SEQUENCE LISTING (166 sequences)

SEQ ID NO: 1 - protein, 4 aa, Artificial Sequence
  Description of Artificial Sequence Synthetic peptide
  Linker
GGGS

SEQ ID NO: 2 - protein, 5 aa, Artificial Sequence
  Description of Artificial Sequence Synthetic peptide
  Linker
GGGGS

SEQ ID NO: 3 - protein, 4 aa, Artificial Sequence
  Description of Artificial Sequence Synthetic peptide
  Linker
GGSG

SEQ ID NO: 4 - protein, 14 aa, Artificial Sequence
  Description of Artificial Sequence Synthetic peptide
  Linker
GSAAAGGSGGSGGS

SEQ ID NO: 5 - protein, 8 aa, Artificial Sequence
  Description of Artificial Sequence Synthetic peptide
  Linker
GGGSGGGS

SEQ ID NO: 6 - protein, 4 aa, Artificial Sequence
  Description of Artificial Sequence Synthetic peptide
  S1 Cleavage Site
  MOD_RES (2)..(2): Any amino acid
RXRR

SEQ ID NO: 7 - protein, 4 aa, Artificial Sequence
  Description of Artificial Sequence Synthetic peptide
  S1 Cleavage Site
  MOD_RES (2)..(2): Any amino acid
RXKR

SEQ ID NO: 8 - protein, 8 aa, Homo sapiens
VGCGVLLS

SEQ ID NO: 9 - protein, 7 aa, Homo sapiens
GCGVLLS

SEQ ID NO: 10 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 11 - protein, 548 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 12 - protein, 482 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 13 - protein, 448 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQRPPDPAPAPLGAPGLPNGLLSGDEDFSSIADMDFSALLSQISS

SEQ ID NO: 14 - protein, 480 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 15 - protein, 445 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNHDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 16 - DNA, 2595 bp, Homo sapiens
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaaaaaaaaaaaaaaaaaa

SEQ ID NO: 17 - DNA, 2586 bp, Homo sapiens
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaaaaaaaaaaaaaaaaaa

SEQ ID NO: 18 - DNA, 2388 bp, Homo sapiens
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaaaaaaaaaaaaaaaaaa

SEQ ID NO: 19 - DNA, 2286 bp, Homo sapiens
agcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctggctagaaaaaaaaaaaaaaaaaa

SEQ ID NO: 20 - DNA, 2341 bp, Homo sapiens
attccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctgg

SEQ ID NO: 21 - DNA, 2236 bp, Homo sapiens
attccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagttcctatagaagagcagcgtggggactacgacctgaatgctgtgcggctctgcttccaggtgacagtgcgggacccatcaggcaggcccctccgcctgccgcctgtcctttctcatcccatctttgacaatcacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctgg

SEQ ID NO: 22 - protein, 50 aa, Artificial Sequence
  Description of Artificial Sequence Synthetic polypeptide
  VP64
DALDDFDLDMLGSDALDDFDLDMLGSDALDDFDLDMLGSDALDDFDLDML

SEQ ID NO: 23 - DNA, 149 bp, Artificial Sequence
  Description of Artificial Sequence Synthetic polynucleotide
  VP64
gacgctcttgatgatttcgatctcgacatgctgggatcagacgctctcgacgacttcgatttggacatgcttggtccgacgctctcgatgatttcgacctcgacatgctcggatccgatgctctggatgactttgatcttgatatgctg

SEQ ID NO: 24 - protein, 318 aa, Artificial Sequence
  Description of Artificial Sequence Synthetic polypeptide
  MyoD
MELLSPPLRDIDLTGPDGSLCSFETADDFYDDPCFDSPDLRFFEDLDPRLVHMGALLKPEEHAHFPTAVHPGPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAFYAPGPLPPGRGSEHYSGDSDASSPRSNCSDGMMDYSGPPSGPRRQNGYDTAYYSEAARESRPGKSAAVSSLDCLSSIVERISTDSPAAPALLLADAPPESPPGPPEGASLSDTEQGTQTPSPDAAPQCPAGSNPNAIYQVL

SEQ ID NO: 25 - protein, 147 aa, Artificial Sequence
  Description of Artificial Sequence Synthetic polypeptide
  GAL4
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 26 - DNA, 441 bp, Artificial Sequence
  Description of Artificial Sequence Synthetic polynucleotide
  GAL4
atgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagt

SEQ ID NO: 27 - protein, 8 aa, Artificial Sequence
  Description of Artificial Sequence Synthetic peptide
  Linker
GGGSGGGS

SEQ ID NO: 28 - protein, 205 aa, Artificial Sequence
  Description of Artificial Sequence Synthetic polypeptide
  Example Transcriptional Regulatory Domain
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 29 - DNA, 24 bp, Artificial Sequence
  Description of Artificial Sequence Synthetic oligonucleotide
  Linker
ggcggtggaagcggaggaggttcc

SEQ ID NO: 30 - DNA, 614 bp, Artificial Sequence
  Description of Artificial Sequence Synthetic polynucleotide
  Example Transcriptional Regulatory Domain
atgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggtccgacgctctcgatgatttcgacctcgacatgctcggatccgatgctctggatgactttgatcttgatatgctg

SEQ ID NO: 31 - DNA, 8601 bp, Artificial Sequence
  Description of Artificial Sequence Synthetic polynucleotide
  Example synPTPR
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggtaataaggccagggggaaagaagaagtacaagctaaagcacatcgtatgggcaagcagggagctagaacgattcgcagttaatcctggccttttagagacatcagaaggcgccgctgatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaatttaaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctcctgcaggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgatctcgacacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttgggtcgaggatggtaccacgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaagccaccatgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcgatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaactttacctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactacccccgggaaacctacctgagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatggggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctgtgactacgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggttaattaactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcattgtctgagtaggtgtcattctattctggggggtggggtggggcaggacagcaagggggaggattgggaagacaatagcaggcatgctggggagtatgatcggtccacgatcagctagattatctagtcagcttgatcatggtcatagctgtttcctgaggctcaatactgaccatttaaatcatacctgacctccatagcagaaagtcaaaagcctccgaccggaggcttttgacttgatcggcacctaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgaatactcaccagtcacagaaaagcatctcacggatggcatgacagtaagagaattatgcagtgctgccataaccatgagtgataacactgcggccaacttacttctggcaaccatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtggctctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggccctgaggacctaaatgtaatcacctggctcaccttcgggtgggcctttctgcgttgctggcgtttttccataggctccgcccccctgacgagcatcacaaaaatcggtgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggcccacccgtgtaaaacgacggccagtttatctagtcagcttgattctagctgatcgtggaccggaaggtgagccag

SEQ ID NO: 32 - protein, 797 aa, Artificial Sequence
  Description of Artificial Sequence Synthetic polypeptide
  Example synPTPR
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDVPGPVPVKSLQGTSFENKIFLNWKEPLDPNGIITQYEISYSSIRSFDPAVPVAGPPQTVSNLWNSTHHVFMHLHPGTTYQFFIRASTVKGFGPATAINVTTNISAPTLPDYEGVDASLNETATTITVLLRPAQAKGAPISAYQIVVEELHPHRTKREAGAMECYQVPVTYQNAMSGGAPYYFAAELPPGNLPEPAPFTVGDNRTYQGFWNPPLAPRKGYNIYFQAMSSVEKETKTQCVRIATKAAATEEPEVIPDPAKQTDRVVKIAGISAGILVFILLLLVVILIVKKSKLAKKRKDAMGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 33 - DNA, 906 bp, Artificial Sequence
  Description of Artificial Sequence Synthetic polynucleotide
  Example synPTPR Core Construct
gatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaactttacctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactacccccgggaaacctacctgagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatgggg

SEQ ID NO: 34 - protein, 302 aa, Artificial Sequence
  Description of Artificial Sequence Synthetic polypeptide
  Example synPTPR Core Construct
DVPGPVPVKSLQGTSFENKIFLNWKEPLDPNGIITQYEISYSSIRSFDPAVPVAGPPQTVSNLWNSTHHVFMHLHPGTTYQFFIRASTVKGFGPATAINVTTNISAPTLPDYEGVDASLNETATTITVLLRPAQAKGAPISAYQIVVEELHPHRTKREAGAMECYQVPVTYQNAMSGGAPYYFAAELPPGNLPEPAPFTVGDNRTYQGFWNPPLAPRKGYNIYFQAMSSVEKETKTQCVRIATKAAATEEPEVIPDPAKQTDRVVKIAGISAGILVFILLLLVVILIVKKSKLAKKRKDAMG

SEQ ID NO: 35 - DNA, 303 bp, Homo sapiens
gatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcacc

SEQ ID NO: 36 - protein, 101 aa, Homo sapiens
DVPGPVPVKSLQGTSFENKIFLNWKEPLDPNGIITQYEISYSSIRSFDPAVPVAGPPQTVSNLWNSTHHVFMHLHPGTTYQFFIRASTVKGFGPATAINVT

SEQ ID NO: 37 - DNA, 252 bp, Homo sapiens
cctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactacccccgggaaacctacct

SEQ ID NO: 38 - protein, 84 aa, Homo sapiens
PDYEGVDASLNETATTITVLLRPAQAKGAPISAYQIVVEELHPHRTKREAGAMECYQVPVTYQNAMSGGAPYYFAAELPPGNLP

SEQ ID NO: 39 - protein, 784 aa, Artificial Sequence
  Description of Artificial Sequence Synthetic polypeptide
  pCDL1932
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSKNFHVKAVMKTSVLLSWEIPENYNSAMPFKILYDDGKMVEEVDGRATQKLIVNLKPEKSYSFVLTNRGNSAGGLQHRVTAKTAPDVLRTKPAFIGKTNLDGMITVQLPEVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMEHAESKMYATSPYSDPVVSMDLDPQPITDEEEGLIWVVGPVLAVVFIICIVIAILLYKRKRAESDSRKSSGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 40 - DNA, 2352 bp, Artificial Sequence
  Description of Artificial Sequence Synthetic polynucleotide
  pCDL1932
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggtggaggaggctctaaaaatttccacgtgaaggctgttatgaaaacatccgttctcctgtcatgggaaatcccggaaaactataattctgctatgcctttcaagatattgtatgatgatggcaagatggttgaagaggtcgacggtcgggcgacacaaaaactgatcgttaacctcaaacctgagaaatcatattcattcgtcctcaccaatcgcggtaatagtgctggtggcctccagcaccgggtaaccgcaaaaactgcgcctgatgtgctcaggacgaagcccgcgtttataggcaagaccaatcttgatggcatgatcactgttcagctcccggaagttcccgccaacgagaatatcaagggttattatattattatcgtaccgctcaagaagtctcgaggcaaatttatcaaaccttgggagtcaccagatgaaatggagcttgatgagttgctcaaagagatcagcagaaagcggcggtccataaggtacggcagggaggtcgagctcaagccatacattgcggctcatttcgatgtgttgccgacggagttcacgctcggggatgataaacactacggcggcttcacaaacaaacagctccaatcagggcaggagtatgtcttcttcgtgcttgctgtcatggaacacgccgaatccaaaatgtatgcaacaagcccttactccgatccggttgtttctatggatctggacccgcagccgataacagatgaagaagaagggctcatttgggtggttggccctgtgctggccgtggtgtttattatctgtatcgttattgcgattcttctctataagcggaagcgagcggagagtgactctcgaaaatcatccgggggtggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 41 - protein, 22 aa, Homo sapiens
MLLLVTSLLLCELPHPAFLLIP

SEQ ID NO: 42 - DNA, 66 bp, Homo sapiens
atgcttctcctggtgacaagccttctgctctgtgagttaccacacccagcattcctcttgatccct

SEQ ID NO: 43 - protein, 10 aa, Artificial Sequence
  Description of Artificial Sequence Synthetic peptide
  Myc tag
EQKLISEEDL

SEQ ID NO: 44 - DNA, 30 bp, Artificial Sequence
  Description of Artificial Sequence Synthetic oligonucleotide
  Myc tag
gaacaaaagctgatcagcgaggaggatctc

SEQ ID NO: 45 - protein, 242 aa, Artificial Sequence
  Description of Artificial Sequence Synthetic polypeptide
  Anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 46 - DNA, 726 bp, Artificial Sequence
  Description of Artificial Sequence Synthetic polynucleotide
  Anti-CD19 scFv
gacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagc

SEQ ID NO: 47 - DNA, 15 bp, Artificial Sequence
  Description of Artificial Sequence Synthetic oligonucleotide
  Linker
ggtggaggaggctct

SEQ ID NO: 48 - DNA, 15 bp, Artificial Sequence
  Description of Artificial Sequence Synthetic oligonucleotide
  Linker
ggtggtgggggctcc

SEQ ID NO: 49 - DNA, 15 bp, Artificial Sequence
  Description of Artificial Sequence Synthetic oligonucleotide
  Linker
ggaggtggtgggagt

SEQ ID NO: 50 - DNA, 15 bp, Artificial Sequence
  Description of Artificial Sequence Synthetic oligonucleotide
  Linker
ggcggaggcgggagc

SEQ ID NO: 51 - DNA, 15 bp, Artificial Sequence
  Description of Artificial Sequence Synthetic oligonucleotide
  Linker
ggcggtggaggttcc

SEQ ID NO: 52 - DNA, 15 bp, Artificial Sequence
  Description of Artificial Sequence Synthetic oligonucleotide
  Linker
gggggaggtgggagt

SEQ ID NO: 53 - DNA, 15 bp, Artificial Sequence
  Description of Artificial Sequence Synthetic oligonucleotide
  Linker
ggcgggggagggagc

SEQ ID NO: 54 - protein, 85 aa, Homo sapiens
KNFHVKAVMKTSVLLSWEIPENYNSAMPFKILYDDGKMVEEVDGRATQKLIVNLKPEKSYSFVLTNRGNSAGGLQHRVTAKTAPD

SEQ ID NO: 55 - DNA, 255 bp, Homo sapiens
aaaaatttccacgtgaaggctgttatgaaaacatccgttctcctgtcatgggaaatcccggaaaactataattctgctatgcctttcaagatattgtatgatgatggcaagatggttgaagaggtcgacggtcgggcgacacaaaaactgatcgttaacctcaaacctgagaaatcatattcattcgtcctcaccaatcgcggtaatagtgctggtggcctccagcaccgggtaaccgcaaaaactgcgcctgat

SEQ ID NO: 56 - DNA, 852 bp, Artificial Sequence
  Description of Artificial Sequence Synthetic polynucleotide
  Sequence encoding PTPR Delta
aaaaatttccacgtgaaggctgttatgaaaacatccgttctcctgtcatgggaaatcccggaaaactataattctgctatgcctttcaagatattgtatgatgatggcaagatggttgaagaggtcgacggtcgggcgacacaaaaactgatcgttaacctcaaacctgagaaatcatattcattcgtcctcaccaatcgcggtaatagtgctggtggcctccagcaccgggtaaccgcaaaaactgcgcctgatgtgctcaggacgaagcccgcgtttataggcaagaccaatcttgatggcatgatcactgttcagctcccggaagttcccgccaacgagaatatcaagggttattatattattatcgtaccgctcaagaagtctcgaggcaaatttatcaaaccttgggagtcaccagatgaaatggagcttgatgagttgctcaaagagatcagcagaaagcggcggtccataaggtacggcagggaggtcgagctcaagccatacattgcggctcatttcgatgtgttgccgacggagttcacgctcggggatgataaacactacggcggcttcacaaacaaacagctccaatcagggcaggagtatgtcttcttcgtgcttgctgtcatggaacacgccgaatccaaaatgtatgcaacaagcccttactccgatccggttgtttctatggatctggacccgcagccgataacagatgaagaagaagggctcatttgggtggttggccctgtgctggccgtggtgtttattatctgtatcgttattgcgattcttctctataagcggaagcgagcggagagtgactctcgaaaatcatccgggggt

SEQ ID NO: 57 - protein, 289 aa, Artificial Sequence
  Description of Artificial Sequence Synthetic polypeptide
  Sequence encoding PTPR Delta
AKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGG

SEQ ID NO: 58 - protein, 9 aa, Artificial Sequence
  Description of Artificial Sequence Synthetic peptide
  c-Myc NLS
PAAKRVKLD

SEQ ID NO: 59 - DNA, 27 bp, Artificial Sequence
  Description of Artificial Sequence Synthetic oligonucleotide
  c-Myc NLS
cccgccgccaagagagtgaagctggac

SEQ ID NO: 60 - protein, 147 aa, Artificial Sequence; identical to SEQ ID NO: 25
  Description of Artificial Sequence Synthetic polypeptide
  GAL4

SEQ ID NO: 61 - DNA, 441 bp, Artificial Sequence; identical to SEQ ID NO: 26
  Description of Artificial Sequence Synthetic polynucleotide
  GAL4

SEQ ID NO: 62 - DNA, 150 bp, Artificial Sequence
  Description of Artificial Sequence Synthetic polynucleotide
  VP64
gacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 63 - protein, 84 aa, Homo sapiens
AKNFRVAAAMKTSVLLSWEVPDSYKSAVPFKILYNGQSVEVDGHSMRKLIADLQPNTEYSFVLMNRGSSAGGLQHLVSIRTAPD

SEQ ID NO: 64 - DNA, 252 bp, Homo sapiens
gccaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgat

SEQ ID NO: 65 - DNA, 252 bp, Homo sapiens
gcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgat

SEQ ID NO: 66 - protein, 789 aa, Artificial Sequence
  Description of Artificial Sequence Synthetic polypeptide
  pCDL1933
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 67 - DNA, 2367 bp, Artificial Sequence
  Description of Artificial Sequence Synthetic polynucleotide
  pCDL1933
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggaggtggtgggagtgccaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggagggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 68 - protein, 289 aa, Artificial Sequence; identical to SEQ ID NO: 57
  Description of Artificial Sequence Synthetic polypeptide
  Sequence from PTPR LAR

SEQ ID NO: 69 - DNA, 867 bp, Artificial Sequence
  Description of Artificial Sequence Synthetic polynucleotide
  Sequence from PTPR LAR
gccaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggaggg

SEQ ID NO: 70 - protein, 89 aa, Homo sapiens
IFLQWREPTQTYGVITLYEITYKAVSSFDPEIDLSNQSGRVSKLGNETHFLFFGLYPGTTYSFTIRASTAKGFGPPATNQFTTKISAPS

SEQ ID NO: 71 - DNA, 267 bp, Homo sapiens
atatttctgcaatggcgagagcctacacaaacatacggagtcataacgttgtacgaaattacgtacaaggccgtgtcatcattcgatcccgaaattgatctttctaaccagtcagggcgcgtaagtaaactcggcaacgagacccactttttgttcttcggcctgtatccgggcactacgtacagtttcaccatccgcgcatctacggccaagggttttggcccacccgctacgaaccagtttactacgaagatttctgctccttca

SEQ ID NO: 72 - protein, 83 aa, Homo sapiens
PAYELETPLNQTDNTVTVMLKPAHSRGAPVSVYQIVVEEERPRRTKKTTEILKCYPVPIHFQNASLLNSQYYFAAEFPADSLQ

SEQ ID NO: 73 - DNA, 249 bp, Homo sapiens
ccagcttatgaactcgaaactccactgaaccaaactgacaacacagttactgtgatgctgaagcccgcgcatagccgaggtgccccagtttctgtgtatcaaattgtggtagaagaagaacggccacgccgcacaaagaagacgacggaaatactgaaatgttatccagtccctattcacttccagaacgctagtttgcttaactcacagtattatttcgcggcagaattccccgccgattctctgcag

SEQ ID NO: 74 - protein, 284 aa, Artificial Sequence
  Description of Artificial Sequence Synthetic polypeptide
  Sequence from PTPR Mu
IFLQWREPTQTYGVITLYEITYKAVSSFDPEIDLSNQSGRVSKLGNETHFLFFGLYPGTTYSFTIRASTAKGFGPPATNQFTTKISAPSMPAYELETPLNQTDNTVTVMLKPAHSRGAPVSVYQIVVEEERPRRTKKTTEILKCYPVPIHFQNASLLNSQYYFAAEFPADSLQAAQPFTIGDNKTYNGYWNTPLLPYKSYRIYFQAASRANGETKIDCVQVATKGAATPKPVPEPEKQTDHTVKIAGVIAGILLFVIIFLGVVLVMKKRKLAKKRKETMSSTGG

SEQ ID NO: 75 - DNA, 852 bp, Artificial Sequence
  Description of Artificial Sequence Synthetic polynucleotide
  Sequence from PTPR Mu
atatttctgcaatggcgagagcctacacaaacatacggagtcataacgttgtacgaaattacgtacaaggccgtgtcatcattcgatcccgaaattgatctttctaaccagtcagggcgcgtaagtaaactcggcaacgagacccactttttgttcttcggcctgtatccgggcactacgtacagtttcaccatccgcgcatctacggccaagggttttggcccacccgctacgaaccagtttactacgaagatttctgctccttcaatgccagcttatgaactcgaaactccactgaaccaaactgacaacacagttactgtgatgctgaagcccgcgcatagccgaggtgccccagtttctgtgtatcaaattgtggtagaagaagaacggccacgccgcacaaagaagacgacggaaatactgaaatgttatccagtccctattcacttccagaacgctagtttgcttaactcacagtattatttcgcggcagaattccccgccgattctctgcaggcggcacagccctttacaataggggacaacaagacttacaatggctattggaacacccccttgcttccttacaagagctacaggatctactttcaagcggcctcccgcgcaaacggtgaaacgaaaattgactgtgtgcaggtagccacaaagggtgcagcgactccgaagcccgtaccggagccagagaagcaaactgatcacacagtcaagattgccggcgtcatagcaggtattctgttgttcgtgataatctttctcggcgtcgtcctcgttatgaagaagaggaaactcgcaaagaagcggaaggaaacaatgtcatccactggtgga

SEQ ID NO: 76 - protein, 784 aa, Artificial Sequence
  Description of Artificial Sequence Synthetic polypeptide
  pCDL1934
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSIFLQWREPTQTYGVITLYEITYKAVSSFDPEIDLSNQSGRVSKLGNETHFLFFGLYPGTTYSFTIRASTAKGFGPPATNQFTTKISAPSMPAYELETPLNQTDNTVTVMLKPAHSRGAPVSVYQIVVEEERPRRTKKTTEILKCYPVPIHFQNASLLNSQYYFAAEFPADSLQAAQPFTIGDNKTYNGYWNTPLLPYKSYRIYFQAASRANGETKIDCVQVATKGAATPKPVPEPEKQTDHTVKIAGVIAGILLFVIIFLGVVLVMKKRKLAKKRKETMSSTGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 77 - DNA, 2352 bp, Artificial Sequence
  Description of Artificial Sequence Synthetic polynucleotide
  pCDL1934
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggcggaggcgggagcatatttctgcaatggcgagagcctacacaaacatacggagtcataacgttgtacgaaattacgtacaaggccgtgtcatcattcgatcccgaaattgatctttctaaccagtcagggcgcgtaagtaaactcggcaacgagacccactttttgttcttcggcctgtatccgggcactacgtacagtttcaccatccgcgcatctacggccaagggttttggcccacccgctacgaaccagtttactacgaagatttctgctccttcaatgccagcttatgaactcgaaactccactgaaccaaactgacaacacagttactgtgatgctgaagcccgcgcatagccgaggtgccccagtttctgtgtatcaaattgtggtagaagaagaacggccacgccgcacaaagaagacgacggaaatactgaaatgttatccagtccctattcacttccagaacgctagtttgcttaactcacagtattatttcgcggcagaattccccgccgattctctgcaggcggcacagccctttacaataggggacaacaagacttacaatggctattggaacacccccttgcttccttacaagagctacaggatctactttcaagcggcctcccgcgcaaacggtgaaacgaaaattgactgtgtgcaggtagccacaaagggtgcagcgactccgaagcccgtaccggagccagagaagcaaactgatcacacagtcaagattgccggcgtcatagcaggtattctgttgttcgtgataatctttctcggcgtcgtcctcgttatgaagaagaggaaactcgcaaagaagcggaaggaaacaatgtcatccactggtggaggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 78 - protein, 807 aa, Artificial Sequence
  Description of Artificial Sequence Synthetic polypeptide
  pCDL1935
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSEDVPSGIAAESLTFTPLEDMIFLKWEEPQEPNGLITQYEISYQSIESSDPAVNVPGPRRTISKLRNETYHVFSNLHPGTTYLFSVRARTGKGFGQAALTEITTNISAPSFDYADMPSPLGESENTITVLLRPAQGRGAPISVYQVIVEEERARRLRREPGGQDCFPVPLTFEAALARGLVHYFGAELAASSLPEAMPFTVGDNQTYRGFWNPPLEPRKAYLIYFQAASHLKGETRLNCIRIARKAACKESKRPLEVSQRSEEMGLILGICAGGLAVLILLLGAIIVIIRKGRDHYAYSYYPKPVNMTGGGGGGSPAAKRVKLDMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 79 - DNA, 2427 bp, Artificial Sequence
  Description of Artificial Sequence Synthetic polynucleotide
  pCDL1935
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggcggtggaggttccgaagatgtcccatccggtatagcggctgaaagcttgacatttaccccattggaagatatgattttcctgaaatgggaagaaccacaagagcccaatggtctcattactcaatatgagatcagttaccagagcattgagtcaagtgatcccgctgtcaatgtaccgggacctcgcaggactatctctaagctgcggaacgaaacgtaccatgtattcagcaacctgcaccccggcaccacgtacttgttttccgtacgcgcgagaactggcaagggattcgggcaggctgcccttacagaaataactacgaacatttctgctccttcattcgactacgcagacatgccttcaccgctcggtgaatctgagaacaccattacggtcctgcttaggcctgcacagggaaggggtgctcccatttccgtctaccaggtaatcgttgaagaggaacgcgcccggcggctcagacgggaacccggtgggcaagactgtttcccggtccctctgacctttgaggcggccttggccagaggtctggtgcattacttcggagccgagttggccgcaagctcactgcctgaggcgatgcccttcaccgtgggggacaatcagacctacaggggattttggaatccacctcttgaacctcgcaaagcgtacctgatctatttccaggctgcgtcacacctgaaaggggaaaccaggttgaattgcatccgcatagctaggaaagccgcctgtaaagagtccaaaaggccacttgaagtctctcagcgcagtgaagaaatgggtctgatccttggaatttgcgcgggagggctggctgtacttatccttctcctcggagctataatcgttataatcaggaaaggcagagaccactacgcctactcttactatcctaaaccggtgaacatgacggggggaggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 80 - protein, 107 aa, Homo sapiens
VPSGIAAESLTFTPLEDMIFLKWEEPQEPNGLITQYEISYQSIESSDPAVNVPGPRRTISKLRNETYHVFSNLHPGTTYLFSVRARTGKGFGQAALTEITTNISAPS

SEQ ID NO: 81 - DNA, 321 bp, Homo sapiens
gtcccatccggtatagcggctgaaagcttgacatttaccccattggaagatatgattttcctgaaatgggaagaaccacaagagcccaatggtctcattactcaatatgagatcagttaccagagcattgagtcaagtgatcccgctgtcaatgtaccgggacctcgcaggactatctctaagctgcggaacgaaacgtaccatgtattcagcaacctgcaccccggcaccacgtacttgttttccgtacgcgcgagaactggcaagggattcgggcaggctgcccttacagaaataactacgaacatttctgctccttca

SEQ ID NO: 82 - protein, 83 aa, Homo sapiens
FDYADMPSPLGESENTITVLLRPAQGRGAPISVYQVIVEEERARRLRREPGGQDCFPVPLTFEAALARGLVHYFGAELAASSL

SEQ ID NO: 83 - DNA, 249 bp, Homo sapiens
ttcgactacgcagacatgccttcaccgctcggtgaatctgagaacaccattacggtcctgcttaggcctgcacagggaaggggtgctcccatttccgtctaccaggtaatcgttgaagaggaacgcgcccggcggctcagacgggaacccggtgggcaagactgtttcccggtccctctgacctttgaggcggccttggccagaggtctggtgcattacttcggagccgagttggccgcaagctcactg

SEQ ID NO: 84 - protein, 309 aa, Artificial Sequence
  Description of Artificial Sequence Synthetic polypeptide
  Sequence from PTPR Psi
EDVPSGIAAESLTFTPLEDMIFLKWEEPQEPNGLITQYEISYQSIESSDPAVNVPGPRRTISKLRNETYHVFSNLHPGTTYLFSVRARTGKGFGQAALTEITTNISAPSFDYADMPSPLGESENTITVLLRPAQGRGAPISVYQVIVEEERARRLRREPGGQDCFPVPLTFEAALARGLVHYFGAELAASSLPEAMPFTVGDNQTYRGFWNPPLEPRKAYLIYFQAASHLKGETRLNCIRIARKAACKESKRPLEVSQRSEEMGLILGICAGGLAVLILLLGAIIVIIRKGRDHYAYSYYPKPVNMTGG

SEQ ID NO: 85 - DNA, 927 bp, Artificial Sequence
  Description of Artificial Sequence Synthetic polynucleotide
  Sequence from PTPR Psi
gaagatgtcccatccggtatagcggctgaaagcttgacatttaccccattggaagatatgattttcctgaaatgggaagaaccacaagagcccaatggtctcattactcaatatgagatcagttaccagagcattgagtcaagtgatcccgctgtcaatgtaccgggacctcgcaggactatctctaagctgcggaacgaaacgtaccatgtattcagcaacctgcaccccggcaccacgtacttgttttccgtacgcgcgagaactggcaagggattcgggcaggctgcccttacagaaataactacgaacatttctgctccttcattcgactacgcagacatgccttcaccgctcggtgaatctgagaacaccattacggtcctgcttaggcctgcacagggaaggggtgctcccatttccgtctaccaggtaatcgttgaagaggaacgcgcccggcggctcagacgggaacccggtgggcaagactgtttcccggtccctctgacctttgaggcggccttggccagaggtctggtgcattacttcggagccgagttggccgcaagctcactgcctgaggcgatgcccttcaccgtgggggacaatcagacctacaggggattttggaatccacctcttgaacctcgcaaagcgtacctgatctatttccaggctgcgtcacacctgaaaggggaaaccaggttgaattgcatccgcatagctaggaaagccgcctgtaaagagtccaaaaggccacttgaagtctctcagcgcagtgaagaaatgggtctgatccttggaatttgcgcgggagggctggctgtacttatccttctcctcggagctataatcgttataatcaggaaaggcagagaccactacgcctactcttactatcctaaaccggtgaacatgacgggggga

SEQ ID NO: 86 - protein, 792 aa, Artificial Sequence
  Description of Artificial Sequence Synthetic polypeptide
  pCDL1936
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSKIYIQWKPPNETNGVITLYEINYKAVGSLDPSADLSSQRGKVFKLRNETHHLFVGLYPGTTYSFTIKASTAKGFGPPVTTRIATKISAPSMPEYDTDTPLNETDTTITVMLKPAQSRGAPVSVYQLVVKEERLQKSRRAADIIECFSVPVSYRNASSLDSLHYFAAELKPANLPVTQPFTVGDNKTYNGYWNPPLSPLKSYSIYFQALSKANGETKINCVRLATKGASTQNSNTVEPEKQVDNTVKMAGVIAGLLMFIIILLGVMLTIKRRRNAYSYSYYLKLAKKQKETGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 87 - DNA, 2376 bp, Artificial Sequence
  Description of Artificial Sequence Synthetic polynucleotide
  pCDL1936
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcgggggaggtgggagtaaaatctatattcaatggaagccgccaaatgaaaccaatggagttataacgctctatgaaatcaactacaaggcggttggctcccttgatccctctgccgacctttcctcacagcggggtaaagtgttcaagctgaggaacgaaacgcaccaccttttcgtggggttgtatccaggaacgacctacagttttactattaaggcttccacagccaaaggctttgggccccctgtaaccactaggattgctactaaaatctccgcgccatctatgcccgaatatgatacggacaccccattgaacgaaacagatacaacaataactgtcatgctgaagcctgcgcaatcacgcggagcccctgtcagcgtatatcaacttgtagtcaaagaagaaagactgcaaaaatcccgacgcgctgccgacattattgagtgcttctcagtacccgtgagctacagaaacgctagtagcttggattctttgcattatttcgcggccgaacttaagcccgcgaatcttccggtgactcaaccgtttacagtgggtgacaataaaacttacaatggctattggaacccgccgttgtcaccgctgaagagctactcaatatatttccaggccctgagtaaagctaacggtgagacaaaaatcaactgtgtgagacttgcaactaaaggagccagcacacagaattctaatactgtggagcccgagaagcaagttgacaacactgtgaaaatggctggggtaattgcaggactgcttatgttcatcataatcctgcttggggttatgcttactatcaagcgacggcgcaacgcctacagctatagctactatttgaaattggcaaaaaagcagaaggaaactggagggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 88 - protein, 90 aa, Homo sapiens
KIYIQWKPPNETNGVITLYEINYKAVGSLDPSADLSSQRGKVFKLRNETHHLFVGLYPGTTYSFTIKASTAKGFGPPVTTRIATKISAPS

SEQ ID NO: 89 - DNA, 270 bp, Homo sapiens
aaaatctatattcaatggaagccgccaaatgaaaccaatggagttataacgctctatgaaatcaactacaaggcggttggctcccttgatccctctgccgacctttcctcacagcggggtaaagtgttcaagctgaggaacgaaacgcaccaccttttcgtggggttgtatccaggaacgacctacagttttactattaaggcttccacagccaaaggctttgggccccctgtaaccactaggattgctactaaaatctccgcgccatct

SEQ ID NO: 90 - protein, 136 aa, Homo sapiens
MPEYDTDTPLNETDTTITVMLKPAQSRGAPVSVYQLVVKEERLQKSRRAADIIECFSVPVSYRNASSLDSLHYFAAELKPANLPVTQPFTVGDNKTYNGYWNPPLSPLKSYSIYFQALSKANGETKINCVRLATKG

SEQ ID NO: 91 - DNA, 408 bp, Homo sapiens
atgcccgaatatgatacggacaccccattgaacgaaacagatacaacaataactgtcatgctgaagcctgcgcaatcacgcggagcccctgtcagcgtatatcaacttgtagtcaaagaagaaagactgcaaaaatcccgacgcgctgccgacattattgagtgcttctcagtacccgtgagctacagaaacgctagtagcttggattctttgcattatttcgcggccgaacttaagcccgcgaatcttccggtgactcaaccgtttacagtgggtgacaataaaacttacaatggctattggaacccgccgttgtcaccgctgaagagctactcaatatatttccaggccctgagtaaagctaacggtgagacaaaaatcaactgtgtgagacttgcaactaaagga

SEQ ID NO: 92 - protein, 21 aa, Homo sapiens
AGVIAGLLMFIIILLGVMLTI

SEQ ID NO: 93 - DNA, 63 bp, Homo sapiens
gctggggtaattgcaggactgcttatgttcatcataatcctgcttggggttatgcttactatc

SEQ ID NO: 94 - protein, 292 aa, Artificial Sequence
  Description of Artificial Sequence Synthetic polypeptide
  Sequence from PTPR Pho
KIYIQWKPPNETNGVITLYEINYKAVGSLDPSADLSSQRGKVFKLRNETHHLFVGLYPGTTYSFTIKASTAKGFGPPVTTRIATKISAPSMPEYDTDTPLNETDTTITVMLKPAQSRGAPVSVYQLVVKEERLQKSRRAADIIECFSVPVSYRNASSLDSLHYFAAELKPANLPVTQPFTVGDNKTYNGYWNPPLSPLKSYSIYFQALSKANGETKINCVRLATKGASTQNSNTVEPEKQVDNTVKMAGVIAGLLMFIIILLGVMLTIKRRRNAYSYSYYLKLAKKQKETGG

SEQ ID NO: 95 - DNA, 876 bp, Artificial Sequence
  Description of Artificial Sequence Synthetic polynucleotide
  Sequence from PTPR Pho
aaaatctatattcaatggaagccgccaaatgaaaccaatggagttataacgctctatgaaatcaactacaaggcggttggctcccttgatccctctgccgacctttcctcacagcggggtaaagtgttcaagctgaggaacgaaacgcaccaccttttcgtggggttgtatccaggaacgacctacagttttactattaaggcttccacagccaaaggctttgggccccctgtaaccactaggattgctactaaaatctccgcgccatctatgcccgaatatgatacggacaccccattgaacgaaacagatacaacaataactgtcatgctgaagcctgcgcaatcacgcggagcccctgtcagcgtatatcaacttgtagtcaaagaagaaagactgcaaaaatcccgacgcgctgccgacattattgagtgcttctcagtacccgtgagctacagaaacgctagtagcttggattctttgcattatttcgcggccgaacttaagcccgcgaatcttccggtgactcaaccgtttacagtgggtgacaataaaacttacaatggctattggaacccgccgttgtcaccgctgaagagctactcaatatatttccaggccctgagtaaagctaacggtgagacaaaaatcaactgtgtgagacttgcaactaaaggagccagcacacagaattctaatactgtggagcccgagaagcaagttgacaacactgtgaaaatggctggggtaattgcaggactgcttatgttcatcataatcctgcttggggttatgcttactatcaagcgacggcgcaacgcctacagctatagctactatttgaaattggcaaaaaagcagaaggaaactggaggg

SEQ ID NO: 96 - protein, 789 aa, Artificial Sequence
  Description of Artificial Sequence Synthetic polypeptide
  pCDL1937
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 97 - DNA, 2367 bp, Artificial Sequence
  Description of Artificial Sequence Synthetic polynucleotide
  pCDL1937
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggcgggggagggagcagtccgaagaacttcaaagtcaagatgatcatgaagacatctgtgctcttgagttgggagtttcccgataattataacagccccacgccttacaagattcagtacaatggactcacccttgatgtagatggccgcacgacaaagaagctgattactcacctcaaaccgcatactttctataatttcgtgctgacgaatcggggttcttccctgggaggtctccagcaaactgtaacggcgtggactgcgtttaatttgctgaacggtaagccctcagtggcccccaaaccggatgccgacggatttataatggtgtaccttccagatggtcagagtccggtccccgtacagagctacttcattgtcatggtgcccctcaggaaatcccgaggtggtcaatttctcacaccattgggtagtccggaggacatggatctggaagaactgatccaggatattagccgcctgcaacgcagatcacttagacatagtagacagctggaggtgccgaggccgtacatcgctgcgcgattctccgtactcccgccaacctttcacccaggggatcagaaacaatacggcggttttgataatcgagggcttgaaccaggacatagatacgtgctttttgtgttggctgtgctccagaaatctgaaccgacgtttgccgcaagcccctttagcgacccatttcagctggataaccctgaccctcagccgatagtcgatggcgaggaggggctgatatgggtgattgggcccgtactcgcggtagtgtttattatctgtatcgtaattgctatactgctttataagaacaagccggacagtaaaaggaaggattctgagcctaggactaaaggcggtggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 98 - protein, 85 aa, Homo sapiens
SPKNFKVKMIMKTSVLLSWEFPDNYNSPTPYKIQYNGLTLDVDGRTTKKLITHLKPHTFYNFVLTNRGSSLGGLQQTVTAWTAFN

SEQ ID NO: 99 - DNA, 255 bp, Homo sapiens
agtccgaagaacttcaaagtcaagatgatcatgaagacatctgtgctcttgagttgggagtttcccgataattataacagccccacgccttacaagattcagtacaatggactcacccttgatgtagatggccgcacgacaaagaagctgattactcacctcaaaccgcatactttctataatttcgtgctgacgaatcggggttcttccctgggaggtctccagcaaactgtaacggcgtggactgcgtttaat

SEQ ID NO: 100 - protein, 289 aa, Artificial Sequence
  Description of Artificial Sequence Synthetic polypeptide
  Sequence from PTPR Sigma
SPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKGG

SEQ ID NO: 101 - DNA, 867 bp, Artificial Sequence
  Description of Artificial Sequence Synthetic polynucleotide
  Sequence from PTPR Sigma
agtccgaagaacttcaaagtcaagatgatcatgaagacatctgtgctcttgagttgggagtttcccgataattataacagccccacgccttacaagattcagtacaatggactcacccttgatgtagatggccgcacgacaaagaagctgattactcacctcaaaccgcatactttctataatttcgtgctgacgaatcggggttcttccctgggaggtctccagcaaactgtaacggcgtggactgcgtttaatttgctgaacggtaagccctcagtggcccccaaaccggatgccgacggatttataatggtgtaccttccagatggtcagagtccggtccccgtacagagctacttcattgtcatggtgcccctcaggaaatcccgaggtggtcaatttctcacaccattgggtagtccggaggacatggatctggaagaactgatccaggatattagccgcctgcaacgcagatcacttagacatagtagacagctggaggtgccgaggccgtacatcgctgcgcgattctccgtactcccgccaacctttcacccaggggatcagaaacaatacggcggttttgataatcgagggcttgaaccaggacatagatacgtgctttttgtgttggctgtgctccagaaatctgaaccgacgtttgccgcaagcccctttagcgacccatttcagctggataaccctgaccctcagccgatagtcgatggcgaggaggggctgatatgggtgattgggcccgtactcgcggtagtgtttattatctgtatcgtaattgctatactgctttataagaacaagccggacagtaaaaggaaggattctgagcctaggactaaaggcggt

SEQ ID NO: 102 - protein, 797 aa, Artificial Sequence; identical to SEQ ID NO: 32
  Description of Artificial Sequence Synthetic polypeptide
  pCDL1541

SEQ ID NO: 103 - DNA, 2391 bp, Artificial Sequence
  Description of Artificial Sequence Synthetic polynucleotide
  pCDL1541
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcgatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaactttacctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactacccccgggaaacctacctgagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatggggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 104 - protein, 105 aa, Homo sapiens
GPVPVKSLQGTSFENKIFLNWKEPLDPNGIITQYEISYSSIRSFDPAVPVAGPPQTVSNLWNSTHHVFMHLHPGTTYQFFIRASTVKGFGPATAINVTTNISAPT

SEQ ID NO: 105 - DNA, 315 bp, Homo sapiens
ggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaact

SEQ ID NO: 106 - protein, 84 aa, Homo sapiens; identical to SEQ ID NO: 38

SEQ ID NO: 107 - DNA, 252 bp, Homo sapiens; identical to SEQ ID NO: 37

SEQ ID NO: 108 - protein, 302 aa, Artificial Sequence; identical to SEQ ID NO: 34
  Description of Artificial Sequence Synthetic polypeptide
  Sequence from PTPR Kappa

SEQ ID NO: 109 - DNA, 906 bp, Artificial Sequence; identical to SEQ ID NO: 33
  Description of Artificial Sequence Synthetic polynucleotide
  Sequence from PTPR Kappa

SEQ ID NO: 110 - protein, 1061 aa, Artificial Sequence
  Description of Artificial Sequence Synthetic polypeptide
  pCDL2243
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSKNFHVKAVMKTSVLLSWEIPENYNSAMPFKILYDDGKMVEEVDGRATQKLIVNLKPEKSYSFVLTNRGNSAGGLQHRVTAKTAPDVLRTKPAFIGKTNLDGMITVQLPEVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMEHAESKMYATSPYSDPVVSMDLDPQPITDEEEGLIWVVGPVLAVVFIICIVIAILLYKRKRAESDSRKSSGGGGGGSPAAKRVKLDGSMVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMGGGSGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 111 - DNA, 3183 bp, Artificial Sequence
  Description of Artificial Sequence Synthetic polynucleotide
  pCDL2243
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggtggaggaggctctaaaaatttccacgtgaaggctgttatgaaaacatccgttctcctgtcatgggaaatcccggaaaactataattctgctatgcctttcaagatattgtatgatgatggcaagatggttgaagaggtcgacggtcgggcgacacaaaaactgatcgttaacctcaaacctgagaaatcatattcattcgtcctcaccaatcgcggtaatagtgctggtggcctccagcaccgggtaaccgcaaaaactgcgcctgatgtgctcaggacgaagcccgcgtttataggcaagaccaatcttgatggcatgatcactgttcagctcccggaagttcccgccaacgagaatatcaagggttattatattattatcgtaccgctcaagaagtctcgaggcaaatttatcaaaccttgggagtcaccagatgaaatggagcttgatgagttgctcaaagagatcagcagaaagcggcggtccataaggtacggcagggaggtcgagctcaagccatacattgcggctcatttcgatgtgttgccgacggagttcacgctcggggatgataaacactacggcggcttcacaaacaaacagctccaatcagggcaggagtatgtcttcttcgtgcttgctgtcatggaacacgccgaatccaaaatgtatgcaacaagcccttactccgatccggttgtttctatggatctggacccgcagccgataacagatgaagaagaagggctcatttgggtggttggccctgtgctggccgtggtgtttattatctgtatcgttattgcgattcttctctataagcggaagcgagcggagagtgactctcgaaaatcatccgggggtggtggtgggggctcccccgccgccaagagagtgaagctggacggatccatggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgaaacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagactctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatgggcggtggaagcggaggaggttccgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 112 - protein, 283 aa, Homo sapiens
MVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAM

SEQ ID NO: 113 - DNA, 849 bp, Artificial Sequence
  Description of Artificial Sequence Synthetic polynucleotide
  HNF1 alpha
atggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgaaacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagactctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatg

SEQ ID NO: 114 - protein, 191 aa, Artificial Sequence
  Description of Artificial Sequence Synthetic polypeptide
  P65 Transcriptional Activation Domain
DEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 115 - DNA, 573 bp, Artificial Sequence
  Description of Artificial Sequence Synthetic polynucleotide
  P65 Transcriptional Activation Domain
gatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 116 - protein, 1066 aa, Artificial Sequence
  Description of Artificial Sequence Synthetic polypeptide
  pCDL2244
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGGGGGGSPAAKRVKLDGSMVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMGGGSGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 117 - DNA, 3198 bp, Artificial Sequence
  Description of Artificial Sequence Synthetic polynucleotide
  pCDL2244
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggaggtggtgggagtgccaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggagggggtggtgggggctcccccgccgccaagagagtgaagctggacggatccatggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgaaacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagactctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatgggcggtggaagcggaggaggttccgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 118 - protein, 1069 aa, Artificial Sequence
  Description of Artificial Sequence Synthetic polypeptide
  pCDL2245
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSKIYIQWKPPNETNGVITLYEINYKAVGSLDPSADLSSQRGKVFKLRNETHHLFVGLYPGTTYSFTIKASTAKGFGPPVTTRIATKISAPSMPEYDTDTPLNETDTTITVMLKPAQSRGAPVSVYQLVVKEERLQKSRRAADIIECFSVPVSYRNASSLDSLHYFAAELKPANLPVTQPFTVGDNKTYNGYWNPPLSPLKSYSIYFQALSKANGETKINCVRLATKGASTQNSNTVEPEKQVDNTVKMAGVIAGLLMFIIILLGVMLTIKRRRNAYSYSYYLKLAKKQKETGGGGGGSPAAKRVKLDGSMVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMGGGSGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 119 - DNA, 3207 bp, Artificial Sequence
  Description of Artificial Sequence Synthetic polynucleotide
  pCDL2245
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcgggggaggtgggagtaaaatctatattcaatggaagccgccaaatgaaaccaatggagttataacgctctatgaaatcaactacaaggcggttggctcccttgatccctctgccgacctttcctcacagcggggtaaagtgttcaagctgaggaacgaaacgcaccaccttttcgtggggttgtatccaggaacgacctacagttttactattaaggcttccacagccaaaggctttgggccccctgtaaccactaggattgctactaaaatctccgcgccatctatgcccgaatatgatacggacaccccattgaacgaaacagatacaacaataactgtcatgctgaagcctgcgcaatcacgcggagcccctgtcagcgtatatcaacttgtagtcaaagaagaaagactgcaaaaatcccgacgcgctgccgacattattgagtgcttctcagtacccgtgagctacagaaacgctagtagcttggattctttgcattatttcgcggccgaacttaagcccgcgaatcttccggtgactcaaccgtttacagtgggtgacaataaaacttacaatggctattggaacccgccgttgtcaccgctgaagagctactcaatatatttccaggccctgagtaaagctaacggtgagacaaaaatcaactgtgtgagacttgcaactaaaggagccagcacacagaattctaatactgtggagcccgagaagcaagttgacaacactgtgaaaatggctggggtaattgcaggactgcttatgttcatcataatcctgcttggggttatgcttactatcaagcgacggcgcaacgcctacagctatagctactatttgaaattggcaaaaaagcagaaggaaactggagggggtggtgggggctcccccgccgccaagagagtgaagctggacggatccatggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgaaacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagactctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatgggcggtggaagcggaggaggttccgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 120 - protein, 1066 aa, Artificial Sequence
  Description of Artificial Sequence Synthetic polypeptide
  pCDL2246
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSSPKNFKVKMIMKTSVLLSWEFPDNYNSPTPYKIQYNGLTLDVDGRTTKKLITHLKPHTFYNFVLTNRGSSLGGLQQTVTAWTAFNLLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKGGGGGGSPAAKRVKLDGSMVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMGGGSGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 121 - DNA, 3198 bp, Artificial Sequence
  Description of Artificial Sequence Synthetic polynucleotide
  pCDL2246
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcggcgggggagggagcagtccgaagaacttcaaagtcaagatgatcatgaagacatctgtgctcttgagttgggagtttcccgataattataacagccccacgccttacaagattcagtacaatggactcacccttgatgtagatggccgcacgacaaagaagctgattactcacctcaaaccgcatactttctataatttcgtgctgacgaatcggggttcttccctgggaggtctccagcaaactgtaacggcgtggactgcgtttaatttgctgaacggtaagccctcagtggcccccaaaccggatgccgacggatttataatggtgtaccttccagatggtcagagtccggtccccgtacagagctacttcattgtcatggtgcccctcaggaaatcccgaggtggtcaatttctcacaccattgggtagtccggaggacatggatctggaagaactgatccaggatattagccgcctgcaacgcagatcacttagacatagtagacagctggaggtgccgaggccgtacatcgctgcgcgattctccgtactcccgccaacctttcacccaggggatcagaaacaatacggcggttttgataatcgagggcttgaaccaggacatagatacgtgctttttgtgttggctgtgctccagaaatctgaaccgacgtttgccgcaagcccctttagcgacccatttcagctggataaccctgaccctcagccgatagtcgatggcgaggaggggctgatatgggtgattgggcccgtactcgcggtagtgtttattatctgtatcgtaattgctatactgctttataagaacaagccggacagtaaaaggaaggattctgagcctaggactaaaggcggtggtggtgggggctcccccgccgccaagagagtgaagctggacggatccatggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgaaacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagactctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatgggcggtggaagcggaggaggttccgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 122 - protein, 1075 aa, Artificial Sequence
  Description of Artificial Sequence Synthetic polypeptide
  pCDL2247
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDVPLGPVPVKSLQGTSFENKIFLNWKEPLDPNGIITQYEISYSSIRSFDPAVPVAGPPQTVSNLWNSTHHVFMHLHPGTTYQFFIRASTVKGFGPATAINVTTNISAPTLPDYEGVDASLNETATTITVLLRPAQAKGAPISAYQIVVEELHPHRTKREAGAMECYQVPVTYQNAMSGGAPYYFAAELPPGNLPEPAPFTVGDNRTYQGFWNPPLAPRKGYNIYFQAMSSVEKETKTQCVRIATKAAATEEPEVIPDPAKQTDRVVKIAGISAGILVFILLLLVVILIVKKSKLAKKRKDAMGGGGGSPAAKRVKLDGSMVSKLSQLQTELLAALLESGLSKEALIQALGEPGPYLLAGEGPLDKGESCGGGRGELAELPNGLGETRGSEDETDDDGEDFTPPILKELENLSPEEAAHQKAVVETLLQEDPWRVAKMVKSYLQQHNIPQREVVDTTGLNQSHLSQHLNKGTPMKTQKRAALYTWYVRKQREVAQQFTHAGQGGLIEEPTGDELPTKKGRRNRFKWGPASQQILFQAYERQKNPSKEERETLVEECNRAECIQRGVSPSQAQGLGSNLVTEVRVYNWFANRRKEEAFRHKLAMGGGSGGGSDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 123 - DNA, 3222 bp, Artificial Sequence
  Description of Artificial Sequence Synthetic polynucleotide
  pCDL2247
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcgatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaactttacctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactacccccgggaaacctacctgagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatggggggtggtgggggctcccccgccgccaagagagtgaagctggacggatccatggtttctaaactgagccagctgcagacggagctcctggcggccctgctcgagtcagggctgagcaaagaggcactgatccaggcactgggtgagccggggccctacctcctggctggagaaggccccctggacaagggggagtcctgcggcggcggtcgaggggagctggctgagctgcccaatgggctgggggagactcggggctccgaggacgaaacggacgacgatggggaagacttcacgccacccatcctcaaagagctggagaacctcagccctgaggaggcggcccaccagaaagccgtggtggagacccttctgcaggaggacccgtggcgtgtggcgaagatggtcaagtcctacctgcagcagcacaacatcccacagcgggaggtggtcgataccactggcctcaaccagtcccacctgtcccaacacctcaacaagggcactcccatgaagacgcagaagcgggccgccctgtacacctggtacgtccgcaagcagcgagaggtggcgcagcagttcacccatgcagggcagggagggctgattgaagagcccacaggtgatgagctaccaaccaagaaggggcggaggaaccgtttcaagtggggcccagcatcccagcagatcctgttccaggcctatgagaggcagaagaaccctagcaaggaggagcgagagactctagtggaggagtgcaatagggcggaatgcatccagagaggggtgtccccatcacaggcacaggggctgggctccaacctcgtcacggaggtgcgtgtctacaactggtttgccaaccggcgcaaagaagaagccttccggcacaagctggccatgggcggtggaagcggaggaggttccgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcc

SEQ ID NO: 124 - protein, 1190 aa, Artificial Sequence
  Description of Artificial Sequence Synthetic polypeptide
  pCDL2762
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSPPQKVMCVSMGSTTVRVSWVPPPADSRNGVITQYSVAYEAVDGEDRGRHVVDGISREHSSWDLVGLEKWTEYRVWVRAHTDVGPGPESSPVLVRTDEDVPSGPPRKVEVEPLNSTAVHVYWKLPVPSKQHGQIRGYQVTYVRLENGEPRGLPIIQDVMLAEAQWRPEESEDYETTISGLTPETTYSVTVAAYTTKGDGARSKPKIVTTTGAVPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 125 - DNA, 3570 bp, Artificial Sequence
  Description of Artificial Sequence Synthetic polynucleotide
  pCDL2762
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcccaccgcaaaaagttatgtgtgtgagcatgggatctaccaccgtccgcgtttcttgggttccaccgcctgccgatagtcggaatggcgtcattactcaatacagcgtagcctacgaggccgtggatggagaggatagaggtcgccatgtcgtagatggaattagccgcgagcatagttcatgggacttagtcgggttggaaaagtggactgaataccgtgtttgggtccgagctcacacggatgttggcccaggaccagagtccagtcccgttctcgttcggacggacgaggacgttccgtccggtccaccccgaaaagttgaggtagagcccctgaatagtacggcagtacatgtctattggaagctccctgttccatcaaagcaacatgggcagattagggggtaccaagttacatatgtgcgattggaaaacggggaaccacggggcctcccgatcatccaagatgttatgctcgcagaggcacagtggcgccccgaggagtccgaagattatgaaactactataagtgggcttaccccagagacaacctactctgtaactgtagctgcttacacgaccaaaggggatggcgcccgaagcaagcccaaaattgttaccacaacaggggctgtacccggcagaccgacaatgatgatcagcacgacagcgatgaacacggcacttctgcaatggcatccgcccaaggaattgccaggggagttgctcggctaccggctgcaatactgtagggcagatgaagcaaggccgaacacaatcgatttcggaaaggatgatcagcactttactgttaccggacttcataaagggacaacttatatcttccggctggcggctaaaaatagagcgggcttgggagaagaattcgagaaggagattagaaccccggaggatttgcctagtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggagggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 126 - protein, 98 aa, Homo sapiens
PPQKVMCVSMGSTTVRVSWVPPPADSRNGVITQYSVAYEAVDGEDRGRHVVDGISREHSSWDLVGLEKWTEYRVWVRAHTDVGPGPESSPVLVRTDED

SEQ ID NO: 127 - DNA, 294 bp, Homo sapiens
ccaccgcaaaaagttatgtgtgtgagcatgggatctaccaccgtccgcgtttcttgggttccaccgcctgccgatagtcggaatggcgtcattactcaatacagcgtagcctacgaggccgtggatggagaggatagaggtcgccatgtcgtagatggaattagccgcgagcatagttcatgggacttagtcgggttggaaaagtggactgaataccgtgtttgggtccgagctcacacggatgttggcccaggaccagagtccagtcccgttctcgttcggacggacgaggac

SEQ ID NO: 128 - protein, 109 aa, Homo sapiens
PPRKVEVEPLNSTAVHVYWKLPVPSKQHGQIRGYQVTYVRLENGEPRGLPIIQDVMLAEAQWRPEESEDYETTISGLTPETTYSVTVAAYTTKGDGARSKPKIVTTTGA

SEQ ID NO: 129 - DNA, 327 bp, Homo sapiens
ccaccccgaaaagttgaggtagagcccctgaatagtacggcagtacatgtctattggaagctccctgttccatcaaagcaacatgggcagattagggggtaccaagttacatatgtgcgattggaaaacggggaaccacggggcctcccgatcatccaagatgttatgctcgcagaggcacagtggcgccccgaggagtccgaagattatgaaactactataagtgggcttaccccagagacaacctactctgtaactgtagctgcttacacgaccaaaggggatggcgcccgaagcaagcccaaaattgttaccacaacaggggct

SEQ ID NO: 130 - protein, 95 aa, Homo sapiens
VPGRPTMMISTTAMNTALLQWHPPKELPGELLGYRLQYCRADEARPNTIDFGKDDQHFTVTGLHKGTTYIFRLAAKNRAGLGEEFEKEIRTPEDL

SEQ ID NO: 131 - DNA, 285 bp, Homo sapiens
gtacccggcagaccgacaatgatgatcagcacgacagcgatgaacacggcacttctgcaatggcatccgcccaaggaattgccaggggagttgctcggctaccggctgcaatactgtagggcagatgaagcaaggccgaacacaatcgatttcggaaaggatgatcagcactttactgttaccggacttcataaagggacaacttatatcttccggctggcggctaaaaatagagcgggcttgggagaagaattcgagaaggagattagaaccccggaggatttg

SEQ ID NO: 132 - protein, 93 aa, Homo sapiens
FPQNLHVTGLTTSTTELAWDPPVLAERNGRIISYTVVFRDINSQQELQNITTDTRFTLTGLKPDTTYDIKVRAWTSKGSGPLSPSIQSRTMPV

SEQ ID NO: 133 - DNA, 279 bp, Homo sapiens
ttcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagtt

SEQ ID NO: 134 - protein, 695 aa, Artificial Sequence
  Description of Artificial Sequence Synthetic polypeptide
  Sequence from PTPR LAR
PPQKVMCVSMGSTTVRVSWVPPPADSRNGVITQYSVAYEAVDGEDRGRHVVDGISREHSSWDLVGLEKWTEYRVWVRAHTDVGPGPESSPVLVRTDEDVPSGPPRKVEVEPLNSTAVHVYWKLPVPSKQHGQIRGYQVTYVRLENGEPRGLPIIQDVMLAEAQWRPEESEDYETTISGLTPETTYSVTVAAYTTKGDGARSKPKIVTTTGAVPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGG

SEQ ID NO: 135 - DNA, 2085 bp, Artificial Sequence
  Description of Artificial Sequence Synthetic polynucleotide
  Sequence from PTPR LAR
ccaccgcaaaaagttatgtgtgtgagcatgggatctaccaccgtccgcgtttcttgggttccaccgcctgccgatagtcggaatggcgtcattactcaatacagcgtagcctacgaggccgtggatggagaggatagaggtcgccatgtcgtagatggaattagccgcgagcatagttcatgggacttagtcgggttggaaaagtggactgaataccgtgtttgggtccgagctcacacggatgttggcccaggaccagagtccagtcccgttctcgttcggacggacgaggacgttccgtccggtccaccccgaaaagttgaggtagagcccctgaatagtacggcagtacatgtctattggaagctccctgttccatcaaagcaacatgggcagattagggggtaccaagttacatatgtgcgattggaaaacggggaaccacggggcctcccgatcatccaagatgttatgctcgcagaggcacagtggcgccccgaggagtccgaagattatgaaactactataagtgggcttaccccagagacaacctactctgtaactgtagctgcttacacgaccaaaggggatggcgcccgaagcaagcccaaaattgttaccacaacaggggctgtacccggcagaccgacaatgatgatcagcacgacagcgatgaacacggcacttctgcaatggcatccgcccaaggaattgccaggggagttgctcggctaccggctgcaatactgtagggcagatgaagcaaggccgaacacaatcgatttcggaaaggatgatcagcactttactgttaccggacttcataaagggacaacttatatcttccggctggcggctaaaaatagagcgggcttgggagaagaattcgagaaggagattagaaccccggaggatttgcctagtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggaggg

SEQ ID NO: 136 - protein, 1092 aa, Artificial Sequence
  Description of Artificial Sequence Synthetic polypeptide
  pCDL2763
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVPSGPPRKVEVEPLNSTAVHVYWKLPVPSKQHGQIRGYQVTYVRLENGEPRGLPIIQDVMLAEAQWRPEESEDYETTISGLTPETTYSVTVAAYTTKGDGARSKPKIVTTTGAVPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 137 - DNA, 3276 bp, Artificial Sequence
  Description of Artificial Sequence Synthetic polynucleotide
  pCDL2763
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcgttccgtccggtccaccccgaaaagttgaggtagagcccctgaatagtacggcagtacatgtctattggaagctccctgttccatcaaagcaacatgggcagattagggggtaccaagttacatatgtgcgattggaaaacggggaaccacggggcctcccgatcatccaagatgttatgctcgcagaggcacagtggcgccccgaggagtccgaagattatgaaactactataagtgggcttaccccagagacaacctactctgtaactgtagctgcttacacgaccaaaggggatggcgcccgaagcaagcccaaaattgttaccacaacaggggctgtacccggcagaccgacaatgatgatcagcacgacagcgatgaacacggcacttctgcaatggcatccgcccaaggaattgccaggggagttgctcggctaccggctgcaatactgtagggcagatgaagcaaggccgaacacaatcgatttcggaaaggatgatcagcactttactgttaccggacttcataaagggacaacttatatcttccggctggcggctaaaaatagagcgggcttgggagaagaattcgagaaggagattagaaccccggaggatttgcctagtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggagggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 138 - protein, 597 aa, Artificial Sequence
  Description of Artificial Sequence Synthetic polypeptide
  Sequence from PTPR LAR
VPSGPPRKVEVEPLNSTAVHVYWKLPVPSKQHGQIRGYQVTYVRLENGEPRGLPIIQDVMLAEAQWRPEESEDYETTISGLTPETTYSVTVAAYTTKGDGARSKPKIVTTTGAVPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGG

SEQ ID NO: 139 - DNA, 1791 bp, Artificial Sequence
  Description of Artificial Sequence Synthetic polynucleotide
  Sequence from PTPR LAR
gttccgtccggtccaccccgaaaagttgaggtagagcccctgaatagtacggcagtacatgtctattggaagctccctgttccatcaaagcaacatgggcagattagggggtaccaagttacatatgtgcgattggaaaacggggaaccacggggcctcccgatcatccaagatgttatgctcgcagaggcacagtggcgccccgaggagtccgaagattatgaaactactataagtgggcttaccccagagacaacctactctgtaactgtagctgcttacacgaccaaaggggatggcgcccgaagcaagcccaaaattgttaccacaacaggggctgtacccggcagaccgacaatgatgatcagcacgacagcgatgaacacggcacttctgcaatggcatccgcccaaggaattgccaggggagttgctcggctaccggctgcaatactgtagggcagatgaagcaaggccgaacacaatcgatttcggaaaggatgatcagcactttactgttaccggacttcataaagggacaacttatatcttccggctggcggctaaaaatagagcgggcttgggagaagaattcgagaaggagattagaaccccggaggatttgcctagtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggaggg

SEQ ID NO: 140 - protein, 979 aa, Artificial Sequence
  Description of Artificial Sequence Synthetic polypeptide
  pCDL2764
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSVPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 141 - DNA, 2937 bp, Artificial Sequence
  Description of Artificial Sequence Synthetic polynucleotide
  pCDL2764
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcgtacccggcagaccgacaatgatgatcagcacgacagcgatgaacacggcacttctgcaatggcatccgcccaaggaattgccaggggagttgctcggctaccggctgcaatactgtagggcagatgaagcaaggccgaacacaatcgatttcggaaaggatgatcagcactttactgttaccggacttcataaagggacaacttatatcttccggctggcggctaaaaatagagcgggcttgggagaagaattcgagaaggagattagaaccccggaggatttgcctagtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggagggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 142 - protein, 484 aa, Artificial Sequence
  Description of Artificial Sequence Synthetic polypeptide
  Sequence from PTPR LAR
VPGRPTMMISTTAMNTALLQWHPPKELPGELLGYRLQYCRADEARPNTIDFGKDDQHFTVTGLHKGTTYIFRLAAKNRAGLGEEFEKEIRTPEDLPSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGG

SEQ ID NO: 143 - DNA, 1452 bp, Artificial Sequence
  Description of Artificial Sequence Synthetic polynucleotide
  Sequence from PTPR LAR
gtacccggcagaccgacaatgatgatcagcacgacagcgatgaacacggcacttctgcaatggcatccgcccaaggaattgccaggggagttgctcggctaccggctgcaatactgtagggcagatgaagcaaggccgaacacaatcgatttcggaaaggatgatcagcactttactgttaccggacttcataaagggacaacttatatcttccggctggcggctaaaaatagagcgggcttgggagaagaattcgagaaggagattagaaccccggaggatttgcctagtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggaggg

SEQ ID NO: 144 - protein, 883 aa, Artificial Sequence
  Description of Artificial Sequence Synthetic polypeptide
  pCDL2765
MLLLVTSLLLCELPHPAFLLIPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGGGGGGSPAAKRVKLDGSMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGGSGGGSDALDDFDLDMLGSDALDDFDLDMLGSDALDDFDLDMLGSDALDDFDLDML

SEQ ID NO: 145 - DNA, 2649 bp, Artificial Sequence
  Description of Artificial Sequence Synthetic polynucleotide
  pCDL2765
atgcttctcctggtgacaagccttctgctctgtgagttaccacacccagcattcctcttgatccctgaacaaaagctgatcagcgaggaggatctcgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagtggcggagggggatctgaagtgaaactgcaggaaagcggccctggcctggtggccccatctcagtctctgagcgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctagcagtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggagggggtggtgggggctcccccgccgccaagagagtgaagctggacggatccatgaaactccttagcagcatcgaacaggcttgcgacatctgcaggttgaaaaaactcaagtgctcaaaagaaaagcctaagtgcgcaaagtgccttaaaaacaattgggaatgtcgctatagccccaagacaaagcggagccctctcacgagagcacacctgactgaggtagaatctcgcttggagaggctggaacagcttttcctgcttatctttccacgcgaggatctcgatatgatcctcaaaatggactccctccaggacatcaaagctctgctgactggactgtttgtacaggataatgtgaacaaggacgctgtgacagacagattggcaagcgtggaaacggatatgcccctgacccttagacagcaccggatcagtgccacctcttctagcgaggaaagttcaaataaaggacagcgccagctgacggtgagtggcggtggaagcggaggaggttccgacgctcttgatgatttcgatctcgacatgctgggatcagacgctctcgacgacttcgatttggacatgcttggatccgacgctctcgatgatttcgacctcgacatgctcggatccgatgctctggatgactttgatcttgatatgctg

SEQ ID NO: 146 - protein, 388 aa, Artificial Sequence
  Description of Artificial Sequence Synthetic polypeptide
  Sequence from PTPR LAR
SGFPQNLHVTGLTTSTTELAWDPPVLAERNGRIISYTVVFRDINSQQELQNITTDTRFTLTGLKPDTTYDIKVRAWTSKGSGPLSPSIQSRTMPVEQVFAKNFRVAAAMKTSVLLSWEVPDSYKSAVPFKILYNGQSVEVDGHSMRKLIADLQPNTEYSFVLMNRGSSAGGLQHLVSIRTAPDLLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGG

SEQ ID NO: 147 - DNA, 1164 bp, Artificial Sequence
  Description of Artificial Sequence Synthetic polynucleotide
  Sequence from PTPR LAR
agtggattcccgcaaaatctccatgtcacgggactcactacttcaacgaccgaactcgcttgggatcctccagtccttgccgaaagaaacggaaggattataagctacaccgtagtcttccgagatattaactcacaacaagaactccagaatattactaccgatacgcgattcacattgacaggacttaagcctgacacgacttatgatataaaagtccgggcttggacttccaaaggaagcggaccactgtctccctcaatccaatcgaggactatgccagttgagcaagtgtttgcaaaaaacttcagagtagcggcggcgatgaaaacttctgtccttctgagctgggaagttccggattcctataaatccgctgtccccttcaagatattgtacaatggtcaaagtgtcgaggttgatggacactccatgaggaaactcattgccgacctccaaccgaacacagaatacagttttgttttgatgaatcgcggttcctcagccgggggcctgcagcacctcgtgagtattaggaccgctcccgatcttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggaggg

SEQ ID NO: 148 - protein, 199 aa, Artificial Sequence
  Description of Artificial Sequence Synthetic polypeptide
  Sequence from PTPR Delta
VLRTKPAFIGKTNLDGMITVQLPEVPANENIKGYYIIIVPLKKSRGKFIKPWESPDEMELDELLKEISRKRRSIRYGREVELKPYIAAHFDVLPTEFTLGDDKHYGGFTNKQLQSGQEYVFFVLAVMEHAESKMYATSPYSDPVVSMDLDPQPITDEEEGLIWVVGPVLAVVFIICIVIAILLYKRKRAESDSRKSSGG

SEQ ID NO: 149 - DNA, 597 bp, Artificial Sequence
  Description of Artificial Sequence Synthetic polynucleotide
  Sequence from PTPR Delta
gtgctcaggacgaagcccgcgtttataggcaagaccaatcttgatggcatgatcactgttcagctcccggaagttcccgccaacgagaatatcaagggttattatattattatcgtaccgctcaagaagtctcgaggcaaatttatcaaaccttgggagtcaccagatgaaatggagcttgatgagttgctcaaagagatcagcagaaagcggcggtccataaggtacggcagggaggtcgagctcaagccatacattgcggctcatttcgatgtgttgccgacggagttcacgctcggggatgataaacactacggcggcttcacaaacaaacagctccaatcagggcaggagtatgtcttcttcgtgcttgctgtcatggaacacgccgaatccaaaatgtatgcaacaagcccttactccgatccggttgtttctatggatctggacccgcagccgataacagatgaagaagaagggctcatttgggtggttggccctgtgctggccgtggtgtttattatctgtatcgttattgcgattcttctctataagcggaagcgagcggagagtgactctcgaaaatcatccgggggt

SEQ ID NO: 150 - DNA, 6 bp, Artificial Sequence
  Description of Artificial Sequence Synthetic oligonucleotide
  Linker
ggatcc

SEQ ID NO: 151 - protein, 205 aa, Artificial Sequence
  Description of Artificial Sequence Synthetic polypeptide
  Sequence from PTPR LAR
LLPHKPLPASAYIEDGRFDLSMPHVQDPSLVRWFYIVVVPIDRVGGSMLTPRWSTPEELELDELLEAIEQGGEEQRRRRRQAERLKPYVAAQLDVLPETFTLGDKKNYRGFYNRPLSPDLSYQCFVLASLKEPMDQKRYASSPYSDEIVVQVTPAQQQEEPEMLWVTGPVLAVILIILIVIAILLFKRKRTHSPSSKDEQSIGGG

SEQ ID NO: 152 - DNA, 615 bp, Artificial Sequence
  Description of Artificial Sequence Synthetic polynucleotide
  Sequence from PTPR LAR
cttcttccacataagcctctccccgcatctgcgtacatagaagacgggcggttcgacctgtcaatgccccacgtacaggaccctagtcttgtaagatggttttatatcgtggtcgttcctatagaccgggttggaggctctatgctcaccccgcgatggagtaccccggaagaacttgagctggatgagttgttggaagcaatcgaacaggggggcgaggaacagaggcggcgccgccgccaagcagagcgactgaaaccgtatgttgccgctcagttggatgtgttgcccgaaacgtttactttgggtgacaaaaaaaattatcgggggttctacaatagacctttgagtcctgatcttagttatcagtgttttgtgttggcatctttgaaggaaccgatggaccagaaacgatacgcttcctccccctatagtgacgagattgtcgttcaagtaacacccgcacaacagcaggaggagccggaaatgctttgggttacagggccggtacttgcggtgatcttgataatactcattgtcatagccattctgcttttcaagcgcaaacggacccactcaccctcttccaaggacgagcagagcatagggggaggg

SEQ ID NO: 153 - protein, 111 aa, Artificial Sequence
  Description of Artificial Sequence Synthetic polypeptide
  Sequence from PTPR Mu
AAQPFTIGDNKTYNGYWNTPLLPYKSYRIYFQAASRANGETKIDCVQVATKGAATPKPVPEPEKQTDHTVKIAGVIAGILLFVIIFLGVVLVMKKRKLAKKRKETMSSTGG

SEQ ID NO: 154 - DNA, 333 bp, Artificial Sequence
  Description of Artificial Sequence Synthetic polynucleotide
  Sequence from PTPR Mu
gcggcacagccctttacaataggggacaacaagacttacaatggctattggaacacccccttgcttccttacaagagctacaggatctactttcaagcggcctcccgcgcaaacggtgaaacgaaaattgactgtgtgcaggtagccacaaagggtgcagcgactccgaagcccgtaccggagccagagaagcaaactgatcacacagtcaagattgccggcgtcatagcaggtattctgttgttcgtgataatctttctcggcgtcgtcctcgttatgaagaagaggaaactcgcaaagaagcggaaggaaacaatgtcatccactggtgga

SEQ ID NO: 155 - protein, 117 aa, Artificial Sequence
  Description of Artificial Sequence Synthetic polypeptide
  Sequence from PTPR Psi
PEAMPFTVGDNQTYRGFWNPPLEPRKAYLIYFQAASHLKGETRLNCIRIARKAACKESKRPLEVSQRSEEMGLILGICAGGLAVLILLLGAIIVIIRKGRDHYAYSYYPKPVNMTGG

SEQ ID NO: 156 - DNA, 351 bp, Artificial Sequence
  Description of Artificial Sequence Synthetic polynucleotide
  Sequence from PTPR Psi
cctgaggcgatgcccttcaccgtgggggacaatcagacctacaggggattttggaatccacctcttgaacctcgcaaagcgtacctgatctatttccaggctgcgtcacacctgaaaggggaaaccaggttgaattgcatccgcatagctaggaaagccgcctgtaaagagtccaaaaggccacttgaagtctctcagcgcagtgaagaaatgggtctgatccttggaatttgcgcgggagggctggctgtacttatccttctcctcggagctataatcgttataatcaggaaaggcagagaccactacgcctactcttactatcctaaaccggtgaacatgacgggggga

SEQ ID NO: 157 - protein, 66 aa, Artificial Sequence
  Description of Artificial Sequence Synthetic polypeptide
  Sequence from PTPR Rho
ASTQNSNTVEPEKQVDNTVKMAGVIAGLLMFIIILLGVMLTIKRRRNAYSYSYYLKLAKKQKETGG

SEQ ID NO: 158 - DNA, 198 bp, Artificial Sequence
  Description of Artificial Sequence Synthetic polynucleotide
  Sequence from PTPR Rho
gccagcacacagaattctaatactgtggagcccgagaagcaagttgacaacactgtgaaaatggctggggtaattgcaggactgcttatgttcatcataatcctgcttggggttatgcttactatcaagcgacggcgcaacgcctacagctatagctactatttgaaattggcaaaaaagcagaaggaaactggaggg

SEQ ID NO: 159 - protein, 204 aa, Artificial Sequence
  Description of Artificial Sequence Synthetic polypeptide
  Sequence from PTPR Sigma
LLNGKPSVAPKPDADGFIMVYLPDGQSPVPVQSYFIVMVPLRKSRGGQFLTPLGSPEDMDLEELIQDISRLQRRSLRHSRQLEVPRPYIAARFSVLPPTFHPGDQKQYGGFDNRGLEPGHRYVLFVLAVLQKSEPTFAASPFSDPFQLDNPDPQPIVDGEEGLIWVIGPVLAVVFIICIVIAILLYKNKPDSKRKDSEPRTKGG

SEQ ID NO: 160 - DNA, 612 bp, Artificial Sequence
  Description of Artificial Sequence Synthetic polynucleotide
  Sequence from PTPR Sigma
ttgctgaacggtaagccctcagtggcccccaaaccggatgccgacggatttataatggtgtaccttccagatggtcagagtccggtccccgtacagagctacttcattgtcatggtgcccctcaggaaatcccgaggtggtcaatttctcacaccattgggtagtccggaggacatggatctggaagaactgatccaggatattagccgcctgcaacgcagatcacttagacatagtagacagctggaggtgccgaggccgtacatcgctgcgcgattctccgtactcccgccaacctttcacccaggggatcagaaacaatacggcggttttgataatcgagggcttgaaccaggacatagatacgtgctttttgtgttggctgtgctccagaaatctgaaccgacgtttgccgcaagcccctttagcgacccatttcagctggataaccctgaccctcagccgatagtcgatggcgaggaggggctgatatgggtgattgggcccgtactcgcggtagtgtttattatctgtatcgtaattgctatactgctttataagaacaagccggacagtaaaaggaaggattctgagcctaggactaaaggcggt

SEQ ID NO: 161 - protein, 109 aa, Artificial Sequence
  Description of Artificial Sequence Synthetic polypeptide
  Sequence from PTPR Kappa
EPAPFTVGDNRTYQGFWNPPLAPRKGYNIYFQAMSSVEKETKTQCVRIATKAAATEEPEVIPDPAKQTDRVVKIAGISAGILVFILLLLVVILIVKKSKLAKKRKDAMG

SEQ ID NO: 162 - DNA, 327 bp, Artificial Sequence
  Description of Artificial Sequence Synthetic polynucleotide
  Sequence from PTPR Kappa
gagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatgggg

SEQ ID NO: 163 - protein, 4 aa, Artificial Sequence
  Description of Artificial Sequence Synthetic peptide
  Sequence from PTPR LAR
VPSG

SEQ ID NO: 164 - protein, 4 aa, Artificial Sequence
  Description of Artificial Sequence Synthetic peptide
  Sequence from PTPR LAR
EQVF

SEQ ID NO: 165 - DNA, 12 bp, Artificial Sequence
  Description of Artificial Sequence Synthetic oligonucleotide
  Sequence from PTPR LAR
gttccgtccggt

SEQ ID NO: 166 - DNA, 12 bp, Artificial Sequence
  Description of Artificial Sequence Synthetic oligonucleotide
  Sequence from PTPR LAR
gagcaagtgttt

(19) United States
(12) Patent Application Publication
(10) Pub. No.: US 2020/0384030 A1
(43) Pub. Date: Dec. 10, 2020

(54) CHIMERIC TRANSMEMBRANE RECEPTORS AND USES THEREOF

(71) Applicant: CELL DESIGN LABS, INC., Emeryville, CA (US)

(72) Inventors: Peter Emtage, Lafayette, CA (US); Andrew Glibicky, Mountain View, CA (US); Spencer Scott, San Francisco, CA (US)

(21) Appl. No.: 16/969,805

(22) PCT Filed: Feb. 20, 2019
(86) PCT No.: PCT/US2019/018813

§ 371 Date: Aug. 13, 2020

Related U.S. Application Data
(60) Provisional application No. 62/633,543, filed on Feb. 21, 2018.

(51) Int. Cl. A61K 35/17; C07K 16/28; C07K 14/725; C12N 9/16

(52) U.S. Cl. CPC A61K 35/17; C07K 16/2803; C07K 2317/622; C12N 9/16; C12Y 301/03048; C07K 14/7051

(57) ABSTRACT

Provided herein are chimeric transmembrane receptors and methods of using them to regulate selective gene expression in cells (e.g., immune cells). For example, chimeric transmembrane receptors provided herein may be synthetic receptor-like protein tyrosine phosphatases (“synPTPRs”) that can regulate transcription of a heterologous target gene in a cell when bound by a target antigen present on a target cell.

### CROSS-REFERENCE TO RELATED APPLICATION

This application claims priority to U.S. Provisional Patent Application Ser. No. 62/633,543, filed Feb. 21, 2018, the entire contents of which are herein incorporated by reference.

### TECHNICAL FIELD

The present invention relates to molecular biology, and particularly to methods and compositions for regulating selective gene expression in cells (e.g., cells of the monocyte/macrophage lineage), and applications thereof.

### BACKGROUND

A variety of new tools for treating cancer have been developed in recent years. For example, expression vectors encoding chimeric antigen receptors (CARs) and engineered T cell receptors (TCRs) that target certain cancer antigens present on cancer cells have been introduced into immune cells, which engineered immune cells are then administered to a subject having cancer. Regulating the expression, activity, or both, of such engineered immune cells remains an active area of endeavor. A variety of regulatory mechanisms to control the expression, activity, or both, of chimeric antigen receptors, for example, are known in the art. See, e.g., Roybal et al, “Precision Tumor Recognition by T Cells With Combinatorial Antigen-Sensing Circuits”, Cell 2016; Wu et al, “Remote control of therapeutic T cells through a small molecule-gated chimeric receptor”, Science 2015; Sakemura et al, “A Tet-On Inducible System for Controlling CD19-Chimeric Antigen Receptor Expression upon Drug Administration”, Cancer Immunol Res 2016; Rakhit et al, “Chemical biology strategies for posttranslational control of protein function”, Chem Biol 2014; Navarro et al, “A Novel Destabilizing Domain Based on a Small-Molecule Dependent Fluorophore”, ACS Chem Biol 2016, each of which is incorporated herein by reference in its entirety. Improved compositions and methods for regulating engineered immune cells expressing, for example, chimeric antigen receptors or T cell receptors are needed.

### SUMMARY OF THE INVENTION

Provided herein are methods and compositions for regulating selective expression of a protein (e.g., a therapeutic protein, e.g., a chimeric antigen receptor or T-cell receptor) in cells (e.g., immune cells), and applications thereof.

In some embodiments, provided herein are chimeric transmembrane receptors that include: an extracellular antigen-binding domain that is capable of specifically binding to a target antigen; an extracellular integrin ligand-binding domain that includes an S2 protease cleavage site; a transmembrane domain; an intracellular regulatory domain that includes a gamma-secretase protease cleavage site; and an intracellular transcriptional regulatory domain; wherein, when the chimeric transmembrane receptor is expressed in a mammalian cell, binding of the extracellular antigen-binding domain to the target antigen induces (1) cleavage of the extracellular integrin-ligand binding domain at the S2 protease cleavage site and (2) cleavage of the intracellular regulatory domain at the gamma-secretase protease cleavage site, thereby releasing the intracellular transcriptional regulatory domain from the transmembrane domain.

In some embodiments, chimeric transmembrane receptors provided herein include an antigen-binding domain that is an antibody or an antibody fragment. In some embodiments, a chimeric transmembrane receptor includes an antigen-binding domain that an antibody, wherein the antibody is selected from the group consisting of: a Fab fragment, an Fv fragment, a scFv fragment, an Fd fragment, a chimeric antibody, a humanized antibody, a fully-human antibody, a single-chain antibody (scAb), a single domain antibody (dAb), a single domain heavy chain antibody, a single domain light chain antibody, a nanobody, a bi-specific antibody, and a multi-specific antibody.

In some embodiments, chimeric transmembrane receptors provided herein include an antigen-binding domain that binds a target antigen selected from the group consisting of: BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, IL-13, IL-6, insulin-like growth factor I receptor, integrin α5β1, integrin ανβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

In some embodiments, chimeric transmembrane receptors provided herein include an extracellular integrin ligand-binding domain that is a human fibronectin III domain or a mouse fibronectin III domain. In some embodiments, chimeric transmembrane receptors provided herein include an extracellular integrin ligand-binding domain that includes a sequence at least 80% identical to a sequence of a wild type human fibronectin III domain or a sequence of a wild type mouse fibronectin III domain. In some embodiments, chimeric transmembrane receptors provided herein include an additional extracellular integrin ligand-binding domain. In some embodiments, an additional extracellular integrin ligand-binding domain of a chimeric transmembrane receptor provided herein includes a wild type human fibronectin type III domain or a wild type mouse fibronectin type III domain. In some embodiments, an additional extracellular integrin ligand-binding domain of a chimeric transmembrane receptor provided herein includes comprises a sequence at least 80% identical to a sequence of a wild type human fibronectin III domain or a sequence of a wild type mouse fibronectin III domain.

In some embodiments, chimeric transmembrane receptors provided herein include a transmembrane domain that is present in a receptor-like tyrosine phosphatase. In some embodiments, chimeric transmembrane receptors provided herein include a transmembrane domain that is at least 80% identical to a sequence of a transmembrane domain present in a receptor-like tyrosine phosphatase. In some embodiments, chimeric transmembrane receptors provided herein include a transmembrane domain that is present in a polypeptide selected from the group consisting of: CD28, CD3 epsilon, CD4, CD5, CD6, CD8a, CD9, CD16, CD22, CD33, CD37, CD 45, CD64, CD80, CD86, CD134, 4-1BB, GITR, NGFR, and CD154. In some embodiments, chimeric transmembrane receptors provided herein include a transmembrane domain that includes a sequence that is at least 80% identical to the sequence of a transmembrane domain present in a polypeptide selected from the group consisting of: CD28, CD3 epsilon, CD4, CD5, CD6, CD8a, CD9, CD16, CD22, CD33, CD37, CD 45, CD64, CD80, CD86, CD134, 4-1BB, GITR, NGFR, and CD154.

In some embodiments, chimeric transmembrane receptors provided herein include a gamma-secretase cleavage site that includes a Gly-Val dipeptide amino acid sequence.

In some embodiments, chimeric transmembrane receptors provided herein include an intracellular transcriptional regulatory domain is a transcriptional activator. In some embodiments, chimeric transmembrane receptors provided herein include an intracellular transcriptional regulatory domain is a transcriptional repressor. In some embodiments, chimeric transmembrane receptors provided herein include an intracellular transcriptional regulatory domain that is present in a polypeptide selected from the group consisting of: VP64, RelA (p65), YAP, WWTR1(TAZ), CREB3(LZIP), and MyoD. In some embodiments, chimeric transmembrane receptors provided herein include an intracellular transcriptional regulatory domain that includes a sequence that is at least 80% identical to a sequence of a transcriptional activation domain present in a polypeptide selected from the group consisting of: VP64, RelA (p65), YAP, WWTR1(TAZ), CREB3(LZIP), and MyoD.

Also provided herein are nucleic acids that encode any of the chimeric transmembrane receptors described herein. Also provided herein are vectors that include any of the nucleic acids encoding any of the chimeric transmembrane receptors described herein. Also provided herein are mammalian cells that include any of the nucleic acids encoding any of the chimeric transmembrane receptors described herein described herein or any of the vectors described herein. In some embodiments, the mammalian cell is an immune cell. For example, the immune cell can be selected from the group consisting of: a CD4+ T cell, a CD8+ T cell, a B cell, a monocyte, a natural killer cell, a dendritic cell, a macrophage, a regulatory T cell, and a helper T cell. In some embodiments, the mammalian cell further includes a heterologous target gene that includes (i) a transcription regulatory sequence that is capable of being specifically recognized by the intracellular transcriptional regulatory domain and (ii) a nucleic acid sequence that encodes a recombinant protein, wherein the nucleic acid sequence that encodes the recombinant protein is operably linked to the transcription regulatory sequence.

In some embodiments, a recombinant protein encoded by a heterologous target gene is a secreted polypeptide.

In some embodiments, a recombinant protein encoded by a heterologous target gene is a chimeric antigen receptor (CAR). For example, a CAR can include an antigen-binding domain capable of specifically binding to an antigen selected from the group consisting of: BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, IL-13, IL-6, insulin-like growth factor I receptor, integrin α5β1, integrin ανβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-0, TRAIL-R1, TRAIL-R2, tumor antigen CTAA16.88, VEGF-A, VEGFR-1, VEGFR2, and vimentin.

In some embodiments, a recombinant protein encoded by a heterologous target gene is a T cell receptor (TCR). For example, a TCR can include an antigen-binding domain capable of specifically binding to an antigen selected from the group consisting of: BCMA, CD11a, CD19, CD20, CD22, CD30, CD38, CD52, Her2/neu, ENPP3, EGFR, MAGE-A1, IL-13R-a2, GD2, alpha-integrin, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), or high molecular weight-melanoma associated antigen (HMW-MAA).

Also provided herein are pharmaceutical compositions that include any of the mammalian cells described herein. Also provided herein are pharmaceutical compositions that include any of the nucleic acids or vectors described herein. In some embodiments, pharmaceutical compositions that include any of the nucleic acids or vectors described herein can further include a heterologous target gene that includes (i) a transcription regulatory sequence that is capable of being specifically recognized by the intracellular transcriptional regulatory domain and (ii) a nucleic acid sequence that encodes a recombinant protein, wherein the nucleic acid sequence that encodes the recombinant protein is operably linked to the transcription regulatory sequence.

Also provided herein are methods of treating disease in a subject in need thereof that include administering a therapeutically effective amount of any of the pharmaceutical compositions described herein to the subject. In some embodiments, the disease is cancer. In some embodiments, the pharmaceutical composition includes a mammalian cell that is autologous to the subject. In some embodiments, the pharmaceutical composition includes a mammalian cell that is allogenic to the subject.

Also provided herein are nucleic acids encoding a chimeric transmembrane receptor that include: a first nucleic acid segment that encodes an extracellular antigen-binding domain that is capable of specifically binding to a target antigen; a second nucleic acid segment that encodes an extracellular integrin ligand-binding domain that includes an S1 protease cleavage site, an S2 protease cleavage site, or both; a third nucleic acid segment that encodes a transmembrane domain; a fourth nucleic acid segment that encodes an intracellular regulatory domain that includes a gamma-secretase protease cleavage site; and a fifth nucleic acid segment that encodes an intracellular transcriptional regulatory domain; wherein, when the chimeric transmembrane receptor is expressed in a mammalian cell, binding of the extracellular antigen-binding domain to the target antigen induces (1) cleavage of the extracellular integrin-ligand binding domain at the S2 protease cleavage site and (2) cleavage of the intracellular regulatory domain at the gamma-secretase protease cleavage site, thereby releasing the intracellular transcriptional regulatory domain from the transmembrane domain. Also provided herein are vectors that include any of the nucleic acids encoding a chimeric transmembrane receptor described herein. In some embodiments of vectors that include a nucleic acid encoding a chimeric transmembrane receptor, the nucleic acid encoding a chimeric transmembrane receptor is operably linked to a transcription regulatory sequence.

Also provided herein are mammalian cells that include any of the nucleic acids encoding a chimeric transmembrane receptor or any of the vectors described herein. In some embodiments, the mammalian cell is an immune cell. For example, the mammalian cell can be selected from the group consisting of: a CD4+ T cell, a CD8+ T cell, a B cell, a monocyte, a natural killer cell, a dendritic cell, a macrophage, a regulatory T cell, and a helper T cell. In some embodiments, the mammalian cell further includes a heterologous target gene that includes (i) a transcription regulatory sequence that is capable of being specifically recognized by the intracellular transcriptional regulatory domain and (ii) a nucleic acid sequence that encodes a recombinant protein, wherein the nucleic acid sequence that encodes the recombinant protein is operably linked to the transcription regulatory sequence. In some embodiments, a recombinant protein encoded by a heterologous target gene is a secreted polypeptide. In some embodiments, a recombinant protein encoded by a heterologous target gene is a chimeric antigen receptor (CAR). For example, a CAR can include an antigen-binding domain capable of specifically binding to an antigen selected from the group consisting of: BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, IL-13, IL-6, insulin-like growth factor I receptor, integrin α5β1, integrin ανβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. In some embodiments, a recombinant protein encoded by a heterologous target gene is a T cell receptor (TCR). For example, a TCR can include an antigen-binding domain capable of specifically binding to an antigen selected from the group consisting of: BCMA, CD11a, CD19, CD20, CD22, CD30, CD38, CD52, Her2/neu, ENPP3, EGFR, MAGE-A1, IL-13R-a2, GD2, alpha-integrin, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), or high molecular weight-melanoma associated antigen (HMW-MAA).

Other features and advantages of the invention will be apparent from the following Detailed Description of the Invention, and from the claims. It should be understood, however, that the detailed description and the specific examples, while indicating preferred embodiments of the invention, are given by way of illustration only, since various changes and modifications within the spirit and scope of the invention will become apparent to those skilled in the art from this detailed description. All publications mentioned herein, including patents, patent application publications, and scientific papers, are incorporated by reference in their entirety.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic diagram of an exemplary synPTPR based on the receptor-like protein tyrosine phosphatase type-K (PTPRK). PTPRK is composed of a MAM domain, Ig domain, four fibronectin type-III (FN-III) domains, and two intracellular phosphatase domains. In the embodiment of a synPTPR shown, only the transmembrane region and the first two most membrane-proximal fibronectin domains were kept from PTPR. An anti-CD19 scFv was fused to the N-terminus of the PTPR-core, and a transcription factor was fused to the intracellular, C-terminus of the PTPR-core. Without wishing to be bound by theory, association of the anti-CD19 scFv with its cognate ligand is hypothesized to cause a protease from the ADAM family (e.g., ADAM10 or ADAM17) to cleave the S2 cleavage site and gamma-secretase processing of the PTPR-core, releasing the intracellular transcription factor to shuttle to the nucleus and affect transcription of a nucleic acid sequence encoding a protein (e.g., a therapeutic protein, e.g., a chimeric antigen receptor or a T-cell receptor).

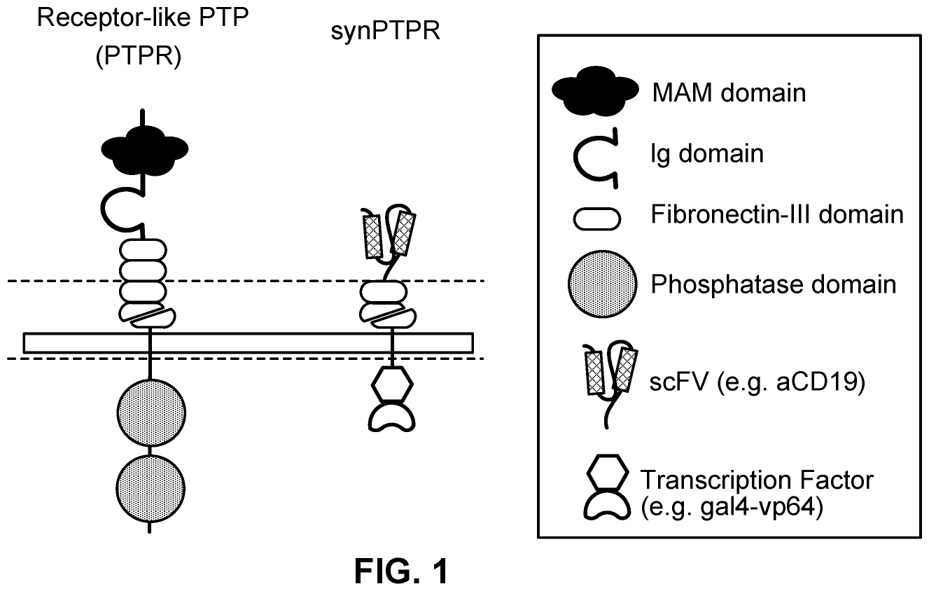

FIG. 2 is a schematic diagram of the constructs used to assess the functionality of exemplary synPTPRs as an antigen-sensing platform. An exemplary synPTPR (FIG. 2A) is composed of an aCD19 scFv, the PTPR-core, and a gal4-vp64 transcription factor. The reporter construct (FIG. 2B) includes a constitutive mCherry marker, and an inducible promoter driving GFP with multiple gal4 binding sites. In the presence of gal4-vp64, the reporter will upregulate the production of GFP. As a positive control, a synthetic Notch protein with the same aCD19 scFv and gal4-vp64 transcription factor was used (FIG. 2C). The synthetic Notch protein also used the same reporter (FIG. 2D).

FIG. 3 is a graph showing GFP expression in cells expressing a Notch1 positive control and synPTPR in the presence of CD19-expressing cells. The exemplary synPTPR used in this experiment upregulated GFP expression in the presence of both low and high antigen levels of CD19, with minimal basal expression in the absence of CD19.

FIG. 4 is a schematic showing wildtype PTPR proteins and the different chimeric transmembrane receptor that each include a portion of one of the wildtype PTPR proteins that were tested in Example 3.

FIG. 5 is a schematic showing the pairs of nucleic acid constructs encoding different chimeric transmembrane receptors and reporter nucleic acids that were tested in Example 3.

FIG. 6 is a graph showing the percentage of GFP-positive cells in a population of CD3⁺ cells transduced with pCDL1932, pCDL1933, pCDL1934, pCDL1935, pCDL1936, pCDL1937, or pCDL1541, and their corresponding reporter nucleic acid (as depicted in FIG. 5) upon co-culture with CD19⁻ K562 cells (un-stimulated) or CD19⁺ Raji cells (stimulated).

FIG. 7 is a graph showing the mean fluorescence intensity in GFP⁺ and mCherry⁺ cells in a population of CD3⁺ cells transduced with pCDL1932, pCDL1933, pCDL1934, pCDL1935, pCDL1936, pCDL1937, or pCDL1541, and their corresponding reporter nucleic acid (as depicted in FIG. 5) upon co-culture with CD19− K562 cells (un-stimulated) or CD19+ Raji cells (stimulated).

FIG. 8 is a graph showing the percentage of myc-positive cells in a population of CD3⁺ cells transduced with pCDL1933, pCDL2243, pCDL2244, pCDL2246, or pCDL2244, and their corresponding reporter nucleic acid (as depicted in FIG. 5) upon co-culture with CD19⁻K562 cells (un-stimulated) or CD19⁺ Raji cells (stimulated).

FIG. 9 is a graph showing the mean fluorescence intensity in GFP⁺ and mCherry⁺ cells in a population of CD3⁺ cells transduced with pCDL1933, pCDL2243, pCDL2244, pCDL2246, or pCDL2244, and their corresponding reporter nucleic acid (as depicted in FIG. 5) upon co-culture with CD19− K562 cells (un-stimulated) or CD19+ Raji cells (stimulated).

FIG. 10 shows the percentage of myc⁺ positive cells in a population of CD3⁺ cells transduced with pCDL2762, pCDL2763, pCDL2764, pCDL2765, or pCDL1933.

FIG. 11 is a graph showing the mean fluorescence intensity in GFP⁺ and mCherry⁺ cells in a population of CD3⁺ cells transduced with pCDL2762, pCDL2763, pCDL2764, pCDL2765, or pCDL1933, and their corresponding reporter nucleic acid (as depicted in FIG. 5) upon co-culture with CD19− K562 cells (un-stimulated) or CD19+ Raji cells (stimulated).

### DETAILED DESCRIPTION OF THE INVENTION

Provided herein are chimeric transmembrane receptors that include an extracellular antigen-binding domain that is capable of specifically binding to a target antigen, an extracellular integrin ligand-binding domain comprising an S2 protease cleavage site, a transmembrane domain, an intracellular regulatory domain comprising a gamma-secretase protease cleavage site, and an intracellular transcriptional regulatory domain. In some embodiments, chimeric transmembrane receptors provided herein include one or more linkers between their various domains. In some embodiments, when the chimeric transmembrane receptor is expressed in a mammalian cell, binding of the extracellular antigen-binding domain to the target antigen induces (1) cleavage of the extracellular integrin-ligand binding domain at the S2 protease cleavage site and (2) cleavage of the intracellular regulatory domain at the gamma-secretase protease cleavage site, thereby releasing the intracellular transcriptional regulatory domain from the transmembrane domain. In some embodiments, release of the intracellular regulatory domain modulates an activity of a cell. For example, an intracellular regulatory domain can include a DNA-binding domain (e.g., any of the DNA-binding domains described herein or known in the art) and a transcriptional activation domain. (e.g., any of the transcriptional activation domains described herein or known in the art) When the intracellular regulatory domain is released upon binding of the extracellular antigen-binding domain to the target antigen, it can translocate to the nucleus of the cell where it can regulate the transcription of an mRNA encoding a polypeptide (e.g., a recombinant polypeptide, e.g., a chimeric antigen receptor or a T-cell receptor) under control of a regulatory element that is regulated by the intracellular regulatory domain (e.g., a promoter that is bound by the DNA-binding domain of the intracellular regulatory domain).

Chimeric transmembrane receptors provided herein exhibit a number of advantages over existing technology. For example, chimeric transmembrane receptors provided herein are more sensitive to activation (e.g., resulting in stronger gene regulation in the presence of a lower concentration of antigen) than other engineered receptors that are designed to regulate gene transcription upon binding a target antigen. Moreover, chimeric transmembrane receptors provided herein are smaller in size than other engineered receptors. For example, synNotch receptors such as those described in U.S. Pat. Nos. 9,670,281 and 9,834,608, each of which is incorporated herein by reference in its entirety, are limited both by sensitivity of response which is a function of the mechanism by which they occlude their S2 cleavage sites, as well as by their size. Chimeric transmembrane receptors provided herein are smaller than other engineered receptors that are designed to regulate gene transcription upon binding a target antigen, thus providing a variety of benefits over existing technology.

Various non-limiting aspects of chimeric transmembrane receptors are described herein, and can be used in any combination without limitation. Additional aspects of various components of chimeric transmembrane receptors are known in the art.

As used herein, the word “a” before a noun refers to one or more of the particular noun.

As used herein, the term “antigen” refers generally to a binding partner specifically recognized by an extracellular antigen-binding domain described herein. Exemplary antigens include different classes of molecules, such as, but not limited to, polypeptides and peptide fragments thereof, small molecules, lipids, carbohydrates, and nucleic acids. Non-limiting examples of antigen or antigens that can be specifically bound by any of the extracellular antigen-binding domains are described herein. Additional examples of antigen or antigens that can be specifically bound by any of the extracellular antigen-binding domains are known in the art.

The terms “chimeric antigen receptor” and “CAR”, used interchangeably herein, refer to artificial multi-module molecules capable of triggering or inhibiting the activation of an immune cell, which generally but not exclusively include an extracellular domain (e.g., a ligand/antigen binding domain), a transmembrane domain and one or more intracellular signaling domains. The term CAR is not limited specifically to CAR molecules but also includes CAR variants, i.e., CAR variants are described, e.g., in PCT Application No. US2014/016527; Fedorov et al., Sci Transl. Med. 5(215):215ra172, 2013; Glienke et al., Front. Pharmacol. 6:21, 2015; Kakarla & Gottschalk, Cancer J. 20(2):151-155, 2014; Riddell et al., Cancer J. 20(2):141-144, 2014; Pegram et al., Cancer J. 20(2):127-33, 2014; Cheadle et al., Immunol Rev. 257(1):91-106, 2014; Barrett et al., Ann. Rev. Med. 65:333-347, 2014; Sadelain et al., Cancer Discov. 3(4):388-98, 2013; and Cartellieri et al., J. Biomed. Biotechnol. 956304, 2010; the disclosures of which are incorporated herein by reference in their entirety.

The term “extracellular antigen-binding domain” means a domain that is present on the extracellular side of the plasma membrane and binds specifically to a target antigen. In some examples, an extracellular antigen-binding domain can be formed from the amino acids present within a single-chain polypeptide. In other examples, an extracellular antigen-binding domain can be formed from amino acids present within a first single-chain polypeptide and the amino acids present in one or more additional single-chain polypeptides (e.g., a second single-chain polypeptide). Non-limiting examples of extracellular antigen-binding domains are described in more detail herein, including, without limitation, scFvs, or LBDs (Ligand Binding Domains) of growth factors. Additional examples of extracellular antigen-binding domains are known in the art.

The phrase “extracellular side of the plasma membrane” when used to describe the location of a transmembrane polypeptide means that the polypeptide includes at least one transmembrane domain that traverses the plasma membrane and at least one domain (e.g., at least one extracellular antigen-binding domain) that is located in the extracellular space.

“GFP” or green fluorescent protein (GFP) is a commonly used reporter of gene expression. Arun et al., J. Pharmacol. Toxicol. Methods 51(1):1-23, 2005.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified to greater than 90%, greater than 95%, or greater than 98%.

“Linkers” are amino acid sequences that separate multiple domains in a single protein, and, generally, can be classified into three groups: flexible, rigid and cleavable. Chen, X., et al., 2013, Adv. Drug Deliv. Rev., 65, 1357-1369. Linkers can be natural or synthetic. A number of linkers are employed to realize the subject invention including “flexible linkers.” The latter are rich in glycine. Klein et al., Protein Engineering, Design & Selection Vol. 27, No. 10, pp. 325-330, 2014; Priyanka et al., Protein Sci., 2013 February; 22(2): 153-167. In some embodiments, the linker is a synthetic linker. A synthetic linker can have a length of from about 10 amino acids to about 200 amino acids, e.g., from 10 to 25 amino acids, from 25 to 50 amino acids, from 50 to 75 amino acids, from 75 to 100 amino acids, from 100 to 125 amino acids, from 125 to 150 amino acids, from 150 to 175 amino acids, or from 175 to 200 amino acids. A synthetic linker can have a length of from 10 to 30 amino acids, e.g., 10, 11, 12, 13, 14, 15, 16, 17, 18, 19, 20, 21, 22, 23, 24, 25, 26, 27, 28, 29, or 30 amino acids. A synthetic linker can have a length of from 30 to 50 amino acids, e.g., from 30 to 35 amino acids, from 35 to 40 amino acids, from 40 to 45 amino acids, or from 45 to 50 amino acids. In some embodiments, the linker is a flexible linker. In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS, SEQ ID NO: 1) sequences, e.g., 1, 2, 3, 4, 5, 6, 7, 8, 9, or 10 or more GGGS sequences. In some embodiments, the linker has one or more Gly-Gly-Gly-Gly-Ser (GGGGS, SEQ ID NO: 2) sequences, e.g., 1, 2, 3, 4, 5, 6, 7, 8, 9, or 10 or more GGGGS sequences. In some embodiments, the linker has one or more Gly-Gly-Ser-Gly (GGSG, SEQ ID NO: 3) sequences, e.g., 1, 2, 3, 4, 5, 6, 7, 8, 9, or 10 or more GGSG sequences. In some embodiments, the linker is or comprises GSAAAGGSGGSGGS (SEQ ID NO: 4). In some embodiments, the linker is or comprises GGGSGGGS (SEQ ID NO: 5).

In some examples, a Gly-Gly-Gly-Gly-Ser (SEQ ID NO: 2) linker can be encoded by the nucleic acid sequence of: GGTGGAGGAGGCTCT (SEQ ID NO: 47), GGTGGTGGGGGCTCC (SEQ ID NO: 48), GGAGGTGGTGGGAGT (SEQ ID NO: 49), GGCGGAGGCGGGAGC (SEQ ID NO: 50), GGCGGTGGAGGTTCC (SEQ ID NO: 51), GGGGGAGGTGGGAGT (SEQ ID NO: 52), or GGCGGGGGAGGGAGC (SEQ ID NO: 53).

In some examples, the GGGSGGGS (SEQ ID NO: 5) linker is encoded by the nucleic acid sequence of GGCGGTGGAAGCGGAGGAGGTTCC (SEQ ID NO: 29).

The terms “polypeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, a “portion” of a polypeptide or protein refers at least 10 amino acids of the reference sequence, e.g., 10 to 200, 25 to 300, 50 to 400, 100 to 500, 200 to 600, 300 to 700, 400 to 800, 500 to 900, or 600 to 1000 or more amino acids of the reference sequence. In some embodiments, the portion of a polypeptide or protein is functional.

The term “subject” refers to any mammal. In some embodiments, the subject or “subject suitable for treatment” may be a canine (e.g., a dog), feline (e.g., a cat), equine (e.g., a horse), ovine, bovine, porcine, caprine, primate, e.g., a simian (e.g., a monkey (e.g., marmoset, baboon), or an ape (e.g., a gorilla, chimpanzee, orangutan, or gibbon) or a human; or rodent (e.g., a mouse, a guinea pig, a hamster, or a rat). In some embodiments, the subject or “subject suitable for treatment” may be a non-human mammal, especially mammals that are conventionally used as models for demonstrating therapeutic efficacy in humans (e.g., murine, lapine, porcine, canine or primate animals) may be employed.

The term “synNotch” refers to any of the variety of synthetic receptor-like polypeptides that use endogenous or modified Notch domains to effect intracellular signaling. Exemplary synNotch polypeptides are described in U.S. Pat. Nos. 9,670,281 and 9,834,608, and generally comprise, from N-terminal to C-terminal an extracellular antigen-binding domain, one or more ligand-inducible proteolytic cleavage sites, and an intracellular domain, wherein binding of extracellular antigen-binding domain to its target induces cleavage of the Notch receptor polypeptide at the one or more ligand-inducible proteolytic cleavage sites, thereby releasing the intracellular domain. As will be clear to one of ordinary skill in the art upon reading the present disclosure, “synPTPR” constructs provided herein exhibit certain advantages over synNotch constructs.

The term “synPTPR” refers to any of the variety of chimeric transmembrane receptor described herein. In general, synPTPRs described herein have had a substantial part of their wild type extracellular domains replaced with an extracellular antigen-binding domain. In some embodiments, synPTPRs described herein have an extracellular antigen-binding domain in place of the MAM domain, the Ig domain, and one or more FN-III domains that are endogenously present in a PTPR. In some embodiments, synPTPRs described herein have an intracellular regulatory domain comprising a gamma-secretase protease cleavage site in place of the phosphatase domains that are endogenously present in a PTPR. In some embodiments, synPTPRs described herein have one or more (e.g., one or two) extracellular integrin ligand-binding domain(s), which integrin ligand-binding domain(s) are cleaved upon the extracellular antigen-binding domain of the binding of the chimeric transmembrane receptor to its target ligand. In some embodiments, such cleavage results in cleavage of the gamma-secretase protease cleavage site, resulting in release of the intracellular transcriptional regulatory domain from the transmembrane domain.

The term “TCR” refers to a T cell receptor, a multi-module molecule capable of triggering or inhibiting the activation of an immune cell which generally but not exclusively includes an extracellular domain (e.g., a ligand/antigen binding domain), a transmembrane domain and one or more intracellular signaling domains. Wild type TCRs are heterodimers, the majority of which include an alpha and a beta chain. A smaller portion of TCRs include a gamma and a delta chain. TCRs as used herein refer to both TCRs having wild type nucleic acid and/or amino acid sequences, as well as engineered TCRs having one or more modifications in their nucleic acid and/or amino acid sequence as compared to a nucleic acid and/or amino acid sequence of a wild type TCR.

### Extracellular Antigen-Binding Domains

In some embodiments, chimeric transmembrane receptors provided herein include at least one extracellular antigen-binding domain that specifically binds to a target antigen. In some embodiments, the extracellular antigen-binding domain is selected from the group consisting of: a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2 scFv-IgG, IgG-2 scFv, scFv4-Ig, Zybody, DVI-IgG, Diabody-CH3, a triple body, a miniantibody, a minibody, a TriBi minibody, a nanobody, scFv-CH3 KIH, Fab-scFv, a F(ab′)₂-scFv₂, a scFv-KIH, a Fab-scFv-Fc, a tetravalent HCAb, a scDiabody-Fc, a Diabody-Fc, a tandem scFv-Fc, an Intrabody, a dock and lock, a ImmTAC, an IgG-IgG conjugate, a Cov-X-Body, and a scFv1-PEG-scFv₂. See, e.g., Spiess et al., Mol. Immunol. 67:95-106, 2015, incorporated in its entirety herewith, for a description of these elements. In some embodiments, the extracellular antigen-binding domain is selected from the group consisting of: a Fab fragment, an Fv fragment, a scFv fragment, an Fd fragment, a chimeric antibody, a humanized antibody, a fully-human antibody, a single-chain antibody (scAb), a single domain antibody (dAb), a single domain heavy chain antibody, a single domain light chain antibody, a nanobody, a bi-specific antibody, and a multi-specific antibody.

In some embodiments, chimeric transmembrane receptors provided herein include at least one extracellular antigen-binding domain that includes an antibody, an antibody fragment, or an antibody derivative. Such antibodies, antibody fragments, and antibody derivatives can be of any antibody isotype or subtype, or can be derived from any antibody isotype or subtype. For example, the light chains of antibodies (immunoglobulins) from any vertebrate species can be assigned to one of two distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b. Without limitation, chimeric transmembrane receptors provided herein can include at least one extracellular antigen-binding domain that includes an antibody, an antibody fragment, or an antibody derivative, wherein the antibody, antibody fragment, or antibody derivative is of any of the light and heavy chain types or classes described herein.

In some embodiments, an extracellular antigen-binding domain is humanized or fully human. “Humanized” as used herein refers to an antibody comprising portions of antibodies of different origin, wherein at least one portion comprises amino acid sequences of human origin.

For example, a humanized antibody can comprise portions derived from an antibody of nonhuman origin with the requisite specificity, such as a mouse, and from antibody sequences of human origin (e.g., chimeric antibody), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized antibody is an antibody containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized antibody. See, e.g., Cabilly et al., U.S. Pat. No. 4,816,567; Boss et al., U.S. Pat. No. 4,816,397; Neuberger, M. S. et al., WO 86/01533; Winter, U.S. Pat. No. 5,225,539; See also, Ladner et al., U.S. Pat. No. 4,946,778; Huston, U.S. Pat. No. 5,476,786; and Bird, R. E. et al., Science, 242: 423-426 (1988)), regarding single chain antibodies.

Antibody fragments that can be used as extracellular antigen-binding domains in chimeric transmembrane receptors provided herein include a portion of an intact antibody, for example, the antigen binding or variable region of the intact antibody, which portion retains the capability of specifically binding to an antigen. Non-limiting examples of antibody fragments that can be used as an extracellular antigen-binding domain of an chimeric transmembrane receptor include an Fv fragment, a Fab fragment, a F(ab′)₂ fragment, and a Fab′ fragment. Additional examples of an antigen-binding fragment of an antibody include an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM). Additional examples of antibody fragments that can be used in antigen-binding domains of chimeric transmembrane receptors provided herein are known in the art.

A Fv fragment is the minimum antibody fragment that contains a complete antigen-recognition and binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

A scFv (also referred to as a “single-chain Fv” or a “sFv”) is an antibody fragment that includes the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, Vol. 113, Rosenburg and Moore eds., Springer-Verlag, New York, pp. 269-315 (1994).

A Fab fragment includes the constant domain of the light chain and the first constant domain (CH1) of the heavy chain, in addition to the heavy and light chain variable domains of the Fv fragment. Papain digestion of antibodies produces two identical Fab antigen-binding fragments, each with a single antigen-binding site, and a residual “Fc” fragment, a designation reflecting the ability to crystallize readily. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

A F(ab′)₂ fragment includes two Fab fragments joined, near the hinge region, by disulfide bonds. Pepsin treatment yields an F(ab′)₂ fragment that has two antigen combining sites and is still capable of cross-linking antigen.

A nanobody (Nb) is the smallest antigen binding fragment or single variable domain (V.sub.HH) derived from naturally occurring heavy chain antibody. They are derived from heavy chain only antibodies, seen in camelids. In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, an engineered immune cell includes a single antigen-binding domain. In some embodiments, a single antigen-binding domain is a “dual variable domain immunoglobulin” or “DVD-Ig”. A dual variable domain immunoglobulin is a multivalent and multispecific binding protein as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety. In some embodiments, a single antigen-binding domain present in an engineered immune cell is a DART. DARTs are described in, e.g., Garber, Nature Reviews Drug Discovery 13:799-801, 2014.

Diabodies are small antibody fragments with two antigen-binding sites, which fragments comprise a heavy-chain variable domain (VH) connected to a light-chain variable domain (VL) in the same polypeptide chain (VH-VL). Diabodies are described in EP 404,097; WO 93/11161; and Hollinger et al., Proc. Natl. Acad. Sci. U.S.A. 90:6444-6448, 1993.

In some embodiments, an extracellular antigen-binding domain of a chimeric transmembrane receptor provided herein binds to a target antigen selected from the group consisting of: BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, IL-13, IL-6, insulin-like growth factor I receptor, integrin α5β1, integrin ανβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

In some embodiments, an extracellular antigen-binding domain of a chimeric transmembrane receptor provided herein is bi-specific or multi-specific in that it binds (e.g., is capable of binding) to more than one different target antigen. In some embodiments, a chimeric transmembrane receptor provided herein includes two or more extracellular antigen-binding domains, each of which binds (e.g., is capable of binding) to two or more different target antigens. For example, a chimeric transmembrane receptor can include two or more scFv domains, wherein each scFv domain binds or is capable of binding to different target antigens (e.g., CD19 and CD20).

The amino acid of an exemplary extracellular antigen-binding domain that binds specifically to human CD19 is shown below. Also shown below is the cDNA sequence that encodes this exemplary antigen-binding domain. In some embodiments, an extracellular antigen-binding domain can include a sequence that is at least 80%, at least 82%, at least 84%, at least 86%, at least 88%, at least 90%, at least 92%, at least 94%, at least 96%, at least 98%, at least 99%, or 100% identical to SEQ ID NO: 45. In some embodiments, an extracellular antigen-binding domain can be encoded by a nucleic acid including a sequence that is at least 80%, at least 82%, at least 84%, at least 86%, at least 88%, at least 90%, at least 92%, at least 94%, at least 96%, at least 98%, at least 99%, or 100% to SEQ ID NO: 45.

Exemplary Anti-Human CD19 scFv
(SEQ ID NO: 45)
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSS
cDNA Sequence Encoding an Exemplary Anti-Human
CD19 scFv
(SEQ ID NO: 46)
gacatccagatgacccagaccaccagcagcctgagcgccagcctgggcga
tagagtgaccatcagctgcagagccagccaggacatcagcaagtacctga
actggtatcagcagaaacccgacggcaccgtgaagctgctgatctaccac
accagcagactgcacagcggcgtgcccagcagattttctggcagcggctc
cggcaccgactacagcctgaccatctccaacctggaacaggaagatatcg
ctacctacttctgtcagcaaggcaacaccctgccctacaccttcggcgga
ggcaccaagctggaaatcacaggcggcggaggatctggcggaggcggaag
tggcggagggggatctgaagtgaaactgcaggaaagcggccctggcctgg
tggccccatctcagtctctgagcgtgacctgtaccgtgtccggcgtgtcc
ctgcctgactatggcgtgtcctggatcagacagccccccagaaagggcct
ggaatggctgggagtgatctggggcagcgagacaacctactacaacagcg
ccctgaagtcccggctgaccatcatcaaggacaactccaagagccaggtg
ttcctgaagatgaacagcctgcagaccgacgacaccgccatctactactg
cgccaagcactactactacggcggcagctacgccatggactactggggcc
agggcacaagcgtgaccgtgtctagc

Those of ordinary skill in the art will be able to select appropriate target antigens for use in chimeric transmembrane receptors described herein, including chimeric transmembrane receptors that bind more than one target antigen.

### Integrin Ligand-Binding Domains

In some embodiments, chimeric transmembrane receptors provided herein include at least one integrin ligand-binding domain. For example, certain chimeric transmembrane receptors provided herein include a single integrin ligand-binding domain or include (at most) a single integrin ligand-binding domain. Alternatively, certain chimeric transmembrane receptors provided herein include more than one integrin ligand-binding domain (e.g., at most two integrin ligand-binding domains). In some embodiments, one or more integrin ligand-binding domains in a chimeric transmembrane receptor provided herein is cleaved (e.g., at an S2 protease cleavage site) upon binding of the extracellular antigen-binding domain of the chimeric transmembrane receptor to its target ligand. In some embodiments, such cleavage of the integrin ligand-binding domain results in cleavage of a gamma-secretase protease cleavage site, resulting in release of the intracellular transcriptional regulatory domain from the remainder of the chimeric transmembrane receptor (e.g., intracellular transcriptional regulatory domain is liberated from the transmembrane domain, permitting it to travel to the nucleus to regulate transcription of a heterologous target gene). In some embodiments, an integrin ligand-binding domain of a chimeric transmembrane receptor includes a S2 proteolytic cleavage site, which S2 proteolytic cleavage site includes an Ala-Val dipeptide sequence. In some embodiments, an integrin ligand-binding domain of a chimeric transmembrane receptor includes a S2 proteolytic cleavage site, which S2 proteolytic cleavage site is capable of being cleaved by a protease from the ADAM family (e.g., ADAM10 or ADAM17).

Integrins are transmembrane proteins that play a role in cell-extracellular matrix (ECM) adhesion. Upon ligand binding, integrins activate signal transduction pathways that mediate a variety of cellular signals, such as, e.g., regulation of the cell cycle, organization of the intracellular cytoskeleton, and movement of new receptors to the cell membrane. Examples of integrin ligands include, without limitation, fibronectin, vitronectin, collagen, and laminin. Those of ordinary skill in the art will be aware of other integrin ligands and their corresponding integrin ligand-binding domains that can be used in accordance with the chimeric transmembrane receptors provided herein.

Any of a variety of integrin ligand-binding domains can be used in accordance with the chimeric transmembrane receptors and methods described herein. In some embodiments, chimeric transmembrane receptors provided herein include at least one (e.g., only one or only two) integrin ligand-binding domain that is present in a protein in receptor-like protein tyrosine phosphatase, including without limitation, a receptor-like protein tyrosine phosphatase in the Type IIa or Type IIb sub-families. For example, chimeric transmembrane receptors provided herein can include at least one (e.g., only one or only two) integrin ligand-binding domain that is present in RPTP(mu), RPTP(delta), RPTP(kappa), LAR, or RPTP(gamma). Exemplary RPTP(mu) polypeptide sequences are shown in NCBI Reference Sequence: NP_001098714.1 and NCBI Reference Sequence: NP 002836.3 (found at URLs www.ncbi.nlm.nih.gov/protein/NP_001098714 and www.ncbi.nlm.nih.gov/protein/NP_002836, respectively). Exemplary RPTP(delta) polypeptide sequences are shown in NCBI Reference Sequence: NP 001035802.1 and NCBI Reference Sequence: NP 001164496.1 (found at URLs www.ncbi.nlm.nih.gov/protein/NP_001035802 and www.ncbi.nlm.nih.gov/protein/NP_001164496, respectively). Exemplary RPTP(kappa) polypeptide sequences are shown in NCBI Reference Sequence: NP 001129120.1 and NCBI Reference Sequence: NP 001278910.1 (found at URLs www.ncbi.nlm.nih.gov/protein/NP_001129120 and www.ncbi.nlm.nih.gov/protein/NP_001278910, respectively). Exemplary LAR polypeptide sequences are shown in NCBI Reference Sequence: NP_001316066.1 and NCBI Reference Sequence: NP_001316067.1 (found at URLs www.ncbi.nlm.nih.gov/protein/NP_001316066 and www.ncbi.nlm.nih.gov/protein/NP_001316067, respectively). An exemplary RPTP(gamma) polypeptide sequences is shown in NCBI Reference Sequence: NP 002832.3 (found at URL www.ncbi.nlm.nih.gov/protein/NP_002832). Those of ordinary skill in the art will be aware of other suitable RPTP(mu), RPTP(delta), RPTP(kappa), LAR, or RPTP(gamma) polypeptide sequences that can be used in accordance with materials and methods disclosed herein, as well as nucleic acid sequences encoding them.

Full-length RPTP(kappa) (also known as PTPRK) is composed of a MAM domain, Ig domain, four fibronectin type-III (FN-III) domains, and two intracellular phosphatase domains. The full-length PTPRK protein gets processed by Furin cleavage at an 51 cleavage site during production and maturation of the PTPRK, giving rise to the mature transmembrane protein that is composed of the E-subunit and the P-subunit, which mature transmembrane protein is expressed on the surface as a bipartite molecule. Exemplary 51 cleavage sites include the amino acid sequences RXRR (SEQ ID NO: 6) or RXKR (SEQ ID NO: 7), where X is any amino acid. After ligand interaction, presumably caused by high cell-density, a protease from the ADAM family (e.g., ADAM10 or ADAM17) is recruited to cleave at the S2 site releasing the E-subunit and the extracellular stalk of the P-subunit. The membrane bound P-subunit is then processed by gamma-secretase and is shuttled to the nucleus where it can regulate gene transcription.

In some embodiments, chimeric transmembrane receptors provided herein include a “core” portion of a receptor-like protein tyrosine phosphatase (e.g., PTPRK), which core portion includes at least one (e.g., only one or only two) integrin ligand-binding domain (e.g., at least one fibronectin domain (e.g., a fibronectin type-III (FN-III) domain)) comprising an S2 cleavage site, a transmembrane domain, and/or an intracellular regulatory domain comprising a gamma-secretase protease cleavage site.

In some embodiments, an integrin ligand-binding domain is a fibronectin domain (e.g., a fibronectin type-III (FN-III) domain). Fibronectin domains are found in a wide variety of extracellular proteins including other extracellular-matrix molecules, cell-surface receptors, enzymes, and muscle proteins. The FN-III domain is an evolutionary conserved protein domain that is found in a variety of proteins. The FN-III domain is approximately 100 amino acids long and possesses a conserved beta sandwich fold with one beta sheet containing four strands and the other sheet containing three strands. In contrast to the two other fibronectin-type domains, the FN-III domain is the only one without disulfide bonding present. Sites of interaction with other molecules, including integrins, have been mapped to short stretch of amino acids such as the Arg-Gly-Asp (RGD) sequence found in various FN-III domains.

In some embodiments, chimeric transmembrane receptors provided herein include at least one FN-III domain (e.g., one or two FN-III domains) as the integrin ligand-binding domain. In some embodiments, one or more FN-III domains in a chimeric transmembrane receptor provided herein are cleaved upon the extracellular antigen-binding domain of the binding of the chimeric transmembrane receptor to its target ligand. In some embodiments, such cleavage results in cleavage of the S2 protease cleavage site and subsequent cleavage of the gamma-secretase cleavage site, resulting in release of the intracellular transcriptional regulatory domain from remainder of the chimeric transmembrane receptor (e.g., release from the transmembrane domain).

In some embodiments, an integrin ligand-binding domain for use in chimeric transmembrane receptors provided herein comprises portions of integrin ligand-binding domains present in two or more endogenous proteins, such that the integrin ligand-binding domain retains the ability to be cleaved at the S2 cleavage site. In some embodiments, chimeric transmembrane receptors provided herein include an integrin ligand-binding domain that differs from an integrin ligand-binding domain present in an endogenous protein by one or more amino acids, e.g., 1, 2, 3, 4, 5, 6, 7, 8, 9, 10, or more amino acids.

In some embodiments, chimeric transmembrane receptors provided herein include an integrin ligand-binding domain that shares a degree of amino acid sequence identity to an integrin ligand-binding domain present in an endogenous protein. For example, an integrin ligand-binding domain for use in an chimeric transmembrane receptor provided herein can share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with an integrin ligand-binding domain present in an endogenous protein. As will be appreciated by those of ordinary skill the art, an integrin ligand-binding domain that differs from an integrin ligand-binding domain present in an endogenous protein by one or more amino acids should still retain the ability to be cleaved at the S2 cleavage site. Methods of identifying and/or testing such modified integrin ligand-binding domains are known in the art.

In some embodiments, an integrin ligand-binding domain (e.g., a fibronectin-type III domain) is encoded by a nucleic acid sequence of SEQ ID NO: 35, shown below:

[SEQ ID NO: 35]
gatgtgcctggtcccgtaccagtaaaatctcttcaaggaacatcctttga
aaataagatcttcttgaactggaaagaacctttggatccaaatggaatca
tcactcaatatgagatcagctatagcagtataagatcatttgatcctgca
gttccagtggctggacctccccagactgtatcaaatttatggaacagtac
acaccatgtctttatgcatctccaccctggaaccacgtaccagtttttca
taagagccagcacggtcaaaggctttggtccagccacagccatcaatgtc
acc.

In some embodiments, an integrin ligand-binding domain (e.g., a fibronectin type III domain) includes a polypeptide sequence of SEQ ID NO: 36, shown below:

[SEQ ID NO: 36]
DVPGPVPVKSLQGTSFENKIFLNWKEPLDPNGIITQYEISYSSIRSFDPA
VPVAGPPQTVSNLWNSTHHVFMHLHPGTTYQFFIRASTVKGFGPATAINV
T.

In some embodiments, an integrin ligand-binding domain (e.g., a fibronectin type III domain) is encoded by a nucleic acid sequence of SEQ ID NO: 37, shown below:

[SEQ ID NO: 37]
cctgactatgaaggagttgatgcctctctcaatgaaactgccaccacaat
aactgtattgttgagaccagcacaagccaaaggtgctcctatcagtgctt
atcagattgttgtggaagaactgcacccacaccgaaccaagagagaagcc
ggagccatggaatgctaccaggttcctgtcacataccaaaatgccatgag
tgggggtgcaccgtattactttgctgcagaactacccccgggaaacctac
ct.

In some embodiments, an integrin ligand-binding domain (e.g., a fibronectin type III domain) includes a polypeptide sequence of SEQ ID NO: 38, shown below:

PDYEGVDASLNETATTITVLLRPAQAKGAPISAYQIVVEELHPHRTKREAGAME CYQVPVTYQNAMSGGAPYYFAAELPPGNLP [SEQ ID NO: 38]. In some embodiments, an integrin ligand-binding domain (e.g., a fibronectin-type III domain) is encoded by a nucleic acid sequence of:

(SEQ ID NO: 55)
AAAAATTTCCACGTGAAGGCTGTTATGAAAACATCCGTTCTCCTGTCATG
GGAAATCCCGGAAAACTATAATTCTGCTATGCCTTTCAAGATATTGTATG
ATGATGGCAAGATGGTTGAAGAGGTCGACGGTCGGGCGACACAAAAACTG
ATCGTTAACCTCAAACCTGAGAAATCATATTCATTCGTCCTCACCAATCG
CGGTAATAGTGCTGGTGGCCTCCAGCACCGGGTAACCGCAAAAACTGCGC
CTGAT.

In some embodiments, an integrin ligand-binding domain (e.g., a fibronectin type III domain) includes an amino acid sequence of:

(SEQ ID NO: 54)
KNFHVKAVMKTSVLLSWEIPENYNSAMPFKILYDDGKMVEEVDGRATQKL
IVNLKPEKSYSFVLTNRGNSAGGLQHRVTAKTAPD.

In some embodiments, an integrin ligand-binding domain (e.g., a fibronectin-type III domain) is encoded by a nucleic acid sequence of:

(SEQ ID NO: 64)
GCCAAAAACTTCAGAGTAGCGGCGGCGATGAAAACTTCTGTCCTTCTGAG
CTGGGAAGTTCCGGATTCCTATAAATCCGCTGTCCCCTTCAAGATATTGT
ACAATGGTCAAAGTGTCGAGGTTGATGGACACTCCATGAGGAAACTCATT
GCCGACCTCCAACCGAACACAGAATACAGTTTTGTTTTGATGAATCGCGG
TTCCTCAGCCGGGGGCCTGCAGCACCTCGTGAGTATTAGGACCGCTCCCG
AT.

In some embodiments, an integrin ligand-binding domain (e.g., a fibronectin-type III domain) is encoded by a nucleic acid sequence of:

(SEQ ID NO: 65)
GCAAAAAACTTCAGAGTAGCGGCGGCGATGAAAACTTCTGTCCTTCTGAG
CTGGGAAGTTCCGGATTCCTATAAATCCGCTGTCCCCTTCAAGATATTGT
ACAATGGTCAAAGTGTCGAGGTTGATGGACACTCCATGAGGAAACTCATT
GCCGACCTCCAACCGAACACAGAATACAGTTTTGTTTTGATGAATCGCGG
TTCCTCAGCCGGGGGCCTGCAGCACCTCGTGAGTATTAGGACCGCTCCCG
AT.

In some embodiments, an integrin ligand-binding domain (e.g., a fibronectin type III domain) includes an amino acid sequence of:

(SEQ ID NO: 63)
AKNFRVAAAMKTSVLLSWEVPDSYKSAVPFKILYNGQSVEVDGHSMRKLI
ADLQPNTEYSFVLMNRGSSAGGLQHLVSIRTAPD.

In some embodiments, an integrin ligand-binding domain (e.g., a fibronectin-type III domain) is encoded by a nucleic acid sequence of:

(SEQ ID NO: 71)
ATATTTCTGCAATGGCGAGAGCCTACACAAACATACGGAGTCATAACGTT
GTACGAAATTACGTACAAGGCCGTGTCATCATTCGATCCCGAAATTGATC
TTTCTAACCAGTCAGGGCGCGTAAGTAAACTCGGCAACGAGACCCACTTT
TTGTTCTTCGGCCTGTATCCGGGCACTACGTACAGTTTCACCATCCGCGC
ATCTACGGCCAAGGGTTTTGGCCCACCCGCTACGAACCAGTTTACTACGA
AGATTTCTGCTCCTTCA.

In some embodiments, an integrin ligand-binding domain (e.g., a fibronectin type III domain) includes an amino acid sequence of:

(SEQ ID NO: 70)
IFLQWREPTQTYGVITLYEITYKAVSSFDPEIDLSNQSGRVSKLGNETHF
LFFGLYPGTTYSFTIRASTAKGFGPPATNQFTTKISAPS.

In some embodiments, an integrin ligand-binding domain (e.g., a fibronectin-type III domain) is encoded by a nucleic acid sequence of:

(SEQ ID NO: 73)
CCAGCTTATGAACTCGAAACTCCACTGAACCAAACTGACAACACAGTTAC
TGTGATGCTGAAGCCCGCGCATAGCCGAGGTGCCCCAGTTTCTGTGTATC
AAATTGTGGTAGAAGAAGAACGGCCACGCCGCACAAAGAAGACGACGGAA
ATACTGAAATGTTATCCAGTCCCTATTCACTTCCAGAACGCTAGTTTGCT
TAACTCACAGTATTATTTCGCGGCAGAATTCCCCGCCGATTCTCTGCAG.

In some embodiments, an integrin ligand-binding domain (e.g., a fibronectin type III domain) includes an amino acid sequence of:

(SEQ ID NO: 72)
PAYELETPLNQTDNTVTVIVILKPAHSRGAPVSVYQIVVEEERPRRTKKT
TEILKCYPVPIEIFQNASLLNSQYYFAAEFPADSLQ.

In some embodiments, an integrin ligand-binding domain (e.g., a fibronectin-type III domain) is encoded by a nucleic acid sequence of:

(SEQ ID NO: 81)
GTCCCATCCGGTATAGCGGCTGAAAGCTTGACATTTACCCCATTGGAAGA
TATGATTTTCCTGAAATGGGAAGAACCACAAGAGCCCAATGGTCTCATTA
CTCAATATGAGATCAGTTACCAGAGCATTGAGTCAAGTGATCCCGCTGTC
AATGTACCGGGACCTCGCAGGACTATCTCTAAGCTGCGGAACGAAACGTA
CCATGTATTCAGCAACCTGCACCCCGGCACCACGTACTTGTTTTCCGTAC
GCGCGAGAACTGGCAAGGGATTCGGGCAGGCTGCCCTTACAGAAATAACT
ACGAACATTTCTGCTCCTTCA.

In some embodiments, an integrin ligand-binding domain (e.g., a fibronectin type III domain) includes an amino acid sequence of:

(SEQ ID NO: 80)
VPSGIAAESLTFTPLEDMIFLKWEEPQEPNGLITQYEISYQSIESSDPAV
NVPGPRRTISKLRNETYHVFSNLHPGTTYLFSVRARTGKGFGQAALTEIT
TNISAPS.

In some embodiments, an integrin ligand-binding domain (e.g., a fibronectin-type III domain) is encoded by a nucleic acid sequence of:

(SEQ ID NO: 83)
TTCGACTACGCAGACATGCCTTCACCGCTCGGTGAATCTGAGAACACCAT
TACGGTCCTGCTTAGGCCTGCACAGGGAAGGGGTGCTCCCATTTCCGTCT
ACCAGGTAATCGTTGAAGAGGAACGCGCCCGGCGGCTCAGACGGGAACCC
GGTGGGCAAGACTGTTTCCCGGTCCCTCTGACCTTTGAGGCGGCCTTGGC
CAGAGGTCTGGTGCATTACTTCGGAGCCGAGTTGGCCGCAAGCTCACTG.

In some embodiments, an integrin ligand-binding domain (e.g., a fibronectin type III domain) includes an amino acid sequence of:

(SEQ ID NO: 82)
FDYADNIPSPLGESENTITVLLRPAQGRGAPISVYQVIVEEERARRLRRE
PGGQDCFPVPLTFEAALARGLVHYFGAELAASSL.

In some embodiments, an integrin ligand-binding domain (e.g., a fibronectin-type III domain) is encoded by a nucleic acid sequence of:

(SEQ ID NO: 89)
AAAATCTATATTCAATGGAAGCCGCCAAATGAAACCAATGGAGTTATAAC
GCTCTATGAAATCAACTACAAGGCGGTTGGCTCCCTTGATCCCTCTGCCG
ACCTTTCCTCACAGCGGGGTAAAGTGTTCAAGCTGAGGAACGAAACGCAC
CACCTTTTCGTGGGGTTGTATCCAGGAACGACCTACAGTTTTACTATTAA
GGCTTCCACAGCCAAAGGCTTTGGGCCCCCTGTAACCACTAGGATTGCTA
CTAAAATCTCCGCGCCATCT.

In some embodiments, an integrin ligand-binding domain (e.g., a fibronectin type III domain) includes an amino acid sequence of:

(SEQ ID NO: 88)
KIYIQWKPPNETNGVITLYEINYKAVGSLDPSADLSSQRGKVFKLRNETH
ELFVGLYPGTTYSFTIKASTAKGFGPPVTTRIATKISAPS.

In some embodiments, an integrin ligand-binding domain (e.g., a fibronectin-type III domain) is encoded by a nucleic acid sequence of:

(SEQ ID NO: 91)
ATGCCCGAATATGATACGGACACCCCATTGAACGAAACAGATACAACAAT
AACTGTCATGCTGAAGCCTGCGCAATCACGCGGAGCCCCTGTCAGCGTAT
ATCAACTTGTAGTCAAAGAAGAAAGACTGCAAAAATCCCGACGCGCTGCC
GACATTATTGAGTGCTTCTCAGTACCCGTGAGCTACAGAAACGCTAGTAG
CTTGGATTCTTTGCATTATTTCGCGGCCGAACTTAAGCCCGCGAATCTTC
CGGTGACTCAACCGTTTACAGTGGGTGACAATAAAACTTACAATGGCTAT
TGGAACCCGCCGTTGTCACCGCTGAAGAGCTACTCAATATATTTCCAGGC
CCTGAGTAAAGCTAACGGTGAGACAAAAATCAACTGTGTGAGACTTGCAA
CTAAAGGA.

In some embodiments, an integrin ligand-binding domain (e.g., a fibronectin type III domain) includes an amino acid sequence of:

(SEQ ID NO: 90)
MPEYDTDTPLNETDTTITVIVILKPAQSRGAPVSVYQLVVKEERLQKSRR
AADIIECFSVPVSYRNASSLDSLHYFAAELKPANLPVTQPFTVGDNKTYN
GYWNPPLSPLKSYSIYFQALSKANGETKINCVRLATKG.

In some embodiments, an integrin ligand-binding domain (e.g., a fibronectin-type III domain) is encoded by a nucleic acid sequence of:

(SEQ ID NO: 99)
AGTCCGAAGAACTTCAAAGTCAAGATGATCATGAAGACATCTGTGCTCTT
GAGTTGGGAGTTTCCCGATAATTATAACAGCCCCACGCCTTACAAGATTC
AGTACAATGGACTCACCCTTGATGTAGATGGCCGCACGACAAAGAAGCTG
ATTACTCACCTCAAACCGCATACTTTCTATAATTTCGTGCTGACGAATCG
GGGTTCTTCCCTGGGAGGTCTCCAGCAAACTGTAACGGCGTGGACTGCGT
TTAAT.

In some embodiments, an integrin ligand-binding domain (e.g., a fibronectin type III domain) includes an amino acid sequence of:

(SEQ ID NO: 98)
SPKNFKVKMIMKTSVLLSWEFPDNYNSPTPYKIQYNGLTLDVDGRTTKKL
ITHLKPHTFYNFVLTNRGSSLGGLQQTVTAWTAFN.

In some embodiments, an integrin ligand-binding domain (e.g., a fibronectin-type III domain) is encoded by a nucleic acid sequence of:

(SEQ ID NO: 105)
GGTCCCGTACCAGTAAAATCTCTTCAAGGAACATCCTTTGAAAATAAGATC
TTCTTGAACTGGAAAGAACCTTTGGATCCAAATGGAATCATCACTCAATAT
GAGATCAGCTATAGCAGTATAAGATCATTTGATCCTGCAGTTCCAGTGGCT
GGACCTCCCCAGACTGTATCAAATTTATGGAACAGTACACACCATGTCTTT
ATGCATCTCCACCCTGGAACCACGTACCAGTTTTTCATAAGAGCCAGCACG
GTCAAAGGCTTTGGTCCAGCCACAGCCATCAATGTCACCACCAATATCTCA
GCTCCAACT.

In some embodiments, an integrin ligand-binding domain (e.g., a fibronectin type III domain) includes an amino acid sequence of:

(SEQ ID NO: 104)
GPVPVKSLQGTSFENKIFLNWKEPLDPNGIITQYEISYSSIRSFDPAVPVA
GPPQTVSNLWNSTHEIVFMEILHPGTTYQFFIRASTVKGFGPATAINVTTN
ISAPT.

In some embodiments, an integrin ligand-binding domain (e.g., a fibronectin-type III domain) is encoded by a nucleic acid sequence of:

(SEQ ID NO: 107)
CCTGACTATGAAGGAGTTGATGCCTCTCTCAATGAAACTGCCACCACAATA
ACTGTATTGTTGAGACCAGCACAAGCCAAAGGTGCTCCTATCAGTGCTTAT
CAGATTGTTGTGGAAGAACTGCACCCACACCGAACCAAGAGAGAAGCCGGA
GCCATGGAATGCTACCAGGTTCCTGTCACATACCAAAATGCCATGAGTGGG
GGTGCACCGTATTACTTTGCTGCAGAACTACCCCCGGGAAACCTACCT.

In some embodiments, an integrin ligand-binding domain (e.g., a fibronectin type III domain) includes an amino acid sequence of:

(SEQ ID NO: 106)
PDYEGVDASLNETATTITVLLRPAQAKGAPISAYQIVVEELHPHRTKREAG
AMECYQVPVTYQNAMSGGAPYYFAAELPPGNLP.

In some embodiments, an integrin ligand-binding domain (e.g., a fibronectin-type III domain) is encoded by a nucleic acid sequence of:

(SEQ ID NO: 127)
CCACCGCAAAAAGTTATGTGTGTGAGCATGGGATCTACCACCGTCCGCGTT
TCTTGGGTTCCACCGCCTGCCGATAGTCGGAATGGCGTCATTACTCAATAC
AGCGTAGCCTACGAGGCCGTGGATGGAGAGGATAGAGGTCGCCATGTCGTA
GATGGAATTAGCCGCGAGCATAGTTCATGGGACTTAGTCGGGTTGGAAAAG
TGGACTGAATACCGTGTTTGGGTCCGAGCTCACACGGATGTTGGCCCAGGA
CCAGAGTCCAGTCCCGTTCTCGTTCGGACGGACGAGGAC.

In some embodiments, an integrin ligand-binding domain (e.g., a fibronectin type III domain) includes an amino acid sequence of:

(SEQ ID NO: 126)
PPQKVIVICVSMGSTTVRVSWVPPPADSRNGVITQYSVAYEAVDGEDRGRH
VVDGISREHSSWDLVGLEKWTEYRVWVRAHTDVGPGPESSPVLVRTDED.

In some embodiments, an integrin ligand-binding domain (e.g., a fibronectin-type III domain) is encoded by a nucleic acid sequence of:

(SEQ ID NO: 129)
CCACCCCGAAAAGTTGAGGTAGAGCCCCTGAATAGTACGGCAGTACATGTC
TATTGGAAGCTCCCTGTTCCATCAAAGCAACATGGGCAGATTAGGGGGTAC
CAAGTTACATATGTGCGATTGGAAAACGGGGAACCACGGGGCCTCCCGATC
ATCCAAGATGTTATGCTCGCAGAGGCACAGTGGCGCCCCGAGGAGTCCGAA
GATTATGAAACTACTATAAGTGGGCTTACCCCAGAGACAACCTACTCTGTA
ACTGTAGCTGCTTACACGACCAAAGGGGATGGCGCCCGAAGCAAGCCCAAA
ATTGTTACCACAACAGGGGCT.

In some embodiments, an integrin ligand-binding domain (e.g., a fibronectin type III domain) includes an amino acid sequence of:

(SEQ ID NO: 128)
PPRKVEVEPLNSTAVHVYWKLPVPSKQHGQIRGYQVTYVRLENGEPRGLPI
IQDVMLAEAQWRPEESEDYETTISGLTPETTYSVTVAAYTTKGDGARSKPK
IVTTTGA.

In some embodiments, an integrin ligand-binding domain (e.g., a fibronectin-type III domain) is encoded by a nucleic acid sequence of:

(SEQ ID NO: 131)
GTACCCGGCAGACCGACAATGATGATCAGCACGACAGCGATGAACACGGCA
CTTCTGCAATGGCATCCGCCCAAGGAATTGCCAGGGGAGTTGCTCGGCTAC
CGGCTGCAATACTGTAGGGCAGATGAAGCAAGGCCGAACACAATCGATTTC
GGAAAGGATGATCAGCACTTTACTGTTACCGGACTTCATAAAGGGACAACT
TATATCTTCCGGCTGGCGGCTAAAAATAGAGCGGGCTTGGGAGAAGAATTC
GAGAAGGAGATTAGAACCCCGGAGGATTTG.

In some embodiments, an integrin ligand-binding domain (e.g., a fibronectin type III domain) includes an amino acid sequence of:

(SEQ ID NO: 130)
VPGRPTIVIMISTTAIVINTALLQWHPPKELPGELLGYRLQYCRADEARPN
TIDFGKDDQHFTVTGLHKGTTYIFRLAAKNRAGLGEEFEKEIRTPEDL.

In some embodiments, an integrin ligand-binding domain (e.g., a fibronectin-type III domain) is encoded by a nucleic acid sequence of:

(SEQ ID NO: 133)
TTCCCGCAAAATCTCCATGTCACGGGACTCACTACTTCAACGACCGAACTC
GCTTGGGATCCTCCAGTCCTTGCCGAAAGAAACGGAAGGATTATAAGCTAC
ACCGTAGTCTTCCGAGATATTAACTCACAACAAGAACTCCAGAATATTACT
ACCGATACGCGATTCACATTGACAGGACTTAAGCCTGACACGACTTATGAT
ATAAAAGTCCGGGCTTGGACTTCCAAAGGAAGCGGACCACTGTCTCCCTCA
ATCCAATCGAGGACTATGCCAGTT.

In some embodiments, an integrin ligand-binding domain (e.g., a fibronectin type III domain) includes an amino acid sequence of:

(SEQ ID NO: 132)
FPQNLHVTGLTTSTTELAWDPPVLAERNGRIISYTVVFRDINSQQELQNIT
TDTRFTLTGLKPDTTYDIKVRAWTSKGSGPLSPSIQSRTMPV.

In some embodiments, an integrin ligand-binding domain (e.g., a fibronectin type III domain) can be encoded by a nucleic acid sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to any one of SEQ ID NOs: 35, 37, 55, 64, 65, 71, 73, 81, 83, 89, 91, 99, 105, 107, 127, 129, 131, and 133.

In some embodiments, an integrin ligand-binding domain (e.g., a fibronectin type III domain) can include a sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to any one of SEQ ID NOs: 36, 38, 54, 63, 70, 72, 80, 82, 88, 90, 98, 104, 106, 126, 128, 130, and 132. In some embodiments, an integrin ligand-binding domain (e.g., fibronectin type III domain) can include a sequence that is identical to any one of SEQ ID NOs: 36, 38, 54, 63, 70, 72, 80, 82, 88, 90, 98, 104, 106, 126, 128, 130, and 132, except that it includes 1, 2, 3, 4, 5, 6, 7, 8, 9, 10, 11, 12, 13, 14, or 15 amino acid substitutions or deletions.

As one skilled in the art, amino acids that are conserved between different related protein domains are more likely to contribute to the function of the protein, and therefore, should not be substituted, while amino acids that are not conserved between different protein domains are less likely to contribute to the function of the protein, and it is likely that substitutions at these amino acid positions will not result in a loss in the activity of the protein.

### Transmembrane Domains

Chimeric transmembrane receptors provided herein include a transmembrane domain. The term “transmembrane domain” refers to a domain of a polypeptide that includes at least one contiguous amino acid sequence that traverses a lipid bilayer when present in the corresponding endogenous polypeptide when expressed in a mammalian cell. For example, a transmembrane domain can include one, two, three, four, five, six, seven, eight, nine, or ten contiguous amino acid sequences that each traverse a lipid bilayer when present in the corresponding endogenous polypeptide when expressed in a mammalian cell. As is known in the art, a transmembrane domain can, e.g., include at least one (e.g., two, three, four, five, six, seven, eight, nine, or ten) contiguous amino acid sequence (that traverses a lipid bilayer when present in the corresponding endogenous polypeptide when expressed in a mammalian cell) that has α-helical secondary structure in the lipid bilayer. In some embodiments, a transmembrane domain can include two or more contiguous amino acid sequences (that each traverse a lipid bilayer when present in the corresponding endogenous polypeptide when expressed in a mammalian cell) that form a β-barrel secondary structure in the lipid bilayer.

Any of a variety of transmembrane domains can be used in accordance with the chimeric transmembrane receptors and methods described herein. In some embodiments, chimeric transmembrane receptors can include a transmembrane domain that is present in an endogenous polypeptide. In some embodiments, chimeric transmembrane receptors provided herein include at least one transmembrane domain that is present in a protein in receptor-like protein tyrosine phosphatase, including without limitation, a receptor-like protein tyrosine phosphatase in the Type IIa or Type IIb sub-families. For example, chimeric transmembrane receptors provided herein can include at least one transmembrane domain that is present in RTPT(mu), RPTP(delta), RPTP(kappa), LAR, or RPTP(gamma). Other non-limiting examples of polypeptides having transmembrane domains that are suitable for use in chimeric transmembrane receptors provided herein include CD28, CD3 epsilon, CD4, CD5, CD6, CD8a, CD9, CD16, CD22, CD33, CD37, CD 45, CD64, CD80, CD86, CD134, 4-1BB, GITR, NGFR, and CD154. Additional examples of transmembrane domains are known in the art.

In some embodiments, a transmembrane domain for use in chimeric transmembrane receptors provided herein comprises portions of transmembrane domains present in two or more endogenous proteins, such that the chimeric transmembrane domain retains the ability to fold correctly and traverse the cell membrane. In some embodiments, chimeric transmembrane receptors provided herein include a transmembrane domain that differs from a transmembrane domain present in an endogenous protein by one or more amino acids, e.g., 1, 2, 3, 4, 5, 6, 7, 8, 9, 10, or more amino acids.

In some embodiments, chimeric transmembrane receptors provided herein include a transmembrane domain that shares a degree of amino acid sequence identity to a transmembrane domain present in an endogenous protein. For example, a transmembrane domain for use in an chimeric transmembrane receptor provided herein can share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with a transmembrane domain present in an endogenous protein. As will be appreciated by those of ordinary skill the art, a transmembrane domain that differs from a transmembrane domain present in an endogenous protein by one or more amino acids should still retain the ability to fold correctly and traverse the cell membrane. Methods of identifying and/or testing such modified transmembrane domains are known in the art.

A non-limiting example of a transmembrane domain is encoded by the nucleic acid sequence of:

(SEQ ID NO: 93)
GCTGGGGTAATTGCAGGACTGCTTATGTTCATCATAATCCTGCTTGGGGTT
ATGCTTACTATC.

A non-limiting example of a transmembrane domain can include the amino acid sequence of: AGVIAGLLMFIIILLGVMLTI (SEQ ID NO: 92). An exemplary transmembrane domain can include a sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to SEQ ID NO: 92. An exemplary transmembrane domain can be encoded by a nucleic acid sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to SEQ ID NO: 93.

### Intracellular Regulatory Domains

In some embodiments, chimeric transmembrane receptors provided herein include an intracellular regulatory domain. As will be appreciated upon reading the present specification, a function of the intracellular regulatory domain is to mediate release of the intracellular transcriptional regulatory domain from the remainder of the chimeric transmembrane receptor (e.g., via cleavage of the intracellular regulatory domain upon the chimeric transmembrane receptor binding a target antigen via its extracellular antigen-binding domain). In some embodiments, chimeric transmembrane receptors provided herein having: an extracellular antigen-binding domain that is capable of specifically binding to a target antigen, an extracellular integrin ligand-binding domain comprising an S2 protease cleavage site, a transmembrane domain, an intracellular regulatory domain comprising a gamma-secretase protease cleavage site, and an intracellular transcriptional regulatory domain, can regulate transcription of a heterologous target gene. For example, when the extracellular antigen-binding domain binds the target antigen, the integrin ligand-binding domain is cleaved at its S2 protease cleavage site and the intracellular regulatory domain is cleaved at its gamma-secretase protease cleavage site, releasing the intracellular transcriptional regulatory domain that can translocate to the nucleus and regulate transcription of the heterologous target gene.

Any of a variety of intracellular regulatory domains can be used in accordance with the chimeric transmembrane receptors and methods described herein. In some embodiments, chimeric transmembrane receptors provided herein include at least one intracellular regulatory domain that is present in a protein in receptor-like protein tyrosine phosphatase, including without limitation, a receptor-like protein tyrosine phosphatase in the Type IIa or Type I % sub-families. For example, chimeric transmembrane receptors provided herein can include at least one intracellular regulatory domain that is present in RPTP(mu), RPTP(delta), RPTP(kappa), LAR, or RPTP(gamma). In some embodiments, chimeric transmembrane receptors provided herein include at least one intracellular regulatory domain that is present in a Notch protein.

In some embodiments, an intracellular regulatory domain for use in chimeric transmembrane receptors provided herein comprises portions of intracellular regulatory domains present in two or more endogenous proteins, such that the chimeric transmembrane receptor retains the ability to be cleaved at the gamma-secretase cleavage site. In some embodiments, chimeric transmembrane receptors provided herein include an intracellular regulatory domain that differs from an intracellular regulatory domain present in an endogenous protein by one or more amino acids, e.g., 1, 2, 3, 4, 5, 6, 7, 8, 9, 10, or more amino acids.

In some embodiments, chimeric transmembrane receptors provided herein include an intracellular regulatory domain that shares a degree of amino acid sequence identity to an intracellular regulatory domain present in an endogenous protein. For example, an intracellular regulatory domain for use in an chimeric transmembrane receptor provided herein can share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with an intracellular regulatory domain present in an endogenous protein (e.g., any of the exemplary PTPR proteins described herein or any of the exemplary portions of PTPR proteins described herein). As will be appreciated by those of ordinary skill the art, an intracellular regulatory domain that differs from an intracellular regulatory domain present in an endogenous protein by one or more amino acids should still retain the ability to be cleaved at the gamma-secretase cleavage site. Methods of identifying and/or testing such modified intracellular regulatory domains are known in the art.

In some embodiments, an intracellular regulatory domain of a chimeric transmembrane receptor provided herein includes a gamma-secretase (γ-secretase) cleavage site. A γ-secretase cleavage site can comprise a Gly-Val dipeptide sequence (e.g., a chimeric transmembrane receptor can include a gamma-secretase (γ-secretase) cleavage site having the sequence VGCGVLLS (SEQ ID NO: 8) or GCGVLLS (SEQ ID NO: 9)). Those of ordinary skill in the art will be aware of other suitable γ-secretase cleavage sites and/or amino acid sequences having such a γ-secretase cleavage site that can be used in accordance with the chimeric transmembrane receptors and methods provided herein.

### Intracellular Transcriptional Regulatory Domains

In some embodiments, chimeric transmembrane receptors provided herein include at least one intracellular transcriptional regulatory domain. In some embodiments, the intracellular transcriptional regulatory domain regulates transcription of a heterologous target gene. In some embodiments, the intracellular transcriptional regulatory domain includes a nuclear localization signal such that upon release from the chimeric transmembrane receptor, the intracellular transcriptional regulatory domain translocates to the nucleus where it regulates transcription of a heterologous target gene. In some embodiments, a heterologous target gene includes a transcription regulatory sequence (e.g., a promoter) that is operably linked to an expression sequence encoding a polypeptide (e.g., a recombinant protein). In some embodiments, an intracellular transcriptional regulatory domain includes a DNA binding domain and a transcriptional activation domain. In some embodiments, a DNA binding domain of an intracellular transcriptional regulatory binds a transcription regulatory sequence (e.g., a promoter) that is operably linked to a sequence encoding a polypeptide (e.g., a recombinant protein). In some embodiments, a heterologous target gene includes an expression sequence encoding a polypeptide to be expressed in a cell that expresses the chimeric transmembrane receptor (e.g., after the extracellular antigen-binding domain of the chimeric transmembrane receptor binds its target antigen, resulting in release of the intracellular transcriptional regulatory domain from the transmembrane domain). Non-limiting examples of such polypeptides include chimeric antigen receptors (CARs), T cell receptors (TCRs), and cytokines. Those of ordinary skill in the art will be aware of other suitable polypeptides that can be expressed a cell that expresses any of the variety of chimeric transmembrane receptors described herein.

In some embodiments, an intracellular transcriptional regulatory domain is a transcriptional activator. In some embodiments, an intracellular transcriptional regulatory domain is an engineered protein that includes a DNA binding domain (e.g., a zinc finger or TALE based DNA binding domain) and a transcriptional effector domain (e.g., VP16 or VP64). In some embodiments, an intracellular transcriptional regulatory domain is a GAL4-VP16 fusion protein. In some embodiments, an intracellular transcriptional regulatory domain is a GAL4-VP64 fusion protein. Those of ordinary skill in the art will be aware of other suitable proteins that include a DNA binding domain and/or a transcriptional effector domain that can be used in accordance with intracellular transcriptional regulatory domains and methods provided herein.

In some embodiments, an intracellular transcriptional regulatory domain represses transcription of heterologous target gene. In some embodiments, an intracellular transcriptional regulatory domain includes a DNA binding domain and a transcriptional repressor domain.

An intracellular transcriptional regulatory domain can include amino acid sequences from any of a variety of polypeptides. Non-limiting examples of such polypeptides include: transcriptional activators, transcriptional repressors, transcriptional co-activators, transcriptional co-repressors, DNA binding polypeptides, RNA binding polypeptides, and translational regulatory polypeptides.

In some cases, an intracellular transcriptional regulatory domain can include one or more amino acid sequences from one or more polypeptides that affect transcription. For example, an intracellular transcriptional regulatory domain can include amino acid sequences from one or more of the following exemplary transcriptional regulators: ABT1, ACYP2, AEBP1, AEBP2, AES, AFF1, AFF3, AHR, ANK1, ANK2, ANKFY1, ANKIB1, ANKRD1, ANKRD10, ANKRD2, ANKRD32, ANKRD46, ANKRD49, ANKRD56, ANKRD57, ANKS4B, AR, ARHGAP17, ARID1A, ARID1B, ARID3A, ARID4A, ARID5B, ARNT, ARNT2, ARNTL, ARNTL2, ARX, ASB10, ASB11, ASB12, ASB15, ASB2, ASB5, ASB8, ASB9, ASH1L, ASH2L, ASXL1, ASZ1, ATF1, ATF3, ATF4, ATF4, ATF5, ATF6, ATF7, ATF7IP, ATM, ATOH1, ATXN3, 1300003B13RIK, B3GAT3, B930041F14RIK, BACH1, BACH2, BARX1, BARX2, BATF, BATF2, BATF3, BAZ2A, BBX, BC003267, BCL11A, BCL11B, BCL3, BCL6, BCL6B, BCLAF1, BCOR, BHLHA15, BHLHE40, BHLHE41, BLZF1, BMYC, BNC1, BNC2, BPNT1, BRCA1, BRWD1, BTBD11, BTF3, 6030408C04RIK, CAMK4, CARHSP1, CARM1, CBX4, CBX7, CCNC, CCNH, CCNT1, CCNT2, CDC5L, CDK2, CDK4, CDK9, CDKN2C, CDX1, CDX1, CDX2, CEBPA, CEBPB, CEBPD, CEBPG, CEBPG, CEBPZ, CHD4, CHD7, CHGB, CIC, CIITA, CITED1, CITED2, CITED4, CLOCK, CLPB, CML3, CNOT7, COPS2, CREB1, CREB3, CREB3L1, CREB3L1, CREB3L2, CREB3L3, CREB5, CREBBP, CREBL2, CREM, CSDA, CSDA, CSDC2, CSDE1, CTBP2, CTCF, CTCFL, CTNNB1, CTNNBL1, CXXC1, D11BWG0517E, 2300002D11RIK, DACH1, DAXX, DBP, DDIT3, DDX20, DDX54, DDX58, DEAF1, DEK, DIDO1, DLX2, DMRT1, DMRT2, DMRTB1, DNMT1, DNMT3A, DR1, DRG1, DUSP26, DYSFIP1, E2F1, E2F2, E2F3, E2F5, E2F6, EBF1, EBF2, EBF3, EBF3, EED, EGR1, EGR2, EGR3, EHF, EHMT2, EID2, ELAVL2, ELF1, ELF1, ELF2, ELF3, ELF4, ELF5, ELK3, ELK4, ELL2, EMX2, EMX2, EN2, ENPP2, EOMES, EP300, EPAS1, ERF, ERG, ESR1, ESRRA, ESRRB, ESRRG, ETS1, ETS2, ETV1, ETV3, ETV4, ETV5, ETV6, EVIL EWSR1, EZH1, EZH2, FAH, FBXL10, FBXL11, FBXW7, FEM1A, FEM1B, FEM1C, FHL2, FLI1, FMNL2, FOS, FOSB, FOSL1, FOSL2, FOXA1, FOXA2, FOXA3, FOXC1, FOXD1, FOXD2, FOXD3, FOXF1, FOXF1A, FOXF2, FOXG1, FOXI1, FOXJ2, FOXJ3, FOXK1, FOXK2, FOXL1, FOXL2, FOXM1, FOXN1, FOXN2, FOXN3, FOXO1, FOXO3, FOXP1, FOXP2, FOXP3, FOXP4, FOXQ1, FUS, FUSIP1, 2810021G02RIK, GABPA, GABPB1, GARNL1, GAS7, GATA1, GATA2, GATA3, GATA4, GATA5, GATA5, GATA6, GBX2, GCDH, GCM1, GFI1, GFI1B, GLI2, GLI3, GLIS1, GLIS2, GLIS3, GLS2, GMEB1, GMEB2, GRHL1, GRHL2, GRHL3, GRLF1, GTF2A1, GTF2B, GTF2E2, GTF2F1, GTF2F2, GTF2H2, GTF2H4, GTF2I, GTF2IRD1, GTF2IRD1, GZFL HAND2, HBP1, HCLS1, HDAC10, HDAC11, HDAC2, HDAC5, HDAC9, HELZ, HES1, HES4, HES5, HES6, HEXIM1, HEY2, HEYL, HHEX, HHEX, HIC1, HIC2, HIF1A, HIF1AN, HIPK2, HIVEP1, HIVEP2, HIVEP2, HIVEP3, HLF, HLTF, HLX, HMBOX1, HMG20A, HMGA2, HMGB2, HMGB3, HNF1B, HNF4A, HNF4G, HOMEZ, HOXA10, HOXA11, HOXA13, HOXA2, HOXA3, HOXA4, HOXA5, HOXA6, HOXA7, HOXA9, HOXB1, HOXB2, HOXB3, HOXB4, HOXB6, HOXB7, HOXB8, HOXB9, HOXC10, HOXC10, HOXC11, HOXC5, HOXC6, HOXC8, HOXC9, HOXD8, HOXD9, HR, HSBP1, HSF2BP, HTATIP2, HTATSF1, HUWE1, 5830417I10RIK, ID1, ID2, ID3, ID3, IFNAR2, IKBKB, IKBKG, IKZF1, IKZF2, IKZF3, IKZF4, IL31RA, ILF3, ING1, ING2, ING3, ING4, INSM1, INTS12, IQWD1, IRF1, IRF1, IRF2, IRF3, IRF4, IRF5, IRF6, IRF7, IRF8, IRF8, IRX1, IRX2, IRX3, IRX4, IRX5, ISL1, ISL2, ISX, ISX, IVNS1ABP, 2810021J22RIK, JARID1A, JARID1B, JARID1C, JARID1D, JDP2, JUN, JUNB, JUND, KLF1, KLF10, KLF11, KLF12, KLF13, KLF15, KLF16, KLF2, KLF3, KLF3, KLF4, KLF5, KLF6, KLF7, KLF8, KLF9, KRR1, 6330416L07RIK, L3MBTL2, LASS2, LASS4, LASS6, LBA1, LBH, LBX1, LCOR, LDB1, LDB2, LEFT, LHX1, LHX2, LHX5, LIMD1, LIN28, LMO1, LMO4, LMX1A, LSM11, LSM4, LYL1, 9030612M13RIK, 1810007M14RIK, 3632451006RIK, MAF, MAFA, MAFB, MAFF, MAFG, MAFK, MAGED1, MAP3K12, MAPK1, MAPK3, MAPK8, MAPK81P1, MAX, MAZ, MBD2, MCM2, MCM4, MCM5, MCM6, MCM1, MECOM, MECP2, MED12, MEDS, MEF2A, MEF2B, MEF2C, MEF2D, MEIS1, MEIS1, MEIS2, MEOX2, MESP2, MID1, MITF, MKI67IP, MKL1, MLL1, MLL3, MLLT10, MLLT3, MLX, MLXIP, MLXIPL, MNT, MNX1, MPL, MSC, MSRB2, MSX2, MTA3, MTF1, MTF2, MTPN, MXD1, MXD4, MXI1, MYB, MYBBP1A, MYBL2, MYC, MYCBP, MYCL1, MYCN, MYEF2, MYF6, MYNN, MYOCD, MYOD1, MYOG, MYST3, MYST4, MYT1L, MZF1, NAB1, NAB2, NANOG, NARG1, NCOA1, NCOA2, NCOA3, NCOR1, NCOR2, NDN, NEUROD1, NEUROD4, NEUROD6, NEUROG1, NEUROG2, NFAT5, NFATC1, NFATC2, NFATC2IP, NFATC3, NFATC3, NFATC4, NFE2, NFE2L1, NFE2L2, NFIA, NFIA, NFIB, NFIC, NFIL3, NFIX, NFKB1, NFKB2, NFKBIB, NFKBIE, NFKBIZ, NFX1, NFXL1, NFYA, NFYB, NHLH1, NKX2-2, NKX2-3, NKX2-5, NKX2-6, NKX6-2, NMI, NOTCH1, NOTCH2, NOTCH3, NOTCH4, NPAS1, NPAS2, NPAS3, NROB1, NROB2, NR1D1, NR1D2, NR1H3, NR1H4, NR1I2, NR1I3, NR2C1, NR2C2, NR2E3, NR2F1, NR2F2, NR2F6, NR3C1, NR3C2, NR4A1, NR4A2, NR4A2, NR4A3, NR5A1, NR5A2, NRARP, NRIP1, NRIP2, NSBP1, NSD1, NUDT12, NULL, NUPR1, 1700065013RIK, OLIG1, OLIG2, OLIG2, ONECUT1, ONECUT2, ONECUT3, ORC2L, OSGIN1, OSR1, OSR2, OSTF1, OVOL1, OVOL2, PAPOLA, PAPOLG, PAPPA2, PATZ1, PAWR, PAX2, PAX5, PAX6, PAX7, PAX8, PAX9, PBX1, PBX2, PBX3, PBX4, PCBD1, PCGF6, PDCD11, PDLIM4, PDX1, PEG3, PERI, PFDN1, PGR, PHF′1, PHF10, PHF12, PHF13, PHF14, PHF20, PHF21A, PHF5A, PHF7, PHOX2A, PHOX2B, PIAS2, PIR, PITX1, PITX2, PKNOX1, PKNOX2, PLA2G6, PLAGL1, PLAGL2, PLRG1, PML, POGK, POLR2B, POLR2E, POLR2H, POLR3E, POLR3H, POLRMT, POU1F1, POU2AF1, POU2F1, POU2F2, POU3F2, POU3F3, POU3F3, POU5F1, POU6F1, PPARA, PPARD, PPARG, PPARGC1A, PPARGC1B, PPP1R12C, PPP1R13B, PPP1R16B, PPP1R1B, PPP2R1A, PPP3CB, PQBP1, PRDM1, PRDM14, PRDM15, PRDM16, PRDM2, PRDM4, PRDM5, PRDM6, PRDM8, PREB, PRKAR1A, PRKCBP1, PROX1, PRRX1, PRRX2, PSMC5, PSMD10, PSMD9, PTF1A, PTGES2, PURB, PWP1, RAB11A, RAB11B, RAB15, RAB18, RAB1B, RAB25, RAB8A, RAB8B, RAI14, RARA, RARB, RARG, RASSF7, RB1, RBBP7, RBL1, RBM14, RBM39, RBM9, RBPJ, RBPJL, RCOR2, REL, RELA, RELB, RERE, REST, REXO4, RFC1, RFX1, RFX2, RFX3, RFX5, RFX7, RFX8, RHOX5, RHOX6, RHOX9, RIPK4, RNF12, RNF14, RNF141, RNF38, RNF4, RORA, RORA, RORB, RORC, RPS6KA4, RREB1, RSRC1, RUNX1, RUNX1T1, RUNX2, RUNX2, RUNX3, RUVBL1, RUVBL2, RXRA, RXRG, RYBP, SAFB2, SALL1, SALL1, SALL2, SALL4, SAP30, SAP30BP, SATB1, SATB2, SATB2, SCAND1, SCAP, SCRT2, SEC14L2, SERTAD1, SF1, SFPI1, SFRS5, SH3D19, SH3PXD2B, SHANK3, SHOX2, SHPRH, SIN3A, SIN3B, SIRT2, SIRT3, SIRT5, SIX1, SIX1, SIX2, SIX3, SIX4, SIX5, SKI, SMAD1, SMAD2, SMAD3, SMAD7, SMARCA1, SMARCA2, SMARCA5, SMARCB1, SMYD1, SNAI1, SNAI2, SNAPC2, SNAPC4, SNIP1, SOLH, SOX1, SOX10, SOX11, SOX12, SOX13, SOX15, SOX17, SOX18, SOX2, SOX21, SOX4, SOX5, SOX6, SOX7, SOX8, SOX9, SP1, SP110, SP140L, SP2, SP3, SP4, SP6, SP8, SPDEF, SPEN, SPI1, SPIB, SQSTM1, SREBF1, SREBF2, SREBF2, SRF, SSBP2, SSBP3, SSBP4, SSRP1, ST18, STAG1, STAT1, STAT1, STAT2, STAT3, STAT4, STAT5A, STAT5B, STAT5B, STATE, SUB1, SUZ12, TADA2L, TAF13, TAF5, TAF5L, TAF7, TAF9, TAL1, TAL1, TARDBP, TBPL1, TBR1, TBX1, TBX10, TBX15, TBX18, TBX2, TBX2, TBX20, TBX21, TBX3, TBX4, TBX5, TBX6, TCEA1, TCEA3, TCEAL1, TCEB3, TCERG1, TCF12, TCF15, TCF19, TCF20, TCF21, TCF21, TCF3, TCF4, TCF7, TCF7L2, TCFAP2A, TCFAP2B, TCFAP2C, TCFCP2L1, TCFE2A, TCFE3, TCFEB, TCFEC, TCFL5, TEAD1, TEAD2, TEAD3, TEAD4, TEF, TFAP2A, TFAP2C, TFCP2L1, TFDP2, TFEB, TFEC, TGFB1I1, TGIF1, TGIF2, TGIF2LX, THRA, THRAP3, THRB, THRSP, TIAL1, TLE1, TLE6, TMEM131, TMPO, TNFAIP3, TOB1, TOX4, TP63, TRERF1, TRIB3, TRIM24, TRIM28, TRIM30, TRIP13, TRIP4, TRIPE, TRP53, TRP53BP1, TRP63, TRPS1, TRPS1, TSC22D1, TSC22D2, TSC22D3, TSC22D4, TSHZ1, TSHZ1, TSHZ3, TTRAP, TUB, TULP4, TWIST1, TWIST2, TYSND1, UBE2W, UBN1, UBP1, UBTF, UGP2, UHRF1, UHRF2, UNCX, USF1, USF2, UTF1, VDR, VEZF1, VGLL2, VF64, VSX1, WASL, WHSC1, WHSC2, WT1, WWP1, WWTR1, XBP1, YAF2, YY1, ZBED1, ZBED4, ZBTB1, ZBTB10, ZBTB16, ZBTB16, ZBTB17, ZBTB2, ZBTB20, ZBTB22, ZBTB25, ZBTB32, ZBTB38, ZBTB4, ZBTB43, ZBTB45, ZBTB47, ZBTB7A, ZBTB7B, ZBTB7C, ZCCHC8, ZDHHC13, ZDHHC16, ZDHHC21, ZDHHC5, ZDHHC6, ZEB2, ANK2ZEB2, ZFHX2, ZFHX3, ZFHX4, ZFP105, ZFP110, ZFP143, ZFP148, ZFP161, ZFP192, ZFP207, ZFP219, ZFP238, ZFP263, ZFP275, ZFP277, ZFP281, ZFP287, ZFP292, ZFP35, ZFP354C, ZFP36, ZFP36L1, ZFP386, ZFP407, ZFP42, ZFP423, ZFP426, ZFP445, ZFP451, ATF5ZFP451, ZFP467, ZFP52, ZFP57, ZFP592, ZFP593, ZFP597, ZFP612, ZFP637, ZFP64, ZFP647, ZFP748, ZFP810, ZFP9, ZFP91, ZFPM1, ZFPM2, ZFX, ZHX2, ZHX3, ZIC1, ZIC2, ZIC3, ZIC4, ZIC5, ZKSCAN1, ZKSCAN3, ZMYND11, ZNF143, ZNF160, ZNF175, ZNF184, ZNF192, ZNF213, ZNF217, ZNF219, ZNF22, ZNF238, ZNF24, ZNF267, ZNF273, ZNF276, ZNF280D, ZNF281, ZNF292, ZNF311, ZNF331, ZNF335, ZNF337, ZNF33B, ZNF366, ZNF394, ZNF398, ZNF41, ZNF410, ZNF415, ZNF423, ZNF436, ZNF444, ZNF445, ZNF451, ZNF460, ZNF496, ZNF498, ZNF516, ZNF521, ZNF532, ZNF536, ZNF546, ZNF552, ZNF563, ZNF576, ZNF580, ZNF596, ZNF621, ZNF628, ZNF648, ZNF649, ZNF652, ZNF655, ZNF664, ZNF668, ZNF687, ZNF692, ZNF696, ZNF697, ZNF710, ZNF80, ZNF91, ZNF92, ZNRD1, ZSCAN10, ZSCAN16, ZSCAN20, ZSCAN21, ZXDC, and ZZZ3. Additionally or alternatively, an intracellular transcriptional regulatory domain can include one or more amino acid sequences from one or more of the following exemplary transcriptional regulators: ASCL1, BRN2, CDX2, CDX4, CTNNB1, EOMES, JUN, FOS, HNF4a, HOXAs (e.g., HOXA1, HOXA2, HOXA3, HOXA4, HOXA5, HOXA10, HOXA11, HOXA13), HOXBs (e.g., HOXB9), HOXCs (e.g., HOXC4, HOXC5, HOXC6, HOXC8, HOXC9, HOXC10, HOXC11, HOXC12, HOXC13), HOXDs (e.g., HOXD1, HOXD3, HOXD4, HOXD8, HOXD9, HOXD10, HOXD11, HOXD12, HOXD13), SNAI1-3, MYOD1, MYOG, NEUROD1-6 (e.g., NEUROD1, NEUROD2, NEUROD4, NEUROD6), PDX1, PU.1, SOX2, Nanog, Klf4, BCL-6, SOX9, STAT1-6, TBET, TCF, TEAD1-4 (e.g., TEAD1, TEAD2, TEAD3, TEAD4), TAF6L, CLOCK, CREB, GATA3, IRF7, MycC, NFkB, RORyt, RUNX1, SRF, TBX21, NFAT, MEF2D, and FoxP3.

In some embodiments, an intracellular transcriptional regulatory domain includes a DNA binding domain and/or a transcriptional effector domain that shares a degree of amino acid sequence identity to a DNA binding domain and/or a transcriptional effector domain present in an endogenous protein. For example, an intracellular transcriptional regulatory domain for use in an chimeric transmembrane receptor provided herein can share at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more sequence identity with a DNA binding domain and/or a transcriptional effector domain present in an endogenous protein. As will be appreciated by those of ordinary skill the art, an intracellular transcriptional regulatory domain having a DNA binding domain and/or a transcriptional effector domain that differs from a DNA binding domain and/or a transcriptional effector domain present in an endogenous protein by one or more amino acids should still retain the ability to fold correctly and bind DNA and/or affect transcription. Methods of identifying and/or testing such modified DNA binding domains and/or transcriptional effector domains are known in the art.

In some embodiments, an intracellular transcriptional regulatory domain includes a transcriptional activation domain present in a polypeptide selected from the group consisting of: VP64, RelA (p65) (Wang, Weixin, et al. “The nuclear factor-KB RelA transcription factor is constitutively activated in human pancreatic adenocarcinoma cells.” Clinical Cancer Research 5.1 (1999): 119-127), YAP (Lian, Ian, et al. “The role of YAP transcription coactivator in regulating stem cell self-renewal and differentiation.” Genes & development 24.11 (2010): 1106-1118, doi:10.1101/gad.1903310), WWTR1(TAZ) (Hong, Jeong-Ho, et al. “TAZ, a transcriptional modulator of mesenchymal stem cell differentiation.” Science 309.5737 (2005): 1074-1078, doi: 10.1126/science.1110955), CREB3(LZIP) (Omori, Yoshihiro, et al. “CREB-H: a novel mammalian transcription factor belonging to the CREB/ATF family and functioning via the box-B element with a liver-specific expression.” Nucleic acids research 29.10 (2001): 2154-2162, doi.org/10.1093/nar/29.10.2154), and MyoD (Weintraub, Harold, and Robert Davis. “The myoD gene family: nodal point during specification of the muscle cell lineage.” Science 251.4995 (1991): 761, doi: 10.1126/science.1846704).

In some embodiments, an intracellular transcriptional regulatory domain includes a transcriptional activation domain present in a RelA (p65) polypeptide (e.g., a Rel-A (p65) polypeptide described in accession numbers NCBI No. NP 068810.3, NP 001138610.1, NP_001230913.1, NP_001230914.1, XP_011543508.1, or XP 011543509.1). In some embodiments, the amino acid sequence of Rel-A (p65) is or comprises all or a portion of SEQ ID NO: 10, SEQ ID NO: 11, SEQ ID NO: 12, SEQ ID NO: 13, SEQ ID NO: 14, or SEQ ID NO: 15. In some embodiments, the amino acid sequence of the intracellular transcriptional regulatory domain of the chimeric transmembrane receptor comprises all or a portion of transcription factor p65 isoform 1 (NP_068810.3), transcription factor p65 isoform 2 (NP_001138610.1), transcription factor p65 isoform 3 (NP_001230913.1), transcription factor p65 isoform 4 (NP_001230914.1), transcription factor p65 isoform X1 (XP 011543508.1), or transcription factor p65 isoform X2 (XP_011543509.1). In some embodiments, the amino acid sequence of the intracellular transcriptional regulatory domain of the chimeric transmembrane receptor comprises all or a portion of SEQ ID NO: 10, SEQ ID NO: 11, SEQ ID NO: 12, SEQ ID NO: 13, SEQ ID NO: 14, or SEQ ID NO: 15. In some embodiments, the amino acid sequence of the intracellular transcriptional regulatory domain of the chimeric transmembrane receptor is or comprises amino acids 1-551 of SEQ ID NO: 10.

Human transcription factor p65 isoform 1 NP_068810.3
(SEQ ID NO: 10)
MDELFPLIFPAEPAQASGPYVEIIEQPKQRGMRFRYKCEGRSAGSIPGERSTDTTKTHPTIKIN
GYTGPGTVRISLVTKDPPHRPHPHELVGKDCRDGFYEAELCPDRCIHSFQNLGIQCVKKRDLEQ
AISQRIQTNNNPFQVPIEEQRGDYDLNAVRLCFQVTVRDPSGRPLRLPPVLSHPIFDNRAPNTA
ELKICRVNRNSGSCLGGDEIFLLCDKVQKEDIEVYFTGPGWEARGSFSQADVHRQVAIVFRTPP
YADPSLQAPVRVSMQLRRPSDRELSEPMEFQYLPDTDDRHRIEEKRKRTYETFKSIMKKSPFSG
PTDPRPPPRRIAVPSRSSASVPKPAPQPYPFTSSLSTINYDEFPTMVFPSGQISQASALAPAPP
QVLPQAPAPAPAPAMVSALAQAPAPVPVLAPGPPQAVAPPAPKPTQAGEGTLSEALLQLQFDDE
DLGALLGNSTDPAVFTDLASVDNSEFQQLLNQGIPVAPHTTEPMLMEYPEAITRLVTGAQRPPD
PAPAPLGAPGLPNGLLSGDEDFSSIADMDFSALLSQISS
Human transcription factor p65 isoform 2 NP_001138610.1
(SEQ ID NO: 11)
MDELFPLIFPAEPAQASGPYVEIIEQPKQRGMRFRYKCEGRSAGSIPGERSTDTTKTHPTIKIN
GYTGPGTVRISLVTKDPPHRPHPHELVGKDCRDGFYEAELCPDRCIHSFQNLGIQCVKKRDLEQ
AISQRIQTNNNPFQEEQRGDYDLNAVRLCFQVTVRDPSGRPLRLPPVLSHPIFDNRAPNTAELK
ICRVNRNSGSCLGGDEIFLLCDKVQKEDIEVYFTGPGWEARGSFSQADVHRQVAIVFRTPPYAD
PSLQAPVRVSMQLRRPSDRELSEPMEFQYLPDTDDRHRIEEKRKRTYETFKSIMKKSPFSGPTD
PRPPPRRIAVPSRSSASVPKPAPQPYPFTSSLSTINYDEFPTMVFPSGQISQASALAPAPPQVL
PQAPAPAPAPAMVSALAQAPAPVPVLAPGPPQAVAPPAPKPTQAGEGTLSEALLQLQFDDEDLG
ALLGNSTDPAVFTDLASVDNSEFQQLLNQGIPVAPHTTEPMLMEYPEAITRLVTGAQRPPDPAP
APLGAPGLPNGLLSGDEDFSSIADMDFSALLSQISS
Human transcription factor p65 isoform 3 NP_001230913.1
(SEQ ID NO: 12)
MDELFPLIFPAEPAQASGPYVEIIEQPKQRGMRFRYKCEGRSAGSIPGERSTDTTKTHPTIKIN
GYTGPGTVRISLVTKDPPHRPHPHELVGKDCRDGFYEAELCPDRCIHSFQNLGIQCVKKRDLEQ
AISQRIQTNNNPFQVPIEEQRGDYDLNAVRLCFQVTVRDPSGRPLRLPPVLSHPIFDNRAPNTA
ELKICRVNRNSGSCLGGDEIFLLCDKVQKEDIEVYFTGPGWEARGSFSQADVHRQVAIVFRTPP
YADPSLQAPVRVSMQLRRPSDRELSEPMEFQYLPDTDDRHRIEEKRKRTYETFKSIMKKSPFSG
PTDPRPPPRRIAVPSRSSASVPKPAPGPPQAVAPPAPKPTQAGEGTLSEALLQLQFDDEDLGAL
LGNSTDPAVFTDLASVDNSEFQQLLNQGIPVAPHTTEPMLMEYPEAITRLVTGAQRPPDPAPAP
LGAPGLPNGLLSGDEDFSSIADMDFSALLSQISS
Human transcription factor p65 isoform 4 NP_001230914.1
(SEQ ID NO: 13)
MDELFPLIFPAEPAQASGPYVEIIEQPKQRGMRFRYKCEGRSAGSIPGERSTDTTKTHPTIKIN
GYTGPGTVRISLVTKDPPHRPHPHELVGKDCRDGFYEAELCPDRCIHSFQNLGIQCVKKRDLEQ
AISQRIQTNNNPFQVPIEEQRGDYDLNAVRLCFQVTVRDPSGRPLRLPPVLSHPIFDNRAPNTA
ELKICRVNRNSGSCLGGDEIFLLCDKVQKEDIEVYFTGPGWEARGSFSQADVHRQVAIVFRTPP
YADPSLQAPVRVSMQLRRPSDRELSEPMEFQYLPDTDDRHRIEEKRKRTYETFKSIMKKSPFSG
PTDPRPPPRRIAVPSRSSASVPKPAPQPYPFTSSLSTINYDEFPTMVFPSGQISQASALAPAPP
QVLPQAPAPAPAPAMVSALAQRPPDPAPAPLGAPGLPNGLLSGDEDFSSIADMDFSALLSQISS
Human transcription factor p65 isoform X1 XP_011543508.1
(SEQ ID NO: 14)
MDELFPLIFPAEPAQASGPYVEIIEQPKQRGMRFRYKCEGRSAGSIPGERSTDTTKTHPTIKIN
GYTGPGTVRISLVTKDPPHRPHPHELVGKDCRDGFYEAELCPDRCIHSFQNLGIQCVKKRDLEQ
AISQRIQTNNNPFQVPIEEQRGDYDLNAVRLCFQVTVRDPSGRPLRLPPVLSHPIFDNRAPNTA
ELKICRVNRNSGSCLGGDEIFLLCDKVQKDDRHRIEEKRKRTYETFKSIMKKSPFSGPTDPRPP
PRRIAVPSRSSASVPKPAPQPYPFTSSLSTINYDEFPTMVFPSGQISQASALAPAPPQVLPQAP
APAPAPAMVSALAQAPAPVPVLAPGPPQAVAPPAPKPTQAGEGTLSEALLQLQFDDEDLGALLG
NSTDPAVFTDLASVDNSEFQQLLNQGIPVAPHTTEPMLMEYPEAITRLVTGAQRPPDPAPAPLG
APGLPNGLLSGDEDFSSIADMDFSALLSQISS
Human transcription factor p65 isoform X2 XP_011543509.1
(SEQ ID NO: 15)
MDELFPLIFPAEPAQASGPYVEIIEQPKQRGMRFRYKCEGRSAGSIPGERSTDTTKTHPTIKIN
GYTGPGTVRISLVTKDPPHRPHPHELVGKDCRDGFYEAELCPDRCIHSFQNLGIQCVKKRDLEQ
AISQRIQTNNNPFQVPIEEQRGDYDLNAVRLCFQVTVRDPSGRPLRLPPVLSHPIFDNHDRHRI
EEKRKRTYETFKSIMKKSPFSGPTDPRPPPRRIAVPSRSSASVPKPAPQPYPFTSSLSTINYDE
FPTMVFPSGQISQASALAPAPPQVLPQAPAPAPAPAMVSALAQAPAPVPVLPAGPPQAVAPPAP
KPTQAGEGTLSEALLQLQFDDEDLGALLGNSTDPAVFTDLASVDNSEFQQLLNQGIPVAPHTTE
PMLMEYPEAITRLVTGAQRPPDPAPAPLGAPGLPNGLLSGDEDFSSIADMDFSALLSQISS

In some embodiments, the amino acid sequence of Rel-A (p65), as described herein, is at least 80% identical to the amino acid sequence of SEQ ID NO: 10, SEQ ID NO:11, SEQ ID NO: 12, SEQ ID NO: 13, SEQ ID NO: 14, or SEQ ID NO: 15. In some embodiments, the amino acid sequence of Rel-A (p65) is 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 10, SEQ ID NO:11, SEQ ID NO: 12, SEQ ID NO: 13, SEQ ID NO: 14, or SEQ ID NO: 15. In some embodiments, the amino acid sequence of Rel-A (p65), as described herein, can vary from the amino acid sequence of SEQ ID NO: 10, SEQ ID NO:11, SEQ ID NO: 12, SEQ ID NO: 13, SEQ ID NO: 14, or SEQ ID NO: 15 by 1 amino acid, 2 amino acids, 3 amino acids, 4 amino acids, 5 amino acids, 6 amino acids, 7 amino acids, 8 amino acids, 9 amino acids, or 10 or more amino acids.

In some embodiments, the nucleic acid sequence encoding Rel-A (p65) is provided by NCBI No. NM_021975.3, NM_001145138.1, NM_001243984.1, NM_001243985.1, XM_011545206.1, or XM_0115452071 In some embodiments, the nucleic acid sequence encoding Rel-A (p65) is or comprises SEQ ID NO: 16, SEQ ID NO: 17, SEQ ID NO: 18, SEQ ID NO: 19, SEQ ID NO: 20, or SEQ ID NO: 21.

Human RELA proto-oncogene, NF-kB subunit (RELA), transcript
variant 1, mRNA NM_021975.3
(SEQ ID NO: 16)
AGCGCGCAGGCGCGGCCGGATTCCGGGCAGTGACGCGACGGCGGGCCGCGCGGCGCATTTCCGC
CTCTGGCGAATGGCTCGTCTGTAGTGCACGCCGCGGGCCCAGCTGCGACCCCGGCCCCGCCCCC
GGGACCCCGGCCATGGACGAACTGTTCCCCCTCATCTTCCCGGCAGAGCCAGCCCAGGCCTCTG
GCCCCTATGTGGAGATCATTGAGCAGCCCAAGCAGCGGGGCATGCGCTTCCGCTACAAGTGCGA
GGGGCGCTCCGCGGGCAGCATCCCAGGCGAGAGGAGCACAGATACCACCAAGACCCACCCCACC
ATCAAGATCAATGGCTACACAGGACCAGGGACAGTGCGCATCTCCCTGGTCACCAAGGACCCTC
CTCACCGGCCTCACCCCCACGAGCTTGTAGGAAAGGACTGCCGGGATGGCTTCTATGAGGCTGA
GCTCTGCCCGGACCGCTGCATCCACAGTTTCCAGAACCTGGGAATCCAGTGTGTGAAGAAGCGG
GACCTGGAGCAGGCTATCAGTCAGCGCATCCAGACCAACAACAACCCCTTCCAAGTTCCTATAG
AAGAGCAGCGTGGGGACTACGACCTGAATGCTGTGCGGCTCTGCTTCCAGGTGACAGTGCGGGA
CCCATCAGGCAGGCCCCTCCGCCTGCCGCCTGTCCTTTCTCATCCCATCTTTGACAATCGTGCC
CCCAACACTGCCGAGCTCAAGATCTGCCGAGTGAACCGAAACTCTGGCAGCTGCCTCGGTGGGG
ATGAGATCTTCCTACTGTGTGACAAGGTGCAGAAAGAGGACATTGAGGTGTATTTCACGGGACC
AGGCTGGGAGGCCCGAGGCTCCTTTTCGCAAGCTGATGTGCACCGACAAGTGGCCATTGTGTTC
CGGACCCCTCCCTACGCAGACCCCAGCCTGCAGGCTCCTGTGCGTGTCTCCATGCAGCTGCGGC
GGCCTTCCGACCGGGAGCTCAGTGAGCCCATGGAATTCCAGTACCTGCCAGATACAGACGATCG
TCACCGGATTGAGGAGAAACGTAAAAGGACATATGAGACCTTCAAGAGCATCATGAAGAAGAGT
CCTTTCAGCGGACCCACCGACCCCCGGCCTCCACCTCGACGCATTGCTGTGCCTTCCCGCAGCT
CAGCTTCTGTCCCCAAGCCAGCACCCCAGCCCTATCCCTTTACGTCATCCCTGAGCACCATCAA
CTATGATGAGTTTCCCACCATGGTGTTTCCTTCTGGGCAGATCAGCCAGGCCTCGGCCTTGGCC
CCGGCCCCTCCCCAAGTCCTGCCCCAGGCTCCAGCCCCTGCCCCTGCTCCAGCCATGGTATCAG
CTCTGGCCCAGGCCCCAGCCCCTGTCCCAGTCCTAGCCCCAGGCCCTCCTCAGGCTGTGGCCCC
ACCTGCCCCCAAGCCCACCCAGGCTGGGGAAGGAACGCTGTCAGAGGCCCTGCTGCAGCTGCAG
TTTGATGATGAAGACCTGGGGGCCTTGCTTGGCAACAGCACAGACCCAGCTGTGTTCACAGACC
TGGCATCCGTCGACAACTCCGAGTTTCAGCAGCTGCTGAACCAGGGCATACCTGTGGCCCCCCA
CACAACTGAGCCCATGCTGATGGAGTACCCTGAGGCTATAACTCGCCTAGTGACAGGGGCCCAG
AGGCCCCCCGACCCAGCTCCTGCTCCACTGGGGGCCCCGGGGCTCCCCAATGGCCTCCTTTCAG
GAGATGAAGACTTCTCCTCCATTGCGGACATGGACTTCTCAGCCCTGCTGAGTCAGATCAGCTC
CTAAGGGGGTGACGCCTGCCCTCCCCAGAGCACTGGGTTGCAGGGGATTGAAGCCCTCCAAAAG
CACTTACGGATTCTGGTGGGGTGTGTTCCAACTGCCCCCAACTTTGTGGATGTCTTCCTTGGAG
GGGGGAGCCATATTTTATTCTTTTATTGTCAGTATCTGTATCTCTCTCTCTTTTTGGAGGTGCT
TAAGCAGAAGCATTAACTTCTCTGGAAAGGGGGGAGCTGGGGAAACTCAAACTTTTCCCCTGTC
CTGATGGTCAGCTCCCTTCTCTGTAGGGAACTCTGGGGTCCCCCATCCCCATCCTCCAGCTTCT
GGTACTCTCCTAGAGACAGAAGCAGGCTGGAGGTAAGGCCTTTGAGCCCACAAAGCCTTATCAA
GTGTCTTCCATCATGGATTCATTACAGCTTAATCAAAATAACGCCCCAGATACCAGCCCCTGTA
TGGCACTGGCATTGTCCCTGTGCCTAACACCAGCGTTTGAGGGGCTGGCCTTCCTGCCCTACAG
AGGTCTCTGCCGGCTCTTTCCTTGCTCAACCATGGCTGAAGGAAACCAGTGCAACAGCACTGGC
TCTCTCCAGGATCCAGAAGGGGTTTGGTCTGGGACTTCCTTGCTCTCCCTCTTCTCAAGTGCCT
TAATAGTAGGGTAAGTTGTTAAGAGTGGGGGAGAGCAGGCTGGCAGCTCTCCAGTCAGGAGGCA
TAGTTTTTACTGAACAATCAAAGCACTTGGACTCTTGCTCTTTCTACTCTGAACTAATAAATCT
GTTGCCAGCTGGCTAGAAA
Human RELA proto-oncogene, NF-kB subunit (RELA), transcript
variant 2, mRNA NM_001145138.1
(SEQ ID NO: 17)
AGCGCGCAGGCGCGGCCGGATTCCGGGCAGTGACGCGACGGCGGGCCGCGCGGCGCATTTCCGC
CTCTGGCGAATGGCTCGTCTGTAGTGCACGCCGCGGGCCCAGCTGCGACCCCGGCCCCGCCCCC
GGGACCCCGGCCATGGACGAACTGTTCCCCCTCATCTTCCCGGCAGAGCCAGCCCAGGCCTCTG
GCCCCTATGTGGAGATCATTGAGCAGCCCAAGCAGCGGGGCATGCGCTTCCGCTACAAGTGCGA
GGGGCGCTCCGCGGGCAGCATCCCAGGCGAGAGGAGCACAGATACCACCAAGACCCACCCCACC
ATCAAGATCAATGGCTACACAGGACCAGGGACAGTGCGCATCTCCCTGGTCACCAAGGACCCTC
CTCACCGGCCTCACCCCCACGAGCTTGTAGGAAAGGACTGCCGGGATGGCTTCTATGAGGCTGA
GCTCTGCCCGGACCGCTGCATCCACAGTTTCCAGAACCTGGGAATCCAGTGTGTGAAGAAGCGG
GACCTGGAGCAGGCTATCAGTCAGCGCATCCAGACCAACAACAACCCCTTCCAAGAAGAGCAGC
GTGGGGACTACGACCTGAATGCTGTGCGGCTCTGCTTCCAGGTGACAGTGCGGGACCCATCAGG
CAGGCCCCTCCGCCTGCCGCCTGTCCTTTCTCATCCCATCTTTGACAATCGTGCCCCCAACACT
GCCGAGCTCAAGATCTGCCGAGTGAACCGAAACTCTGGCAGCTGCCTCGGTGGGGATGAGATCT
TCCTACTGTGTGACAAGGTGCAGAAAGAGGACATTGAGGTGTATTTCACGGGACCAGGCTGGGA
GGCCCGAGGCTCCTTTTCGCAAGCTGATGTGCACCGACAAGTGGCCATTGTGTTCCGGACCCCT
CCCTACGCAGACCCCAGCCTGCAGGCTCCTGTGCGTGTCTCCATGCAGCTGCGGCGGCCTTCCG
ACCGGGAGCTCAGTGAGCCCATGGAATTCCAGTACCTGCCAGATACAGACGATCGTCACCGGAT
TGAGGAGAAACGTAAAAGGACATATGAGACCTTCAAGAGCATCATGAAGAAGAGTCCTTTCAGC
GGACCCACCGACCCCCGGCCTCCACCTCGACGCATTGCTGTGCCTTCCCGCAGCTCAGCTTCTG
TCCCCAAGCCAGCACCCCAGCCCTATCCCTTTACGTCATCCCTGAGCACCATCAACTATGATGA
GTTTCCCACCATGGTGTTTCCTTCTGGGCAGATCAGCCAGGCCTCGGCCTTGGCCCCGGCCCCT
CCCCAAGTCCTGCCCCAGGCTCCAGCCCCTGCCCCTGCTCCAGCCATGGTATCAGCTCTGGCCC
AGGCCCCAGCCCCTGTCCCAGTCCTAGCCCCAGGCCCTCCTCAGGCTGTGGCCCCACCTGCCCC
CAAGCCCACCCAGGCTGGGGAAGGAACGCTGTCAGAGGCCCTGCTGCAGCTGCAGTTTGATGAT
GAAGACCTGGGGGCCTTGCTTGGCAACAGCACAGACCCAGCTGTGTTCACAGACCTGGCATCCG
TCGACAACTCCGAGTTTCAGCAGCTGCTGAACCAGGGCATACCTGTGGCCCCCCACACAACTGA
GCCCATGCTGATGGAGTACCCTGAGGCTATAACTCGCCTAGTGACAGGGGCCCAGAGGCCCCCC
GACCCAGCTCCTGCTCCACTGGGGGCCCCGGGGCTCCCCAATGGCCTCCTTTCAGGAGATGAAG
ACTTCTCCTCCATTGCGGACATGGACTTCTCAGCCCTGCTGAGTCAGATCAGCTCCTAAGGGGG
TGACGCCTGCCCTCCCCAGAGCACTGGGTTGCAGGGGATTGAAGCCCTCCAAAAGCACTTACGG
ATTCTGGTGGGGTGTGTTCCAACTGCCCCCAACTTTGTGGATGTCTTCCTTGGAGGGGGGAGCC
ATATTTTATTCTTTTATTGTCAGTATCTGTATCTCTCTCTCTTTTTGGAGGTGCTTAAGCAGAA
GCATTAACTTCTCTGGAAAGGGGGGAGCTGGGGAAACTCAAACTTTTCCCCTGTCCTGATGGTC
AGCTCCCTTCTCTGTAGGGAACTCTGGGGTCCCCCATCCCCATCCTCCAGCTTCTGGTACTCTC
CTAGAGACAGAAGCAGGCTGGAGGTAAGGCCTTTGAGCCCACAAAGCCTTATCAAGTGTCTTCC
ATCATGGATTCATTACAGCTTAATCAAAATAACGCCCCAGATACCAGCCCCTGTATGGCACTGG
CATTGTCCCTGTGCCTAACACCAGCGTTTGAGGGGCTGGCCTTCCTGCCCTACAGAGGTCTCTG
CCGGCTCTTTCCTTGCTCAACCATGGCTGAAGGAAACCAGTGCAACAGCACTGGCTCTCTCCAG
GATCCAGAAGGGGTTTGGTCTGGGACTTCCTTGCTCTCCCTCTTCTCAAGTGCCTTAATAGTAG
GGTAAGTTGTTAAGAGTGGGGGAGAGCAGGCTGGCAGCTCTCCAGTCAGGAGGCATAGTTTTTA
CTGAACAATCAAAGCACTTGGACTCTTGCTCTTTCTACTCTGAACTAATAAATCTGTTGCCAAG
CTGGCTAGAAA
Human RELA proto-oncogene, NF-kB subunit (RELA), transcript
variant 3, mRNA NM_001243984.1
(SEQ ID NO: 18)
AGCGCGCAGGCGCGGCCGGATTCCGGGCAGTGACGCGACGGCGGGCCGCGCGGCGCATTTCCGC
CTCTGGCGAATGGCTCGTCTGTAGTGCACGCCGCGGGCCCAGCTGCGACCCCGGCCCCGCCCCC
GGGACCCCGGCCATGGACGAACTGTTCCCCCTCATCTTCCCGGCAGAGCCAGCCCAGGCCTCTG
GCCCCTATGTGGAGATCATTGAGCAGCCCAAGCAGCGGGGCATGCGCTTCCGCTACAAGTGCGA
GGGGCGCTCCGCGGGCAGCATCCCAGGCGAGAGGAGCACAGATACCACCAAGACCCACCCCACC
ATCAAGATCAATGGCTACACAGGACCAGGGACAGTGCGCATCTCCCTGGTCACCAAGGACCCTC
CTCACCGGCCTCACCCCCACGAGCTTGTAGGAAAGGACTGCCGGGATGGCTTCTATGAGGCTGA
GCTCTGCCCGGACCGCTGCATCCACAGTTTCCAGAACCTGGGAATCCAGTGTGTGAAGAAGCGG
GACCTGGAGCAGGCTATCAGTCAGCGCATCCAGACCAACAACAACCCCTTCCAAGTTCCTATAG
AAGAGCAGCGTGGGGACTACGACCTGAATGCTGTGCGGCTCTGCTTCCAGGTGACAGTGCGGGA
CCCATCAGGCAGGCCCCTCCGCCTGCCGCCTGTCCTTTCTCATCCCATCTTTGACAATCGTGCC
CCCAACACTGCCGAGCTCAAGATCTGCCGAGTGAACCGAAACTCTGGCAGCTGCCTCGGTGGGG
ATGAGATCTTCCTACTGTGTGACAAGGTGCAGAAAGAGGACATTGAGGTGTATTTCACGGGACC
AGGCTGGGAGGCCCGAGGCTCCTTTTCGCAAGCTGATGTGCACCGACAAGTGGCCATTGTGTTC
CGGACCCCTCCCTACGCAGACCCCAGCCTGCAGGCTCCTGTGCGTGTCTCCATGCAGCTGCGGC
GGCCTTCCGACCGGGAGCTCAGTGAGCCCATGGAATTCCAGTACCTGCCAGATACAGACGATCG
TCACCGGATTGAGGAGAAACGTAAAAGGACATATGAGACCTTCAAGAGCATCATGAAGAAGAGT
CCTTTCAGCGGACCCACCGACCCCCGGCCTCCACCTCGACGCATTGCTGTGCCTTCCCGCAGCT
CAGCTTCTGTCCCCAAGCCAGCCCCAGGCCCTCCTCAGGCTGTGGCCCCACCTGCCCCCAAGCC
CACCCAGGCTGGGGAAGGAACGCTGTCAGAGGCCCTGCTGCAGCTGCAGTTTGATGATGAAGAC
CTGGGGGCCTTGCTTGGCAACAGCACAGACCCAGCTGTGTTCACAGACCTGGCATCCGTCGACA
ACTCCGAGTTTCAGCAGCTGCTGAACCAGGGCATACCTGTGGCCCCCCACACAACTGAGCCCAT
GCTGATGGAGTACCCTGAGGCTATAACTCGCCTAGTGACAGGGGCCCAGAGGCCCCCCGACCCA
GCTCCTGCTCCACTGGGGGCCCCGGGGCTCCCCAATGGCCTCCTTTCAGGAGATGAAGACTTCT
CCTCCATTGCGGACATGGACTTCTCAGCCCTGCTGAGTCAGATCAGCTCCTAAGGGGGTGACGC
CTGCCCTCCCCAGAGCACTGGGTTGCAGGGGATTGAAGCCCTCCAAAAGCACTTACGGATTCTG
GTGGGGTGTGTTCCAACTGCCCCCAACTTTGTGGATGTCTTCCTTGGAGGGGGGAGCCATATTT
TATTCTTTTATTGTCAGTATCTGTATCTCTCTCTCTTTTTGGAGGTGCTTAAGCAGAAGCATTA
ACTTCTCTGGAAAGGGGGGAGCTGGGGAAACTCAAACTTTTCCCCTGTCCTGATGGTCAGCTCC
CTTCTCTGTAGGGAACTCTGGGGTCCCCCATCCCCATCCTCCAGCTTCTGGTACTCTCCTAGAG
ACAGAAGCAGGCTGGAGGTAAGGCCTTTGAGCCCACAAAGCCTTATCAAGTGTCTTCCATCATG
GATTCATTACAGCTTAATCAAAATAACGCCCCAGATACCAGCCCCTGTATGGCACTGGCATTGT
CCCTGTGCCTAACACCAGCGTTTGAGGGGCTGGCCTTCCTGCCCTACAGAGGTCTCTGCCGGCT
CTTTCCTTGCTCAACCATGGCTGAAGGAAACCAGTGCAACAGCACTGGCTCTCTCCAGGATCCA
GAAGGGGTTTGGTCTGGGACTTCCTTGCTCTCCCTCTTCTCAAGTGCCTTAATAGTAGGGTAAG
TTGTTAAGAGTGGGGGAGAGCAGGCTGGCAGCTCTCCAGTCAGGAGGCATAGTTTTTACTGAAC
AATCAAAGCACTTGGACTCTTGCTCTTTCTACTCTGAACTAATAAATCTGTTGCCAAGCTGGCT
AGAAA
Human RELA proto-oncogene, NF-kB subunit (RELA), transcript
variant 4, mRNA NM_001243985.1
(SEQ ID NO: 19)
AGCGCGCAGGCGCGGCCGGATTCCGGGCAGTGACGCGACGGCGGGCCGCGCGGCGCATTTCCGC
CTCTGGCGAATGGCTCGTCTGTAGTGCACGCCGCGGGCCCAGCTGCGACCCCGGCCCCGCCCCC
GGGACCCCGGCCATGGACGAACTGTTCCCCCTCATCTTCCCGGCAGAGCCAGCCCAGGCCTCTG
GCCCCTATGTGGAGATCATTGAGCAGCCCAAGCAGCGGGGCATGCGCTTCCGCTACAAGTGCGA
GGGGCGCTCCGCGGGCAGCATCCCAGGCGAGAGGAGCACAGATACCACCAAGACCCACCCCACC
ATCAAGATCAATGGCTACACAGGACCAGGGACAGTGCGCATCTCCCTGGTCACCAAGGACCCTC
CTCACCGGCCTCACCCCCACGAGCTTGTAGGAAAGGACTGCCGGGATGGCTTCTATGAGGCTGA
GCTCTGCCCGGACCGCTGCATCCACAGTTTCCAGAACCTGGGAATCCAGTGTGTGAAGAAGCGG
GACCTGGAGCAGGCTATCAGTCAGCGCATCCAGACCAACAACAACCCCTTCCAAGTTCCTATAG
AAGAGCAGCGTGGGGACTACGACCTGAATGCTGTGCGGCTCTGCTTCCAGGTGACAGTGCGGGA
CCCATCAGGCAGGCCCCTCCGCCTGCCGCCTGTCCTTTCTCATCCCATCTTTGACAATCGTGCC
CCCAACACTGCCGAGCTCAAGATCTGCCGAGTGAACCGAAACTCTGGCAGCTGCCTCGGTGGGG
ATGAGATCTTCCTACTGTGTGACAAGGTGCAGAAAGAGGACATTGAGGTGTATTTCACGGGACC
AGGCTGGGAGGCCCGAGGCTCCTTTTCGCAAGCTGATGTGCACCGACAAGTGGCCATTGTGTTC
CGGACCCCTCCCTACGCAGACCCCAGCCTGCAGGCTCCTGTGCGTGTCTCCATGCAGCTGCGGC
GGCCTTCCGACCGGGAGCTCAGTGAGCCCATGGAATTCCAGTACCTGCCAGATACAGACGATCG
TCACCGGATTGAGGAGAAACGTAAAAGGACATATGAGACCTTCAAGAGCATCATGAAGAAGAGT
CCTTTCAGCGGACCCACCGACCCCCGGCCTCCACCTCGACGCATTGCTGTGCCTTCCCGCAGCT
CAGCTTCTGTCCCCAAGCCAGCACCCCAGCCCTATCCCTTTACGTCATCCCTGAGCACCATCAA
CTATGATGAGTTTCCCACCATGGTGTTTCCTTCTGGGCAGATCAGCCAGGCCTCGGCCTTGGCC
CCGGCCCCTCCCCAAGTCCTGCCCCAGGCTCCAGCCCCTGCCCCTGCTCCAGCCATGGTATCAG
CTCTGGCCCAGAGGCCCCCCGACCCAGCTCCTGCTCCACTGGGGGCCCCGGGGCTCCCCAATGG
CCTCCTTTCAGGAGATGAAGACTTCTCCTCCATTGCGGACATGGACTTCTCAGCCCTGCTGAGT
CAGATCAGCTCCTAAGGGGGTGACGCCTGCCCTCCCCAGAGCACTGGGTTGCAGGGGATTGAAG
CCCTCCAAAAGCACTTACGGATTCTGGTGGGGTGTGTTCCAACTGCCCCCAACTTTGTGGATGT
CTTCCTTGGAGGGGGGAGCCATATTTTATTCTTTTATTGTCAGTATCTGTATCTCTCTCTCTTT
TTGGAGGTGCTTAAGCAGAAGCATTAACTTCTCTGGAAAGGGGGGAGCTGGGGAAACTCAAACT
TTTCCCCTGTCCTGATGGTCAGCTCCCTTCTCTGTAGGGAACTCTGGGGTCCCCCATCCCCATC
CTCCAGCTTCTGGTACTCTCCTAGAGACAGAAGCAGGCTGGAGGTAAGGCCTTTGAGCCCACAA
AGCCTTATCAAGTGTCTTCCATCATGGATTCATTACAGCTTAATCAAAATAACGCCCCAGATAC
CAGCCCCTGTATGGCACTGGCATTGTCCCTGTGCCTAACACCAGCGTTTGAGGGGCTGGCCTTC
CTGCCCTACAGAGGTCTCTGCCGGCTCTTTCCTTGCTCAACCATGGCTGAAGGAAACCAGTGCA
ACAGCACTGGCTCTCTCCAGGATCCAGAAGGGGTTTGGTCTGGGACTTCCTTGCTCTCCCTCTT
CTCAAGTGCCTTAATAGTAGGGTAAGTTGTTAAGAGTGGGGGAGAGCAGGCTGGCAGCTCTCCA
GTCAGGAGGCATAGTTTTTACTGAACAATCAAAGCACTTGGACTCTTGCTCTTTCTACTCTGAA
CTAATAAATCTGTTGCCAAGCTGGCTAG
Human RELA proto-oncogene, NF-kB subunit (RELA), transcript
variant X1, mRNA XM_011545206.1
(SEQ ID NO: 20)
ATTCCGGGCAGTGACGCGACGGCGGGCCGCGCGGCGCATTTCCGCCTCTGGCGAATGGCTCGTC
TGTAGTGCACGCCGCGGGCCCAGCTGCGACCCCGGCCCCGCCCCCGGGACCCCGGCCATGGACG
AACTGTTCCCCCTCATCTTCCCGGCAGAGCCAGCCCAGGCCTCTGGCCCCTATGTGGAGATCAT
TGAGCAGCCCAAGCAGCGGGGCATGCGCTTCCGCTACAAGTGCGAGGGGCGCTCCGCGGGCAGC
ATCCCAGGCGAGAGGAGCACAGATACCACCAAGACCCACCCCACCATCAAGATCAATGGCTACA
CAGGACCAGGGACAGTGCGCATCTCCCTGGTCACCAAGGACCCTCCTCACCGGCCTCACCCCCA
CGAGCTTGTAGGAAAGGACTGCCGGGATGGCTTCTATGAGGCTGAGCTCTGCCCGGACCGCTGC
ATCCACAGTTTCCAGAACCTGGGAATCCAGTGTGTGAAGAAGCGGGACCTGGAGCAGGCTATCA
GTCAGCGCATCCAGACCAACAACAACCCCTTCCAAGTTCCTATAGAAGAGCAGCGTGGGGACTA
CGACCTGAATGCTGTGCGGCTCTGCTTCCAGGTGACAGTGCGGGACCCATCAGGCAGGCCCCTC
CGCCTGCCGCCTGTCCTTTCTCATCCCATCTTTGACAATCGTGCCCCCAACACTGCCGAGCTCA
AGATCTGCCGAGTGAACCGAAACTCTGGCAGCTGCCTCGGTGGGGATGAGATCTTCCTACTGTG
TGACAAGGTGCAGAAAGACGATCGTCACCGGATTGAGGAGAAACGTAAAAGGACATATGAGACC
TTCAAGAGCATCATGAAGAAGAGTCCTTTCAGCGGACCCACCGACCCCCGGCCTCCACCTCGAC
GCATTGCTGTGCCTTCCCGCAGCTCAGCTTCTGTCCCCAAGCCAGCACCCCAGCCCTATCCCTT
TACGTCATCCCTGAGCACCATCAACTATGATGAGTTTCCCACCATGGTGTTTCCTTCTGGGCAG
ATCAGCCAGGCCTCGGCCTTGGCCCCGGCCCCTCCCCAAGTCCTGCCCCAGGCTCCAGCCCCTG
CCCCTGCTCCAGCCATGGTATCAGCTCTGGCCCAGGCCCCAGCCCCTGTCCCAGTCCTAGCCCC
AGGCCCTCCTCAGGCTGTGGCCCCACCTGCCCCCAAGCCCACCCAGGCTGGGGAAGGAACGCTG
TCAGAGGCCCTGCTGCAGCTGCAGTTTGATGATGAAGACCTGGGGGCCTTGCTTGGCAACAGCA
CAGACCCAGCTGTGTTCACAGACCTGGCATCCGTCGACAACTCCGAGTTTCAGCAGCTGCTGAA
CCAGGGCATACCTGTGGCCCCCCACACAACTGAGCCCATGCTGATGGAGTACCCTGAGGCTATA
ACTCGCCTAGTGACAGGGGCCCAGAGGCCCCCCGACCCAGCTCCTGCTCCACTGGGGGCCCCGG
GGCTCCCCAATGGCCTCCTTTCAGGAGATGAAGACTTCTCCTCCATTGCGGACATGGACTTCTC
AGCCCTGCTGAGTCAGATCAGCTCCTAAGGGGGTGACGCCTGCCCTCCCCAGAGCACTGGGTTG
CAGGGGATTGAAGCCCTCCAAAAGCACTTACGGATTCTGGTGGGGTGTGTTCCAACTGCCCCCA
ACTTTGTGGATGTCTTCCTTGGAGGGGGGAGCCATATTTTATTCTTTTATTGTCAGTATCTGTA
TCTCTCTCTCTTTTTGGAGGTGCTTAAGCAGAAGCATTAACTTCTCTGGAAAGGGGGGAGCTGG
GGAAACTCAAACTTTTCCCCTGTCCTGATGGTCAGCTCCCTTCTCTGTAGGGAACTCTGGGGTC
CCCCATCCCCATCCTCCAGCTTCTGGTACTCTCCTAGAGACAGAAGCAGGCTGGAGGTAAGGCC
TTTGAGCCCACAAAGCCTTATCAAGTGTCTTCCATCATGGATTCATTACAGCTTAATCAAAATA
ACGCCCCAGATACCAGCCCCTGTATGGCACTGGCATTGTCCCTGTGCCTAACACCAGCGTTTGA
GGGGCTGGCCTTCCTGCCCTACAGAGGTCTCTGCCGGCTCTTTCCTTGCTCAACCATGGCTGAA
GGAAACCAGTGCAACAGCACTGGCTCTCTCCAGGATCCAGAAGGGGTTTGGTCTGGGACTTCCT
TGCTCTCCCTCTTCTCAAGTGCCTTAATAGTAGGGTAAGTTGTTAAGAGTGGGGGAGAGCAGGC
TGGCAGCTCTCCAGTCAGGAGGCATAGTTTTTACTGAACAATCAAAGCACTTGGACTCTTGCTC
TTTCTACTCTGAACTAATAAATCTGTTGCCAAGCTGG
Human RELA proto-oncogene, NF-kB subunit (RELA), transcript
variant X2, mRNA XM_011545207.1
(SEQ ID NO: 21)
ATTCCGGGCAGTGACGCGACGGCGGGCCGCGCGGCGCATTTCCGCCTCTGGCGAATGGCTCGTC
TGTAGTGCACGCCGCGGGCCCAGCTGCGACCCCGGCCCCGCCCCCGGGACCCCGGCCATGGACG
AACTGTTCCCCCTCATCTTCCCGGCAGAGCCAGCCCAGGCCTCTGGCCCCTATGTGGAGATCAT
TGAGCAGCCCAAGCAGCGGGGCATGCGCTTCCGCTACAAGTGCGAGGGGCGCTCCGCGGGCAGC
ATCCCAGGCGAGAGGAGCACAGATACCACCAAGACCCACCCCACCATCAAGATCAATGGCTACA
CAGGACCAGGGACAGTGCGCATCTCCCTGGTCACCAAGGACCCTCCTCACCGGCCTCACCCCCA
CGAGCTTGTAGGAAAGGACTGCCGGGATGGCTTCTATGAGGCTGAGCTCTGCCCGGACCGCTGC
ATCCACAGTTTCCAGAACCTGGGAATCCAGTGTGTGAAGAAGCGGGACCTGGAGCAGGCTATCA
GTCAGCGCATCCAGACCAACAACAACCCCTTCCAAGTTCCTATAGAAGAGCAGCGTGGGGACTA
CGACCTGAATGCTGTGCGGCTCTGCTTCCAGGTGACAGTGCGGGACCCATCAGGCAGGCCCCTC
CGCCTGCCGCCTGTCCTTTCTCATCCCATCTTTGACAATCACGATCGTCACCGGATTGAGGAGA
AACGTAAAAGGACATATGAGACCTTCAAGAGCATCATGAAGAAGAGTCCTTTCAGCGGACCCAC
CGACCCCCGGCCTCCACCTCGACGCATTGCTGTGCCTTCCCGCAGCTCAGCTTCTGTCCCCAAG
CCAGCACCCCAGCCCTATCCCTTTACGTCATCCCTGAGCACCATCAACTATGATGAGTTTCCCA
CCATGGTGTTTCCTTCTGGGCAGATCAGCCAGGCCTCGGCCTTGGCCCCGGCCCCTCCCCAAGT
CCTGCCCCAGGCTCCAGCCCCTGCCCCTGCTCCAGCCATGGTATCAGCTCTGGCCCAGGCCCCA
GCCCCTGTCCCAGTCCTAGCCCCAGGCCCTCCTCAGGCTGTGGCCCCACCTGCCCCCAAGCCCA
CCCAGGCTGGGGAAGGAACGCTGTCAGAGGCCCTGCTGCAGCTGCAGTTTGATGATGAAGACCT
GGGGGCCTTGCTTGGCAACAGCACAGACCCAGCTGTGTTCACAGACCTGGCATCCGTCGACAAC
TCCGAGTTTCAGCAGCTGCTGAACCAGGGCATACCTGTGGCCCCCCACACAACTGAGCCCATGC
TGATGGAGTACCCTGAGGCTATAACTCGCCTAGTGACAGGGGCCCAGAGGCCCCCCGACCCAGC
TCCTGCTCCACTGGGGGCCCCGGGGCTCCCCAATGGCCTCCTTTCAGGAGATGAAGACTTCTCC
TCCATTGCGGACATGGACTTCTCAGCCCTGCTGAGTCAGATCAGCTCCTAAGGGGGTGACGCCT
GCCCTCCCCAGAGCACTGGGTTGCAGGGGATTGAAGCCCTCCAAAAGCACTTACGGATTCTGGT
GGGGTGTGTTCCAACTGCCCCCAACTTTGTGGATGTCTTCCTTGGAGGGGGGAGCCATATTTTA
TTCTTTTATTGTCAGTATCTGTATCTCTCTCTCTTTTTGGAGGTGCTTAAGCAGAAGCATTAAC
TTCTCTGGAAAGGGGGGAGCTGGGGAAACTCAAACTTTTCCCCTGTCCTGATGGTCAGCTCCCT
TCTCTGTAGGGAACTCTGGGGTCCCCCATCCCCATCCTCCAGCTTCTGGTACTCTCCTAGAGAC
AGAAGCAGGCTGGAGGTAAGGCCTTTGAGCCCACAAAGCCTTATCAAGTGTCTTCCATCATGGA
TTCATTACAGCTTAATCAAAATAACGCCCCAGATACCAGCCCCTGTATGGCACTGGCATTGTCC
CTGTGCCTAACACCAGCGTTTGAGGGGCTGGCCTTCCTGCCCTACAGAGGTCTCTGCCGGCTCT
TTCCTTGCTCAACCATGGCTGAAGGAAACCAGTGCAACAGCACTGGCTCTCTCCAGGATCCAGA
AGGGGTTTGGTCTGGGACTTCCTTGCTCTCCCTCTTCTCAAGTGCCTTAATAGTAGGGTAAGTT
GTTAAGAGTGGGGGAGAGCAGGCTGGCAGCTCTCCAGTCAGGAGGCATAGTTTTTACTGAACAA
TCAAAGCACTTGGACTCTTGCTCTTTCTACTCTGAACTAATAAATCTGTTGCCAAGCTGG

In some embodiments, the nucleic acid sequence encoding Rel-A (p65), as described herein, is at least 80% identical to the sequence of SEQ ID NO: 16, SEQ ID NO: 17, SEQ ID NO: 18. SEQ ID NO: 19, SEQ ID NO: 20, or SEQ ID NO: 21. In some embodiments, the nucleic acid sequence encoding Rel-A (p65) is 80%, 81%, 82%, 83%, 84%, 85%, 86%, 8′7%, 88%, 89%, 90%, 91%, 92%, 93%, 94%, 95%, 96%, 97%, 98%, or 99% identical to the sequence of SEQ ID NO: 16, SEQ ID NO: 17, SEQ ID NO: 18, SEQ ID NO: 19, SEQ ID NO: 20, or SEQ ID NO: 21. In some embodiments, the nucleic acid encoding Rel-A (p65), as described herein, can vary from the sequence of SEQ ID NO: 16, SEQ ID NO: 17, SEQ ID NO: 18, SEQ ID NO: 19, SEQ ID NO: 20, or SEQ ID NO: 21 by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more nucleotides.

In some embodiments, an intracellular transcriptional regulatory domain includes a transcriptional activation domain present in a VP64 polypeptide. In some embodiments, the amino acid sequence of VP64 is or comprises all or a portion of SEQ ID NO: 22. In some embodiments, the amino acid sequence of the intracellular transcriptional regulatory domain of the chimeric transmembrane receptor comprises all or a portion of SEQ ID NO: 22. In some embodiments, the amino acid sequence of VP64, as described herein, is at least 80% identical to the amino acid sequence of SEQ ID NO: 22. In some embodiments, the amino acid sequence of VP64 is 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 22. In some embodiments, the amino acid sequence of VP64, as described herein, can vary from the amino acid sequence of SEQ ID NO: 22 by 1 amino acid, 2 amino acids, 3 amino acids, 4 amino acids, 5 amino acids, 6 amino acids, 7 amino acids, 8 amino acids, 9 amino acids, or 10 or more amino acids.

VP64 Polypeptide Sequence
(SEQ ID NO: 22)
DALDDFDLDMLGSDALDDFDLDMLGSDALDDFDLDMLGSDALDDFDLDML

In some embodiments, the nucleic acid sequence encoding VP64 is or comprises SEQ ID NO: 23. In some embodiments, the nucleic acid sequence encoding VP64, as described herein, is at least 80% identical to the sequence of SEQ ID NO: 23. In some embodiments, the nucleic acid sequence encoding VP64 is 80%, 81%, 82%, 83%, 84%, 85%, 86%, 87%, 88%, 89%, 90%, 91%, 92%, 93%, 94%, 95%, 96%, 97%, 98%, or 99% identical to the sequence of SEQ ID NO: 23. In some embodiments, the nucleic acid encoding VP64, as described herein, can vary from the sequence of SEQ ID NO: 23 by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more nucleotides.

VP64 Nucleotide Sequence
(SEQ ID NO: 23)
gacgctcttgatgatttcgatctcgacatgctgggatcagacgctctcga
cgacttcgatttggacatgcttggtccgacgctctcgatgatttcgacct
cgacatgctcggatccgatgctctggatgactttgatcttgatatgctg

In some embodiments, an intracellular transcriptional regulatory domain includes a transcriptional activation domain present in a MyoD polypeptide. In some embodiments, the amino acid sequence of MyoD is or comprises all or a portion of SEQ ID NO: 24. In some embodiments, the amino acid sequence of the intracellular transcriptional regulatory domain of the chimeric transmembrane receptor comprises all or a portion of SEQ ID NO: 24. In some embodiments, the amino acid sequence of MyoD, as described herein, is at least 80% identical to the amino acid sequence of SEQ ID NO: 24. In some embodiments, the amino acid sequence of VP64 is 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 24. In some embodiments, the amino acid sequence of MyoD, as described herein, can vary from the amino acid sequence of SEQ ID NO: 24 by 1 amino acid, 2 amino acids, 3 amino acids, 4 amino acids, 5 amino acids, 6 amino acids, 7 amino acids, 8 amino acids, 9 amino acids, or 10 or more amino acids.

MyoD Polypeptide Sequence
(SEQ ID NO: 24)
MELLSPPLRDIDLTGPDGSLCSFETADDFYDDPCFDSPDLRFFEDLDPRL
VHMGALLKPEEHAHFPTAVHPGPGAREDEHVRAPSGHHQAGRCLLWACKA
CKRKTTNADRRKAATMRERRRLSKVNEAFETLKRCTSSNPNQRLPKVEIL
RNAIRYIEGLQALLRDQDAAPPGAAAFYAPGPLPPGRGSEHYSGDSDASS
PRSNCSDGMMDYSGPPSGPRRQNGYDTAYYSEAARESRPGKSAAVSSLDC
LSSIVERISTDSPAAPALLLADAPPESPPGPPEGASLSDTEQGTQTPSPD
AAPQCPAGSNPNAIYQVL

Those of ordinary skill in the art will be aware of other suitable transcriptional activation domains that can be included in intracellular transcriptional regulatory domains of chimeric transmembrane receptors provided herein.

Any of a variety of DNA binding domains can be included in intracellular transcription regulatory domains of chimeric transmembrane receptors provided herein. In some embodiments, an intracellular transcriptional regulatory domain includes a DNA binding domain present in a GAL4 polypeptide. In some embodiments, the amino acid sequence of the GAL4 DNA binding domain is or comprises all or a portion of SEQ ID NO: 25. In some embodiments, the amino acid sequence of the intracellular DNA binding domain of the chimeric transmembrane receptor comprises all or a portion of SEQ ID NO: 25. In some embodiments, the amino acid sequence of the GAL4 DNA binding domain, as described herein, is at least 80% identical to the amino acid sequence of SEQ ID NO: 25. In some embodiments, the amino acid sequence of the GAL4 DNA binding domain is 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 25. In some embodiments, the amino acid sequence of the GAL4 DNA binding domain, as described herein, can vary from the amino acid sequence of SEQ ID NO: 25 by 1 amino acid, 2 amino acids, 3 amino acids, 4 amino acids, 5 amino acids, 6 amino acids, 7 amino acids, 8 amino acids, 9 amino acids, or 10 or more amino acids.

GAL4 DNA Binding Domain Polypeptide Sequence
(SEQ ID NO: 25)
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVS

In some embodiments, the nucleic acid sequence encoding the GAL4 DNA binding domain is or comprises SEQ ID NO: 26. In some embodiments, the nucleic acid sequence encoding the GAL4 DNA binding domain, as described herein, is at least 80% identical to the sequence of SEQ ID NO: 26. In some embodiments, the nucleic acid sequence encoding the GAL4 DNA binding domain is 80%, 81%, 82%, 83%, 84%, 85%, 86%, 87%, 88%, 89%, 90%, 91%, 92%, 93%, 94%, 95%, 96%, 97%, 98%, or 99% identical to the sequence of SEQ ID NO: 26. In some embodiments, the nucleic acid encoding the GAL4 DNA binding domain, as described herein, can vary from the sequence of SEQ ID NO: 26 by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more nucleotides.

GAL4 DNA Binding Domain Nucleotide Sequence
(SEQ ID NO: 26)
atgaaactccttagcagcatcgaacaggcttgcgacatctgcaggttgaa
aaaactcaagtgetcaaaagaaaagectaagtgcgcaaagtgccttaaaa
acaattgggaatgtcgctatagccccaagacaaagcggagccctctcacg
agagcacacctgactgaggtagaatctcgcttggagaggctggaacagct
tttcctgcttatctttccacgcgaggatctcgatatgatcctcaaaatgg
actccctccaggacatcaaagctctgctgactggactgtttgtacaggat
aatgtgaacaaggacgctgtgacagacagattggcaagcgtggaaacgga
tatgcccctgacccttagacagcaccggatcagtgccacctcttctagcg
aggaaagttcaaataaaggacagcgccagctgacggtgagt

Those of ordinary skill in the art will be aware of other suitable DNA binding domains that can be included in intracellular transcriptional regulatory domains of chimeric transmembrane receptors provided herein.

In some embodiments, an intracellular transcriptional regulatory domain includes a GAL4 DNA binding domain (e.g., the GAL4 DNA binding domain shown in SEQ ID NO: 25, or a variant thereof) and a VP64 transcriptional activation domain (e.g., the VP64 transcriptional action domain shown in SEQ ID NO: 22, or a variant thereof). In some embodiments, an intracellular transcriptional regulatory domain includes a linker sequence between the DNA binding domain and the transcriptional activation domain. Any of the variety of linker sequences disclosed herein can be included in an intracellular transcriptional regulatory domain. One non-limiting example of such a linker sequence is GGGSGGGS (SEQ ID NO: 27). In some embodiments, an intracellular transcriptional regulatory domain comprises the following polypeptide sequence:

(SEQ ID NO: 28)
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVSGGG
SGGGSDALDDFDLDMLGSDALDDFDLDMLGSDALDDFDLDMLGSDALDDF
DLDML

In some embodiments, an intracellular transcriptional regulatory domain includes a nucleic acid sequence encoding a GAL4 DNA binding domain (e.g., the nucleic acid sequence encoding the GAL4 DNA binding domain shown in SEQ ID NO: 26, or a variant thereof) and a nucleic acid sequence encoding a VP64 transcriptional activation domain (e.g., the nucleic acid sequence encoding the VP64 transcriptional action domain shown in SEQ ID NO: 23, or a variant thereof). In some embodiments, an intracellular transcriptional regulatory domain includes a nucleic acid sequence encoding a linker sequence between the DNA binding domain and the transcriptional activation domain. A nucleic acid sequence can encode any of the variety of linker sequences disclosed herein. One non-limiting example of a nucleic acid sequence encoding a linker sequence is ggcggtggaagcggaggaggttcc (SEQ ID NO: 29). In some embodiments, an intracellular transcriptional regulatory domain comprises the following nucleic acid sequence:

(SEQ ID NO: 30)
atgaaactccttagcagcatcgaacaggcttgcgacatctgcaggttgaa
aaaactcaagtgctcaaaagaaaagcctaagtgcgcaaagtgccttaaaa
acaattgggaatgtcgctatagccccaagacaaagcggagccctctcacg
agagcacacctgactgaggtagaatctcgcttggagaggctggaacagct
tttcctgcttatctttccacgcgaggatctcgatatgatcctcaaaatgg
actccctccaggacatcaaagctctgctgactggactgtttgtacaggat
aatgtgaacaaggacgctgtgacagacagattggcaagcgtggaaacgga
tatgcccctgacccttagacagcaccggatcagtgccacctcttctagcg
aggaaagttcaaataaaggacagcgccagctgacggtgagtggcggtgga
agcggaggaggttccgacgctcttgatgatttcgatctcgacatgctggg
atcagacgctctcgacgacttcgatttggacatgcttggtccgacgctct
cgatgatttcgacctcgacatgctcggatccgatgctctggatgactttg
atcttgatatgctg

In some embodiments, an intracellular transcriptional regulatory domain can include a GAL4 DNA-binding domain (e.g., any of the exemplary GAL4 DNA-binding domains described herein, such as those described herein) operably linked (e.g., optionally through the use of any of the linkers described herein to a VP64 transcriptional activation domain (e.g., any of the exemplary VP64 transcriptional activation domains described herein, such as those described below). In some embodiments, a GAL4 DNA-binding domain can include an amino acid sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to SEQ ID NO: 60. In some embodiments a GAL4 DNA-binding domain can be encoded by a nucleic acid that includes a sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% to SEQ ID NO: 61.

In some embodiments, a VP64 transcriptional activation domain can include an amino acid sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to SEQ ID NO: 22. In some embodiments a VP64 transcriptional activation domain can be encoded by a nucleic acid that includes a sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% to SEQ ID NO: 62.

Exemplary GAL4 DNA-Binding Domain
(SEQ ID NO: 60)
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVSGGG
SGGGS
Exemplary cDNA Encoding a GAL4 DNA-Binding Domain
(SEQ ID NO: 61)
ATGAAACTCCTTAGCAGCATCGAACAGGCTTGCGACATCTGCAGGTTGAA
AAAACTCAAGTGCTCAAAAGAAAAGCCTAAGTGCGCAAAGTGCCTTAAAA
ACAATTGGGAATGTCGCTATAGCCCCAAGACAAAGCGGAGCCCTCTCACG
AGAGCACACCTGACTGAGGTAGAATCTCGCTTGGAGAGGCTGGAACAGCT
TTTCCTGCTTATCTTTCCACGCGAGGATCTCGATATGATCCTCAAAATGG
ACTCCCTCCAGGACATCAAAGCTCTGCTGACTGGACTGTTTGTACAGGAT
AATGTGAACAAGGACGCTGTGACAGACAGATTGGCAAGCGTGGAAACGGA
TATGCCCCTGACCCTTAGACAGCACCGGATCAGTGCCACCTCTTCTAGCG
AGGAAAGTTCAAATAAAGGACAGCGCCAGCTGACGGTGAGT
Exemplary VP64 Transcriptional Activation Domain
(SEQ ID NO: 22)
DALDDFDLDMLGSDALDDFDLDMLGSDALDDFDLDMLGSDALDDFDLDML
Exemplary cDNA Encoding a VP64 Transcriptional
Activation Domain
(SEQ ID NO: 62)
GACGCTCTTGATGATTTCGATCTCGACATGCTGGGATCAGACGCTCTCGA
CGACTTCGATTTGGACATGCTTGGATCCGACGCTCTCGATGATTTCGACC
TCGACATGCTCGGATCCGATGCTCTGGATGACTTTGATCTTGATATGCTG

In some embodiments, an intracellular transcriptional regulatory domain can include a HNF1 alpha DNA-binding domain (e.g., any of the exemplary HNF1 alpha DNA-binding domains described herein, such as those described herein) operably linked (e.g., optionally through the use of any of the linkers described herein to a p65 transcriptional activation domain (e.g., any of the exemplary p65 transcriptional activation domains described herein, such as those described below).

In some embodiments, a HNF1 alpha DNA-binding domain can include an amino acid sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to SEQ ID NO: 112. In some embodiments a HNF1 alpha DNA-binding domain can be encoded by a nucleic acid that includes a sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% to SEQ ID NO: 113.

In some embodiments, a P65 transcriptional activation domain can include an amino acid sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to SEQ ID NO: 114. In some embodiments, a P65 transcriptional activation domain can be encoded by a nucleic acid that includes a sequence that is at least 70%, at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% to SEQ ID NO: 115.

Exemplary HNF1 alpha DNA-Binding Domain
(SEQ ID NO: 112)
MVSKLSQLQTELLAALLESGLSKEALIQALGEPGPYLLAGEGPLDKGESCGGGRGELAE
LPNGLGETRGSEDETDDDGEDFTPPILKELENLSPEEAAHQKAVVETLLQEDPWRVAK
MVKSYLQQHNIPQREVVDTTGLNQSHLSQHLNKGTPMKTQKRAALYTWYVRKQREV
AQQFTHAGQGGLIEEPTGDELPTKKGRRNRFKWGPASQQILFQAYERQKNPSKEERETL
VEECNRAECIQRGVSPSQAQGLGSNLVTEVRVYNWFANRRKEEAFRHKLAM
Exemplary cDNA Encoding a HNF1 alpha DNA-Binding Domain
(SEQ ID NO: 113)
ATGGTTTCTAAACTGAGCCAGCTGCAGACGGAGCTCCTGGCGGCCCTGCTCGAGTC
AGGGCTGAGCAAAGAGGCACTGATCCAGGCACTGGGTGAGCCGGGGCCCTACCTCC
TGGCTGGAGAAGGCCCCCTGGACAAGGGGGAGTCCTGCGGCGGCGGTCGAGGGGA
GCTGGCTGAGCTGCCCAATGGGCTGGGGGAGACTCGGGGCTCCGAGGACGAAACG
GACGACGATGGGGAAGACTTCACGCCACCCATCCTCAAAGAGCTGGAGAACCTCAG
CCCTGAGGAGGCGGCCCACCAGAAAGCCGTGGTGGAGACCCTTCTGCAGGAGGACC
CGTGGCGTGTGGCGAAGATGGTCAAGTCCTACCTGCAGCAGCACAACATCCCACAG
CGGGAGGTGGTCGATACCACTGGCCTCAACCAGTCCCACCTGTCCCAACACCTCAA
CAAGGGCACTCCCATGAAGACGCAGAAGCGGGCCGCCCTGTACACCTGGTACGTCC
GCAAGCAGCGAGAGGTGGCGCAGCAGTTCACCCATGCAGGGCAGGGAGGGCTGAT
TGAAGAGCCCACAGGTGATGAGCTACCAACCAAGAAGGGGCGGAGGAACCGTTTC
AAGTGGGGCCCAGCATCCCAGCAGATCCTGTTCCAGGCCTATGAGAGGCAGAAGAA
CCCTAGCAAGGAGGAGCGAGAGACTCTAGTGGAGGAGTGCAATAGGGCGGAATGC
ATCCAGAGAGGGGTGTCCCCATCACAGGCACAGGGGCTGGGCTCCAACCTCGTCAC
GGAGGTGCGTGTCTACAACTGGTTTGCCAACCGGCGCAAAGAAGAAGCCTTCCGGC
ACAAGCTGGCCATG
Exemplary P65 Transcriptional Activation Domain
(SEQ ID NO: 114)
DEFPTMVFPSGQISQASALAPAPPQVLPQAPAPAPAPAMVSALAQAPAPVPVLAPGPPQ
AVAPPAPKPTQAGEGTLSEALLQLQFDDEDLGALLGNSTDPAVFTDLASVDNSEFQQLL
NQGIPVAPHTTEPMLMEYPEAITRLVTGAQRPPDPAPAPLGAPGLPNGLLSGDEDFSSIA
DMDFSALLSQISS
Exemplary cDNA Encoding a P65 Transcriptional Activation Domain
(SEQ ID NO: 115)
GATGAGTTTCCCACCATGGTGTTTCCTTCTGGGCAGATCAGCCAGGCCTCGGCCTTG
GCCCCGGCCCCTCCCCAAGTCCTGCCCCAGGCTCCAGCCCCTGCCCCTGCTCCAGCC
ATGGTATCAGCTCTGGCCCAGGCCCCAGCCCCTGTCCCAGTCCTAGCCCCAGGCCCT
CCTCAGGCTGTGGCCCCACCTGCCCCCAAGCCCACCCAGGCTGGGGAAGGAACGCT
GTCAGAGGCCCTGCTGCAGCTGCAGTTTGATGATGAAGACCTGGGGGCCTTGCTTG
GCAACAGCACAGACCCAGCTGTGTTCACAGACCTGGCATCCGTCGACAACTCCGAG
TTTCAGCAGCTGCTGAACCAGGGCATACCTGTGGCCCCCCACACAACTGAGCCCAT
GCTGATGGAGTACCCTGAGGCTATAACTCGCCTAGTGACAGGGGCCCAGAGGCCCC
CCGACCCAGCTCCTGCTCCACTGGGGGCCCCGGGGCTCCCCAATGGCCTCCTTTCAG
GAGATGAAGACTTCTCCTCCATTGCGGACATGGACTTCTCAGCCCTGCTGAGTCAGA
TCAGCTCC

### Exemplary Embodiments

In some embodiments of any of the chimeric transmembrane receptors described herein, the extracellular integrin ligand-binding domain comprising an S2 protease cleavage site; the transmembrane domain; and the intracellular regulatory domain including the gamma-secretase protease cleavage site can be a contiguous sequence (or derived from a contiguous sequence) present within a naturally-occurring protein receptor (e.g., any of the exemplary PTPRs described herein).

Non-limiting examples of contiguous amino acid sequences from different PTPRs that include an extracellular integrin-ligand binding domain including an S2 protease cleavage site, a transmembrane domain, and an intracellular regulatory domain including the gamma-secretase protease cleavage site are shown below. Also shown below are non-limiting examples of nucleic acid sequences that encode contiguous amino acid sequences from different PTPRs that include an extracellular integrin-ligand binding domain including an S2 protease cleavage site, a transmembrane domain, and an intracellular regulatory domain including the gamma-secretase protease cleavage site.

Any of the chimeric transmembrane receptors described herein can include an amino acid sequence that is at least 72%, at least 74%, at least 76%, at least 78%, at least 80%, at least 82%, at least 84%, at least 86%, at least 88%, at least 90%, at least 92%, at least 94%, at least 96%, at least 98%, at least 99% or 100% identical to the amino acid sequence of any one of SEQ ID NOs: 56, 68, 74, 84, 94, 100, 108, 134, 138, 142, and 146. In some embodiments, a chimeric transmembrane receptors described herein can include an amino acid sequence that is identical to any of SEQ ID NOs: 56, 68, 74, 84, 94, 100, 108, 134, 138, 142, and 146, except that it includes 1, 2, 3, 4, 5, 6, 7, 8, 9, 10, 11, 12, 13, 14, 15, 16, 17, 18, 19, or 20 amino acid substitutions.

Any of nucleic acids encoding any of the chimeric transmembrane receptors described herein can include a nucleic acid sequence that is at least 72%, at least 74%, at least 76%, at least 78%, at least 80%, at least 82%, at least 84%, at least 86%, at least 88%, at least 90%, at least 92%, at least 94%, at least 96%, at least 98%, at least 99%, or 100% identical to the nucleic acid sequence of any one of SEQ ID NOs: 57, 69, 75, 85, 95, 101, 109, 135, 139, 143, and 147.

Exemplary Sequence from PTPR Delta
(SEQ ID NO: 56)
KNFHVKAVMKTSVLLSWEIPENYNSAMPFKILYDDGKMVEEVDGRATQKLIVNLKPEK
SYSFVLTNRGNSAGGLQHRVTAKTAPDVLRTKPAFIGKTNLDGMITVQLPEVPANENIK
GYYMVPLKKSRGKFIKPWESPDEMELDELLKEISRKRRSIRYGREVELKPYIAAHFDVL
PTEFTLGDDKHYGGFTNKQLQSGQEYVFFVLAVMEHAESKMYATSPYSDPVVSMDLD
PQPITDEEEGLIWVVGPVLAVVFIICIVIAILLYKRKRAESDSRKSSGG
cDNA Sequence Encoding Exemplary Sequence from PTPR Delta
(SEQ ID NO: 57)
AAAAATTTCCACGTGAAGGCTGTTATGAAAACATCCGTTCTCCTGTCATGGGAAATC
CCGGAAAACTATAATTCTGCTATGCCTTTCAAGATATTGTATGATGATGGCAAGATG
GTTGAAGAGGTCGACGGTCGGGCGACACAAAAACTGATCGTTAACCTCAAACCTGA
GAAATCATATTCATTCGTCCTCACCAATCGCGGTAATAGTGCTGGTGGCCTCCAGCA
CCGGGTAACCGCAAAAACTGCGCCTGATGTGCTCAGGACGAAGCCCGCGTTTATAG
GCAAGACCAATCTTGATGGCATGATCACTGTTCAGCTCCCGGAAGTTCCCGCCAAC
GAGAATATCAAGGGTTATTATATTATTATCGTACCGCTCAAGAAGTCTCGAGGCAA
ATTTATCAAACCTTGGGAGTCACCAGATGAAATGGAGCTTGATGAGTTGCTCAAAG
AGATCAGCAGAAAGCGGCGGTCCATAAGGTACGGCAGGGAGGTCGAGCTCAAGCC
ATACATTGCGGCTCATTTCGATGTGTTGCCGACGGAGTTCACGCTCGGGGATGATAA
ACACTACGGCGGCTTCACAAACAAACAGCTCCAATCAGGGCAGGAGTATGTCTTCT
TCGTGCTTGCTGTCATGGAACACGCCGAATCCAAAATGTATGCAACAAGCCCTTACT
CCGATCCGGTTGTTTCTATGGATCTGGACCCGCAGCCGATAACAGATGAAGAAGAA
GGGCTCATTTGGGTGGTTGGCCCTGTGCTGGCCGTGGTGTTTATTATCTGTATCGTTA
TTGCGATTCTTCTCTATAAGCGGAAGCGAGCGGAGAGTGACTCTCGAAAATCATCC
GGGGGT
Exemplary Sequence from PTPR LAR
(SEQ ID NO: 68)
AKNFRVAAAMKTSVLLSWEVPDSYKSAVPFKILYNGQSVEVDGHSMRKLIADLQPNTE
YSFVLMNRGSSAGGLQHLVSIRTAPDLLPHKPLPASAYIEDGRFDLSMPHVQDPSLVRW
FYIVVVPIDRVGGSMLTPRWSTPEELELDELLEAIEQGGEEQRRRRRQAERLKPYVAAQ
LDVLPETFTLGDKKNYRGFYNRPLSPDLSYQCFVLASLKEPMDQKRYASSPYSDEIVVQ
VTPAQQQEEPEMLWVTGPVLAVILIILIVIAILLFKRKRTHSPSSKDEQSIGGG
cDNA Sequence Encoding Exemplary Sequence from PTPR LAR
(SEQ ID NO: 69)
GCCAAAAACTTCAGAGTAGCGGCGGCGATGAAAACTTCTGTCCTTCTGAGCTGGGA
AGTTCCGGATTCCTATAAATCCGCTGTCCCCTTCAAGATATTGTACAATGGTCAAAG
TGTCGAGGTTGATGGACACTCCATGAGGAAACTCATTGCCGACCTCCAACCGAACA
CAGAATACAGTTTTGTTTTGATGAATCGCGGTTCCTCAGCCGGGGGCCTGCAGCACC
TCGTGAGTATTAGGACCGCTCCCGATCTTCTTCCACATAAGCCTCTCCCCGCATCTG
CGTACATAGAAGACGGGCGGTTCGACCTGTCAATGCCCCACGTACAGGACCCTAGT
CTTGTAAGATGGTTTTATATCGTGGTCGTTCCTATAGACCGGGTTGGAGGCTCTATG
CTCACCCCGCGATGGAGTACCCCGGAAGAACTTGAGCTGGATGAGTTGTTGGAAGC
AATCGAACAGGGGGGCGAGGAACAGAGGCGGCGCCGCCGCCAAGCAGAGCGACTG
AAACCGTATGTTGCCGCTCAGTTGGATGTGTTGCCCGAAACGTTTACTTTGGGTGAC
AAAAAAAATTATCGGGGGTTCTACAATAGACCTTTGAGTCCTGATCTTAGTTATCAG
TGTTTTGTGTTGGCATCTTTGAAGGAACCGATGGACCAGAAACGATACGCTTCCTCC
CCCTATAGTGACGAGATTGTCGTTCAAGTAACACCCGCACAACAGCAGGAGGAGCC
GGAAATGCTTTGGGTTACAGGGCCGGTACTTGCGGTGATCTTGATAATACTCATTGT
CATAGCCATTCTGCTTTTCAAGCGCAAACGGACCCACTCACCCTCTTCCAAGGACGA
GCAGAGCATAGGGGGAGGG
Exemplary Sequence from PTPR Mu
(SEQ ID NO: 74)
IFLQWREPTQTYGVITLYEITYKAVSSFDPEIDLSNQSGRVSKLGNETHFLFFGLYPGTTY
SFTIRASTAKGFGPPATNQFTTKISAPSMPAYELETPLNQTDNTVTVMLKPAHSRGAPVS
VYQIVVEEERPRRTKKTTEILKCYPVPIHFQNASLLNSQYYFAAEFPADSLQAAQPFTIG
DNKTYNGYWNTPLLPYKSYRIYFQAASRANGETKIDCVQVATKGAATPKPVPEPEKQT
DHTVKIAGVIAGILLFVIIFLGVVLVMKKRKLAKKRKETMSSTGG
cDNA Sequence Encoding Exemplary Sequence from PTPR Mu
(SEQ ID NO: 75)
ATATTTCTGCAATGGCGAGAGCCTACACAAACATACGGAGTCATAACGTTGTACGA
AATTACGTACAAGGCCGTGTCATCATTCGATCCCGAAATTGATCTTTCTAACCAGTC
AGGGCGCGTAAGTAAACTCGGCAACGAGACCCACTTTTTGTTCTTCGGCCTGTATCC
GGGCACTACGTACAGTTTCACCATCCGCGCATCTACGGCCAAGGGTTTTGGCCCACC
CGCTACGAACCAGTTTACTACGAAGATTTCTGCTCCTTCAATGCCAGCTTATGAACT
CGAAACTCCACTGAACCAAACTGACAACACAGTTACTGTGATGCTGAAGCCCGCGC
ATAGCCGAGGTGCCCCAGTTTCTGTGTATCAAATTGTGGTAGAAGAAGAACGGCCA
CGCCGCACAAAGAAGACGACGGAAATACTGAAATGTTATCCAGTCCCTATTCACTT
CCAGAACGCTAGTTTGCTTAACTCACAGTATTATTTCGCGGCAGAATTCCCCGCCGA
TTCTCTGCAGGCGGCACAGCCCTTTACAATAGGGGACAACAAGACTTACAATGGCT
ATTGGAACACCCCCTTGCTTCCTTACAAGAGCTACAGGATCTACTTTCAAGCGGCCT
CCCGCGCAAACGGTGAAACGAAAATTGACTGTGTGCAGGTAGCCACAAAGGGTGC
AGCGACTCCGAAGCCCGTACCGGAGCCAGAGAAGCAAACTGATCACACAGTCAAG
ATTGCCGGCGTCATAGCAGGTATTCTGTTGTTCGTGATAATCTTTCTCGGCGTCGTCC
TCGTTATGAAGAAGAGGAAACTCGCAAAGAAGCGGAAGGAAACAATGTCATCCAC
TGGTGGA
Exemplary Sequence from PTPR Psi
(SEQ ID NO: 84)
EDVPSGIAAESLTFTPLEDMIFLKWEEPQEPNGLITQYEISYQSIESSDPAVNVPGPRRTIS
KLRNETYHVFSNLHPGTTYLFSVRARTGKGFGQAALTEITTNISAPSFDYADMPSPLGES
ENTITVLLRPAQGRGAPISVYQVIVEEERARRLRREPGGQDCFPVPLTFEAALARGLVHY
FGAELAASSLPEAMPFTVGDNQTYRGFWNPPLEPRKAYLIYFQAASHLKGETRLNCIRI
ARKAACKESKRPLEVSQRSEEMGLILGICAGGLAVLILLLGAIIVIIRKGRDHYAYSYYPK
PVNMTGG
cDNA Sequence Encoding Exemplary Sequence from PTPR Psi
(SEQ ID NO: 85)
GAAGATGTCCCATCCGGTATAGCGGCTGAAAGCTTGACATTTACCCCATTGGAAGA
TATGATTTTCCTGAAATGGGAAGAACCACAAGAGCCCAATGGTCTCATTACTCAAT
ATGAGATCAGTTACCAGAGCATTGAGTCAAGTGATCCCGCTGTCAATGTACCGGGA
CCTCGCAGGACTATCTCTAAGCTGCGGAACGAAACGTACCATGTATTCAGCAACCT
GCACCCCGGCACCACGTACTTGTTTTCCGTACGCGCGAGAACTGGCAAGGGATTCG
GGCAGGCTGCCCTTACAGAAATAACTACGAACATTTCTGCTCCTTCATTCGACTACG
CAGACATGCCTTCACCGCTCGGTGAATCTGAGAACACCATTACGGTCCTGCTTAGGC
CTGCACAGGGAAGGGGTGCTCCCATTTCCGTCTACCAGGTAATCGTTGAAGAGGAA
CGCGCCCGGCGGCTCAGACGGGAACCCGGTGGGCAAGACTGTTTCCCGGTCCCTCT
GACCTTTGAGGCGGCCTTGGCCAGAGGTCTGGTGCATTACTTCGGAGCCGAGTTGG
CCGCAAGCTCACTGCCTGAGGCGATGCCCTTCACCGTGGGGGACAATCAGACCTAC
AGGGGATTTTGGAATCCACCTCTTGAACCTCGCAAAGCGTACCTGATCTATTTCCAG
GCTGCGTCACACCTGAAAGGGGAAACCAGGTTGAATTGCATCCGCATAGCTAGGAA
AGCCGCCTGTAAAGAGTCCAAAAGGCCACTTGAAGTCTCTCAGCGCAGTGAAGAAA
TGGGTCTGATCCTTGGAATTTGCGCGGGAGGGCTGGCTGTACTTATCCTTCTCCTCG
GAGCTATAATCGTTATAATCAGGAAAGGCAGAGACCACTACGCCTACTCTTACTAT
CCTAAACCGGTGAACATGACGGGGGGA
Exemplary Sequence from PTPR Pho
(SEQ ID NO: 94)
KIYIQWKPPNETNGVITLYEINYKAVGSLDPSADLSSQRGKVFKLRNETHELFVGLYPGT
TYSFTIKASTAKGFGPPVTTRIATKISAPSMPEYDTDTPLNETDTTITVMLKPAQSRGAPV
SVYQLVVKEERLQKSRRAADIIECFSVPVSYRNASSLDSLHYFAAELKPANLPVTQPFTV
GDNKTYNGYWNPPLSPLKSYSIYFQALSKANGETKINCVRLATKGASTQNSNTVEPEKQ
VDNTVKMAGVIAGLLMFIIILLGVMLTIKRRRNAYSYSYYLKLAKKQKETGG
cDNA Sequence Encoding Exemplary Sequence from PTPR Pho
(SEQ ID NO: 95)
AAAATCTATATTCAATGGAAGCCGCCAAATGAAACCAATGGAGTTATAACGCTCTA
TGAAATCAACTACAAGGCGGTTGGCTCCCTTGATCCCTCTGCCGACCTTTCCTCACA
GCGGGGTAAAGTGTTCAAGCTGAGGAACGAAACGCACCACCTTTTCGTGGGGTTGT
ATCCAGGAACGACCTACAGTTTTACTATTAAGGCTTCCACAGCCAAAGGCTTTGGGC
CCCCTGTAACCACTAGGATTGCTACTAAAATCTCCGCGCCATCTATGCCCGAATATG
ATACGGACACCCCATTGAACGAAACAGATACAACAATAACTGTCATGCTGAAGCCT
GCGCAATCACGCGGAGCCCCTGTCAGCGTATATCAACTTGTAGTCAAAGAAGAAAG
ACTGCAAAAATCCCGACGCGCTGCCGACATTATTGAGTGCTTCTCAGTACCCGTGAG
CTACAGAAACGCTAGTAGCTTGGATTCTTTGCATTATTTCGCGGCCGAACTTAAGCC
CGCGAATCTTCCGGTGACTCAACCGTTTACAGTGGGTGACAATAAAACTTACAATG
GCTATTGGAACCCGCCGTTGTCACCGCTGAAGAGCTACTCAATATATTTCCAGGCCC
TGAGTAAAGCTAACGGTGAGACAAAAATCAACTGTGTGAGACTTGCAACTAAAGGA
GCCAGCACACAGAATTCTAATACTGTGGAGCCCGAGAAGCAAGTTGACAACACTGT
GAAAATGGCTGGGGTAATTGCAGGACTGCTTATGTTCATCATAATCCTGCTTGGGGT
TATGCTTACTATCAAGCGACGGCGCAACGCCTACAGCTATAGCTACTATTTGAAATT
GGCAAAAAAGCAGAAGGAAACTGGAGGG
Exemplary Sequence from PTPR Sigma
(SEQ ID NO: 100)
SPKNFKVKMIMKTSVLLSWEFPDNYNSPTPYKIQYNGLTLDVDGRTTKKLITHLKPHTF
YNFVLTNRGSSLGGLQQTVTAWTAFNLLNGKPSVAPKPDADGFIMVYLPDGQSPVPVQ
SYFIVMVPLRKSRGGQFLTPLGSPEDMDLEELIQDISRLQRRSLRHSRQLEVPRPYIAARF
SVLPPTFHPGDQKQYGGFDNRGLEPGHRYVLFVLAVLQKSEPTFAASPFSDPFQLDNPD
PQPIVDGEEGLIWVIGPVLAVVFIICIVIAILLYKNKPDSKRKDSEPRTKGG
cDNA Sequence Encoding Exemplary Sequence from PTPR Sigma
(SEQ ID NO: 101)
AGTCCGAAGAACTTCAAAGTCAAGATGATCATGAAGACATCTGTGCTCTTGAGTTG
GGAGTTTCCCGATAATTATAACAGCCCCACGCCTTACAAGATTCAGTACAATGGACT
CACCCTTGATGTAGATGGCCGCACGACAAAGAAGCTGATTACTCACCTCAAACCGC
ATACTTTCTATAATTTCGTGCTGACGAATCGGGGTTCTTCCCTGGGAGGTCTCCAGC
AAACTGTAACGGCGTGGACTGCGTTTAATTTGCTGAACGGTAAGCCCTCAGTGGCC
CCCAAACCGGATGCCGACGGATTTATAATGGTGTACCTTCCAGATGGTCAGAGTCC
GGTCCCCGTACAGAGCTACTTCATTGTCATGGTGCCCCTCAGGAAATCCCGAGGTGG
TCAATTTCTCACACCATTGGGTAGTCCGGAGGACATGGATCTGGAAGAACTGATCC
AGGATATTAGCCGCCTGCAACGCAGATCACTTAGACATAGTAGACAGCTGGAGGTG
CCGAGGCCGTACATCGCTGCGCGATTCTCCGTACTCCCGCCAACCTTTCACCCAGGG
GATCAGAAACAATACGGCGGTTTTGATAATCGAGGGCTTGAACCAGGACATAGATA
CGTGCTTTTTGTGTTGGCTGTGCTCCAGAAATCTGAACCGACGTTTGCCGCAAGCCC
CTTTAGCGACCCATTTCAGCTGGATAACCCTGACCCTCAGCCGATAGTCGATGGCGA
GGAGGGGCTGATATGGGTGATTGGGCCCGTACTCGCGGTAGTGTTTATTATCTGTAT
CGTAATTGCTATACTGCTTTATAAGAACAAGCCGGACAGTAAAAGGAAGGATTCTG
AGCCTAGGACTAAAGGCGGT
Exemplary Sequence from PTPR Kappa
(SEQ ID NO: 108)
DVPGPVPVKSLQGTSFENKIFLNWKEPLDPNGIITQYEISYSSIRSFDPAVPVAGPPQTVS
NLWNSTHEIVFIVIEILHPGTTYQFFIRASTVKGFGPATAINVTTNISAPTLPDYEGVDASLN
ETATTITVLLRPAQAKGAPISAYQIVVEELHPHRTKREAGAMECYQVPVTYQNAIVISGG
APYYFAAELPPGNLPEPAPFTVGDNRTYQGFWNPPLAPRKGYNIYFQAMSSVEKETKTQ
CVRIATKAAATEEPEVIPDPAKQTDRVVKIAGISAGILVFILLLLVVILIVKKSKLAKKRK
DAMG
cDNA Sequence Encoding Exemplary Sequence from PTPR Kappa
(SEQ ID NO: 109)
GATGTGCCTGGTCCCGTACCAGTAAAATCTCTTCAAGGAACATCCTTTGAAAATAAG
ATCTTCTTGAACTGGAAAGAACCTTTGGATCCAAATGGAATCATCACTCAATATGAG
ATCAGCTATAGCAGTATAAGATCATTTGATCCTGCAGTTCCAGTGGCTGGACCTCCC
CAGACTGTATCAAATTTATGGAACAGTACACACCATGTCTTTATGCATCTCCACCCT
GGAACCACGTACCAGTTTTTCATAAGAGCCAGCACGGTCAAAGGCTTTGGTCCAGC
CACAGCCATCAATGTCACCACCAATATCTCAGCTCCAACTTTACCTGACTATGAAGG
AGTTGATGCCTCTCTCAATGAAACTGCCACCACAATAACTGTATTGTTGAGACCAGC
ACAAGCCAAAGGTGCTCCTATCAGTGCTTATCAGATTGTTGTGGAAGAACTGCACC
CACACCGAACCAAGAGAGAAGCCGGAGCCATGGAATGCTACCAGGTTCCTGTCACA
TACCAAAATGCCATGAGTGGGGGTGCACCGTATTACTTTGCTGCAGAACTACCCCC
GGGAAACCTACCTGAGCCTGCCCCGTTCACTGTGGGTGACAATCGGACCTACCAAG
GCTTTTGGAACCCTCCTTTGGCTCCGCGCAAAGGATACAACATCTATTTCCAGGCGA
TGAGCAGTGTGGAGAAGGAAACTAAAACCCAGTGCGTACGCATTGCTACAAAAGC
AGCAGCAACAGAAGAACCAGAAGTGATCCCAGATCCCGCCAAGCAGACAGACAGA
GTGGTGAAAATAGCAGGAATTAGTGCTGGAATTTTGGTGTTCATCC TCCTTCTCCTA
GTTGTCATATTAATTGTAAAAAAGAGCAAACTTGCTAAAAAACGCAAAGATGCCAT
GGGG
Exemplary Sequence from PTPR LAR
(SEQ ID NO: 134)
PPQKVMCVSMGSTTVRVSWVPPPADSRNGVITQYSVAYEAVDGEDRGRHVVDGISRE
HSSWDLVGLEKWTEYRVWVRAHTDVGPGPESSPVLVRTDEDVPSGPPRKVEVEPLNST
AVHVYWKLPVPSKQHGQIRGYQVTYVRLENGEPRGLPIIQDVMLAEAQWRPEESEDYE
TTIS GLTPETTYSVTVAAYTTKGDGARSKPKIVTTTGAVPGRPTMMISTTAMNTALLQW
HPPKELPGELLGYRLQYCRADEARPNTIDFGKDDQHFTVTGLHKGTTYIFRLAAKNRAG
LGEEFEKEIRTPEDLPSGFPQNLHVTGLTTSTTELAWDPPVLAERNGRIISYTVVFRDINS
QQELQNITTDTRFTLTGLKPDTTYDIKVRAWTSKGSGPLSPSIQSRTMPVEQVFAKNFRV
AAAMKTSVLLSWEVPDSYKSAVPFKILYNGQSVEVDGHSMRKLIADLQPNTEYSFVLM
NRGSSAGGLQHLVSIRTAPDLLPHKPLPASAYIEDGRFDLSMPHVQDPSLVRWFYIVVV
PIDRVGGSMLTPRWSTPEELELDELLEAIEQGGEEQRRRRRQAERLKPYVAAQLDVLPE
TFTLGDKKNYRGFYNRPLSPDLSYQCFVLASLKEPMDQKRYASSPY SDEIVVQVTPAQQ
QEEPEMLWVTGPVLAVILIILIVIAILLFKRKRTHSPSSKDEQSIGGG
cDNA Sequence Encoding Exemplary Sequence from PTPR LAR
(SEQ ID NO: 135)
CCACCGCAAAAAGTTATGTGTGTGAGCATGGGATCTACCACCGTCCGCGTTTCTTGG
GTTCCACCGCCTGCCGATAGTCGGAATGGCGTCATTACTCAATACAGCGTAGCCTAC
GAGGCCGTGGATGGAGAGGATAGAGGTCGCCATGTCGTAGATGGAATTAGCCGCG
AGCATAGTTCATGGGACTTAGTCGGGTTGGAAAAGTGGACTGAATACCGTGTTTGG
GTCCGAGCTCACACGGATGTTGGCCCAGGACCAGAGTCCAGTCCCGTTCTCGTTCGG
ACGGACGAGGACGTTCCGTCCGGTCCACCCCGAAAAGTTGAGGTAGAGCCCCTGAA
TAGTACGGCAGTACATGTCTATTGGAAGCTCCCTGTTCCATCAAAGCAACATGGGC
AGATTAGGGGGTACCAAGTTACATATGTGCGATTGGAAAACGGGGAACCACGGGG
CCTCCCGATCATCCAAGATGTTATGCTCGCAGAGGCACAGTGGCGCCCCGAGGAGT
CCGAAGATTATGAAACTACTATAAGTGGGCTTACCCCAGAGACAACCTACTCTGTA
ACTGTAGCTGCTTACACGACCAAAGGGGATGGCGCCCGAAGCAAGCCCAAAATTGT
TACCACAACAGGGGCTGTACCCGGCAGACCGACAATGATGATCAGCACGACAGCG
ATGAACACGGCACTTCTGCAATGGCATCCGCCCAAGGAATTGCCAGGGGAGTTGCT
CGGCTACCGGCTGCAATACTGTAGGGCAGATGAAGCAAGGCCGAACACAATCGATT
TCGGAAAGGATGATCAGCACTTTACTGTTACCGGACTTCATAAAGGGACAACTTAT
ATCTTCCGGCTGGCGGCTAAAAATAGAGCGGGCTTGGGAGAAGAATTCGAGAAGG
AGATTAGAACCCCGGAGGATTTGCCTAGTGGATTCCCGCAAAATCTCCATGTCACG
GGACTCACTACTTCAACGACCGAACTCGCTTGGGATCCTCCAGTCCTTGCCGAAAGA
AACGGAAGGATTATAAGCTACACCGTAGTCTTCCGAGATATTAACTCACAACAAGA
ACTCCAGAATATTACTACCGATACGCGATTCACATTGACAGGACTTAAGCCTGACA
CGACTTATGATATAAAAGTCCGGGCTTGGACTTCCAAAGGAAGCGGACCACTGTCT
CCCTCAATCCAATCGAGGACTATGCCAGTTGAGCAAGTGTTTGCaAAAAACTTCAGA
GTAGCGGCGGCGATGAAAACTTCTGTCCTTCTGAGCTGGGAAGTTCCGGATTCCTAT
AAATCCGCTGTCCCCTTCAAGATATTGTACAATGGTCAAAGTGTCGAGGTTGATGGA
CACTCCATGAGGAAACTCATTGCCGACCTCCAACCGAACACAGAATACAGTTTTGTT
TTGATGAATCGCGGTTCCTCAGCCGGGGGCCTGCAGCACCTCGTGAGTATTAGGAC
CGCTCCCGATCTTCTTCCACATAAGCCTCTCCCCGCATCTGCGTACATAGAAGACGG
GCGGTTCGACCTGTCAATGCCCCACGTACAGGACCCTAGTCTTGTAAGATGGTTTTA
TATCGTGGTCGTTCCTATAGACCGGGTTGGAGGCTCTATGCTCACCCCGCGATGGAG
TACCCCGGAAGAACTTGAGCTGGATGAGTTGTTGGAAGCAATCGAACAGGGGGGCG
AGGAACAGAGGCGGCGCCGCCGCCAAGCAGAGCGACTGAAACCGTATGTTGCCGC
TCAGTTGGATGTGTTGCCCGAAACGTTTACTTTGGGTGACAAAAAAAATTATCGGG
GGTTCTACAATAGACCTTTGAGTCCTGATCTTAGTTATCAGTGTTTTGTGTTGGCATC
TTTGAAGGAACCGATGGACCAGAAACGATACGCTTCCTCCCCCTATAGTGACGAGA
TTGTCGTTCAAGTAACACCCGCACAACAGCAGGAGGAGCCGGAAATGCTTTGGGTT
ACAGGGCCGGTACTTGCGGTGATCTTGATAATACTCATTGTCATAGCCATTCTGCTT
TTCAAGCGCAAACGGACCCACTCACCCTCTTCCAAGGACGAGCAGAGCATAGGGGG
AGGG
Exemplary Sequence from PTPR LAR
(SEQ ID NO: 138)
VPSGPPRKVEVEPLNSTAVHVYWKLPVPSKQHGQIRGYQVTYVRLENGEPRGLPIIQDV
MLAEAQWRPEESEDYETTISGLTPETTYSVTVAAYTTKGDGARSKPKIVTTTGAVPGRP
TMMISTTAMNTALLQWHPPKELPGELLGYRLQYCRADEARPNTIDFGKDDQHFTVTGL
HKGTTYIFRLAAKNRAGLGEEFEKEIRTPEDLPSGFPQNLHVTGLTTSTTELAWDPPVLA
ERNGRIISYTVVFRDINSQQELQNITTDTRFTLTGLKPDTTYDIKVRAWTSKGSGPLSPSI
QSRTMPVEQVFAKNFRVAAAMKTSVLLSWEVPDSYKSAVPFKILYNGQSVEVDGHSM
RKLIADLQPNTEYSFVLMNRGSSAGGLQHLVSIRTAPDLLPHKPLPASAYIEDGRFDLSM
PHVQDPSLVRWFYIVVVPIDRVGGSMLTPRWSTPEELELDELLEAIEQGGEEQRRRRRQ
AERLKPYVAAQLDVLPETFTLGDKKNYRGFYNRPLSPDLSYQCFVLASLKEPMDQKRY
ASSPYSDEIVVQVTPAQQQEEPEMLWVTGPVLAVILIILIVIAILLFKRKRTHSPSSKDEQS
IGGG
cDNA Sequence Encoding Exemplary Sequence from PTPR LAR
(SEQ ID NO: 139)
GTTCCGTCCGGTCCACCCCGAAAAGTTGAGGTAGAGCCCCTGAATAGTACGGCAGT
ACATGTCTATTGGAAGCTCCCTGTTCCATCAAAGCAACATGGGCAGATTAGGGGGT
ACCAAGTTACATATGTGCGATTGGAAAACGGGGAACCACGGGGCCTCCCGATCATC
CAAGATGTTATGCTCGCAGAGGCACAGTGGCGCCCCGAGGAGTCCGAAGATTATGA
AACTACTATAAGTGGGCTTACCCCAGAGACAACCTACTCTGTAACTGTAGCTGCTTA
CACGACCAAAGGGGATGGCGCCCGAAGCAAGCCCAAAATTGTTACCACAACAGGG
GCTGTACCCGGCAGACCGACAATGATGATCAGCACGACAGCGATGAACACGGCACT
TCTGCAATGGCATCCGCCCAAGGAATTGCCAGGGGAGTTGCTCGGCTACCGGCTGC
AATACTGTAGGGCAGATGAAGCAAGGCCGAACACAATCGATTTCGGAAAGGATGA
TCAGCACTTTACTGTTACCGGACTTCATAAAGGGACAACTTATATCTTCCGGCTGGC
GGCTAAAAATAGAGCGGGCTTGGGAGAAGAATTCGAGAAGGAGATTAGAACCCCG
GAGGATTTGCCTAGTGGATTCCCGCAAAATCTCCATGTCACGGGACTCACTACTTCA
ACGACCGAACTCGCTTGGGATCCTCCAGTCCTTGCCGAAAGAAACGGAAGGATTAT
AAGCTACACCGTAGTCTTCCGAGATATTAACTCACAACAAGAACTCCAGAATATTA
CTACCGATACGCGATTCACATTGACAGGACTTAAGCCTGACACGACTTATGATATA
AAAGTCCGGGCTTGGACTTCCAAAGGAAGCGGACCACTGTCTCCCTCAATCCAATC
GAGGACTATGCCAGTTGAGCAAGTGTTTGCaAAAAACTTCAGAGTAGCGGCGGCGA
TGAAAACTTCTGTCCTTCTGAGCTGGGAAGTTCCGGATTCCTATAAATCCGCTGTCC
CCTTCAAGATATTGTACAATGGTCAAAGTGTCGAGGTTGATGGACACTCCATGAGG
AAACTCATTGCCGACCTCCAACCGAACACAGAATACAGTTTTGTTTTGATGAATCGC
GGTTCCTCAGCCGGGGGCCTGCAGCACCTCGTGAGTATTAGGACCGCTCCCGATCTT
CTTCCACATAAGCCTCTCCCCGCATCTGCGTACATAGAAGACGGGCGGTTCGACCTG
TCAATGCCCCACGTACAGGACCCTAGTCTTGTAAGATGGTTTTATATCGTGGTCGTT
CCTATAGACCGGGTTGGAGGCTCTATGCTCACCCCGCGATGGAGTACCCCGGAAGA
ACTTGAGCTGGATGAGTTGTTGGAAGCAATCGAACAGGGGGGCGAGGAACAGAGG
CGGCGCCGCCGCCAAGCAGAGCGACTGAAACCGTATGTTGCCGCTCAGTTGGATGT
GTTGCCCGAAACGTTTACTTTGGGTGACAAAAAAAATTATCGGGGGTTCTACAATA
GACCTTTGAGTCCTGATCTTAGTTATCAGTGTTTTGTGTTGGCATCTTTGAAGGAACC
GATGGACCAGAAACGATACGCTTCCTCCCCCTATAGTGACGAGATTGTCGTTCAAGT
AACACCCGCACAACAGCAGGAGGAGCCGGAAATGCTTTGGGTTACAGGGCCGGTA
CTTGCGGTGATCTTGATAATACTCATTGTCATAGCCATTCTGCTTTTCAAGCGCAAA
CGGACCCACTCACCCTCTTCCAAGGACGAGCAGAGCATAGGGGGAGGG
Exemplary Sequence from PTPR LAR
(SEQ ID NO: 142)
VPGRPTMMISTTAMNTALLQWHPPKELPGELLGYRLQYCRADEARPNTIDFGKDDQHF
TVTGLHKGTTYIFRLAAKNRAGLGEEFEKEIRTPEDLPSGFPQNLHVTGLTTSTTELAWD
PPVLAERNGRIISYTVVFRDINSQQELQNITTDTRFTLTGLKPDTTYDIKVRAWTSKGSGP
LSPSIQSRTMPVEQVFAKNFRVAAAMKTSVLLSWEVPDSYKSAVPFKILYNGQSVEVDG
HSMRKLIADLQPNTEYSFVLMNRGSSAGGLQHLVSIRTAPDLLPHKPLPASAYIEDGRFD
LSMPHVQDPSLVRWFYIVVVPIDRVGGSMLTPRWSTPEELELDELLEAIEQGGEEQRRR
RRQAERLKPYVAAQLDVLPETFTLGDKKNYRGFYNRPLSPDLSYQCFVLASLKEPMDQ
KRYASSPYSDEIVVQVTPAQQQEEPEMLWVTGPVLAVILIILIVIAILLFKRKRTHSPSSK
DEQSIGGG
cDNA Sequence Encoding Exemplary Sequence from PTPR LAR
(SEQ ID NO: 143)
GTACCCGGCAGACCGACAATGATGATCAGCACGACAGCGATGAACACGGCACTTCT
GCAATGGCATCCGCCCAAGGAATTGCCAGGGGAGTTGCTCGGCTACCGGCTGCAAT
ACTGTAGGGCAGATGAAGCAAGGCCGAACACAATCGATTTCGGAAAGGATGATCA
GCACTTTACTGTTACCGGACTTCATAAAGGGACAACTTATATCTTCCGGCTGGCGGC
TAAAAATAGAGCGGGCTTGGGAGAAGAATTCGAGAAGGAGATTAGAACCCCGGAG
GATTTGCCTAGTGGATTCCCGCAAAATCTCCATGTCACGGGACTCACTACTTCAACG
ACCGAACTCGCTTGGGATCCTCCAGTCCTTGCCGAAAGAAACGGAAGGATTATAAG
CTACACCGTAGTCTTCCGAGATATTAACTCACAACAAGAACTCCAGAATATTACTAC
CGATACGCGATTCACATTGACAGGACTTAAGCCTGACACGACTTATGATATAAAAG
TCCGGGCTTGGACTTCCAAAGGAAGCGGACCACTGTCTCCCTCAATCCAATCGAGG
ACTATGCCAGTTGAGCAAGTGTTTGCaAAAAACTTCAGAGTAGCGGCGGCGATGAA
AACTTCTGTCCTTCTGAGCTGGGAAGTTCCGGATTCCTATAAATCCGCTGTCCCCTTC
AAGATATTGTACAATGGTCAAAGTGTCGAGGTTGATGGACACTCCATGAGGAAACT
CATTGCCGACCTCCAACCGAACACAGAATACAGTTTTGTTTTGATGAATCGCGGTTC
CTCAGCCGGGGGCCTGCAGCACCTCGTGAGTATTAGGACCGCTCCCGATCTTCTTCC
ACATAAGCCTCTCCCCGCATCTGCGTACATAGAAGACGGGCGGTTCGACCTGTCAA
TGCCCCACGTACAGGACCCTAGTCTTGTAAGATGGTTTTATATCGTGGTCGTTCCTA
TAGACCGGGTTGGAGGCTCTATGCTCACCCCGCGATGGAGTACCCCGGAAGAACTT
GAGCTGGATGAGTTGTTGGAAGCAATCGAACAGGGGGGCGAGGAACAGAGGCGGC
GCCGCCGCCAAGCAGAGCGACTGAAACCGTATGTTGCCGCTCAGTTGGATGTGTTG
CCCGAAACGTTTACTTTGGGTGACAAAAAAAATTATCGGGGGTTCTACAATAGACC
TTTGAGTCCTGATCTTAGTTATCAGTGTTTTGTGTTGGCATCTTTGAAGGAACCGATG
GACCAGAAACGATACGCTTCCTCCCCCTATAGTGACGAGATTGTCGTTCAAGTAAC
ACCCGCACAACAGCAGGAGGAGCCGGAAATGCTTTGGGTTACAGGGCCGGTACTTG
CGGTGATCTTGATAATACTCATTGTCATAGCCATTCTGCTTTTCAAGCGCAAACGGA
CCCACTCACCCTCTTCCAAGGACGAGCAGAGCATAGGGGGAGGG
Exemplary Sequence from PTPR LAR
(SEQ ID NO: 146)
SGFPQNLHVTGLTTSTTELAWDPPVLAERNGRIISYTVVFRDINSQQELQNITTDTRFTLT
GLKPDTTYDIKVRAWTSKGSGPLSPSIQSRTMPVEQVFAKNFRVAAAMKTSVLLSWEV
PDSYKSAVPFKILYNGQSVEVDGHSMRKLIADLQPNTEYSFVLMNRGSSAGGLQHLVSI
RTAPDLLPHKPLPASAYIEDGRFDLSMPHVQDPSLVRWFYIVVVPIDRVGGSMLTPRWS
TPEELELDELLEAIEQGGEEQRRRRRQAERLKPYVAAQLDVLPETFTLGDKKNYRGFYN
RPLSPDLSYQCFVLASLKEPMDQKRYASSPYSDEIVVQVTPAQQQEEPEMLWVTGPVL
AVILIILIVIAILLFKRKRTHSPSSKDEQSIGGG
cDNA Sequence Encoding Exemplary Sequence from PTPR LAR
(SEQ ID NO: 147)
AGTGGATTCCCGCAAAATCTCCATGTCACGGGACTCACTACTTCAACGACCGAACTC
GCTTGGGATCCTCCAGTCCTTGCCGAAAGAAACGGAAGGATTATAAGCTACACCGT
AGTCTTCCGAGATATTAACTCACAACAAGAACTCCAGAATATTACTACCGATACGC
GATTCACATTGACAGGACTTAAGCCTGACACGACTTATGATATAAAAGTCCGGGCT
TGGACTTCCAAAGGAAGCGGACCACTGTCTCCCTCAATCCAATCGAGGACTATGCC
AGTTGAGCAAGTGTTTGCaAAAAACTTCAGAGTAGCGGCGGCGATGAAAACTTCTG
TCCTTCTGAGCTGGGAAGTTCCGGATTCCTATAAATCCGCTGTCCCCTTCAAGATAT
TGTACAATGGTCAAAGTGTCGAGGTTGATGGACACTCCATGAGGAAACTCATTGCC
GACCTCCAACCGAACACAGAATACAGTTTTGTTTTGATGAATCGCGGTTCCTCAGCC
GGGGGCCTGCAGCACCTCGTGAGTATTAGGACCGCTCCCGATCTTCTTCCACATAAG
CCTCTCCCCGCATCTGCGTACATAGAAGACGGGCGGTTCGACCTGTCAATGCCCCAC
GTACAGGACCCTAGTCTTGTAAGATGGTTTTATATCGTGGTCGTTCCTATAGACCGG
GTTGGAGGCTCTATGCTCACCCCGCGATGGAGTACCCCGGAAGAACTTGAGCTGGA
TGAGTTGTTGGAAGCAATCGAACAGGGGGGCGAGGAACAGAGGCGGCGCCGCCGC
CAAGCAGAGCGACTGAAACCGTATGTTGCCGCTCAGTTGGATGTGTTGCCCGAAAC
GTTTACTTTGGGTGACAAAAAAAATTATCGGGGGTTCTACAATAGACCTTTGAGTCC
TGATCTTAGTTATCAGTGTTTTGTGTTGGCATCTTTGAAGGAACCGATGGACCAGAA
ACGATACGCTTCCTCCCCCTATAGTGACGAGATTGTCGTTCAAGTAACACCCGCACA
ACAGCAGGAGGAGCCGGAAATGCTTTGGGTTACAGGGCCGGTACTTGCGGTGATCT
TGATAATACTCATTGTCATAGCCATTCTGCTTTTCAAGCGCAAACGGACCCACTCAC
CCTCTTCCAAGGACGAGCAGAGCATAGGGGGAGGG

In some embodiments, any of the chimeric transmembrane receptors provided herein further include a peptide nuclear localization sequence, e.g., operably linked to the transcriptional regulatory domain, such that upon intracellular cleavage the nuclear localization sequence is operably linked to the transcriptional regulatory domain that is released. An exemplary peptide nuclear localization sequence is shown below. Additional examples of peptide nuclear localization sequences are known in the art.

c-Myc Nuclear Localization Sequence
(SEQ ID NO: 58)
PAAKRVKLD
cDNA Sequence Encoding c-Myc Nuclear Localization
Sequence
(SEQ ID NO: 59)
CCCGCCGCCAAGAGAGTGAAGCTGGAC

In some embodiments, any of the chimeric transmembrane receptors can further include a signal sequence. A non-limiting example of a signal sequence is provided below. Additional examples of signal sequences are known in the art.

CSF2RA Signal Sequence
(SEQ ID NO: 41)
MLLLVTSLLLCELPHPAFLLIP
cDNA Encoding the CSF2RA Signal Sequence
(SEQ ID NO: 42)
ATGCTTCTCCTGGTGACAAGCCTTCTGCTCTGTGAGTTACCACACCCAGC
ATTCCTCTTGATCCCT

In some embodiments, any of the chimeric transmembrane receptors can further include a detectable label or tag. A non-limiting example of a detectable tag is a c-myc tag (e.g., the exemplary sequences below). Additional examples of detectable peptide labels are known in the art.

c-MycTag
(SEQ ID NO: 43)
EQKLISEEDL
cDNA Sequence Encoding a c-MycTag
(SEQ ID NO: 44)
GAACAAAAGCTGATCAGCGAGGAGGATCTC

Non-limiting examples of any of the chimeric transmembrane receptors described herein include an amino acid sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to any one of SEQ ID NOs: 39, 66, 76, 78, 86, 96, 102, 110, 116, 118, 120, 122, 124, 136, 140, and 144. In some examples, a chimeric transmembrane receptor described herein can include a sequence that is identical to any one of SEQ ID NOs: 39, 66, 76, 78, 86, 96, 102, 110, 116, 118, 120, 122, 124, 136, 140, and 144, except that it includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 amino acid substitutions.

Non-limiting examples of any of the chimeric transmembrane receptors described herein are encoded by a nucleic acid sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to any one of SEQ ID NOs: 40, 67, 77, 79, 87, 97, 103, 111, 117, 119, 121, 123, 125, 137, 141, and 145.

### Heterologous Target Genes

In some embodiments, chimeric transmembrane receptors provided herein include at least one intracellular transcriptional regulatory domain, which intracellular transcriptional regulatory domain regulates transcription of a heterologous target gene. In some embodiments, a heterologous target gene includes a transcription regulatory sequence (e.g., a promoter) that is operably linked to an expression sequence encoding a polypeptide (e.g., a recombinant protein). In some embodiments, a DNA binding domain of an intracellular transcriptional regulatory domain binds a transcription regulatory sequence (e.g., a promoter) that is operably linked to a sequence encoding a polypeptide (e.g., a recombinant protein). In some embodiments, an intracellular transcriptional regulatory domain activates transcription of a heterologous target gene. In some embodiments, a heterologous target gene includes an expression sequence encoding a polypeptide to be expressed in a cell that expresses the chimeric transmembrane receptor (e.g., after the extracellular antigen-binding domain of the chimeric transmembrane receptor binds its target antigen, resulting in release of the intracellular transcriptional regulatory domain from the transmembrane domain). In some embodiments, an intracellular transcriptional regulatory domain represses transcription of a heterologous target gene.

In some embodiments, a cell (e.g., an immune cell) expressing a chimeric transmembrane receptor includes a heterologous target gene, which heterologous target gene includes an expression sequence encoding a chimeric antigen receptor (CAR). In some embodiments, the intracellular transcriptional regulatory domain of the chimeric transmembrane receptor is a transcriptional activator that activates transcription of the CAR. In some embodiments, the expression sequence of the heterologous target gene encoding the CAR is operably linked to a transcriptional regulatory domain that is activated by the intracellular transcriptional regulatory domain of the chimeric transmembrane receptor.

In some embodiments, a cell (e.g., an immune cell) expressing a chimeric transmembrane receptor includes a heterologous target gene, which heterologous target gene includes an expression sequence encoding a first CAR polypeptide that is one polypeptide of a multi-polypeptide CAR (e.g., a CAR that includes two or more polypeptides, which together form a multi-polypeptide CAR having CAR activity). In some embodiments, the intracellular transcriptional regulatory domain of the chimeric transmembrane receptor is a transcriptional activator that activates transcription of the first CAR polypeptide. In some embodiments of cells having a heterologous target gene comprising an expression sequence encoding a first CAR polypeptide, one or more additional CAR polypeptides are expressed by the cell (e.g., one or more additional CAR polypeptides of a multi-polypeptide CAR). In some embodiments, the one or more additional CAR polypeptides of a multi-polypeptide CAR are constitutive expressed in the cell such that upon expression of the first CAR polypeptide (e.g., activation of transcription and subsequent translation of the first CAR polypeptide in response to the extracellular antigen-binding domain of the chimeric transmembrane receptor binding the target antigen), a functional multi-chain CAR is formed in the cell. In some embodiments, expression of the one or more additional CAR polypeptides of a multi-polypeptide CAR is regulated. For example, the one or more additional CAR polypeptides can be expressed in response to another chimeric transmembrane receptor that is specific for a different target antigen. As will be appreciated by those of ordinary skill in the art upon reading the present specification, such embodiments can further increase the specificity of a cell expressing the multiple chimeric transmembrane receptors for a cell expressing the multiple target antigens.

In some embodiments, a heterologous target gene including a nucleotide sequence encoding a CAR, a first CAR polypeptide, and/or one or more additional CAR polypeptides is present in the cell in a vector that has been transfected into the cell (e.g., using any of a variety of transfection techniques known in the art). In some embodiments, a heterologous target gene including a nucleotide sequence encoding a CAR, a first CAR polypeptide, and/or one or more additional CAR polypeptides is integrated into the genomic DNA of the cell (e.g., using any of a variety of genetic engineering techniques known in the art). Those of ordinary skill in the art will be aware of a large variety of CARs and polypeptide encoding them, each of which can be used in accordance with the heterologous target genes and methods provided herein.

In some embodiments, a CAR expressed in a cell (e.g. an immune cell, e.g., in response to the extracellular antigen-binding domain of a chimeric transmembrane receptor binding a target antigen) binds to a CAR target antigen selected from the group consisting of: BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, IL-13, IL-6, insulin-like growth factor I receptor, integrin α5β1, integrin ανβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. In some embodiments, the CAR target antigen is the same as the target antigen recognized by extracellular antigen-binding domain of a chimeric transmembrane receptor. In some embodiments, the CAR target antigen is different from the target antigen recognized by extracellular antigen-binding domain of a chimeric transmembrane receptor.

In some embodiments of a cell (e.g., an immune cell) expressing a chimeric transmembrane receptor and a CAR that is expressed in response to the extracellular antigen-binding domain of the chimeric transmembrane receptor binding a target antigen, such a cell recognizes a target cell expressing the target antigen and the CAR target antigen in a more specific manner than either: 1) a cell (e.g., an immune cell) expressing the chimeric transmembrane receptor in the absence of the CAR, or 2) a cell (e.g., an immune cell) expressing the CAR in the absence of the chimeric transmembrane receptor. Such cells expressing a chimeric transmembrane receptor and a CAR that is expressed in response to the extracellular antigen-binding domain of a chimeric transmembrane receptor binding a target antigen are advantageous in a number of ways. For example, such cells can be more specific for target cells (e.g., cancer cells) expressing the target antigen and the CAR target antigen. Additionally or alternatively, such cells can reduce adverse effects in a subject as compared to more conventional cells that, when administered therapeutically, aberrantly target non-cancer cells (e.g., non-target cells that may express low levels of the target antigen or the CAR target antigen, or an antigen that cross-reacts with the extracellular antigen-binding domain of the chimeric immune receptor or the CAR).

In some embodiments, a cell (e.g., an immune cell) expressing a chimeric transmembrane receptor includes a heterologous target gene, which heterologous target gene includes an expression sequence encoding a T cell receptor (TCR). In some embodiments, the intracellular transcriptional regulatory domain of the chimeric transmembrane receptor is a transcriptional activator that activates transcription of the TCR. In some embodiments, the expression sequence of the heterologous target gene encoding the TCR is operably linked to a transcriptional regulatory domain that is activated by the intracellular transcriptional regulatory domain of the chimeric transmembrane receptor.

In some embodiments, a cell (e.g., an immune cell) expressing a chimeric transmembrane receptor includes a heterologous target gene, which heterologous target gene includes an expression sequence encoding a first TCR polypeptide that is one polypeptide of a multi-polypeptide TCR (e.g., a TCR that includes two or more polypeptides, which together form a multi-polypeptide TCR having TCR activity). In some embodiments, the intracellular transcriptional regulatory domain of the chimeric transmembrane receptor is a transcriptional activator that activates transcription of the first TCR polypeptide. In some embodiments of cells having a heterologous target gene comprising an expression sequence encoding a first TCR polypeptide, one or more additional TCR polypeptides are expressed by the cell (e.g., one or more additional TCR polypeptides of a multi-polypeptide TCR). In some embodiments, the one or more additional TCR polypeptides of a multi-polypeptide TCR are constitutive expressed in the cell such that upon expression of the first TCR polypeptide (e.g., activation of transcription and subsequent translation of the first TCR polypeptide in response to the extracellular antigen-binding domain of the chimeric transmembrane receptor binding the target antigen), a functional multi-chain TCR is formed in the cell. In some embodiments, expression of the one or more additional TCR polypeptides of a multi-polypeptide TCR is regulated. For example, the one or more additional TCR polypeptides can be expressed in response to another chimeric transmembrane receptor that is specific for a different target antigen. As will be appreciated by those of ordinary skill in the art upon reading the present specification, such embodiments can further increase the specificity of a cell expressing the multiple chimeric transmembrane receptors for a cell expressing the multiple target antigens.

In some embodiments, a heterologous target gene including a nucleotide sequence encoding a TCR, a first TCR polypeptide, and/or one or more additional TCR polypeptides is present in the cell in a vector that has been transfected into the cell (e.g., using any of a variety of transfection techniques known in the art). In some embodiments, a heterologous target gene including a nucleotide sequence encoding a TCR, a first TCR polypeptide, and/or one or more additional TCR polypeptides is integrated into the genomic DNA of the cell (e.g., using any of a variety of genetic engineering techniques known in the art). Those of ordinary skill in the art will be aware of a large variety of TCRs and polypeptide encoding them, each of which can be used in accordance with the heterologous target genes and methods provided herein.

In some embodiments, a TCR expressed in a cell (e.g. an immune cell, e.g., in response to the extracellular antigen-binding domain of a chimeric transmembrane receptor binding a target antigen) binds to a TCR target antigen selected from the group consisting of: BCMA, MAGE, MUC16, CD19, WT-1, CD22, LI-CAM, ROR-1, CEA, 4-1BB, ETA, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD20, CD125 CD200, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgGl, IL-13, IL-6, insulin-like growth factor I receptor, integrin α5β1, integrin ανβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. In some embodiments, the TCR target antigen is the same as the target antigen recognized by extracellular antigen-binding domain of a chimeric transmembrane receptor. In some embodiments, the TCR target antigen is different from the target antigen recognized by extracellular antigen-binding domain of a chimeric transmembrane receptor.

In some embodiments of a cell (e.g., an immune cell) expressing a chimeric transmembrane receptor and a TCR that is expressed in response to the extracellular antigen-binding domain of the chimeric transmembrane receptor binding a target antigen, such a cell recognizes a target cell expressing the target antigen and the TCR target antigen in a more specific manner than either: 1) a cell (e.g., an immune cell) expressing the chimeric transmembrane receptor in the absence of the TCR, or 2) a cell (e.g., an immune cell) expressing the TCR in the absence of the chimeric transmembrane receptor. Such cells expressing a chimeric transmembrane receptor and a TCR that is expressed in response to the extracellular antigen-binding domain of a chimeric transmembrane receptor binding a target antigen are advantageous in a number of ways. For example, such cells can be more specific for target cells (e.g., cancer cells) expressing the target antigen and the TCR target antigen. Additionally or alternatively, such cells can reduce adverse effects in a subject as compared to more conventional cells that, when administered therapeutically, aberrantly target non-cancer cells (e.g., non-target cells that may express low levels of the target antigen or the TCR target antigen, or an antigen that cross-reacts with the extracellular antigen-binding domain of the chimeric immune receptor or the TCR).

In some embodiments, a cell (e.g., an immune cell) expressing a chimeric transmembrane receptor includes a heterologous target gene, which heterologous target gene includes an expression sequence encoding a secreted polypeptide. In some embodiments, the intracellular transcriptional regulatory domain of the chimeric transmembrane receptor is a transcriptional activator that activates transcription of the secreted polypeptide. In some embodiments, the expression sequence of the heterologous target gene encoding the secreted polypeptide is operably linked to a transcriptional regulatory domain that is activated by the intracellular transcriptional regulatory domain of the chimeric transmembrane receptor.

In some embodiments, a heterologous target gene including a nucleotide sequence encoding a secreted polypeptide is present in the cell in a vector that has been transfected into the cell (e.g., using any of a variety of transfection techniques known in the art). In some embodiments, a heterologous target gene including a nucleotide sequence encoding a secreted polypeptide is integrated into the genomic DNA of the cell (e.g., using any of a variety of genetic engineering techniques known in the art). Those of ordinary skill in the art will be aware of a large variety of secreted polypeptides, each of which can be used in accordance with the heterologous target genes and methods provided herein.

In some embodiments, a heterologous target gene includes an expression sequence encoding a cytokine. Non-limiting examples of cytokines include, e.g., interferons (e.g., an alpha-interferon, a beta-interferon, a gamma-interferon); interleukins (e.g., IL-1, IL-1α, IL-2, IL-3, IL-4, IL-5, IL-6, IL-7, IL-8, IL-9, IL-10 IL-11, IL-12; IL-13, IL-14, IL-15, IL-16, IL-17, IL-17A, IL-18, IL-19, IL-20, IL-24); tumor necrosis factors (e.g., TNF-α); transforming growth factor-beta; and TRAIL. In some embodiments of a cell (e.g., an immune cell) expressing a chimeric transmembrane receptor and a cytokine that is expressed in response to the extracellular antigen-binding domain of the chimeric transmembrane receptor binding a target antigen, such a cell recognizes a target cell expressing the target antigen and effectively acts on the target cell (e.g., by mediating an increased immune response against the target cell) in an increased manner relative to either: 1) a cell (e.g., an immune cell) expressing the chimeric transmembrane receptor in the absence of the cytokine, or 2) a cell (e.g., an immune cell) expressing the cytokine in the absence of the chimeric transmembrane receptor. In addition to more effectively acting on the target cell, such cells expressing a chimeric transmembrane receptor and a cytokine that is expressed in response to the extracellular antigen-binding domain of a chimeric transmembrane receptor binding a target antigen are advantageous in a number of ways. For example, the dosage (e.g., the number of immune cells) of such relatively hyperactive cells can be reduced, thus reducing adverse side effects in a subject.

Other non-limiting examples of a polypeptide encoded by an expression sequence of a heterologous target gene include: an apoptosis inducer, an apoptosis inhibitor, an antibody (e.g., an antibody, an antibody fragment, or an antibody derivative), a chemokine, a chemokine receptor, a cytokine receptor, a differentiation factor, a growth factor, a growth factor receptor, a hormone, a metabolic enzyme, a pathogen derived protein, a proliferation inducer, a receptor, a RNA guided nuclease, a site-specific nuclease, a small molecule second messenger synthesis enzyme, a toxin derived protein, a transcription activator, a transcription repressor, a transcriptional activator, a transcriptional repressor, a translation regulator, a translational activator, and a translational repressor. In some embodiments, a polypeptide encoded by an expression sequence of a heterologous target gene includes a second chimeric immune receptor (e.g., a chimeric immune receptor that binds a different target antigen than the target antigen bound by the chimeric immune receptor that actives transcription of the second chimeric immune receptor).

### Expression of a Chimeric Transmembrane Receptor in a Cell

Also provided herein are methods of generating a recombinant cell that expresses an chimeric transmembrane receptor (e.g., any of the chimeric transmembrane receptors described herein) that include: introducing into a cell a nucleic acid sequence encoding the chimeric transmembrane receptor to produce a recombinant cell; and culturing the recombinant cell under conditions sufficient for the expression of the chimeric transmembrane receptor. In some embodiments, the introducing step includes introducing into a cell an expression vector including a sequence encoding the chimeric transmembrane receptor to produce a recombinant cell. In some embodiments, a sequence encoding the chimeric transmembrane receptor is operably linked to a promoter. Exemplary promoters include those derived from polyoma, Adenovirus 2, cytomegalovirus and SV40.

In some embodiments, a nucleic acid sequence encoding the chimeric transmembrane receptor includes a first nucleic acid segment that encodes an extracellular antigen-binding domain that is capable of specifically binding to a target antigen, a second nucleic acid segment that encodes an extracellular integrin ligand-binding domain comprising an S1 protease cleavage site, an S2 protease cleavage site, or both, a third nucleic acid segment that encodes a transmembrane domain, a fourth nucleic acid segment that encodes an intracellular regulatory domain comprising a gamma-secretase protease cleavage site; and a fifth nucleic acid segment that encodes an intracellular transcriptional regulatory domain. In some embodiments, a nucleic acid sequence encoding the chimeric transmembrane receptor includes a nucleic acid segment that encodes an extracellular integrin ligand-binding domain comprising an S1 protease cleavage site and an S2 protease cleavage site (e.g., the nucleic acid sequence encoding the chimeric transmembrane receptor includes a nucleic acid segment that encodes a single extracellular integrin ligand-binding domain having both an S1 cleavage site and an S2 cleavage site). In some embodiments, a nucleic acid sequence encoding the chimeric transmembrane receptor includes a nucleic acid segment that encodes an extracellular integrin ligand-binding domain comprising an S1 protease cleavage site, and a separate nucleic acid segment that encodes an extracellular integrin ligand-binding domain comprising an S2 protease cleavage site. In some embodiments, a chimeric transmembrane receptor is expressed from a nucleic acid sequence encoding the chimeric transmembrane receptor (e.g. via a promoter that is operably linked to the nucleic acid sequence encoding the chimeric transmembrane receptor). In some embodiments, a chimeric transmembrane receptor that is expressed from a nucleic acid sequence encoding the chimeric transmembrane receptor is processed to produce a mature chimeric transmembrane receptor (e.g., via furin cleavage the S1 cleavage site), which mature chimeric transmembrane receptor is properly expressed on the cell surface such that it can function in accordance with the various embodiments disclosed herein.

Nucleic acid sequences encoding a chimeric transmembrane receptor can be readily prepared by a person of ordinary skill in the art using the information and references contained herein and techniques known in the art. Sambrook, et al., A Molecular Cloning, A Laboratory Manual, Cold Spring Harbor Laboratory Press (1989-2016), and Ausubel et al., Current Protocols in Molecular Biology, John Wiley and Sons, (1994-2016)). Non-limiting examples of such techniques include (i) the use of the polymerase chain reaction (PCR) to amplify samples of such nucleic acid, e.g. from genomic sources, (ii) chemical synthesis, or (iii) preparing cDNA sequences. DNA encoding portions of full-length coding sequences may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the relevant sequence may be made, e.g. using site directed mutagenesis, to lead to the expression of modified peptide or to take account of codon preference in the host cells used to express the nucleic acid.

A chimeric transmembrane receptor described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Mammalian cell lines available in the art for expression of a heterologous polypeptide include Chinese hamster ovary cells, HeLa cells, baby hamster kidney cells, COS cells and a variety of others. As used herein, the term “prokaryotic cell” refers to a cell that does not have a distinct, membrane-bound nucleus. In some embodiments, the prokaryotic cell is a bacterial cell. A common bacterial host is E. coli.

In some embodiments, a chimeric transmembrane receptor is expressed in a cell selected from the group consisting of: a CD4+ T cell, a CD8+ T cell, a B cell, a monocyte, a natural killer cell, a dendritic cell, a macrophage, a regulatory T cell, or a helper T cell. In some embodiments, a chimeric transmembrane receptor is expressed in a cell (e.g., an immune cell) that administered to a subject, which cell is autologous to a subject. For example, an immune cell can be isolated from a subject, transfected with an expression vector encoding the chimeric transmembrane receptor, and subsequently administered back to the subject. In some embodiments, a chimeric transmembrane receptor is expressed in a cell (e.g., an immune cell) that administered to a subject, which cell is allogeneic to a subject. For example, an immune cell can be isolated from a donor (e.g., another human), transfected with an expression vector encoding the chimeric transmembrane receptor, and subsequently administered to the subject. In some embodiments, the immune cell that is isolated from the donor is further manipulated to reduce adverse immune responses in the subject and/or improve therapeutic outcomes. For example, nucleic acids encoding one or more endogenous proteins in the cell that lead to an adverse immune response (or otherwise contribute to a poor therapeutic outcome) when the cell is administered to the subject can be modified such that expression of the endogenous protein(s) is reduced or eliminated. Those of ordinary skill in the art will be aware of other suitable techniques for modifying allogeneic cells from a donor to reduce adverse immune responses in the subject and/or improve therapeutic outcomes.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

In some embodiments, expression of a chimeric transmembrane receptor in a cell is regulated by one or more mechanisms. For example, a nucleic acid comprising a nucleotide sequence encoding a chimeric transmembrane receptor can be operably linked to a promoter, an enhancer, or both. Suitable promoters (e.g., inducible promoters) and enchancers for regulating expression of vectors encoding polypeptides in cells are known to those of ordinary skill in the art. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular cloning: a Laboratory Manual: 4th edition, Green and Sambrook et al., 2012, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al., Eds., John Wiley & Sons, 2016.

Provided herein are methods that further include isolation of the chimeric transmembrane receptor from a cell (e.g., a eukaryotic cell) using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

### Methods of Treatment

Provided herein are methods of treating a cancer that include: administering a therapeutically effective amount of a nucleic acid encoding any of the chimeric transmembrane receptors described herein, any of the mammalian cells (e.g., immune cells) described herein, or any of the pharmaceutical compositions described herein to a subject identified as having a cancer. In some embodiments of any of the methods described herein, the cancer is a primary tumor. In some embodiments of any of the methods described herein, the cancer is a metastasis. In some embodiments of any of the methods described herein, the cancer is a T-cell-infiltrating tumor. In some embodiments of any of the methods described herein, the cancer is a non-T-cell-infiltrating tumor.

Also provided herein are methods of reducing the volume of a tumor in a subject that include: administering a therapeutically effective amount of a nucleic acid encoding any of the chimeric transmembrane receptors described herein, any of the mammalian cells (e.g., immune cells described herein), or any of the pharmaceutical compositions described herein to a subject identified as having a cancer. In some embodiments, methods provided herein can result in an about 1% to about 100% reduction, an about 1% to about 95% reduction, an about 1% to about 90% reduction, an about 1% to about 85% reduction, an about 1% to about 80% reduction, an about 1% to about 75% reduction, an about 1% to about 70% reduction, an about 1% to about 65% reduction, an about 1% to about 60% reduction, an about 1% to about 55% reduction, an about 1% to about 50% reduction, an about 1% to about 45% reduction, an about 1% to about 40% reduction, an about 1% to about 35% reduction, an about 1% to about 30% reduction, an about 1% to about 25% reduction, an about 1% to about 20% reduction, an about 1% to about 15% reduction, an about 1% to about 10% reduction, an about 1% to about 5% reduction, an about 5% to about 100% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 100% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 100% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, an about 15% to about 20% reduction, an about 20% to about 100% reduction, an about 20% to about 95% reduction, an about 20% to about 90% reduction, an about 20% to about 85% reduction, an about 20% to about 80% reduction, an about 20% to about 75% reduction, an about 20% to about 70% reduction, an about 20% to about 65% reduction, an about 20% to about 60% reduction, an about 20% to about 55% reduction, an about 20% to about 50% reduction, an about 20% to about 45% reduction, an about 20% to about 40% reduction, an about 20% to about 35% reduction, an about 20% to about 30% reduction, an about 20% to about 25% reduction, an about 25% to about 100% reduction, an about 25% to about 95% reduction, an about 25% to about 90% reduction, an about 25% to about 85% reduction, an about 25% to about 80% reduction, an about 25% to about 75% reduction, an about 25% to about 70% reduction, an about 25% to about 65% reduction, an about 25% to about 60% reduction, an about 25% to about 55% reduction, an about 25% to about 50% reduction, an about 25% to about 45% reduction, an about 25% to about 40% reduction, an about 25% to about 35% reduction, an about 25% to about 30% reduction, an about 30% to about 100% reduction, an about 30% to about 95% reduction, an about 30% to about 90% reduction, an about 30% to about 85% reduction, an about 30% to about 80% reduction, an about 30% to about 75% reduction, an about 30% to about 70% reduction, an about 30% to about 65% reduction, an about 30% to about 60% reduction, an about 30% to about 55% reduction, an about 30% to about 50% reduction, an about 30% to about 45% reduction, an about 30% to about 40% reduction, an about 30% to about 35% reduction, an about 35% to about 100% reduction, an about 35% to about 95% reduction, an about 35% to about 90% reduction, an about 35% to about 85% reduction, an about 35% to about 80% reduction, an about 35% to about 75% reduction, an about 35% to about 70% reduction, an about 35% to about 65% reduction, an about 35% to about 60% reduction, an about 35% to about 55% reduction, an about 35% to about 50% reduction, an about 35% to about 45% reduction, an about 35% to about 40% reduction, an about 40% to about 100% reduction, an about 40% to about 95% reduction, an about 40% to about 90% reduction, an about 40% to about 85% reduction, an about 40% to about 80% reduction, an about 40% to about 75% reduction, an about 40% to about 70% reduction, an about 40% to about 65% reduction, an about 40% to about 60% reduction, an about 40% to about 55% reduction, an about 40% to about 50% reduction, an about 40% to about 45% reduction, an about 45% to about 100% reduction, an about 45% to about 95% reduction, an about 45% to about 90% reduction, an about 45% to about 85% reduction, an about 45% to about 80% reduction, an about 45% to about 75% reduction, an about 45% to about 70% reduction, an about 45% to about 65% reduction, an about 45% to about 60% reduction, an about 45% to about 55% reduction, an about 45% to about 50% reduction, an about 50% to about 100% reduction, an about 50% to about 95% reduction, an about 50% to about 90% reduction, an about 50% to about 85% reduction, an about 50% to about 80% reduction, an about 50% to about 75% reduction, an about 50% to about 70% reduction, an about 50% to about 65% reduction, an about 50% to about 60% reduction, an about 50% to about 55% reduction, an about 55% to about 100% reduction, an about 55% to about 95% reduction, an about 55% to about 90% reduction, an about 55% to about 85% reduction, an about 55% to about 80% reduction, an about 55% to about 75% reduction, an about 55% to about 70% reduction, an about 55% to about 65% reduction, an about 55% to about 60% reduction, an about 60% to about 100% reduction, an about 60% to about 95% reduction, an about 60% to about 90% reduction, an about 60% to about 85% reduction, an about 60% to about 80% reduction, an about 60% to about 75% reduction, an about 60% to about 70% reduction, an about 60% to about 65% reduction, an about 65% to about 100% reduction, an about 65% to about 95% reduction, an about 65% to about 90% reduction, an about 65% to about 85% reduction, an about 65% to about 80% reduction, an about 65% to about 75% reduction, an about 65% to about 70% reduction, an about 70% to about 100% reduction, an about 70% to about 95% reduction, an about 70% to about 90% reduction, an about 70% to about 85% reduction, an about 70% to about 80% reduction, an about 70% to about 75% reduction, an about 75% to about 100% reduction, an about 75% to about 95% reduction, an about 75% to about 90% reduction, an about 75% to about 85% reduction, an about 75% to about 80% reduction, an about 80% to about 100% reduction, an about 80% to about 95% reduction, an about 80% to about 90% reduction, an about 80% to about 85% reduction, an about 85% to about 100% reduction, an about 85% to about 95% reduction, an about 85% to about 90% reduction, an about 90% to about 100% reduction, an about 90% to about 95% reduction, or an about 95% to about 100% reduction in the volume of at least one (e.g., at one, two, three, four, or five) tumor (e.g., a liquid tumor or a solid tumor) in a subject (e.g., a human), e.g., as compared to the volume of the at least one tumor prior to the administering.

Also provided herein are methods of inducing cell death in a cancer cell in a subject that include: administering a therapeutically effective amount of a nucleic acid encoding any of the chimeric transmembrane receptors described herein, any of the mammalian cells (e.g., immune cells), or any of the pharmaceutical compositions described herein to a subject identified as having a cancer. In some embodiments, methods provided herein can result in an about 1% to about 100% reduction, an about 1% to about 95% reduction, an about 1% to about 90% reduction, an about 1% to about 85% reduction, an about 1% to about 80% reduction, an about 1% to about 75% reduction, an about 1% to about 70% reduction, an about 1% to about 65% reduction, an about 1% to about 60% reduction, an about 1% to about 55% reduction, an about 1% to about 50% reduction, an about 1% to about 45% reduction, an about 1% to about 40% reduction, an about 1% to about 35% reduction, an about 1% to about 30% reduction, an about 1% to about 25% reduction, an about 1% to about 20% reduction, an about 1% to about 15% reduction, an about 1% to about 10% reduction, an about 1% to about 5% reduction, an about 5% to about 100% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 100% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 100% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, an about 15% to about 20% reduction, an about 20% to about 100% reduction, an about 20% to about 95% reduction, an about 20% to about 90% reduction, an about 20% to about 85% reduction, an about 20% to about 80% reduction, an about 20% to about 75% reduction, an about 20% to about 70% reduction, an about 20% to about 65% reduction, an about 20% to about 60% reduction, an about 20% to about 55% reduction, an about 20% to about 50% reduction, an about 20% to about 45% reduction, an about 20% to about 40% reduction, an about 20% to about 35% reduction, an about 20% to about 30% reduction, an about 20% to about 25% reduction, an about 25% to about 100% reduction, an about 25% to about 95% reduction, an about 25% to about 90% reduction, an about 25% to about 85% reduction, an about 25% to about 80% reduction, an about 25% to about 75% reduction, an about 25% to about 70% reduction, an about 25% to about 65% reduction, an about 25% to about 60% reduction, an about 25% to about 55% reduction, an about 25% to about 50% reduction, an about 25% to about 45% reduction, an about 25% to about 40% reduction, an about 25% to about 35% reduction, an about 25% to about 30% reduction, an about 30% to about 100% reduction, an about 30% to about 95% reduction, an about 30% to about 90% reduction, an about 30% to about 85% reduction, an about 30% to about 80% reduction, an about 30% to about 75% reduction, an about 30% to about 70% reduction, an about 30% to about 65% reduction, an about 30% to about 60% reduction, an about 30% to about 55% reduction, an about 30% to about 50% reduction, an about 30% to about 45% reduction, an about 30% to about 40% reduction, an about 30% to about 35% reduction, an about 35% to about 100% reduction, an about 35% to about 95% reduction, an about 35% to about 90% reduction, an about 35% to about 85% reduction, an about 35% to about 80% reduction, an about 35% to about 75% reduction, an about 35% to about 70% reduction, an about 35% to about 65% reduction, an about 35% to about 60% reduction, an about 35% to about 55% reduction, an about 35% to about 50% reduction, an about 35% to about 45% reduction, an about 35% to about 40% reduction, an about 40% to about 100% reduction, an about 40% to about 95% reduction, an about 40% to about 90% reduction, an about 40% to about 85% reduction, an about 40% to about 80% reduction, an about 40% to about 75% reduction, an about 40% to about 70% reduction, an about 40% to about 65% reduction, an about 40% to about 60% reduction, an about 40% to about 55% reduction, an about 40% to about 50% reduction, an about 40% to about 45% reduction, an about 45% to about 100% reduction, an about 45% to about 95% reduction, an about 45% to about 90% reduction, an about 45% to about 85% reduction, an about 45% to about 80% reduction, an about 45% to about 75% reduction, an about 45% to about 70% reduction, an about 45% to about 65% reduction, an about 45% to about 60% reduction, an about 45% to about 55% reduction, an about 45% to about 50% reduction, an about 50% to about 100% reduction, an about 50% to about 95% reduction, an about 50% to about 90% reduction, an about 50% to about 85% reduction, an about 50% to about 80% reduction, an about 50% to about 75% reduction, an about 50% to about 70% reduction, an about 50% to about 65% reduction, an about 50% to about 60% reduction, an about 50% to about 55% reduction, an about 55% to about 100% reduction, an about 55% to about 95% reduction, an about 55% to about 90% reduction, an about 55% to about 85% reduction, an about 55% to about 80% reduction, an about 55% to about 75% reduction, an about 55% to about 70% reduction, an about 55% to about 65% reduction, an about 55% to about 60% reduction, an about 60% to about 100% reduction, an about 60% to about 95% reduction, an about 60% to about 90% reduction, an about 60% to about 85% reduction, an about 60% to about 80% reduction, an about 60% to about 75% reduction, an about 60% to about 70% reduction, an about 60% to about 65% reduction, an about 65% to about 100% reduction, an about 65% to about 95% reduction, an about 65% to about 90% reduction, an about 65% to about 85% reduction, an about 65% to about 80% reduction, an about 65% to about 75% reduction, an about 65% to about 70% reduction, an about 70% to about 100% reduction, an about 70% to about 95% reduction, an about 70% to about 90% reduction, an about 70% to about 85% reduction, an about 70% to about 80% reduction, an about 70% to about 75% reduction, an about 75% to about 100% reduction, an about 75% to about 95% reduction, an about 75% to about 90% reduction, an about 75% to about 85% reduction, an about 75% to about 80% reduction, an about 80% to about 100% reduction, an about 80% to about 95% reduction, an about 80% to about 90% reduction, an about 80% to about 85% reduction, an about 85% to about 100% reduction, an about 85% to about 95% reduction, an about 85% to about 90% reduction, an about 90% to about 100% reduction, an about 90% to about 95% reduction, or an about 95% to about 100% reduction in the number of cancer cells in a subject (e.g., a human), e.g., as compared to the number of cancer cells in the subject prior to the administering or as compared to a control subject having the same type of cancer and receiving a different treatment.

Provided herein are methods of decreasing the risk of developing a metastasis or decreasing the risk of developing an additional metastasis in a subject having a cancer that include: administering a therapeutically effective amount of a nucleic acid encoding any of the chimeric transmembrane receptors described herein, any of the mammalian cells (e.g., immune cells), or any of the pharmaceutical compositions described herein to a subject identified as having a cancer. In some embodiments, methods provided herein provide for an about 1% to about 100% reduction, an about 1% to about 95% reduction, an about 1% to about 90% reduction, an about 1% to about 85% reduction, an about 1% to about 80% reduction, an about 1% to about 75% reduction, an about 1% to about 70% reduction, an about 1% to about 65% reduction, an about 1% to about 60% reduction, an about 1% to about 55% reduction, an about 1% to about 50% reduction, an about 1% to about 45% reduction, an about 1% to about 40% reduction, an about 1% to about 35% reduction, an about 1% to about 30% reduction, an about 1% to about 25% reduction, an about 1% to about 20% reduction, an about 1% to about 15% reduction, an about 1% to about 10% reduction, an about 1% to about 5% reduction, an about 5% to about 100% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 100% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 100% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, an about 15% to about 20% reduction, an about 20% to about 100% reduction, an about 20% to about 95% reduction, an about 20% to about 90% reduction, an about 20% to about 85% reduction, an about 20% to about 80% reduction, an about 20% to about 75% reduction, an about 20% to about 70% reduction, an about 20% to about 65% reduction, an about 20% to about 60% reduction, an about 20% to about 55% reduction, an about 20% to about 50% reduction, an about 20% to about 45% reduction, an about 20% to about 40% reduction, an about 20% to about 35% reduction, an about 20% to about 30% reduction, an about 20% to about 25% reduction, an about 25% to about 100% reduction, an about 25% to about 95% reduction, an about 25% to about 90% reduction, an about 25% to about 85% reduction, an about 25% to about 80% reduction, an about 25% to about 75% reduction, an about 25% to about 70% reduction, an about 25% to about 65% reduction, an about 25% to about 60% reduction, an about 25% to about 55% reduction, an about 25% to about 50% reduction, an about 25% to about 45% reduction, an about 25% to about 40% reduction, an about 25% to about 35% reduction, an about 25% to about 30% reduction, an about 30% to about 100% reduction, an about 30% to about 95% reduction, an about 30% to about 90% reduction, an about 30% to about 85% reduction, an about 30% to about 80% reduction, an about 30% to about 75% reduction, an about 30% to about 70% reduction, an about 30% to about 65% reduction, an about 30% to about 60% reduction, an about 30% to about 55% reduction, an about 30% to about 50% reduction, an about 30% to about 45% reduction, an about 30% to about 40% reduction, an about 30% to about 35% reduction, an about 35% to about 100% reduction, an about 35% to about 95% reduction, an about 35% to about 90% reduction, an about 35% to about 85% reduction, an about 35% to about 80% reduction, an about 35% to about 75% reduction, an about 35% to about 70% reduction, an about 35% to about 65% reduction, an about 35% to about 60% reduction, an about 35% to about 55% reduction, an about 35% to about 50% reduction, an about 35% to about 45% reduction, an about 35% to about 40% reduction, an about 40% to about 100% reduction, an about 40% to about 95% reduction, an about 40% to about 90% reduction, an about 40% to about 85% reduction, an about 40% to about 80% reduction, an about 40% to about 75% reduction, an about 40% to about 70% reduction, an about 40% to about 65% reduction, an about 40% to about 60% reduction, an about 40% to about 55% reduction, an about 40% to about 50% reduction, an about 40% to about 45% reduction, an about 45% to about 100% reduction, an about 45% to about 95% reduction, an about 45% to about 90% reduction, an about 45% to about 85% reduction, an about 45% to about 80% reduction, an about 45% to about 75% reduction, an about 45% to about 70% reduction, an about 45% to about 65% reduction, an about 45% to about 60% reduction, an about 45% to about 55% reduction, an about 45% to about 50% reduction, an about 50% to about 100% reduction, an about 50% to about 95% reduction, an about 50% to about 90% reduction, an about 50% to about 85% reduction, an about 50% to about 80% reduction, an about 50% to about 75% reduction, an about 50% to about 70% reduction, an about 50% to about 65% reduction, an about 50% to about 60% reduction, an about 50% to about 55% reduction, an about 55% to about 100% reduction, an about 55% to about 95% reduction, an about 55% to about 90% reduction, an about 55% to about 85% reduction, an about 55% to about 80% reduction, an about 55% to about 75% reduction, an about 55% to about 70% reduction, an about 55% to about 65% reduction, an about 55% to about 60% reduction, an about 60% to about 100% reduction, an about 60% to about 95% reduction, an about 60% to about 90% reduction, an about 60% to about 85% reduction, an about 60% to about 80% reduction, an about 60% to about 75% reduction, an about 60% to about 70% reduction, an about 60% to about 65% reduction, an about 65% to about 100% reduction, an about 65% to about 95% reduction, an about 65% to about 90% reduction, an about 65% to about 85% reduction, an about 65% to about 80% reduction, an about 65% to about 75% reduction, an about 65% to about 70% reduction, an about 70% to about 100% reduction, an about 70% to about 95% reduction, an about 70% to about 90% reduction, an about 70% to about 85% reduction, an about 70% to about 80% reduction, an about 70% to about 75% reduction, an about 75% to about 100% reduction, an about 75% to about 95% reduction, an about 75% to about 90% reduction, an about 75% to about 85% reduction, an about 75% to about 80% reduction, an about 80% to about 100% reduction, an about 80% to about 95% reduction, an about 80% to about 90% reduction, an about 80% to about 85% reduction, an about 85% to about 100% reduction, an about 85% to about 95% reduction, an about 85% to about 90% reduction, an about 90% to about 100% reduction, an about 90% to about 95% reduction, or an about 95% to about 100% reduction in the risk of developing a metastasis or developing an additional metastasis in the subject (e.g., a human), e.g., as compared to a subject having a similar cancer and receiving no treatment or receiving a different treatment.

As used herein, treating includes reducing the number, frequency, or severity of one or more (e.g., two, three, four, or five) signs or symptoms of a cancer in a patient having a cancer (e.g., any of the cancers described herein). For example, treatment can reduce cancer progression, reduce the severity of a cancer, or reduce the risk of re-occurrence of a cancer in a subject having the cancer.

In some embodiments, a therapeutically effective amount of a nucleic acid encoding any of the chimeric transmembrane receptors described herein, any of the mammalian cells (e.g., immune cells) described herein, or any of the pharmaceutical compositions described herein is administered to a subject in combination with one or more additional anti-cancer therapies. Such additional anti-cancer therapies include, without limitation, chemotherapy, immunotherapy, surgical resection, and radiation therapy.

Non-limiting examples of cancers than can be treated using compositions and methods described herein include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor. Additional examples of cancer are known in the art.

### Compositions and Kits

Also provided herein are compositions (e.g., pharmaceutical compositions) that include a nucleic acid encoding any of the chimeric transmembrane receptors described herein. In some embodiments, the compositions (e.g., pharmaceutical compositions) can be disposed in a sterile vial or a pre-loaded syringe. In some embodiments, a composition (e.g., a pharmaceutical composition) can further include a nucleic acid including (i) a transcription regulatory sequence that is capable of being specifically recognized by the intracellular transcriptional regulatory domain and (ii) a nucleic acid sequence that encodes a recombinant protein, where the nucleic acid sequence that encodes the recombinant protein is operably linked to the transcription regulatory sequence.

In some embodiments, a pharmaceutical composition can include any of the mammalian cells (e.g., immune cells) described herein.

In some embodiments, the compositions (e.g., pharmaceutical compositions) are formulated for different routes of administration (e.g., intravenous, subcutaneous, intramuscular, or intratumoral). In some embodiments, the compositions (e.g., pharmaceutical compositions) can include a pharmaceutically acceptable carrier (e.g., phosphate buffered saline). Single or multiple administrations of any of the pharmaceutical compositions described herein can be given to a subject depending on, for example: the dosage and frequency as required and tolerated by the patient. A dosage of the pharmaceutical composition should provide a sufficient quantity of the chimeric transmembrane receptors to effectively treat or ameliorate conditions, diseases, or symptoms.

Also provided herein are methods of treating a subject having a cancer (e.g., any of the cancers described herein) that include administering a therapeutically effective amount of at least one of any of the compositions or pharmaceutical compositions provided herein.

Also provided herein are kits that include any of the chimeric transmembrane receptors described herein, any of the nucleic acids described herein, any of the compositions described herein, or any of the pharmaceutical compositions described herein. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein.

The invention is further described in the following examples, which do not limit the scope of the invention described in the claims.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Materials and Methods

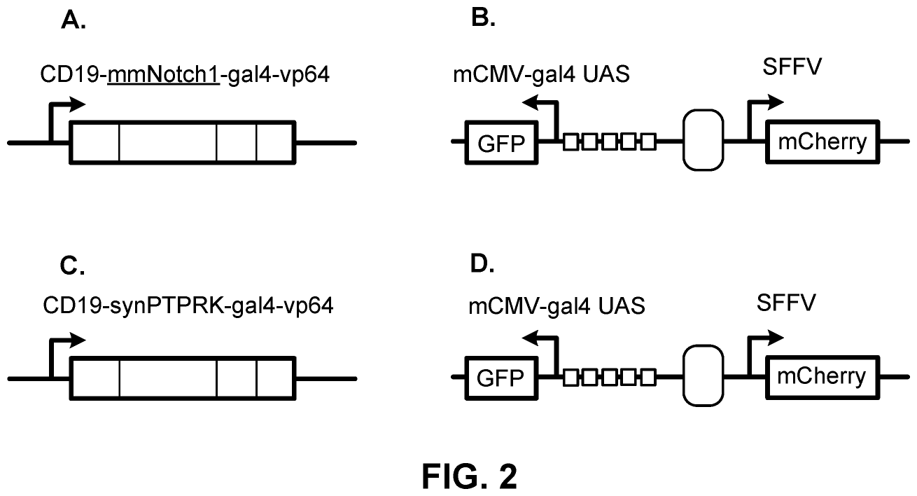

Plasmids constructs encoding synNotch, synPTPR, and the reporter constructs were created by Golden Gate assembly composed of synthetic genes (gBlocks) ordered from IDT and a backbone plasmid with lentiviral compatibility which was created at Cell Design Labs. See FIG. 2 and its description herein for detailed information on the created plasmid constructs.

The plasmids were then transfected into suspension culture (K562 cells) with helper plasmids and incubated to produce high titer virus containing the synNotch or synPTPR constructs.

The constructs were then transduced into Jurkat immortalized human T lymphocyte cells by mixing 75 μL of supernatant for the synNotch or synPTPR constructs and 50 μL of the reporter into 500,000 million cells in 1 mL of media. The cells were then spun in a centrifuge at 1,000 g for 1.5 hours. Afterwards, the cells were incubated for 24 hours before the supernatant including the virus was removed and replaced with fresh media.

After 6 days, the cells were co-cultured overnight with CD19 antigen-expressing cells at a 3:1 (Raji:Jurkat) ratio. Cells were stained with fixable viability dye (Live/Dead Near-IR) and ALEXA-647 anti-myc fluorescently labelled antibodies (both from Thermo Fisher Scientific). The cells were then washed twice and analyzed by flow on a BD-Fortessa.

The nucleotide sequence, including non-coding (e.g. promoter) sequence, of the exemplary synPTPR construct used in these Examples is shown below as SEQ ID NO: 31 (the two fibronectin type-III domains are encoded by nucleotides 4235-4537 and nucleotides 4562-4813, each of which are underlined in the sequence below):

Exemplary synPTPR Construct Nucleotide Sequence
(SEQ ID NO: 31)
gttgacattgattattgactagttattaatagtaatcaattacggggtcattagttcatagcccatatatggagttccgcgttacataacttacggta
aatggcccgcctggctgaccgcccaacgacccccgcccattgacgtcaataatgacgtatgttcccatagtaacgccaatagggactttcc
attgacgtcaatgggtggagtatttacggtaaactgcccacttggcagtacatcaagtgtatcatatgccaagtacgccccctattgacgtcaa
tgacggtaaatggcccgcctggcattatgcccagtacatgaccttatgggactttcctacttggcagtacatctacgtattagtcatcgctatta
ccatggtgatgcggttttggcagtacatcaatgggcgtggatagcggtttgactcacggggatttccaagtctccaccccattgacgtcaatg
ggagtttgttttggcaccaaaatcaacgggactttccaaaatgtcgtaacaactccgccccattgacgcaaatgggcggtaggcgtgtacgg
tgggaggtctatataagcagagctcgtttagtgaaccggggtctctctggttagaccagatctgagcctgggagctctctggctaactaggg
aacccactgcttaagcctcaataaagcttgccttgagtgcttcaagtagtgtgtgcccgtctgttgtgtgactctggtaactagagatccctcag
acccttttagtcagtgtggaaaatctctagcagtggcgcccgaacagggacttgaaagcgaaagggaaaccagaggagctctctcgacgc
aggactcggcttgctgaagcgcgcacggcaagaggcgaggggcggcgactggtgagtacgccaaaaattttgactagcggaggctaga
aggagagagatgggtgcgagagcgtcggtattaagcgggggagaattagataaatgggaaaaaattcggtaataaggccagggggaaa
gaagaagtacaagctaaagcacatcgtatgggcaagcagggagctagaacgattcgcagttaatcctggccttttagagacatcagaagg
cgccgctgatcttcagacctggaggaggcgatatgagggacaattggagaagtgaattatataaatataaagtagtaaaaattgaaccatta
ggagtagcacccaccaaggcaaagagaagagtggtgcagagagaaaaaagagcagtgggaatttaaataggagctttgttccttgggttc
ttgggagcagcaggaagcactatgggcgcagcgtcaatgacgctgacggtacaggccagacaattattgtctgatatagtgcagcagcag
aacaatttgctgagggctattgaggcgcaacagcatctgttgcaactcacagtctggggcatcaaacagctccaggcaagaatcctggctg
tggaaagatacctaaaggatcaacagctcctcctgcaggggatttggggttgctctggaaaactcatttgcaccactgctgtgccttggaatg
ctagttggagtaataaatctctggaacagatttggaatcacacgacctggatggagtgggacagagaaattaacaattacacaagcttaatac
actccttaattgaagaatcgcaaaaccagcaagaaaagaatgaacaagaattattggaattagataaatgggcaagtttgtggaattggttta
acataacaaattggctgtggtatataaaattattcataatgatagtaggaggcttggtaggtttaagaatagtttttgctgtactttctatagtgaat
agagttaggcagggatattcaccattatcgtttcagacccacctcccaaccccgaggggacccgacaggcccgaaggaatagaagaaga
aggtggagagagagacagagacagatccattcgattagtgaacggatctcgacggtatcgatctcgacacaaatggcagtattcatccaca
attttaaaagaaaaggggggattggggggtacagtgcaggggaaagaatagtagacataatagcaacagacatacaaactaaagaattac
aaaaacaaattacaaaaattcaaaattttcgggtttattacagggacagcagagatccagtttgggtcgaggatggtaccacgtgaggctcc
ggtgcccgtcagtgggcagagcgcacatcgcccacagtccccgagaagttggggggaggggtcggcaattgaaccggtgcctagaga
aggtggcgcggggtaaactgggaaagtgatgtcgtgtactggctccgcctttttcccgagggtgggggagaaccgtatataagtgcagta
gtcgccgtgaacgttctttttcgcaacgggtttgccgccagaacacaggtaagtgccgtgtgtggttcccgcgggcctggcctctttacgggt
tatggcccttgcgtgccttgaattacttccacgcccctggctgcagtacgtgattcttgatcccgagcttcgggttggaagtgggtgggagag
ttcgaggccttgcgcttaaggagccccttcgcctcgtgcttgagttgaggcctggcttgggcgctggggccgccgcgtgcgaatctggtgg
caccttcgcgcctgtctcgctgctttcgataagtctctagccatttaaaatttttgatgacctgctgcgacgctttttttctggcaagatagtcttgt
aaatgcgggccaagatctgcacactggtatttcggtttttggggccgcgggcggcgacggggcccgtgcgtcccagcgcacatgttcggc
gaggcggggcctgcgagcgcggccaccgagaatcggacgggggtagtctcaagctggccggcctgctctggtgcctggcctcgcgcc
gccgtgtatcgccccgccctgggcggcaaggctggcccggtcggcaccagttgcgtgagcggaaagatggccgcttcccggccctgct
gcagggagctcaaaatggaggacgcggcgctcgggagagcgggcgggtgagtcacccacacaaaggaaaagggcctttccgtcctca
gccgtcgcttcatgtgactccacggagtaccgggcgccgtccaggcacctcgattagttctcgagcttttggagtacgtcgtctttaggttgg
ggggaggggttttatgcgatggagtttccccacactgagtgggtggagactgaagttaggccagcttggcacttgatgtaattctccttggaa
tttgccctttttgagtttggatcttggttcattctcaagcctcagacagtggttcaaagtttttttcttccatttcaggtgtcgtgaaaactacccctaa
aagccaaagccaccatgcttctcctggtgacaagccttctgctctgtgagttaccacacccagcattcctcttgatccctgaacaaaagctgat
cagcgaggaggatctcgacatccagatgacccagaccaccagcagcctgagcgccagcctgggcgatagagtgaccatcagctgcag
agccagccaggacatcagcaagtacctgaactggtatcagcagaaacccgacggcaccgtgaagctgctgatctaccacaccagcaga
ctgcacagcggcgtgcccagcagattttctggcagcggctccggcaccgactacagcctgaccatctccaacctggaacaggaagatatc
gctacctacttctgtcagcaaggcaacaccctgccctacaccttcggcggaggcaccaagctggaaatcacaggcggcggaggatctgg
cggaggcggaagtggcggagggggatctgaagtgaaactgcaggaaagcggccctggcctggtggccccatctcagtctctgagcgtg
acctgtaccgtgtccggcgtgtccctgcctgactatggcgtgtcctggatcagacagccccccagaaagggcctggaatggctgggagtg
atctggggcagcgagacaacctactacaacagcgccctgaagtcccggctgaccatcatcaaggacaactccaagagccaggtgttcct
gaagatgaacagcctgcagaccgacgacaccgccatctactactgcgccaagcactactactacggcggcagctacgccatggactact
ggggccagggcacaagcgtgaccgtgtctagcgatgtgcctggtcccgtaccagtaaaatctcttcaaggaacatcctttgaaaataagat
cttcttgaactggaaagaacctttggatccaaatggaatcatcactcaatatgagatcagctatagcagtataagatcatttgatcctgcagttc
cagtggctggacctccccagactgtatcaaatttatggaacagtacacaccatgtctttatgcatctccaccctggaaccacgtaccagtttttc
ataagagccagcacggtcaaaggctttggtccagccacagccatcaatgtcaccaccaatatctcagctccaactttacctgactatgaagg
agttgatgcctctctcaatgaaactgccaccacaataactgtattgttgagaccagcacaagccaaaggtgctcctatcagtgcttatcagatt
gttgtggaagaactgcacccacaccgaaccaagagagaagccggagccatggaatgctaccaggttcctgtcacataccaaaatgccat
gagtgggggtgcaccgtattactttgctgcagaactacccccgggaaacctacctgagcctgccccgttcactgtgggtgacaatcggacc
taccaaggcttttggaaccctcctttggctccgcgcaaaggatacaacatctatttccaggcgatgagcagtgtggagaaggaaactaaaac
ccagtgcgtacgcattgctacaaaagcagcagcaacagaagaaccagaagtgatcccagatcccgccaagcagacagacagagtggtg
aaaatagcaggaattagtgctggaattttggtgttcatcctccttctcctagttgtcatattaattgtaaaaaagagcaaacttgctaaaaaacgc
aaagatgccatggggggtggtgggggctcccccgccgccaagagagtgaagctggacggatccatgaaactccttagcagcatcgaac
aggcttgcgacatctgcaggttgaaaaaactcaagtgctcaaaagaaaagcctaagtgcgcaaagtgccttaaaaacaattgggaatgtcg
ctatagccccaagacaaagcggagccctctcacgagagcacacctgactgaggtagaatctcgcttggagaggctggaacagcttttcct
gcttatctttccacgcgaggatctcgatatgatcctcaaaatggactccctccaggacatcaaagctctgctgactggactgtttgtacaggat
aatgtgaacaaggacgctgtgacagacagattggcaagcgtggaaacggatatgcccctgacccttagacagcaccggatcagtgccac
ctcttctagcgaggaaagttcaaataaaggacagcgccagctgacggtgagtggcggtggaagcggaggaggttccgacgctcttgatg
atttcgatctcgacatgctgggatcagacgctctcgacgacttcgatttggacatgcttggatccgacgctctcgatgatttcgacctcgacat
gctcggatccgatgctctggatgactttgatcttgatatgctgtgactacgtcgacaatcaacctctggattacaaaatttgtgaaagattgact
ggtattcttaactatgttgctccttttacgctatgtggatacgctgctttaatgcctttgtatcatgctattgcttcccgtatggctttcattttctcctcc
ttgtataaatcctggttgctgtctctttatgaggagttgtggcccgttgtcaggcaacgtggcgtggtgtgcactgtgtttgctgacgcaacccc
cactggttggggcattgccaccacctgtcagctcctttccgggactttcgctttccccctccctattgccacggcggaactcatcgccgcctg
ccttgcccgctgctggacaggggctcggctgttgggcactgacaattccgtggtgttgtcggggaagctgacgtcctttccatggctgctcg
cctgtgttgccacctggattctgcgcgggacgtccttctgctacgtcccttcggccctcaatccagcggaccttccttcccgcggcctgctgc
cggctctgcggcctcttccgcgtcttcgccttcgccctcagacgagtcggatctccctttgggccgcctccccgcctggttaattaactttaag
accaatgacttacaaggcagctgtagatcttagccactttttaaaagaaaaggggggactggaagggctaattcactcccaacgaagacaa
gatctgctttttgcttgtactgggtctctctggttagaccagatctgagcctgggagctctctggctaactagggaacccactgcttaagcctca
ataaagcttgccttgagtgcttcattgtctgagtaggtgtcattctattctggggggtggggtggggcaggacagcaagggggaggattgg
gaagacaatagcaggcatgctggggagtatgatcggtccacgatcagctagattatctagtcagcttgatcatggtcatagctgtttcctgag
gctcaatactgaccatttaaatcatacctgacctccatagcagaaagtcaaaagcctccgaccggaggcttttgacttgatcggcacctaaga
ggttccaactttcaccataatgaaataagatcactaccgggcgtattttttgagttatcgagattttcaggagctaaggaagctaaaatgagtatt
caacatttccgtgtcgcccttattcccttttttgcggcattttgccttcctgtttttgctcacccagaaacgctggtgaaagtaaaagatgctgaag
atcagttgggtgcacgagtgggttacatcgaactggatctcaacagcggtaagatccttgagagtttacgccccgaagaacgttttccaatg
atgagcacttttaaagttctgctatgtggcgcggtattatcccgtattgacgccgggcaagagcaactcggtcgccgcatacactattctcag
aatgacttggttgaatactcaccagtcacagaaaagcatctcacggatggcatgacagtaagagaattatgcagtgctgccataaccatgag
tgataacactgcggccaacttacttctggcaaccatcggaggaccgaaggagctaaccgcttttttgcacaacatgggggatcatgtaactc
gccttgatcgttgggaaccggagctgaatgaagccataccaaacgacgagcgtgacaccacgatgcctgtagcaatggcaacaacgttg
cgcaaactattaactggcgaactacttactctagcttcccggcaacaattaatagactggatggaggcggataaagttgcaggatcacttctg
cgctcggccctcccggctggctggtttattgctgataaatctggagccggtgagcgtggctctcgcggtatcattgcagcactggggccag
atggtaagccctcccgcatcgtagttatctacacgacggggagtcaggcaactatggatgaacgaaatagacagatcgctgagataggtg
cctcactgattaagcattggtaatgagggccctgaggacctaaatgtaatcacctggctcaccttcgggtgggcctttctgcgttgctggcgtt
tttccataggctccgcccccctgacgagcatcacaaaaatcggtgctcaagtcagaggtggcgaaacccgacaggactataaagatacca
ggcgtttccccctggaagctccctcgtgcgctctcctgttccgaccctgccgcttaccggatacctgtccgcctttctcccttcgggaagcgt
ggcgctttctcatagctcacgctgtaggtatctcagttcggtgtaggtcgttcgctccaagctgggctgtgtgcacgaaccccccgttcagcc
cgaccgctgcgccttatccggtaactatcgtcttgagtccaacccggtaagacacgacttatcgccactggcagcagccactggtaacagg
attagcagagcgaggtatgtaggcggtgctacagagttcttgaagtggtggcctaactacggctacactagaagaacagtatttggtatctg
cgctctgctgaagccagttacctcggaaaaagagttggtagctcttgatccggcaaacaaaccaccgctggtagcggtggtttttttgtttgca
agcagcagattacgcgcagaaaaaaaggatctcaagaagatcctttgattttctaccgaagaaaggcccacccgtgtaaaacgacggcca
gtttatctagtcagcttgattctagctgatcgtggaccggaaggtgagccag

The polypeptide sequence of the exemplary synPTPR construct used in these Examples, including the anti-CD19 extracellular antigen-binding domain, the synPTPRK core having two integrin ligand-binding domains, the transmembrane domain, the intracellular regulatory domain, and the GAL4-VP64 intracellular transcriptional regulatory domain, is shown below as SEQ ID NO: 32 (the 51 cleavage site is in bold, underlined font):

Exemplary synPTPR Construct Polypeptide Sequence
(SEQ ID NO: 32)
MLLLVTSLLLCELPHPAFLLIPEQKLISEEDLDIQMTQTTSSLSASLGDR
VTISCRASQDISKYLNWYQQKPDGTVKLLIYHTSRLHSGVPSRFSGSGSG
TDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLE
WLGVIWGSETTYYNSALKSRLTIIKDNSKSQVFLKMNSLQTDDTAIYYCA
KHYYYGGSYAMDYWGQGTSVTVSSDVPGPVPVKSLQGTSFENKIFLNWKE
PLDPNGIITQYEISYSSIRSFDPAVPVAGPPQTVSNLWNSTHHVFMHLHP
GTTYQFFIRASTVKGFGPATAINVTTNISAPTLPDYEGVDASLNETATTI
TVLLRPAQAKGAPISAYQIVVEELHPHRTKREAGAMECYQVPVTYQNAMS
GGAPYYFAAELPPGNLPEPAPFTVGDNRTYQGFWNPPLAPRKGYNIYFQA
MSSVEKETKTQCVRIATKAAATEEPEVIPDPAKQTDRVVKIAGISAGILV
FILLLLVVILIVKKSKLAKKRKDAMGGGGGSPAAKRVKLDGSMKLLSSIE
QACDICRLKKLKCSKEKPKCAKCLKNNWECRYSPKTKRSPLTRAHLTEVE
SRLERLEQLFLLIFPREDLDMILKMDSLQDIKALLTGLFVQDNVNKDAVT
DRLASVETDMPLTLRQHRISATSSSEESSNKGQRQLTVSGGGSGGGSDAL
DDFDLDMLGSDALDDFDLDMLGSDALDDFDLDMLGSDALDDFDLDML

The nucleotide sequence of the synPTPRK core of the exemplary synPTPR construct used in these Examples is shown below as SEQ ID NO: 33 (the nucleotide sequences encoding the two fibronectin type-III domains are each underlined in the sequence below):

Exemplary synPTPR Core Construct Nucleotide
Sequence
(SEQ ID NO: 33)
gatgtgcctggtcccgtaccagtaaaatctcttcaaggaacatcctttga
aaataagatcttcttgaactggaaagaacctttggatccaaatggaatca
tcactcaatatgagatcagctatagcagtataagatcatttgatcctgca
gttccagtggctggacctccccagactgtatcaaatttatggaacagtac
acaccatgtctttatgcatctccaccctggaaccacgtaccagtttttca
taagagccagcacggtcaaaggctttggtccagccacagccatcaatgtc
accaccaatatctcagctccaactttacctgactatgaaggagttgatgc
ctctctcaatgaaactgccaccacaataactgtattgttgagaccagcac
aagccaaaggtgctcctatcagtgcttatcagattgttgtggaagaactg
cacccacaccgaaccaagagagaagccggagccatggaatgctaccaggt
tcctgtcacataccaaaatgccatgagtgggggtgcaccgtattactttg
ctgcagaactacccccgggaaacctacctgagcctgccccgttcactgtg
ggtgacaatcggacctaccaaggcttttggaaccctcctttggctccgcg
caaaggatacaacatctatttccaggcgatgagcagtgtggagaaggaaa
ctaaaacccagtgcgtacgcattgctacaaaagcagcagcaacagaagaa
ccagaagtgatcccagatcccgccaagcagacagacagagtggtgaaaat
agcaggaattagtgctggaattttggtgttcatcctccttctcctagttg
tcatattaattgtaaaaaagagcaaacttgctaaaaaacgcaaagatgcc
atgggg

The polypeptide sequence of the synPTPRK core of the exemplary synPTPR construct used in these Examples is shown below as SEQ ID NO: 34 (the S1 cleavage site is in bold, underlined font):

Exemplary synPTPR Core Construct Polypeptide
Sequence
(SEQ ID NO: 34)
DVPGPVPVKSLQGTSFENKIFLNWKEPLDPNGIITQYEISYSSIRSFDPA
VPVAGPPQTVSNLWNSTHEIVFMEILHPGTTYQFFIRASTVKGFGPATAI
NVTTNISAPTLPDYEGVDASLNETATTITVLLRPAQAKGAPISAYQIVVE
ELHPHRTKREAGAMECYQVPVTYQNAMSGGAPYYFAAELPPGNLPEPAPF
TVGDNRTYQGFWNPPLAPRKGYNIYFQAMSSVEKETKTQCVRIATKAAAT
EEPEVIPDPAKQTDRVVKIAGISAGILVFILLLLVVILIVKKSKLAKKRK
DAMG

### Example 2: Effects of synPTPR Vs. synNotch on GFP Reporter Expression

Jurkat cells expressing the synPTPR and reporter plasmids constructs shown in FIG. 2 exhibited upregulated GFP expression in the presence of both low (K562 cells) and high (Raji cells) antigen levels of CD19. The extent of stimulation was similar in both scenarios.

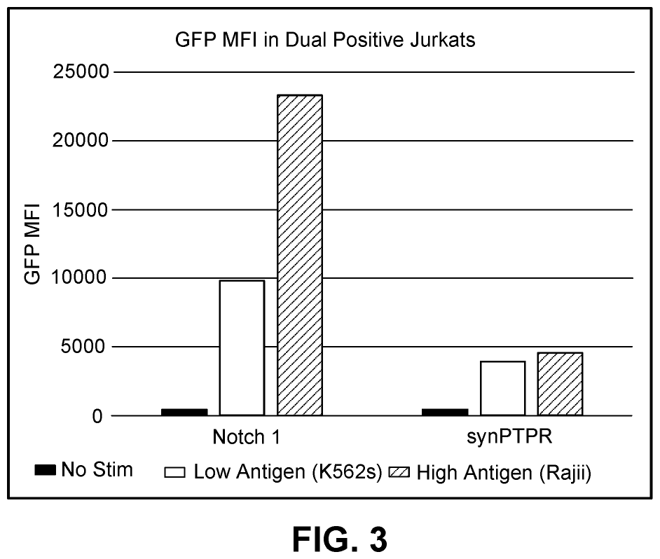

Jurkat cells expression the control synNotch and reporter plasmids constructs shown in FIG. 2 exhibited upregulated GFP expression in the presence of both low (K562 cells) and high (Raji cells) antigen levels of CD19 (FIG. 3). In contrast to the Jurkat cells expressing the synPTPR construct, the extent of stimulation was decreased in cells expressing low antigen levels of CD19 (K562 cells) as compared to cells expressing high levels CD19 (Raji cells) (FIG. 3).

### Example 3

A set of experiments were performed to assess the expression and function of different chimeric transmembrane receptors.

### Materials and Methods

### Tested Constructs

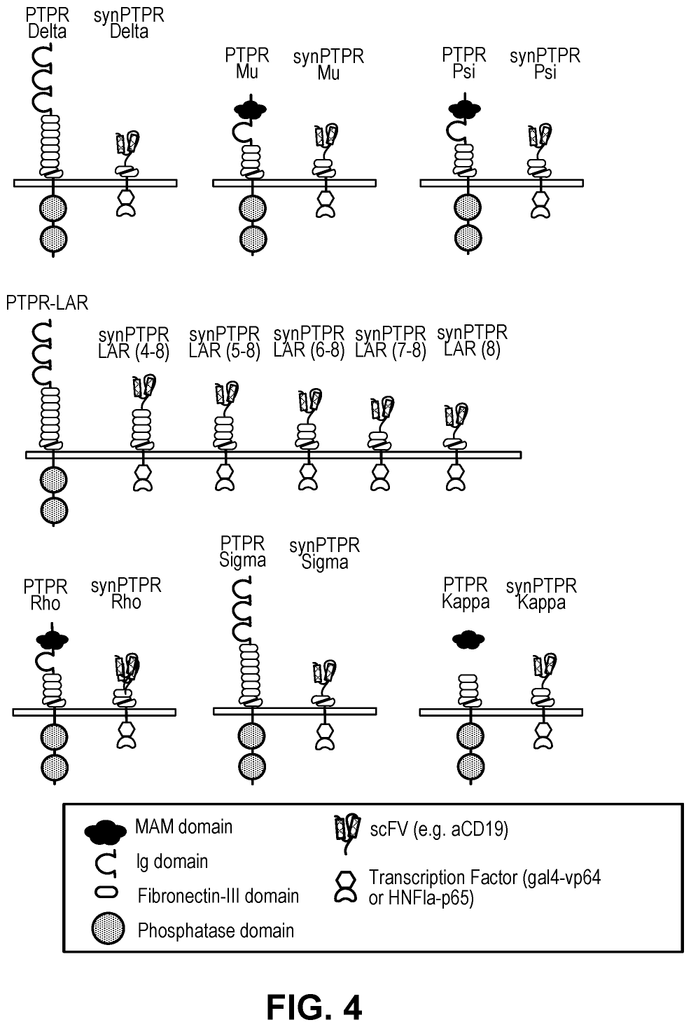

A set of nucleic acids that each encode exemplary different chimeric transmembrane receptors were generated. A schematic showing these different chimeric transmembrane receptors and the corresponding wildtype PTPR proteins are shown in FIG. 4. Table 1 shows each wildtype PTPR protein sequence and each of the nucleic acids tested in these experiments that include a portion of the sequence of each of the wildtype PTPR proteins.

TABLE 1

| | Human PTPR | synPTPR |
|---|---|---|
| PTPR-LAR | P10586 (PTPRF_HUMAN) | pCDL1933, pCDL2762, pCDL2763, |
| (PTPRF) | | pCDL2764, pCDL2765, pCDL2244(HNF1a) |
| PTPR-Delta | P23468 (PTPRD_HUMAN) | pCDL1932, pCDL2243 (HNF1a) |
| PTPR-Mu | P28827 (PTPRM_HUMAN) | pCDL1934 |
| PTPR_Psi | Q92729 (PTPRU_HUMAN) | pCDL1935 |
| PTPR-Kappa | Q15262 (PTPRK_HUMAN) | pCDL1541, pCDL2247(HNF1a) |
| PTPR-Rho | O14522 (PTPRT_HUMAN) | pCDL1936, pCDL2245(HNF1a) |
| PTPR-Sigma | Q13332 (PTPRS_HUMAN) | pCDL1937, pCDL2246(HNF1a) |

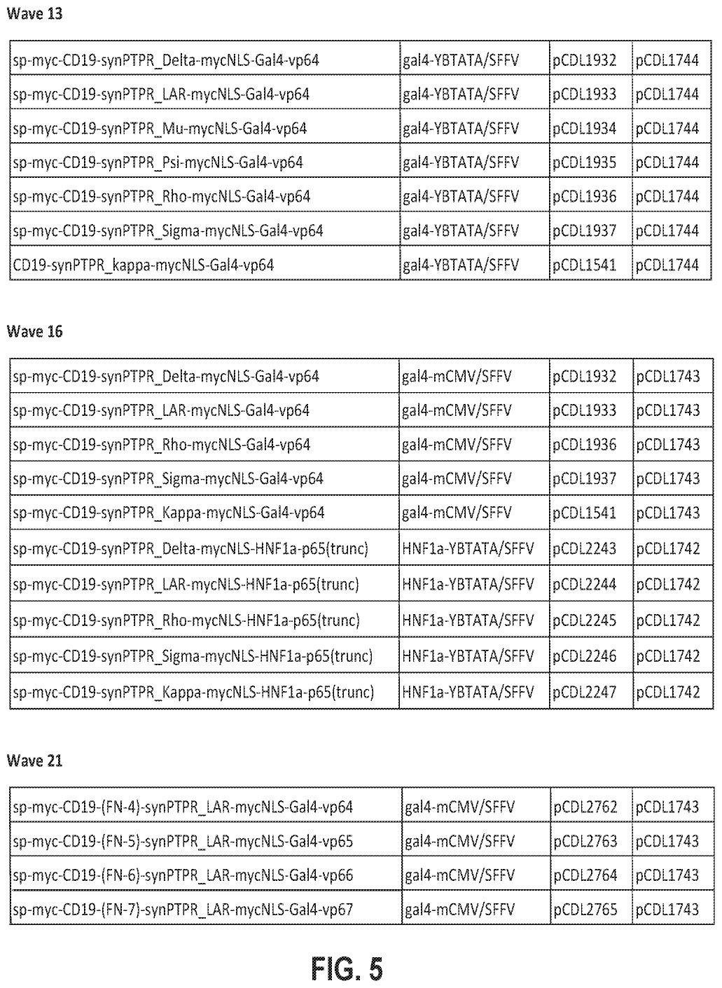

A set of reporter nucleic acid constructs were also generated. The specific pairings of a nucleic acid that encodes a chimeric transmembrane receptor with a reporter nucleic acid construct that were used in these experiments is shown in FIG. 5.

The sequences for each nucleic acid encoding a different chimeric transmembrane receptor is shown below. Also shown below is the chimeric transmembrane receptor encoded by each nucleic acid.

pCDL1932 Protein Sequence (SEQ ID NO: 39) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEITGGGGSGGGGSGGG
GSEVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQGT
SVTVSS;
(linker; SEQ ID NO: 2)
GGGGS;
(FibronectinType III domain; SEQ ID NO: 54)
KNFHVKAVMKTSVLLSWEIPENYNSAMPFKILYDDGKMVEEVDGRATQKLIVNLKPEK
SYSFVLTNRGNSAGGLQHRVTAKTAPD;
(additional portion of PTPR delta; SEQ ID NO: 148)
VLRTKPAFIGKTNLDGMITVQLPEVPANENIKGYYIIIVPLKKSRGKFIKPWESPDEMELD
ELLKEISRKRRSIRYGREVELKPYIAAHFDVLPTEFTLGDDKHYGGFTNKQLQSGQEYVF
FVLAVMEHAESKMYATSPYSDPVVSMDLDPQPITDEEEGLIWVVGPVLAVVFIICIVIAI
LLYKRKRAESDSRKSSGG;
(linker; SEQ ID NO: 2)
GGGGS;
(c-myc NLS; SEQ ID NO: 58)
PAAKRVKLD;
(linker)
GS;
(GAL4 DNA-binding domain; SEQ ID NO: 60)
MKLLSSIEQACDICRLKKLKCSKEKPKCAKCLKNNWECRYSPKTKRSPLTRAHLTEVES
RLERLEQLFLLIFPREDLDMILKMDSLQDIKALLTGLFVQDNVNKDAVTDRLASVETDM;
PLTLRQHRISATSSSEESSNKGQRQLTVS
(linker; SEQ ID NO: 5)
GGGSGGGS;
and
(VP64 Transcriptional Activation Domain; SEQ ID NO: 22)
DALDDFDLDMLGSDALDDFDLDMLGSDALDDFDLDMLGSDALDDFDLDML.

pCDL1932 cDNA Sequence (SEQ ID NO: 40) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCATTCCTCT
TGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGATAGAGT
GACCATCAGCTGCAGAGCCAGCCAGGACATCAGCAAGTACCTGAACTGGTATCAGC
AGAAACCCGACGGCACCGTGAAGCTGCTGATCTACCACACCAGCAGACTGCACAGC
GGCGTGCCCAGCAGATTTTCTGGCAGCGGCTCCGGCACCGACTACAGCCTGACCAT
CTCCAACCTGGAACAGGAAGATATCGCTACCTACTTCTGTCAGCAAGGCAACACCC
TGCCCTACACCTTCGGCGGAGGCACCAAGCTGGAAATCACAGGCGGCGGAGGATCT
GGCGGAGGCGGAAGTGGCGGAGGGGGATCTGAAGTGAAACTGCAGGAAAGCGGCC
CTGGCCTGGTGGCCCCATCTCAGTCTCTGAGCGTGACCTGTACCGTGTCCGGCGTGT
CCCTGCCTGACTATGGCGTGTCCTGGATCAGACAGCCCCCCAGAAAGGGCCTGGAA
TGGCTGGGAGTGATCTGGGGCAGCGAGACAACCTACTACAACAGCGCCCTGAAGTC
CCGGCTGACCATCATCAAGGACAACTCCAAGAGCCAGGTGTTCCTGAAGATGAACA
GCCTGCAGACCGACGACACCGCCATCTACTACTGCGCCAAGCACTACTACTACGGC
GGCAGCTACGCCATGGACTACTGGGGCCAGGGCACAAGCGTGACCGTGTCTAGC;
(linker; SEQ ID NO: 47)
GGTGGAGGAGGCTCT;
(Fibronectin Type III domain; SEQ ID NO: 55)
AAAAATTTCCACGTGAAGGCTGTTATGAAAACATCCGTTCTCCTGTCATGGGAAATC
CCGGAAAACTATAATTCTGCTATGCCTTTCAAGATATTGTATGATGATGGCAAGATG
GTTGAAGAGGTCGACGGTCGGGCGACACAAAAACTGATCGTTAACCTCAAACCTGA
GAAATCATATTCATTCGTCCTCACCAATCGCGGTAATAGTGCTGGTGGCCTCCAGCA
CCGGGTAACCGCAAAAACTGCGCCTGAT;
(additional portion of PTPR delta; SEQ ID NO: 149)
GTGCTCAGGACGAAGCCCGCGTTTATAGGCAAGACCAATCTTGATGGCATGATCAC
TGTTCAGCTCCCGGAAGTTCCCGCCAACGAGAATATCAAGGGTTATTATATTATTAT
CGTACCGCTCAAGAAGTCTCGAGGCAAATTTATCAAACCTTGGGAGTCACCAGATG
AAATGGAGCTTGATGAGTTGCTCAAAGAGATCAGCAGAAAGCGGCGGTCCATAAG
GTACGGCAGGGAGGTCGAGCTCAAGCCATACATTGCGGCTCATTTCGATGTGTTGC
CGACGGAGTTCACGCTCGGGGATGATAAACACTACGGCGGCTTCACAAACAAACAG
CTCCAATCAGGGCAGGAGTATGTCTTCTTCGTGCTTGCTGTCATGGAACACGCCGAA
TCCAAAATGTATGCAACAAGCCCTTACTCCGATCCGGTTGTTTCTATGGATCTGGAC
CCGCAGCCGATAACAGATGAAGAAGAAGGGCTCATTTGGGTGGTTGGCCCTGTGCT
GGCCGTGGTGTTTATTATCTGTATCGTTATTGCGATTCTTCTCTATAAGCGGAAGCG
AGCGGAGAGTGACTCTCGAAAATCATCCGGGGGT;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL 4 DNA-binding domain; SEQ ID NO: 61)
ATGAAACTCCTTAGCAGCATCGAACAGGCTTGCGACATCTGCAGGTTGAAAAAACT
CAAGTGCTCAAAAGAAAAGCCTAAGTGCGCAAAGTGCCTTAAAAACAATTGGGAAT
GTCGCTATAGCCCCAAGACAAAGCGGAGCCCTCTCACGAGAGCACACCTGACTGAG
GTAGAATCTCGCTTGGAGAGGCTGGAACAGCTTTTCCTGCTTATCTTTCCACGCGAG
GATCTCGATATGATCCTCAAAATGGACTCCCTCCAGGACATCAAAGCTCTGCTGACT
GGACTGTTTGTACAGGATAATGTGAACAAGGACGCTGTGACAGACAGATTGGCAAG
CGTGGAAACGGATATGCCCCTGACCCTTAGACAGCACCGGATCAGTGCCACCTCTT
CTAGCGAGGAAAGTTCAAATAAAGGACAGCGCCAGCTGACGGTGAGT;
(linker; SEQ ID NO: 29)
GGCGGTGGAAGCGGAGGAGGTTCC;
and
(VP64 Transcriptional Activation Domain; SEQ ID NO: 62)
GACGCTCTTGATGATTTCGATCTCGACATGCTGGGATCAGACGCTCTCGACGACTTC
GATTTGGACATGCTTGGATCCGACGCTCTCGATGATTTCGACCTCGACATGCTCGGA
TCCGATGCTCTGGATGACTTTGATCTTGATATGCTG.

pCDL1933 Protein Sequence (SEQ ID NO: 66) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEITGGGGSGGGGSGGG
GSEVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQGT
SVTVSS;
(linker; SEQ ID NO: 2)
GGGGS;
(Fibronectin Type III domain; SEQ ID NO: 63)
AKNFRVAAAMKTSVLLSWEVPDSYKSAVPFKILYNGQSVEVDGHSMRKLIADLQPNTE
YSFVLMNRGSSAGGLQHLVSIRTAPD;
(additional portion of PTPR LAR; SEQ ID NO: 151)
LLPHKPLPASAYIEDGRFDLSMPHVQDPSLVRWFYIVVVPIDRVGGSMLTPRWSTPEELE
LDELLEAIEQGGEEQRRRRRQAERLKPYVAAQLDVLPETFTLGDKKNYRGFYNRPLSPD
LSYQCFVLASLKEPMDQKRYASSPYSDEIVVQVTPAQQQEEPEMLWVTGPVLAVILIILI
VIAILLFKRKRTHSPSSKDEQSIGGG;
(linker; SEQ ID NO: 2)
GGGGS;
(c-myc NLS; SEQ ID NO: 58)
PAAKRVKLD;
(linker)
GS;
(GAL 4 DNA-binding domain; SEQ ID NO: 60)
MKLLSSIEQACDICRLKKLKCSKEKPKCAKCLKNNWECRYSPKTKRSPLTRAHLTEVES
RLERLEQLFLLIFPREDLDMILKMDSLQDIKALLTGLFVQDNVNKDAVTDRLASVETDM
PLTLRQHRISATSSSEESSNKGQRQLTVS;
(linker; SEQ ID NO: 5)
GGGSGGGS;
and
(VP64 Transcriptional Activation Domain; SEQ ID NO: 22)
DALDDFDLDMLGSDALDDFDLDMLGSDALDDFDLDMLGSDALDDFDLDML.

pCDL1933 cDNA Sequence (SEQ ID NO: 67) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCATTCCTCT
TGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGATAGAGT
GACCATCAGCTGCAGAGCCAGCCAGGACATCAGCAAGTACCTGAACTGGTATCAGC
AGAAACCCGACGGCACCGTGAAGCTGCTGATCTACCACACCAGCAGACTGCACAGC
GGCGTGCCCAGCAGATTTTCTGGCAGCGGCTCCGGCACCGACTACAGCCTGACCAT
CTCCAACCTGGAACAGGAAGATATCGCTACCTACTTCTGTCAGCAAGGCAACACCC
TGCCCTACACCTTCGGCGGAGGCACCAAGCTGGAAATCACAGGCGGCGGAGGATCT
GGCGGAGGCGGAAGTGGCGGAGGGGGATCTGAAGTGAAACTGCAGGAAAGCGGCC
CTGGCCTGGTGGCCCCATCTCAGTCTCTGAGCGTGACCTGTACCGTGTCCGGCGTGT
CCCTGCCTGACTATGGCGTGTCCTGGATCAGACAGCCCCCCAGAAAGGGCCTGGAA
TGGCTGGGAGTGATCTGGGGCAGCGAGACAACCTACTACAACAGCGCCCTGAAGTC
CCGGCTGACCATCATCAAGGACAACTCCAAGAGCCAGGTGTTCCTGAAGATGAACA
GCCTGCAGACCGACGACACCGCCATCTACTACTGCGCCAAGCACTACTACTACGGC
GGCAGCTACGCCATGGACTACTGGGGCCAGGGCACAAGCGTGACCGTGTCTAGC;
(linker; SEQ ID NO: 49)
GGAGGTGGTGGGAGT;
(Fibronectin Type III domain; SEQ ID NO: 64)
GCCAAAAACTTCAGAGTAGCGGCGGCGATGAAAACTTCTGTCCTTCTGAGCTGGGA
AGTTCCGGATTCCTATAAATCCGCTGTCCCCTTCAAGATATTGTACAATGGTCAAAG
TGTCGAGGTTGATGGACACTCCATGAGGAAACTCATTGCCGACCTCCAACCGAACA
CAGAATACAGTTTTGTTTTGATGAATCGCGGTTCCTCAGCCGGGGGCCTGCAGCACC
TCGTGAGTATTAGGACCGCTCCCGAT;
(additional portion of PTPR LAR; SEQ ID NO: 152)
CTTCTTCCACATAAGCCTCTCCCCGCATCTGCGTACATAGAAGACGGGCGGTTCGAC
CTGTCAATGCCCCACGTACAGGACCCTAGTCTTGTAAGATGGTTTTATATCGTGGTC
GTTCCTATAGACCGGGTTGGAGGCTCTATGCTCACCCCGCGATGGAGTACCCCGGA
AGAACTTGAGCTGGATGAGTTGTTGGAAGCAATCGAACAGGGGGGCGAGGAACAG
AGGCGGCGCCGCCGCCAAGCAGAGCGACTGAAACCGTATGTTGCCGCTCAGTTGGA
TGTGTTGCCCGAAACGTTTACTTTGGGTGACAAAAAAAATTATCGGGGGTTCTACAA
TAGACCTTTGAGTCCTGATCTTAGTTATCAGTGTTTTGTGTTGGCATCTTTGAAGGAA
CCGATGGACCAGAAACGATACGCTTCCTCCCCCTATAGTGACGAGATTGTCGTTCAA
GTAACACCCGCACAACAGCAGGAGGAGCCGGAAATGCTTTGGGTTACAGGGCCGGT
ACTTGCGGTGATCTTGATAATACTCATTGTCATAGCCATTCTGCTTTTCAAGCGCAA
ACGGACCCACTCACCCTCTTCCAAGGACGAGCAGAGCATAGGGGGAGGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL 4 DNA-binding domain; SEQ ID NO: 61)
ATGAAACTCCTTAGCAGCATCGAACAGGCTTGCGACATCTGCAGGTTGAAAAAACT
CAAGTGCTCAAAAGAAAAGCCTAAGTGCGCAAAGTGCCTTAAAAACAATTGGGAAT
GTCGCTATAGCCCCAAGACAAAGCGGAGCCCTCTCACGAGAGCACACCTGACTGAG
GTAGAATCTCGCTTGGAGAGGCTGGAACAGCTTTTCCTGCTTATCTTTCCACGCGAG
GATCTCGATATGATCCTCAAAATGGACTCCCTCCAGGACATCAAAGCTCTGCTGACT
GGACTGTTTGTACAGGATAATGTGAACAAGGACGCTGTGACAGACAGATTGGCAAG
CGTGGAAACGGATATGCCCCTGACCCTTAGACAGCACCGGATCAGTGCCACCTCTT
CTAGCGAGGAAAGTTCAAATAAAGGACAGCGCCAGCTGACGGTGAGT;
(linker; SEQ ID NO: 29)
GGCGGTGGAAGCGGAGGAGGTTCC;
and
(VP64 Transcriptional Activation Domain; SEQ ID NO: 62)
GACGCTCTTGATGATTTCGATCTCGACATGCTGGGATCAGACGCTCTCGACGACTTC
GATTTGGACATGCTTGGATCCGACGCTCTCGATGATTTCGACCTCGACATGCTCGGA
TCCGATGCTCTGGATGACTTTGATCTTGATATGCTG.

pCDL1934 Protein Sequence (SEQ ID NO: 76) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEITGGGGSGGGGSGGG
GSEVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQGT
SVTVSS;
(linker; SEQ ID NO: 2)
GGGGS;
(Fibronectin Type III domain; SEQ ID NO: 70)
IFLQWREPTQTYGVITLYEITYKAVSSFDPEIDLSNQSGRVSKLGNETHFLFFGLYPGTTY
SFTIRASTAKGFGPPATNQFTTKISAPS;
(additional portion of PTPR mu)
M;
(FibronectinType III Domain; SEQ ID NO: 72)
PAYELETPLNQTDNTVTVMLKPAHSRGAPVSVYQIVVEEERPRRTKKTTEILKCYPVPIH
FQNASLLNSQYYFAAEFPADSLQ;
(additional portion of PTPR mu; SEQ ID NO: 153)
AAQPFTIGDNKTYNGYWNTPLLPYKSYRIYFQAASRANGETKIDCVQVATKGAATPKP
VPEPEKQTDHTVKIAGVIAGILLFVIIFLGVVLVMKKRKLAKKRKETMSSTGG;
(linker; SEQ ID NO: 2)
GGGGS;
(c-myc NLS; SEQ ID NO: 58)
PAAKRVKLD;
(linker)
GS;
(GAL4 DNA-binding domain; SEQ ID NO: 60)
MKLLSSIEQACDICRLKKLKCSKEKPKCAKCLKNNWECRYSPKTKRSPLTRAHLTEVES
RLERLEQLFLLIFPREDLDMILKMDSLQDIKALLTGLFVQDNVNKDAVTDRLASVETDM
PLTLRQHRISATSSSEESSNKGQRQLTVS;
(linker; SEQ ID NO: 5)
GGGSGGGS;
(VP64 Transcriptional Activation Domain; SEQ ID NO: 22)
DALDDFDLDMLGSDALDDFDLDMLGSDALDDFDLDMLGSDALDDFDLDML.

pCDL1934 cDNA Sequence (SEQ ID NO: 77) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCATTCCTCT
TGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGATAGAGT
GACCATCAGCTGCAGAGCCAGCCAGGACATCAGCAAGTACCTGAACTGGTATCAGC
AGAAACCCGACGGCACCGTGAAGCTGCTGATCTACCACACCAGCAGACTGCACAGC
GGCGTGCCCAGCAGATTTTCTGGCAGCGGCTCCGGCACCGACTACAGCCTGACCAT
CTCCAACCTGGAACAGGAAGATATCGCTACCTACTTCTGTCAGCAAGGCAACACCC
TGCCCTACACCTTCGGCGGAGGCACCAAGCTGGAAATCACAGGCGGCGGAGGATCT
GGCGGAGGCGGAAGTGGCGGAGGGGGATCTGAAGTGAAACTGCAGGAAAGCGGCC
CTGGCCTGGTGGCCCCATCTCAGTCTCTGAGCGTGACCTGTACCGTGTCCGGCGTGT
CCCTGCCTGACTATGGCGTGTCCTGGATCAGACAGCCCCCCAGAAAGGGCCTGGAA
TGGCTGGGAGTGATCTGGGGCAGCGAGACAACCTACTACAACAGCGCCCTGAAGTC
CCGGCTGACCATCATCAAGGACAACTCCAAGAGCCAGGTGTTCCTGAAGATGAACA
GCCTGCAGACCGACGACACCGCCATCTACTACTGCGCCAAGCACTACTACTACGGC
GGCAGCTACGCCATGGACTACTGGGGCCAGGGCACAAGCGTGACCGTGTCTAGC;
(linker; SEQ ID NO: 50)
GGCGGAGGCGGGAGC;
(Fibronectin Type III domain; SEQ ID NO: 71)
ATATTTCTGCAATGGCGAGAGCCTACACAAACATACGGAGTCATAACGTTGTACGA
AATTACGTACAAGGCCGTGTCATCATTCGATCCCGAAATTGATCTTTCTAACCAGTC
AGGGCGCGTAAGTAAACTCGGCAACGAGACCCACTTTTTGTTCTTCGGCCTGTATCC
GGGCACTACGTACAGTTTCACCATCCGCGCATCTACGGCCAAGGGTTTTGGCCCACC
CGCTACGAACCAGTTTACTACGAAGATTTCTGCTCCTTCA;
(additional portion of PTPR mu)
ATG;
(Fibronectin Type III domain; SEQ ID NO: 73)
CCAGCTTATGAACTCGAAACTCCACTGAACCAAACTGACAACACAGTTACTGTGAT
GCTGAAGCCCGCGCATAGCCGAGGTGCCCCAGTTTCTGTGTATCAAATTGTGGTAG
AAGAAGAACGGCCACGCCGCACAAAGAAGACGACGGAAATACTGAAATGTTATCC
AGTCCCTATTCACTTCCAGAACGCTAGTTTGCTTAACTCACAGTATTATTTCGCGGC
AGAATTCCCCGCCGATTCTCTGCAG;
(additional portion of PTPR mu; SEQ ID NO: 154)
GCGGCACAGCCCTTTACAATAGGGGACAACAAGACTTACAATGGCTATTGGAACAC
CCCCTTGCTTCCTTACAAGAGCTACAGGATCTACTTTCAAGCGGCCTCCCGCGCAAA
CGGTGAAACGAAAATTGACTGTGTGCAGGTAGCCACAAAGGGTGCAGCGACTCCGA
AGCCCGTACCGGAGCCAGAGAAGCAAACTGATCACACAGTCAAGATTGCCGGCGTC
ATAGCAGGTATTCTGTTGTTCGTGATAATCTTTCTCGGCGTCGTCCTCGTTATGAAG
AAGAGGAAACTCGCAAAGAAGCGGAAGGAAACAATGTCATCCACTGGTGGA;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-binding domain; SEQ ID NO: 61)
ATGAAACTCCTTAGCAGCATCGAACAGGCTTGCGACATCTGCAGGTTGAAAAAACT
CAAGTGCTCAAAAGAAAAGCCTAAGTGCGCAAAGTGCCTTAAAAACAATTGGGAAT
GTCGCTATAGCCCCAAGACAAAGCGGAGCCCTCTCACGAGAGCACACCTGACTGAG
GTAGAATCTCGCTTGGAGAGGCTGGAACAGCTTTTCCTGCTTATCTTTCCACGCGAG
GATCTCGATATGATCCTCAAAATGGACTCCCTCCAGGACATCAAAGCTCTGCTGACT
GGACTGTTTGTACAGGATAATGTGAACAAGGACGCTGTGACAGACAGATTGGCAAG
CGTGGAAACGGATATGCCCCTGACCCTTAGACAGCACCGGATCAGTGCCACCTCTT
CTAGCGAGGAAAGTTCAAATAAAGGACAGCGCCAGCTGACGGTGAGT;
(linker; SEQ ID NO: 29)
GGCGGTGGAAGCGGAGGAGGTTCC;
and
(VP64 Transcriptional Activation Domain; SEQ ID NO: 62)
GACGCTCTTGATGATTTCGATCTCGACATGCTGGGATCAGACGCTCTCGACGACTTC
GATTTGGACATGCTTGGATCCGACGCTCTCGATGATTTCGACCTCGACATGCTCGGA
TCCGATGCTCTGGATGACTTTGATCTTGATATGCTG.

pCDL1935 Protein Sequence (SEQ ID NO: 78) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEITGGGGSGGGGSGGG
GSEVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQGT
SVTVSS;
(linker; SEQ ID NO: 2)
GGGGS;
(additional portion of PTPR psi)
ED;
(Fibronectin Type III domain; SEQ ID NO: 80)
VPSGIAAESLTFTPLEDMIFLKWEEPQEPNGLITQYEISYQSIESSDPAVNVPGPRRTISKL
RNETYHVFSNLHPGTTYLFSVRARTGKGFGQAALTEITTNISAPS;
(Fibronectin Type III domain; SEQ ID NO: 82)
FDYADMPSPLGESENTITVLLRPAQGRGAPISVYQVIVEEERARRLRREPGGQDCFPVPL
TFEAALARGLVHYFGAELAASSL;
(additional portion of PTPR psi; SEQ ID NO: 155)
PEAMPFTVGDNQTYRGFWNPPLEPRKAYLIYFQAASHLKGETRLNCIRIARKAACKESK
RPLEVSQRSEEMGLILGICAGGLAVLILLLGAIIVIIRKGRDHYAYSYYPKPVNMTGG;
(linker; SEQ ID NO: 2)
GGGGS;
(c-myc NLS; SEQ ID NO: 58)
PAAKRVKLD;
(linker)
GS;
(GAL4 DNA-binding domain; SEQ ID NO: 60)
MKLLSSIEQACDICRLKKLKCSKEKPKCAKCLKNNWECRYSPKTKRSPLTRAHLTEVES
RLERLEQLFLLIFPREDLDMILKMDSLQDIKALLTGLFVQDNVNKDAVTDRLASVETDM
PLTLRQHRISATSSSEESSNKGQRQLTVS;
(linker; SEQ ID NO: 5)
GGGSGGGS;
and
(VP64 Transcriptional Activation Domain; SEQ ID NO: 22)
DALDDFDLDMLGSDALDDFDLDMLGSDALDDFDLDMLGSDALDDFDLDML.

pCDL1935 cDNA Sequence (SEQ ID NO: 79) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCATTCCTCT
TGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGATAGAGT
GACCATCAGCTGCAGAGCCAGCCAGGACATCAGCAAGTACCTGAACTGGTATCAGC
AGAAACCCGACGGCACCGTGAAGCTGCTGATCTACCACACCAGCAGACTGCACAGC
GGCGTGCCCAGCAGATTTTCTGGCAGCGGCTCCGGCACCGACTACAGCCTGACCAT
CTCCAACCTGGAACAGGAAGATATCGCTACCTACTTCTGTCAGCAAGGCAACACCC
TGCCCTACACCTTCGGCGGAGGCACCAAGCTGGAAATCACAGGCGGCGGAGGATCT
GGCGGAGGCGGAAGTGGCGGAGGGGGATCTGAAGTGAAACTGCAGGAAAGCGGCC
CTGGCCTGGTGGCCCCATCTCAGTCTCTGAGCGTGACCTGTACCGTGTCCGGCGTGT
CCCTGCCTGACTATGGCGTGTCCTGGATCAGACAGCCCCCCAGAAAGGGCCTGGAA
TGGCTGGGAGTGATCTGGGGCAGCGAGACAACCTACTACAACAGCGCCCTGAAGTC
CCGGCTGACCATCATCAAGGACAACTCCAAGAGCCAGGTGTTCCTGAAGATGAACA
GCCTGCAGACCGACGACACCGCCATCTACTACTGCGCCAAGCACTACTACTACGGC
GGCAGCTACGCCATGGACTACTGGGGCCAGGGCACAAGCGTGACCGTGTCTAGC;
(linker; SEQ ID NO: 51)
GGCGGTGGAGGTTCC;
(additional portion of PTPR psi)
GAAGAT;
(Fibronectin Type III Domain; SEQ ID NO: 81)
GTCCCATCCGGTATAGCGGCTGAAAGCTTGACATTTACCCCATTGGAAGATATGATT
TTCCTGAAATGGGAAGAACCACAAGAGCCCAATGGTCTCATTACTCAATATGAGAT
CAGTTACCAGAGCATTGAGTCAAGTGATCCCGCTGTCAATGTACCGGGACCTCGCA
GGACTATCTCTAAGCTGCGGAACGAAACGTACCATGTATTCAGCAACCTGCACCCC
GGCACCACGTACTTGTTTTCCGTACGCGCGAGAACTGGCAAGGGATTCGGGCAGGC
TGCCCTTACAGAAATAACTACGAACATTTCTGCTCCTTCA;
(Fibronectin Type III Domain; SEQ ID NO: 83)
TTCGACTACGCAGACATGCCTTCACCGCTCGGTGAATCTGAGAACACCATTACGGTC
CTGCTTAGGCCTGCACAGGGAAGGGGTGCTCCCATTTCCGTCTACCAGGTAATCGTT
GAAGAGGAACGCGCCCGGCGGCTCAGACGGGAACCCGGTGGGCAAGACTGTTTCC
CGGTCCCTCTGACCTTTGAGGCGGCCTTGGCCAGAGGTCTGGTGCATTACTTCGGAG
CCGAGTTGGCCGCAAGCTCACTG;
(additional portion of PTPR psi; SEQ ID NO: 156)
CCTGAGGCGATGCCCTTCACCGTGGGGGACAATCAGACCTACAGGGGATTTTGGAA
TCCACCTCTTGAACCTCGCAAAGCGTACCTGATCTATTTCCAGGCTGCGTCACACCT
GAAAGGGGAAACCAGGTTGAATTGCATCCGCATAGCTAGGAAAGCCGCCTGTAAA
GAGTCCAAAAGGCCACTTGAAGTCTCTCAGCGCAGTGAAGAAATGGGTCTGATCCT
TGGAATTTGCGCGGGAGGGCTGGCTGTACTTATCCTTCTCCTCGGAGCTATAATCGT
TATAATCAGGAAAGGCAGAGACCACTACGCCTACTCTTACTATCCTAAACCGGTGA
ACATGACGGGGGGA;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-binding domain; SEQ ID NO: 61)
ATGAAACTCCTTAGCAGCATCGAACAGGCTTGCGACATCTGCAGGTTGAAAAAACT
CAAGTGCTCAAAAGAAAAGCCTAAGTGCGCAAAGTGCCTTAAAAACAATTGGGAAT
GTCGCTATAGCCCCAAGACAAAGCGGAGCCCTCTCACGAGAGCACACCTGACTGAG
GTAGAATCTCGCTTGGAGAGGCTGGAACAGCTTTTCCTGCTTATCTTTCCACGCGAG
GATCTCGATATGATCCTCAAAATGGACTCCCTCCAGGACATCAAAGCTCTGCTGACT
GGACTGTTTGTACAGGATAATGTGAACAAGGACGCTGTGACAGACAGATTGGCAAG
CGTGGAAACGGATATGCCCCTGACCCTTAGACAGCACCGGATCAGTGCCACCTCTT
CTAGCGAGGAAAGTTCAAATAAAGGACAGCGCCAGCTGACGGTGAGT;
(linker; SEQ ID NO: 29)
GGCGGTGGAAGCGGAGGAGGTTCC;
and
(VP64 Transcriptional Activation Domain; SEQ ID NO: 62)
GACGCTCTTGATGATTTCGATCTCGACATGCTGGGATCAGACGCTCTCGACGACTTC
GATTTGGACATGCTTGGATCCGACGCTCTCGATGATTTCGACCTCGACATGCTCGGA
TCCGATGCTCTGGATGACTTTGATCTTGATATGCTG.

pCDL1936 Protein Sequence (SEQ ID NO: 86) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHTSRLHSGVPS
RFSGSGSGTDYSLTISNLEQEDIATYFCQQGNTLPYTFGGGTKLEITGGGGSGGGGSGGG
GSEVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQGT
SVTVSS;
(linker; SEQ ID NO: 2)
GGGGS;
(Partial Fibronectin Type III Domain; SEQ ID NO: 88)
KIYIQWKPPNETNGVITLYEINYKAVGSLDPSADLSSQRGKVFKLRNETHEILFVGLYPGT
TYSFTIKASTAKGFGPPVTTRIATKISAPS;
(Fibronectin Type III domain; SEQ ID NO: 90)
MPEYDTDTPLNETDTTITVMLKPAQSRGAPVSVYQLVVKEERLQKSRRAADIIECFSVP
VSYRNASSLDSLHYFAAELKPANLPVTQPFTVGDNKTYNGYWNPPLSPLKSYSIYFQAL
SKANGETKINCVRLATKG;
(additional portion of PTPR rho; SEQ ID NO: 157)
ASTQNSNTVEPEKQVDNTVKMAGVIAGLLMFIIILLGVMLTIKRRRNAYSYSYYLKLAK
KQKETGG;
(linker; SEQ ID NO: 2)
GGGGS;
(c-myc NLS; SEQ ID NO: 58)
PAAKRVKLD;
(linker)
GS;
(GAL4 DNA-binding domain; SEQ ID NO: 60)
MKLLSSIEQACDICRLKKLKCSKEKPKCAKCLKNNWECRYSPKTKRSPLTRAHLTEVES
RLERLEQLFLLIFPREDLDMILKMDSLQDIKALLTGLFVQDNVNKDAVTDRLASVETDM
PLTLRQHRISATSSSEESSNKGQRQLTVS;
(linker; SEQ ID NO: 5)
GGGSGGGS;
and
(VP64 Transcriptional Activation Domain; SEQ ID NO: 22)
DALDDFDLDMLGSDALDDFDLDMLGSDALDDFDLDMLGSDALDDFDLDML.

pCDL1936 cDNA Sequence (SEQ ID NO: 87) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCAC
ACCCAGCATTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCC
TGGGCGATAGAGTGACCATCAGCTGCAGAGCCAGCCAGGACAT
CAGCAAGTACCTGAACTGGTATCAGCAGAAACCCGACGGCACC
GTGAAGCTGCTGATCTACCACACCAGCAGACTGCACAGCGGCG
TGCCCAGCAGATTTTCTGGCAGCGGCTCCGGCACCGACTACAG
CCTGACCATCTCCAACCTGGAACAGGAAGATATCGCTACCTAC
TTCTGTCAGCAAGGCAACACCCTGCCCTACACCTTCGGCGGAG
GCACCAAGCTGGAAATCACAGGCGGCGGAGGATCTGGCGGAGG
CGGAAGTGGCGGAGGGGGATCTGAAGTGAAACTGCAGGAAAGC
GGCCCTGGCCTGGTGGCCCCATCTCAGTCTCTGAGCGTGACCT
GTACCGTGTCCGGCGTGTCCCTGCCTGACTATGGCGTGTCCTG
GATCAGACAGCCCCCCAGAAAGGGCCTGGAATGGCTGGGAGTG
ATCTGGGGCAGCGAGACAACCTACTACAACAGCGCCCTGAAGT
CCCGGCTGACCATCATCAAGGACAACTCCAAGAGCCAGGTGTT
CCTGAAGATGAACAGCCTGCAGACCGACGACACCGCCATCTAC
TACTGCGCCAAGCACTACTACTACGGCGGCAGCTACGCCATGG
ACTACTGGGGCCAGGGCACAAGCGTGACCGTGTCTAGC;
(linker; SEQ ID NO: 52)
GGGGGAGGTGGGAGT;
(Partial Fibronectin Type III Domain;
SEQ ID NO: 89)
AAAATCTATATTCAATGGAAGCCGCCAAATGAAACCAATGGAG
TTATAACGCTCTATGAAATCAACTACAAGGCGGTTGGCTCCCT
TGATCCCTCTGCCGACCTTTCCTCACAGCGGGGTAAAGTGTTC
AAGCTGAGGAACGAAACGCACCACCTTTTCGTGGGGTTGTATC
CAGGAACGACCTACAGTTTTACTATTAAGGCTTCCACAGCCAA
AGGCTTTGGGCCCCCTGTAACCACTAGGATTGCTACTAAAATC
TCCGCGCCATCT;
(Fibronectin Type III Domain;
SEQ ID NO: 91)
ATGCCCGAATATGATACGGACACCCCATTGAACGAAACAGATA
CAACAATAACTGTCATGCTGAAGCCTGCGCAATCACGCGGAGC
CCCTGTCAGCGTATATCAACTTGTAGTCAAAGAAGAAAGACTG
CAAAAATCCCGACGCGCTGCCGACATTATTGAGTGCTTCTCAG
TACCCGTGAGCTACAGAAACGCTAGTAGCTTGGATTCTTTGCA
TTATTTCGCGGCCGAACTTAAGCCCGCGAATCTTCCGGTGACT
CAACCGTTTACAGTGGGTGACAATAAAACTTACAATGGCTATT
GGAACCCGCCGTTGTCACCGCTGAAGAGCTACTCAATATATTT
CCAGGCCCTGAGTAAAGCTAACGGTGAGACAAAAATCAACTGT
GTGAGACTTGCAACTAAAGGA;
(additional portion of PTPR rho;
SEQ ID NO: 158)
GCCAGCACACAGAATTCTAATACTGTGGAGCCCGAGAAGCAAG
TTGACAACACTGTGAAAATGGCTGGGGTAATTGCAGGACTGCT
TATGTTCATCATAATCCTGCTTGGGGTTATGCTTACTATCAAG
CGACGGCGCAACGCCTACAGCTATAGCTACTATTTGAAATTGG
CAAAAAAGCAGAAGGAAACTGGAGGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-binding domain; SEQ ID NO: 61)
ATGAAACTCCTTAGCAGCATCGAACAGGCTTGCGACATCTGCA
GGTTGAAAAAACTCAAGTGCTCAAAAGAAAAGCCTAAGTGCGC
AAAGTGCCTTAAAAACAATTGGGAATGTCGCTATAGCCCCAAG
ACAAAGCGGAGCCCTCTCACGAGAGCACACCTGACTGAGGTAG
AATCTCGCTTGGAGAGGCTGGAACAGCTTTTCCTGCTTATCTT
TCCACGCGAGGATCTCGATATGATCCTCAAAATGGACTCCCTC
CAGGACATCAAAGCTCTGCTGACTGGACTGTTTGTACAGGATA
ATGTGAACAAGGACGCTGTGACAGACAGATTGGCAAGCGTGGA
AACGGATATGCCCCTGACCCTTAGACAGCACCGGATCAGTGCC
ACCTCTTCTAGCGAGGAAAGTTCAAATAAAGGACAGCGCCAGC
TGACGGTGAGT;
(linker; SEQ ID NO: 29)
GGCGGTGGAAGCGGAGGAGGTTCC;
and
(VP64 Transcriptional Activation Domain;
SEQ ID NO: 62)
GACGCTCTTGATGATTTCGATCTCGACATGCTGGGATCAGACG
CTCTCGACGACTTCGATTTGGACATGCTTGGATCCGACGCTCT
CGATGATTTCGACCTCGACATGCTCGGATCCGATGCTCTGGAT
GACTTTGATCTTGATATGCTG.

pCDL1937 Protein Sequence (SEQ ID NO: 96) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGT
VKLLIYHTSRLHSGVPSRFSGSGSGTDYSLTISNLEQEDIATY
FCQQGNTLPYTFGGGTKLEITGGGGSGGGGSGGGGSEVKLQES
GPGLVAPSQSLSVTCTVSGVSLPDYGVSWIRQPPRKGLEWLGV
IWGSETTYYNSALKSRLTIIKDNSKSQVFLKMNSLQTDDTAIY
YCAKHYYYGGSYAMDYWGQGTSVTVSS;
(linker; SEQ ID NO: 2)
GGGGS;
(Fibronectin Type III Domain;
SEQ ID NO: 98)
SPKNFKVKMIMKTSVLLSWEFPDNYNSPTPYKIQYNGLTLDVD
GRTTKKLITHLKPHTFYNFVLTNRGSSLGGLQQTVTAWTAFN;
(additional portion of PTPR sigma;
SEQ ID NO: 159)
LLNGKPSVAPKPDADGFIMVYLPDGQSPVPVQSYFIVMVPLRK
SRGGQFLTPLGSPEDMDLEELIQDISRLQRRSLRHSRQLEVPR
PYIAARFSVLPPTFHPGDQKQYGGFDNRGLEPGHRYVLFVLAV
LQKSEPTFAASPFSDPFQLDNPDPQPIVDGEEGLIWVIGPVLA
VVFIICIVIAILLYKNKPDSKRKDSEPRTKGG;
(linker; SEQ ID NO: 2)
GGGGS;
(c-myc NLS; SEQ ID NO: 58)
PAAKRVKLD;
GS (linker);
(GAL4 DNA-binding domain; SEQ ID NO: 60)
MKLLSSIEQACDICRLKKLKCSKEKPKCAKCLKNNWECRYSPK
TKRSPLTRAHLTEVESRLERLEQLFLLIFPREDLDMILKMDSL
QDIKALLTGLFVQDNVNKDAVTDRLASVETDMPLTLRQHRISA
TSSSEESSNKGQRQLTVS;
(linker; SEQ ID NO: 5)
GGGSGGGS;
and
(VP64 Transcriptional Activation Domain;
SEQ ID NO: 22)
DALDDFDLDMLGSDALDDFDLDMLGSDALDDFDLDMLGSDALD
DFDLDML.

pCDL1937 cDNA Sequence (SEQ ID NO: 97) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCAC
ACCCAGCATTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCC
TGGGCGATAGAGTGACCATCAGCTGCAGAGCCAGCCAGGACAT
CAGCAAGTACCTGAACTGGTATCAGCAGAAACCCGACGGCACC
GTGAAGCTGCTGATCTACCACACCAGCAGACTGCACAGCGGCG
TGCCCAGCAGATTTTCTGGCAGCGGCTCCGGCACCGACTACAG
CCTGACCATCTCCAACCTGGAACAGGAAGATATCGCTACCTAC
TTCTGTCAGCAAGGCAACACCCTGCCCTACACCTTCGGCGGAG
GCACCAAGCTGGAAATCACAGGCGGCGGAGGATCTGGCGGAGG
CGGAAGTGGCGGAGGGGGATCTGAAGTGAAACTGCAGGAAAGC
GGCCCTGGCCTGGTGGCCCCATCTCAGTCTCTGAGCGTGACCT
GTACCGTGTCCGGCGTGTCCCTGCCTGACTATGGCGTGTCCTG
GATCAGACAGCCCCCCAGAAAGGGCCTGGAATGGCTGGGAGTG
ATCTGGGGCAGCGAGACAACCTACTACAACAGCGCCCTGAAGT
CCCGGCTGACCATCATCAAGGACAACTCCAAGAGCCAGGTGTT
CCTGAAGATGAACAGCCTGCAGACCGACGACACCGCCATCTAC
TACTGCGCCAAGCACTACTACTACGGCGGCAGCTACGCCATGG
ACTACTGGGGCCAGGGCACAAGCGTGACCGTGTCTAGC;
(linker; SEQ ID NO: 53)
GGCGGGGGAGGGAGC;
(Fibronectin Type III Domain;
SEQ ID NO: 99)
AGTCCGAAGAACTTCAAAGTCAAGATGATCATGAAGACATCTG
TGCTCTTGAGTTGGGAGTTTCCCGATAATTATAACAGCCCCAC
GCCTTACAAGATTCAGTACAATGGACTCACCCTTGATGTAGAT
GGCCGCACGACAAAGAAGCTGATTACTCACCTCAAACCGCATA
CTTTCTATAATTTCGTGCTGACGAATCGGGGTTCTTCCCTGGG
AGGTCTCCAGCAAACTGTAACGGCGTGGACTGCGTTTAAT;
(additional portion of PTPR sigma;
SEQ ID NO: 160)
TTGCTGAACGGTAAGCCCTCAGTGGCCCCCAAACCGGATGCCG
ACGGATTTATAATGGTGTACCTTCCAGATGGTCAGAGTCCGGT
CCCCGTACAGAGCTACTTCATTGTCATGGTGCCCCTCAGGAAA
TCCCGAGGTGGTCAATTTCTCACACCATTGGGTAGTCCGGAGG
ACATGGATCTGGAAGAACTGATCCAGGATATTAGCCGCCTGCA
ACGCAGATCACTTAGACATAGTAGACAGCTGGAGGTGCCGAGG
CCGTACATCGCTGCGCGATTCTCCGTACTCCCGCCAACCTTTC
ACCCAGGGGATCAGAAACAATACGGCGGTTTTGATAATCGAGG
GCTTGAACCAGGACATAGATACGTGCTTTTTGTGTTGGCTGTG
CTCCAGAAATCTGAACCGACGTTTGCCGCAAGCCCCTTTAGCG
ACCCATTTCAGCTGGATAACCCTGACCCTCAGCCGATAGTCGA
TGGCGAGGAGGGGCTGATATGGGTGATTGGGCCCGTACTCGCG
GTAGTGTTTATTATCTGTATCGTAATTGCTATACTGCTTTATA
AGAACAAGCCGGACAGTAAAAGGAAGGATTCTGAGCCTAGGAC
TAAAGGCGGT;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-binding domain; SEQ ID NO: 61)
ATGAAACTCCTTAGCAGCATCGAACAGGCTTGCGACATCTGCA
GGTTGAAAAAACTCAAGTGCTCAAAAGAAAAGCCTAAGTGCGC
AAAGTGCCTTAAAAACAATTGGGAATGTCGCTATAGCCCCAAG
ACAAAGCGGAGCCCTCTCACGAGAGCACACCTGACTGAGGTAG
AATCTCGCTTGGAGAGGCTGGAACAGCTTTTCCTGCTTATCTT
TCCACGCGAGGATCTCGATATGATCCTCAAAATGGACTCCCTC
CAGGACATCAAAGCTCTGCTGACTGGACTGTTTGTACAGGATA
ATGTGAACAAGGACGCTGTGACAGACAGATTGGCAAGCGTGGA
AACGGATATGCCCCTGACCCTTAGACAGCACCGGATCAGTGCC
ACCTCTTCTAGCGAGGAAAGTTCAAATAAAGGACAGCGCCAGC
TGACGGTGAGT;
(linker; SEQ ID NO: 29)
GGCGGTGGAAGCGGAGGAGGTTCC;
(VP64 Transcriptional Activation Domain;
SEQ ID NO: 62)
GACGCTCTTGATGATTTCGATCTCGACATGCTGGGATCAGACG
CTCTCGACGACTTCGATTTGGACATGCTTGGATCCGACGCTCT
CGATGATTTCGACCTCGACATGCTCGGATCCGATGCTCTGGAT
GACTTTGATCTTGATATGCTG.

pCDL1541 Protein Sequence (SEQ ID NO: 102) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGT
VKLLIYHTSRLHSGVPSRFSGSGSGTDYSLTISNLEQEDIATY
FCQQGNTLPYTFGGGTKLEITGGGGSGGGGSGGGGSEVKLQES
GPGLVAPSQSLSVTCTVSGVSLPDYGVSWIRQPPRKGLEWLGV
IWGSETTYYNSALKSRLTIIKDNSKSQVFLKMNSLQTDDTAIY
YCAKHYYYGGSYAMDYWGQGTSVTVSS;
DVP (additional portion of PTPR kappa);
(Fibronectin Type III Domain;
SEQ ID NO: 104)
GPVPVKSLQGTSFENKIFLNWKEPLDPNGIITQYEISYSSIRS
FDPAVPVAGPPQTVSNLWNSTHEIVFMHLHPGTTYQFFIRAST
VKGFGPATAINVTTNISAPT;
L (additional portion of PTPR kappa);
(Fibronectin Type III Domain;
SEQ ID NO: 106)
PDYEGVDASLNETATTITVLLRPAQAKGAPISAYQIVVEELHP
HRTKREAGAMECYQVPVTYQNAMSGGAPYYFAAELPPGNLP;
(additional portion of PTPR kappa;
SEQ ID NO: 161)
EPAPFTVGDNRTYQGFWNPPLAPRKGYNIYFQAMSSVEKETKT
QCVRIATKAAATEEPEVIPDPAKQTDRVVKIAGISAGILVFIL
LLLVVILIVKKSKLAKKRKDAMG;
(linker; SEQ ID NO: 2)
GGGGS;
(c-myc NLS; SEQ ID NO: 58)
PAAKRVKLD;
GS (linker);
(GAL4 DNA-binding domain; SEQ ID NO: 60)
MKLLSSIEQACDICRLKKLKCSKEKPKCAKCLKNNWECRYSPK
TKRSPLTRAHLTEVESRLERLEQLFLLIFPREDLDMILKMDSL
QDIKALLTGLFVQDNVNKDAVTDRLASVETDMPLTLRQHRISA
TSSSEESSNKGQRQLTVS;
(linker; SEQ ID NO: 5)
GGGSGGGS;
and
(VP64 Transcriptional Activation Domain;
SEQ ID NO: 22)
DALDDFDLDMLGSDALDDFDLDMLGSDALDDFDLDMLGSDALD
DFDLDML.

pCDL1541 cDNA Sequence (SEQ ID NO: 103) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCAC
ACCCAGCATTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCC
TGGGCGATAGAGTGACCATCAGCTGCAGAGCCAGCCAGGACAT
CAGCAAGTACCTGAACTGGTATCAGCAGAAACCCGACGGCACC
GTGAAGCTGCTGATCTACCACACCAGCAGACTGCACAGCGGCG
TGCCCAGCAGATTTTCTGGCAGCGGCTCCGGCACCGACTACAG
CCTGACCATCTCCAACCTGGAACAGGAAGATATCGCTACCTAC
TTCTGTCAGCAAGGCAACACCCTGCCCTACACCTTCGGCGGAG
GCACCAAGCTGGAAATCACAGGCGGCGGAGGATCTGGCGGAGG
CGGAAGTGGCGGAGGGGGATCTGAAGTGAAACTGCAGGAAAGC
GGCCCTGGCCTGGTGGCCCCATCTCAGTCTCTGAGCGTGACCT
GTACCGTGTCCGGCGTGTCCCTGCCTGACTATGGCGTGTCCTG
GATCAGACAGCCCCCCAGAAAGGGCCTGGAATGGCTGGGAGTG
ATCTGGGGCAGCGAGACAACCTACTACAACAGCGCCCTGAAGT
CCCGGCTGACCATCATCAAGGACAACTCCAAGAGCCAGGTGTT
CCTGAAGATGAACAGCCTGCAGACCGACGACACCGCCATCTAC
TACTGCGCCAAGCACTACTACTACGGCGGCAGCTACGCCATGG
ACTACTGGGGCCAGGGCACAAGCGTGACCGTGTCTAGC;
GATGTGCCT (additional portion of PTPR kappa);
(Fibronectin Type III Domain; SEQ ID NO: 105)
GGTCCCGTACCAGTAAAATCTCTTCAAGGAACATCCTTTGAAA
ATAAGATCTTCTTGAACTGGAAAGAACCTTTGGATCCAAATGG
AATCATCACTCAATATGAGATCAGCTATAGCAGTATAAGATCA
TTTGATCCTGCAGTTCCAGTGGCTGGACCTCCCCAGACTGTAT
CAAATTTATGGAACAGTACACACCATGTCTTTATGCATCTCCA
CCCTGGAACCACGTACCAGTTTTTCATAAGAGCCAGCACGGTC
AAAGGCTTTGGTCCAGCCACAGCCATCAATGTCACCACCAATA
TCTCAGCTCCAACT;
TTA (additional portion of PTPR kappa);
(Fibronectin Type III Domain; SEQ ID NO: 107)
CCTGACTATGAAGGAGTTGATGCCTCTCTCAATGAAACTGCCA
CCACAATAACTGTATTGTTGAGACCAGCACAAGCCAAAGGTGC
TCCTATCAGTGCTTATCAGATTGTTGTGGAAGAACTGCACCCA
CACCGAACCAAGAGAGAAGCCGGAGCCATGGAATGCTACCAGG
TTCCTGTCACATACCAAAATGCCATGAGTGGGGGTGCACCGTA
TTACTTTGCTGCAGAACTACCCCCGGGAAACCTACCT;
(additional portion of PTPR kappa;
SEQ ID NO: 162)
GAGCCTGCCCCGTTCACTGTGGGTGACAATCGGACCTACCAAG
GCTTTTGGAACCCTCCTTTGGCTCCGCGCAAAGGATACAACAT
CTATTTCCAGGCGATGAGCAGTGTGGAGAAGGAAACTAAAACC
CAGTGCGTACGCATTGCTACAAAAGCAGCAGCAACAGAAGAAC
CAGAAGTGATCCCAGATCCCGCCAAGCAGACAGACAGAGTGGT
GAAAATAGCAGGAATTAGTGCTGGAATTTTGGTGTTCATCCTC
CTTCTCCTAGTTGTCATATTAATTGTAAAAAAGAGCAAACTTG
CTAAAAAACGCAAAGATGCCATGGGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-binding domain; SEQ ID NO: 61)
ATGAAACTCCTTAGCAGCATCGAACAGGCTTGCGACATCTGCA
GGTTGAAAAAACTCAAGTGCTCAAAAGAAAAGCCTAAGTGCGC
AAAGTGCCTTAAAAACAATTGGGAATGTCGCTATAGCCCCAAG
ACAAAGCGGAGCCCTCTCACGAGAGCACACCTGACTGAGGTAG
AATCTCGCTTGGAGAGGCTGGAACAGCTTTTCCTGCTTATCTT
TCCACGCGAGGATCTCGATATGATCCTCAAAATGGACTCCCTC
CAGGACATCAAAGCTCTGCTGACTGGACTGTTTGTACAGGATA
ATGTGAACAAGGACGCTGTGACAGACAGATTGGCAAGCGTGGA
AACGGATATGCCCCTGACCCTTAGACAGCACCGGATCAGTGCC
ACCTCTTCTAGCGAGGAAAGTTCAAATAAAGGACAGCGCCAGC
TGACGGTGAGT;
(linker; SEQ ID NO: 29)
GGCGGTGGAAGCGGAGGAGGTTCC;
and
(VP64 Transcriptional Activation Domain;
SEQ ID NO: 62)
GACGCTCTTGATGATTTCGATCTCGACATGCTGGGATCAGACG
CTCTCGACGACTTCGATTTGGACATGCTTGGATCCGACGCTCT
CGATGATTTCGACCTCGACATGCTCGGATCCGATGCTCTGGAT
GACTTTGATCTTGATATGCTG.

pCDL2243 Protein Sequence (SEQ ID NO: 110) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGT
VKLLIYHTSRLHSGVPSRFSGSGSGTDYSLTISNLEQEDIATY
FCQQGNTLPYTFGGGTKLEITGGGGSGGGGSGGGGSEVKLQES
GPGLVAPSQSLSVTCTVSGVSLPDYGVSWIRQPPRKGLEWLGV
IWGSETTYYNSALKSRLTIIKDNSKSQVFLKMNSLQTDDTAIY
YCAKHYYYGGSYAMDYWGQGTSVTVSS;
(linker; SEQ ID NO: 2)
GGGGS;
(Fibronectin Type III Domain; SEQ ID NO: 54)
KNFHVKAVMKTSVLLSWEIPENYNSAMPFKILYDDGKMVEEVD
GRATQKLIVNLKPEKSYSFVLTNRGNSAGGLQHRVTAKTAPD;
(additional portion of PTPR delta;
SEQ ID NO: 148)
VLRTKPAFIGKTNLDGMITVQLPEVPANENIKGYYIIIVPLKK
SRGKFIKPWESPDEMELDELLKEISRKRRSIRYGREVELKPYI
AAHFDVLPTEFTLGDDKHYGGFTNKQLQSGQEYVFFVLAVMEH
AESKMYATSPYSDPVVSMDLDPQPITDEEEGLIWVVGPVLAVV
FIICIVIAILLYKRKRAESDSRKSSGG;
(linker; SEQ ID NO: 2)
GGGGS;
(c-myc NLS; SEQ ID NO: 58)
PAAKRVKLD;
GS (linker);
(HNF1 alpha DNA-binding domain;
SEQ ID NO: 112)
MVSKLSQLQTELLAALLESGLSKEALIQALGEPGPYLLAGEGP
LDKGESCGGGRGELAELPNGLGETRGSEDETDDDGEDFTPPIL
KELENLSPEEAAHQKAVVETLLQEDPWRVAKMVKSYLQQHNIP
QREVVDTTGLNQSHLSQHLNKGTPMKTQKRAALYTWYVRKQRE
VAQQFTHAGQGGLJEEPTGDELPTKKGRRNRFKWGPASQQILF
QAYERQKNPSKEERETLVEECNRAECIQRGVSPSQAQGLGSNL
VTEVRVYNWFANRRKEEAFRHKLAM;
(linker; SEQ ID NO: 29)
GGGSGGGS;
and
(p65 Transcriptional Activation Domain;
SEQ ID NO: 114)
DEFPTMVFPSGQISQASALAPAPPQVLPQAPAPAPAPAMVSAL
AQAPAPVPVLAPGPPQAVAPPAPKPTQAGEGTLSEALLQLQFD
DEDLGALLGNSTDPAVFTDLASVDNSEFQQLLNQGIPVAPHTT
EPMLMEYPEAITRLVTGAQRPPDPAPAPLGAPGLPNGLLSGDE
DFSSIADMDFSALLSQISS.

pCDL2243 cDNA Sequence (SEQ ID NO: 111) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACA
CCCAGCATTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCT
GGGCGATAGAGTGACCATCAGCTGCAGAGCCAGCCAGGACATCA
GCAAGTACCTGAACTGGTATCAGCAGAAACCCGACGGCACCGTG
AAGCTGCTGATCTACCACACCAGCAGACTGCACAGCGGCGTGCC
CAGCAGATTTTCTGGCAGCGGCTCCGGCACCGACTACAGCCTGA
CCATCTCCAACCTGGAACAGGAAGATATCGCTACCTACTTCTGT
CAGCAAGGCAACACCCTGCCCTACACCTTCGGCGGAGGCACCAA
GCTGGAAATCACAGGCGGCGGAGGATCTGGCGGAGGCGGAAGTG
GCGGAGGGGGATCTGAAGTGAAACTGCAGGAAAGCGGCCCTGGC
CTGGTGGCCCCATCTCAGTCTCTGAGCGTGACCTGTACCGTGTC
CGGCGTGTCCCTGCCTGACTATGGCGTGTCCTGGATCAGACAGC
CCCCCAGAAAGGGCCTGGAATGGCTGGGAGTGATCTGGGGCAGC
GAGACAACCTACTACAACAGCGCCCTGAAGTCCCGGCTGACCAT
CATCAAGGACAACTCCAAGAGCCAGGTGTTCCTGAAGATGAACA
GCCTGCAGACCGACGACACCGCCATCTACTACTGCGCCAAGCAC
TACTACTACGGCGGCAGCTACGCCATGGACTACTGGGGCCAGGG
CACAAGCGTGACCGTGTCTAGC;
(linker; SEQ ID NO: 47)
GGTGGAGGAGGCTCT;
(Fibronectin Type III Domain; SEQ ID NO: 55)
AAAAATTTCCACGTGAAGGCTGTTATGAAAACATCCGTTCTCCT
GTCATGGGAAATCCCGGAAAACTATAATTCTGCTATGCCTTTCA
AGATATTGTATGATGATGGCAAGATGGTTGAAGAGGTCGACGGT
CGGGCGACACAAAAACTGATCGTTAACCTCAAACCTGAGAAATC
ATATTCATTCGTCCTCACCAATCGCGGTAATAGTGCTGGTGGCC
TCCAGCACCGGGTAACCGCAAAAACTGCGCCTGAT;
(additional portion of PTPR delta;
SEQ ID NO: 149)
GTGCTCAGGACGAAGCCCGCGTTTATAGGCAAGACCAATCTTGA
TGGCATGATCACTGTTCAGCTCCCGGAAGTTCCCGCCAACGAGA
ATATCAAGGGTTATTATATTATTATCGTACCGCTCAAGAAGTCT
CGAGGCAAATTTATCAAACCTTGGGAGTCACCAGATGAAATGGA
GCTTGATGAGTTGCTCAAAGAGATCAGCAGAAAGCGGCGGTCCA
TAAGGTACGGCAGGGAGGTCGAGCTCAAGCCATACATTGCGGCT
CATTTCGATGTGTTGCCGACGGAGTTCACGCTCGGGGATGATAA
ACACTACGGCGGCTTCACAAACAAACAGCTCCAATCAGGGCAGG
AGTATGTCTTCTTCGTGCTTGCTGTCATGGAACACGCCGAATCC
AAAATGTATGCAACAAGCCCTTACTCCGATCCGGTTGTTTCTAT
GGATCTGGACCCGCAGCCGATAACAGATGAAGAAGAAGGGCTCA
TTTGGGTGGTTGGCCCTGTGCTGGCCGTGGTGTTTATTATCTGT
ATCGTTATTGCGATTCTTCTCTATAAGCGGAAGCGAGCGGAGAG
TGACTCTCGAAAATCATCCGGGGGT;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(HNF1 alpha DNA-binding domain;
SEQ ID NO: 113)
ATGGTTTCTAAACTGAGCCAGCTGCAGACGGAGCTCCTGGCGGC
CCTGCTCGAGTCAGGGCTGAGCAAAGAGGCACTGATCCAGGCAC
TGGGTGAGCCGGGGCCCTACCTCCTGGCTGGAGAAGGCCCCCTG
GACAAGGGGGAGTCCTGCGGCGGCGGTCGAGGGGAGCTGGCTGA
GCTGCCCAATGGGCTGGGGGAGACTCGGGGCTCCGAGGACGAAA
CGGACGACGATGGGGAAGACTTCACGCCACCCATCCTCAAAGAG
CTGGAGAACCTCAGCCCTGAGGAGGCGGCCCACCAGAAAGCCGT
GGTGGAGACCCTTCTGCAGGAGGACCCGTGGCGTGTGGCGAAGA
TGGTCAAGTCCTACCTGCAGCAGCACAACATCCCACAGCGGGAG
GTGGTCGATACCACTGGCCTCAACCAGTCCCACCTGTCCCAACA
CCTCAACAAGGGCACTCCCATGAAGACGCAGAAGCGGGCCGCCC
TGTACACCTGGTACGTCCGCAAGCAGCGAGAGGTGGCGCAGCAG
TTCACCCATGCAGGGCAGGGAGGGCTGATTGAAGAGCCCACAGG
TGATGAGCTACCAACCAAGAAGGGGCGGAGGAACCGTTTCAAGT
GGGGCCCAGCATCCCAGCAGATCCTGTTCCAGGCCTATGAGAGG
CAGAAGAACCCTAGCAAGGAGGAGCGAGAGACTCTAGTGGAGGA
GTGCAATAGGGCGGAATGCATCCAGAGAGGGGTGTCCCCATCAC
AGGCACAGGGGCTGGGCTCCAACCTCGTCACGGAGGTGCGTGTC
TACAACTGGTTTGCCAACCGGCGCAAAGAAGAAGCCTTCCGGCA
CAAGCTGGCCATG;
(linker; SEQ ID NO: 29)
GGCGGTGGAAGCGGAGGAGGTTCC;
and
(p65 Transcriptional Activation Domain;
SEQ ID NO: 115)
GATGAGTTTCCCACCATGGTGTTTCCTTCTGGGCAGATCAGCCA
GGCCTCGGCCTTGGCCCCGGCCCCTCCCCAAGTCCTGCCCCAGG
CTCCAGCCCCTGCCCCTGCTCCAGCCATGGTATCAGCTCTGGCC
CAGGCCCCAGCCCCTGTCCCAGTCCTAGCCCCAGGCCCTCCTCA
GGCTGTGGCCCCACCTGCCCCCAAGCCCACCCAGGCTGGGGAAG
GAACGCTGTCAGAGGCCCTGCTGCAGCTGCAGTTTGATGATGAA
GACCTGGGGGCCTTGCTTGGCAACAGCACAGACCCAGCTGTGTT
CACAGACCTGGCATCCGTCGACAACTCCGAGTTTCAGCAGCTGC
TGAACCAGGGCATACCTGTGGCCCCCCACACAACTGAGCCCATG
CTGATGGAGTACCCTGAGGCTATAACTCGCCTAGTGACAGGGGC
CCAGAGGCCCCCCGACCCAGCTCCTGCTCCACTGGGGGCCCCGG
GGCTCCCCAATGGCCTCCTTTCAGGAGATGAAGACTTCTCCTCC
ATTGCGGACATGGACTTCTCAGCCCTGCTGAGTCAGATCAGCTCC.

pCDL2244 Protein Sequence (SEQ ID NO: 116) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKL
LIYHTSRLHSGVPSRFSGSGSGTDYSLTISNLEQEDIATYFCQQGN
TLPYTFGGGTKLEITGGGGSGGGGSGGGGSEVKLQESGPGLVAPSQ
SLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMD
YWGQGTSVTVSS;
(linker; SEQ ID NO: 2)
GGGGS;
(Fibronectin Type III Domain; SEQ ID NO: 63)
AKNFRVAAAMKTSVLLSWEVPDSYKSAVPFKILYNGQSVEVDGHSM
RKLIADLQPNTEYSFVLMNRGSSAGGLQHLVSIRTAPD;
(additional portion of PTPR LAR;
SEQ ID NO: 151)
LLPHKPLPASAYIEDGRFDLSMPHVQDPSLVRWFYIVVVPIDRVGG
SMLTPRWSTPEELELDELLEAIEQGGEEQRRRRRQAERLKPYVAAQ
LDVLPETFTLGDKKNYRGFYNRPLSPDLSYQCFVLASLKEPMDQKR
YASSPYSDEIVVQVTPAQQQEEPEMLWVTGPVLAVILIILIVIAIL
LFKRKRTHSPSSKDEQSIGGG;
(linker; SEQ ID NO: 2)
GGGGS;
(c-myc NLS; SEQ ID NO: 58)
PAAKRVKLD;
GS (linker);
(HNF1 alpha DNA-binding domain;
SEQ ID NO: 112)
MVSKLSQLQTELLAALLESGLSKEALIQALGEPGPYLLAGEGPLDK
GESCGGGRGELAELPNGLGETRGSEDETDDDGEDFTPPILKELENL
SPEEAAHQKAVVETLLQEDPWRVAKMVKSYLQQHNIPQREVVDTTG
LNQSHLSQHLNKGTPMKTQKRAALYTWYVRKQREVAQQFTHAGQGG
LIEEPTGDELPTKKGRRNRFKWGPASQQILFQAYERQKNPSKEERE
TLVEECNRAECIQRGVSPSQAQGLGSNLVTEVRVYNWFANRRKEEA
FRHKLAM;
(linker; SEQ ID NO: 29)
GGGSGGGS;
and
(p65 Transcriptional Activation Domain;
SEQ ID NO: 114)
DEFPTMVFPSGQISQASALAPAPPQVLPQAPAPAPAPAMVSALAQA
PAPVPVLAPGPPQAVAPPAPKPTQAGEGTLSEALLQLQFDDEDLGA
LLGNSTDPAVFTDLASVDNSEFQQLLNQGIPVAPHTTEPMLMEYPE
AITRLVTGAQRPPDPAPAPLGAPGLPNGLLSGDEDFSSIADMDFSA
LLSQISS.

pCDL2244 cDNA Sequence (SEQ ID NO: 117) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACC
CAGCATTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGG
GCGATAGAGTGACCATCAGCTGCAGAGCCAGCCAGGACATCAGCAA
GTACCTGAACTGGTATCAGCAGAAACCCGACGGCACCGTGAAGCTG
CTGATCTACCACACCAGCAGACTGCACAGCGGCGTGCCCAGCAGAT
TTTCTGGCAGCGGCTCCGGCACCGACTACAGCCTGACCATCTCCAA
CCTGGAACAGGAAGATATCGCTACCTACTTCTGTCAGCAAGGCAAC
ACCCTGCCCTACACCTTCGGCGGAGGCACCAAGCTGGAAATCACAG
GCGGCGGAGGATCTGGCGGAGGCGGAAGTGGCGGAGGGGGATCTGA
AGTGAAACTGCAGGAAAGCGGCCCTGGCCTGGTGGCCCCATCTCAG
TCTCTGAGCGTGACCTGTACCGTGTCCGGCGTGTCCCTGCCTGACT
ATGGCGTGTCCTGGATCAGACAGCCCCCCAGAAAGGGCCTGGAATG
GCTGGGAGTGATCTGGGGCAGCGAGACAACCTACTACAACAGCGCC
CTGAAGTCCCGGCTGACCATCATCAAGGACAACTCCAAGAGCCAGG
TGTTCCTGAAGATGAACAGCCTGCAGACCGACGACACCGCCATCTA
CTACTGCGCCAAGCACTACTACTACGGCGGCAGCTACGCCATGGAC
TACTGGGGCCAGGGCACAAGCGTGACCGTGTCTAGC;
(linker; SEQ ID NO: 49)
GGAGGTGGTGGGAGT;
(Fibronectin Type III Domain; SEQ ID NO: 64)
GCCAAAAACTTCAGAGTAGCGGCGGCGATGAAAACTTCTGTCCTTC
TGAGCTGGGAAGTTCCGGATTCCTATAAATCCGCTGTCCCCTTCAA
GATATTGTACAATGGTCAAAGTGTCGAGGTTGATGGACACTCCATG
AGGAAACTCATTGCCGACCTCCAACCGAACACAGAATACAGTTTTG
TTTTGATGAATCGCGGTTCCTCAGCCGGGGGCCTGCAGCACCTCGT
GAGTATTAGGACCGCTCCCGAT;
(additional portion of PTPR LAR;
SEQ ID NO: 152)
CTTCTTCCACATAAGCCTCTCCCCGCATCTGCGTACATAGAAGACG
GGCGGTTCGACCTGTCAATGCCCCACGTACAGGACCCTAGTCTTGT
AAGATGGTTTTATATCGTGGTCGTTCCTATAGACCGGGTTGGAGGC
TCTATGCTCACCCCGCGATGGAGTACCCCGGAAGAACTTGAGCTGG
ATGAGTTGTTGGAAGCAATCGAACAGGGGGGCGAGGAACAGAGGCG
GCGCCGCCGCCAAGCAGAGCGACTGAAACCGTATGTTGCCGCTCAG
TTGGATGTGTTGCCCGAAACGTTTACTTTGGGTGACAAAAAAAATT
ATCGGGGGTTCTACAATAGACCTTTGAGTCCTGATCTTAGTTATCA
GTGTTTTGTGTTGGCATCTTTGAAGGAACCGATGGACCAGAAACGA
TACGCTTCCTCCCCCTATAGTGACGAGATTGTCGTTCAAGTAACAC
CCGCACAACAGCAGGAGGAGCCGGAAATGCTTTGGGTTACAGGGCC
GGTACTTGCGGTGATCTTGATAATACTCATTGTCATAGCCATTCTG
CTTTTCAAGCGCAAACGGACCCACTCACCCTCTTCCAAGGACGAGC
AGAGCATAGGGGGAGGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(HNF1 alpha DNA-binding domain;
SEQ ID NO: 113)
ATGGTTTCTAAACTGAGCCAGCTGCAGACGGAGCTCCTGGCGGCCC
TGCTCGAGTCAGGGCTGAGCAAAGAGGCACTGATCCAGGCACTGGG
TGAGCCGGGGCCCTACCTCCTGGCTGGAGAAGGCCCCCTGGACAAG
GGGGAGTCCTGCGGCGGCGGTCGAGGGGAGCTGGCTGAGCTGCCCA
ATGGGCTGGGGGAGACTCGGGGCTCCGAGGACGAAACGGACGACGA
TGGGGAAGACTTCACGCCACCCATCCTCAAAGAGCTGGAGAACCTC
AGCCCTGAGGAGGCGGCCCACCAGAAAGCCGTGGTGGAGACCCTTC
TGCAGGAGGACCCGTGGCGTGTGGCGAAGATGGTCAAGTCCTACCT
GCAGCAGCACAACATCCCACAGCGGGAGGTGGTCGATACCACTGGC
CTCAACCAGTCCCACCTGTCCCAACACCTCAACAAGGGCACTCCCA
TGAAGACGCAGAAGCGGGCCGCCCTGTACACCTGGTACGTCCGCAA
GCAGCGAGAGGTGGCGCAGCAGTTCACCCATGCAGGGCAGGGAGGG
CTGATTGAAGAGCCCACAGGTGATGAGCTACCAACCAAGAAGGGGC
GGAGGAACCGTTTCAAGTGGGGCCCAGCATCCCAGCAGATCCTGTT
CCAGGCCTATGAGAGGCAGAAGAACCCTAGCAAGGAGGAGCGAGAG
ACTCTAGTGGAGGAGTGCAATAGGGCGGAATGCATCCAGAGAGGGG
TGTCCCCATCACAGGCACAGGGGCTGGGCTCCAACCTCGTCACGGA
GGTGCGTGTCTACAACTGGTTTGCCAACCGGCGCAAAGAAGAAGCC
TTCCGGCACAAGCTGGCCATG;
(linker; SEQ ID NO: 29)
GGCGGTGGAAGCGGAGGAGGTTCC;
(p65 Transcriptional Activation Domain;
SEQ ID NO: 115)
GATGAGTTTCCCACCATGGTGTTTCCTTCTGGGCAGATCAGCCAGG
CCTCGGCCTTGGCCCCGGCCCCTCCCCAAGTCCTGCCCCAGGCTCC
AGCCCCTGCCCCTGCTCCAGCCATGGTATCAGCTCTGGCCCAGGCC
CCAGCCCCTGTCCCAGTCCTAGCCCCAGGCCCTCCTCAGGCTGTGG
CCCCACCTGCCCCCAAGCCCACCCAGGCTGGGGAAGGAACGCTGTC
AGAGGCCCTGCTGCAGCTGCAGTTTGATGATGAAGACCTGGGGGCC
TTGCTTGGCAACAGCACAGACCCAGCTGTGTTCACAGACCTGGCAT
CCGTCGACAACTCCGAGTTTCAGCAGCTGCTGAACCAGGGCATACC
TGTGGCCCCCCACACAACTGAGCCCATGCTGATGGAGTACCCTGAG
GCTATAACTCGCCTAGTGACAGGGGCCCAGAGGCCCCCCGACCCAG
CTCCTGCTCCACTGGGGGCCCCGGGGCTCCCCAATGGCCTCCTTTC
AGGAGATGAAGACTTCTCCTCCATTGCGGACATGGACTTCTCAGCC
CTGCTGAGTCAGATCAGCTCC.

pCDL2245 Protein Sequence (SEQ ID NO: 118) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKL
LIYHTSRLHSGVPSRFSGSGSGTDYSLTISNLEQEDIATYFCQQGN
TLPYTFGGGTKLEITGGGGSGGGGSGGGGSEVKLQESGPGLVAPSQ
SLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMD
YWGQGTSVTVSS;
(linker; SEQ ID NO: 2)
GGGGS;
(Partial Fibronectin Type III Domain;
SEQ ID NO: 88)
KIYIQWKPPNETNGVITLYEINYKAVGSLDPSADLSSQRGKVFKLR
NETHEILFVGLYPGTTYSFTIKASTAKGFGPPVTTRIATKISAPS;
(Fibronectin Type III Domain; SEQ ID NO: 90)
MPEYDTDTPLNETDTTITVMLKPAQSRGAPVSVYQLVVKEERLQKS
RRAADIIECFSVPVSYRNASSLDSLHYFAAELKPANLPVTQPFTVG
DNKTYNGYWNPPLSPLKSYSIYFQALSKANGETKINCVRLATKG;
(additional portion of PTPR Rho;
SEQ ID NO: 157)
ASTQNSNTVEPEKQVDNTVKMAGVIAGLLMFIIILLGVMLTIKRRR
NAYSYSYYLKLAKKQKETGG;
(linker; SEQ ID NO: 2)
GGGGS;
(c-myc NLS; SEQ ID NO: 58)
PAAKRVKLD;
GS (linker);
(HNF1 alpha DNA-binding domain;
SEQ ID NO: 112)
MVSKLSQLQTELLAALLESGLSKEALIQALGEPGPYLLAGEGPLDK
GESCGGGRGELAELPNGLGETRGSEDETDDDGEDFTPPILKELENL
SPEEAAHQKAVVETLLQEDPWRVAKMVKSYLQQHNIPQREVVDTTG
LNQSHLSQHLNKGTPMKTQKRAALYTWYVRKQREVAQQFTHAGQGG
LJEEPTGDELPTKKGRRNRFKWGPASQQILFQAYERQKNPSKEERE
TLVEECNRAECIQRGVSPSQAQGLGSNLVTEVRVYNWFANRRKEEA
FRHKLAM;
(linker; SEQ ID NO: 5)
GGGSGGGS;
and
(p65 Transcriptional Activation Domain;
SEQ ID NO: 114)
DEFPTMVFPSGQISQASALAPAPPQVLPQAPAPAPAPAMVSALAQA
PAPVPVLAPGPPQAVAPPAPKPTQAGEGTLSEALLQLQFDDEDLGA
LLGNSTDPAVFTDLASVDNSEFQQLLNQGIPVAPHTTEPMLMEYPE
AITRLVTGAQRPPDPAPAPLGAPGLPNGLLSGDEDFSSIADMDFSA
LLSQISS.

pCDL2245 cDNA Sequence (SEQ ID NO: 119) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACC
CAGCATTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGG
GCGATAGAGTGACCATCAGCTGCAGAGCCAGCCAGGACATCAGCAA
GTACCTGAACTGGTATCAGCAGAAACCCGACGGCACCGTGAAGCTG
CTGATCTACCACACCAGCAGACTGCACAGCGGCGTGCCCAGCAGAT
TTTCTGGCAGCGGCTCCGGCACCGACTACAGCCTGACCATCTCCAA
CCTGGAACAGGAAGATATCGCTACCTACTTCTGTCAGCAAGGCAAC
ACCCTGCCCTACACCTTCGGCGGAGGCACCAAGCTGGAAATCACAG
GCGGCGGAGGATCTGGCGGAGGCGGAAGTGGCGGAGGGGGATCTGA
AGTGAAACTGCAGGAAAGCGGCCCTGGCCTGGTGGCCCCATCTCAG
TCTCTGAGCGTGACCTGTACCGTGTCCGGCGTGTCCCTGCCTGACT
ATGGCGTGTCCTGGATCAGACAGCCCCCCAGAAAGGGCCTGGAATG
GCTGGGAGTGATCTGGGGCAGCGAGACAACCTACTACAACAGCGCC
CTGAAGTCCCGGCTGACCATCATCAAGGACAACTCCAAGAGCCAGG
TGTTCCTGAAGATGAACAGCCTGCAGACCGACGACACCGCCATCTA
CTACTGCGCCAAGCACTACTACTACGGCGGCAGCTACGCCATGGAC
TACTGGGGCCAGGGCACAAGCGTGACCGTGTCTAGC;
(linker; SEQ ID NO: 52)
GGGGGAGGTGGGAGT;
(Partial Fibronectin III Domain; SEQ ID NO: 89)
AAAATCTATATTCAATGGAAGCCGCCAAATGAAACCAATGGAGTTA
TAACGCTCTATGAAATCAACTACAAGGCGGTTGGCTCCCTTGATCC
CTCTGCCGACCTTTCCTCACAGCGGGGTAAAGTGTTCAAGCTGAGG
AACGAAACGCACCACCTTTTCGTGGGGTTGTATCCAGGAACGACCT
ACAGTTTTACTATTAAGGCTTCCACAGCCAAAGGCTTTGGGCCCCC
TGTAACCACTAGGATTGCTACTAAAATCTCCGCGCCATCT;
(Fibronectin Type III Domain; SEQ ID NO: 91)
ATGCCCGAATATGATACGGACACCCCATTGAACGAAACAGATACAA
CAATAACTGTCATGCTGAAGCCTGCGCAATCACGCGGAGCCCCTGT
CAGCGTATATCAACTTGTAGTCAAAGAAGAAAGACTGCAAAAATCC
CGACGCGCTGCCGACATTATTGAGTGCTTCTCAGTACCCGTGAGCT
ACAGAAACGCTAGTAGCTTGGATTCTTTGCATTATTTCGCGGCCGA
ACTTAAGCCCGCGAATCTTCCGGTGACTCAACCGTTTACAGTGGGT
GACAATAAAACTTACAATGGCTATTGGAACCCGCCGTTGTCACCGC
TGAAGAGCTACTCAATATATTTCCAGGCCCTGAGTAAAGCTAACGG
TGAGACAAAAATCAACTGTGTGAGACTTGCAACTAAAGGA;
(additional portion of PTPR Rho;
SEQ ID NO: 158)
GCCAGCACACAGAATTCTAATACTGTGGAGCCCGAGAAGCAAGTTG
ACAACACTGTGAAAATGGCTGGGGTAATTGCAGGACTGCTTATGTT
CATCATAATCCTGCTTGGGGTTATGCTTACTATCAAGCGACGGCGC
AACGCCTACAGCTATAGCTACTATTTGAAATTGGCAAAAAAGCAGA
AGGAAACTGGAGGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(HNF1 alpha DNA-binding domain;
SEQ ID NO: 113)
ATGGTTTCTAAACTGAGCCAGCTGCAGACGGAGCTCCTGGCGGCCC
TGCTCGAGTCAGGGCTGAGCAAAGAGGCACTGATCCAGGCACTGGG
TGAGCCGGGGCCCTACCTCCTGGCTGGAGAAGGCCCCCTGGACAAG
GGGGAGTCCTGCGGCGGCGGTCGAGGGGAGCTGGCTGAGCTGCCCA
ATGGGCTGGGGGAGACTCGGGGCTCCGAGGACGAAACGGACGACGA
TGGGGAAGACTTCACGCCACCCATCCTCAAAGAGCTGGAGAACCTC
AGCCCTGAGGAGGCGGCCCACCAGAAAGCCGTGGTGGAGACCCTTC
TGCAGGAGGACCCGTGGCGTGTGGCGAAGATGGTCAAGTCCTACCT
GCAGCAGCACAACATCCCACAGCGGGAGGTGGTCGATACCACTGGC
CTCAACCAGTCCCACCTGTCCCAACACCTCAACAAGGGCACTCCCA
TGAAGACGCAGAAGCGGGCCGCCCTGTACACCTGGTACGTCCGCAA
GCAGCGAGAGGTGGCGCAGCAGTTCACCCATGCAGGGCAGGGAGGG
CTGATTGAAGAGCCCACAGGTGATGAGCTACCAACCAAGAAGGGGC
GGAGGAACCGTTTCAAGTGGGGCCCAGCATCCCAGCAGATCCTGTT
CCAGGCCTATGAGAGGCAGAAGAACCCTAGCAAGGAGGAGCGAGAG
ACTCTAGTGGAGGAGTGCAATAGGGCGGAATGCATCCAGAGAGGGG
TGTCCCCATCACAGGCACAGGGGCTGGGCTCCAACCTCGTCACGGA
GGTGCGTGTCTACAACTGGTTTGCCAACCGGCGCAAAGAAGAAGCC
TTCCGGCACAAGCTGGCCATG;
(linker; SEQ ID NO: 29)
GGCGGTGGAAGCGGAGGAGGTTCC;
and
(p65 Transcriptional Activation Domain;
SEQ ID NO: 115)
GATGAGTTTCCCACCATGGTGTTTCCTTCTGGGCAGATCAGCCAGG
CCTCGGCCTTGGCCCCGGCCCCTCCCCAAGTCCTGCCCCAGGCTCC
AGCCCCTGCCCCTGCTCCAGCCATGGTATCAGCTCTGGCCCAGGCC
CCAGCCCCTGTCCCAGTCCTAGCCCCAGGCCCTCCTCAGGCTGTGG
CCCCACCTGCCCCCAAGCCCACCCAGGCTGGGGAAGGAACGCTGTC
AGAGGCCCTGCTGCAGCTGCAGTTTGATGATGAAGACCTGGGGGCC
TTGCTTGGCAACAGCACAGACCCAGCTGTGTTCACAGACCTGGCAT
CCGTCGACAACTCCGAGTTTCAGCAGCTGCTGAACCAGGGCATACC
TGTGGCCCCCCACACAACTGAGCCCATGCTGATGGAGTACCCTGAG
GCTATAACTCGCCTAGTGACAGGGGCCCAGAGGCCCCCCGACCCAG
CTCCTGCTCCACTGGGGGCCCCGGGGCTCCCCAATGGCCTCCTTTC
AGGAGATGAAGACTTCTCCTCCATTGCGGACATGGACTTCTCAGCC
CTGCTGAGTCAGATCAGCTCC.

pCDL2246 Protein Sequence (SEQ ID NO: 120) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKL
LIYHTSRLHSGVPSRFSGSGSGTDYSLTISNLEQEDIATYFCQQGN
TLPYTFGGGTKLEITGGGGSGGGGSGGGGSEVKLQESGPGLVAPSQ
SLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMD
YWGQGTSVTVSS;
(linker; SEQ ID NO: 2)
GGGGS;
(Fibronectin Type III Domain; SEQ ID NO: 98)
SPKNFKVKMIMKTSVLLSWEFPDNYNSPTPYKIQYNGLTLDVDGRT
TKKLITHLKPHTFYNFVLTNRGSSLGGLQQTVTAWTAFN;
(additional portion of PTPR sigma;
SEQ ID NO: 159)
LLNGKPSVAPKPDADGFIIV1VYLPDGQSPVPVQSYFIVMVPLRKS
RGGQFLTPLGSPEDMDLEELIQDISRLQRRSLRHSRQLEVPRPYIA
ARFSVLPPTFHPGDQKQYGGFDNRGLEPGHRYVLFVLAVLQKSEPT
FAASPFSDPFQLDNPDPQPIVDGEEGLIWVIGPVLAVVFIICIVIA
ILLYKNKPDSKRKDSEPRTKGG;
(linker; SEQ ID NO: 2)
GGGGS;
(c-myc NLS; SEQ ID NO: 58)
PAAKRVKLD;
GS (linker);
(HNF1 alpha DNA-binding domain;
SEQ ID NO: 112)
MVSKLSQLQTELLAALLESGLSKEALIQALGEPGPYLLAGEGPLDK
GESCGGGRGELAELPNGLGETRGSEDETDDDGEDFTPPILKELENL
SPEEAAHQKAVVETLLQEDPWRVAKMVKSYLQQHNIPQREVVDTTG
LNQSHLSQHLNKGTPMKTQKRAALYTWYVRKQREVAQQFTHAGQGG
LIEEPTGDELPTKKGRRNRFKWGPASQQILFQAYERQKNPSKEERE
TLVEECNRAECIQRGVSPSQAQGLGSNLVTEVRVYNWFANRRKEEA
FRHKLAM;
(linker; SEQ ID NO: 5)
GGGSGGGS;
and
(p65 Transcriptional Activation Domain;
SEQ ID NO: 114)
DEFPTMVFPSGQISQASALAPAPPQVLPQAPAPAPAPAMVSALAQA
PAPVPVLAPGPPQAVAPPAPKPTQAGEGTLSEALLQLQFDDEDLGA
LLGNSTDPAVFTDLASVDNSEFQQLLNQGIPVAPHTTEPMLMEYPE
AITRLVTGAQRPPDPAPAPLGAPGLPNGLLSGDEDFSSIADMDFSA
LLSQISS.

pCDL2246 cDNA Sequence (SEQ ID NO: 121) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCA
TTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGAT
AGAGTGACCATCAGCTGCAGAGCCAGCCAGGACATCAGCAAGTACCTGAAC
TGGTATCAGCAGAAACCCGACGGCACCGTGAAGCTGCTGATCTACCACACC
AGCAGACTGCACAGCGGCGTGCCCAGCAGATTTTCTGGCAGCGGCTCCGGC
ACCGACTACAGCCTGACCATCTCCAACCTGGAACAGGAAGATATCGCTACC
TACTTCTGTCAGCAAGGCAACACCCTGCCCTACACCTTCGGCGGAGGCACC
AAGCTGGAAATCACAGGCGGCGGAGGATCTGGCGGAGGCGGAAGTGGCGGA
GGGGGATCTGAAGTGAAACTGCAGGAAAGCGGCCCTGGCCTGGTGGCCCCA
TCTCAGTCTCTGAGCGTGACCTGTACCGTGTCCGGCGTGTCCCTGCCTGAC
TATGGCGTGTCCTGGATCAGACAGCCCCCCAGAAAGGGCCTGGAATGGCTG
GGAGTGATCTGGGGCAGCGAGACAACCTACTACAACAGCGCCCTGAAGTCC
CGGCTGACCATCATCAAGGACAACTCCAAGAGCCAGGTGTTCCTGAAGATG
AACAGCCTGCAGACCGACGACACCGCCATCTACTACTGCGCCAAGCACTAC
TACTACGGCGGCAGCTACGCCATGGACTACTGGGGCCAGGGCACAAGCGTG
ACCGTGTCTAGC;
(linker; SEQ ID NO: 53)
GGCGGGGGAGGGAGC;
(Fibronectin Type III Domain; SEQ ID NO: 99)
AGTCCGAAGAACTTCAAAGTCAAGATGATCATGAAGACATCTGTGCTCTTG
AGTTGGGAGTTTCCCGATAATTATAACAGCCCCACGCCTTACAAGATTCAG
TACAATGGACTCACCCTTGATGTAGATGGCCGCACGACAAAGAAGCTGATT
ACTCACCTCAAACCGCATACTTTCTATAATTTCGTGCTGACGAATCGGGGT
TCTTCCCTGGGAGGTCTCCAGCAAACTGTAACGGCGTGGACTGCGTTTAA
T;
(additional portion of PTPR sigma; SEQ ID NO: 160)
TTGCTGAACGGTAAGCCCTCAGTGGCCCCCAAACCGGATGCCGACGGATTT
ATAATGGTGTACCTTCCAGATGGTCAGAGTCCGGTCCCCGTACAGAGCTAC
TTCATTGTCATGGTGCCCCTCAGGAAATCCCGAGGTGGTCAATTTCTCACA
CCATTGGGTAGTCCGGAGGACATGGATCTGGAAGAACTGATCCAGGATATT
AGCCGCCTGCAACGCAGATCACTTAGACATAGTAGACAGCTGGAGGTGCCG
AGGCCGTACATCGCTGCGCGATTCTCCGTACTCCCGCCAACCTTTCACCCA
GGGGATCAGAAACAATACGGCGGTTTTGATAATCGAGGGCTTGAACCAGGA
CATAGATACGTGCTTTTTGTGTTGGCTGTGCTCCAGAAATCTGAACCGACG
TTTGCCGCAAGCCCCTTTAGCGACCCATTTCAGCTGGATAACCCTGACCCT
CAGCCGATAGTCGATGGCGAGGAGGGGCTGATATGGGTGATTGGGCCCGTA
CTCGCGGTAGTGTTTATTATCTGTATCGTAATTGCTATACTGCTTTATAAG
AACAAGCCGGACAGTAAAAGGAAGGATTCTGAGCCTAGGACTAAAGGCGG
T;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(HNF1 alpha DNA-binding domain; SEQ ID NO: 113)
ATGGTTTCTAAACTGAGCCAGCTGCAGACGGAGCTCCTGGCGGCCCTGCTC
GAGTCAGGGCTGAGCAAAGAGGCACTGATCCAGGCACTGGGTGAGCCGGGG
CCCTACCTCCTGGCTGGAGAAGGCCCCCTGGACAAGGGGGAGTCCTGCGGC
GGCGGTCGAGGGGAGCTGGCTGAGCTGCCCAATGGGCTGGGGGAGACTCGG
GGCTCCGAGGACGAAACGGACGACGATGGGGAAGACTTCACGCCACCCATC
CTCAAAGAGCTGGAGAACCTCAGCCCTGAGGAGGCGGCCCACCAGAAAGCC
GTGGTGGAGACCCTTCTGCAGGAGGACCCGTGGCGTGTGGCGAAGATGGTC
AAGTCCTACCTGCAGCAGCACAACATCCCACAGCGGGAGGTGGTCGATACC
ACTGGCCTCAACCAGTCCCACCTGTCCCAACACCTCAACAAGGGCACTCCC
ATGAAGACGCAGAAGCGGGCCGCCCTGTACACCTGGTACGTCCGCAAGCAG
CGAGAGGTGGCGCAGCAGTTCACCCATGCAGGGCAGGGAGGGCTGATTGAA
GAGCCCACAGGTGATGAGCTACCAACCAAGAAGGGGCGGAGGAACCGTTTC
AAGTGGGGCCCAGCATCCCAGCAGATCCTGTTCCAGGCCTATGAGAGGCAG
AAGAACCCTAGCAAGGAGGAGCGAGAGACTCTAGTGGAGGAGTGCAATAGG
GCGGAATGCATCCAGAGAGGGGTGTCCCCATCACAGGCACAGGGGCTGGGC
TCCAACCTCGTCACGGAGGTGCGTGTCTACAACTGGTTTGCCAACCGGCGC
AAAGAAGAAGCCTTCCGGCACAAGCTGGCCATG;
(linker; SEQ ID NO: 29)
GGCGGTGGAAGCGGAGGAGGTTCC;
and
(p65 Transcriptional Activation
Domain; SEQ ID NO: 115)
GATGAGTTTCCCACCATGGTGTTTCCTTCTGGGCAGATCAGCCAGGCCTCG
GCCTTGGCCCCGGCCCCTCCCCAAGTCCTGCCCCAGGCTCCAGCCCCTGCC
CCTGCTCCAGCCATGGTATCAGCTCTGGCCCAGGCCCCAGCCCCTGTCCCA
GTCCTAGCCCCAGGCCCTCCTCAGGCTGTGGCCCCACCTGCCCCCAAGCCC
ACCCAGGCTGGGGAAGGAACGCTGTCAGAGGCCCTGCTGCAGCTGCAGTTT
GATGATGAAGACCTGGGGGCCTTGCTTGGCAACAGCACAGACCCAGCTGTG
TTCACAGACCTGGCATCCGTCGACAACTCCGAGTTTCAGCAGCTGCTGAAC
CAGGGCATACCTGTGGCCCCCCACACAACTGAGCCCATGCTGATGGAGTAC
CCTGAGGCTATAACTCGCCTAGTGACAGGGGCCCAGAGGCCCCCCGACCCA
GCTCCTGCTCCACTGGGGGCCCCGGGGCTCCCCAATGGCCTCCTTTCAGGA
GATGAAGACTTCTCCTCCATTGCGGACATGGACTTCTCAGCCCTGCTGAGT
CAGATCAGCTCC.

pCDL2247 Protein Sequence (SEQ ID NO: 122) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKM
NSLQTDDTAIYYCAKHYYYGGSYAMDYWGQGTSVTVSS;
DVP L (additional portion of PTPR kappa);
(Partial Fibronectin Type III
Domain; SEQ ID NO: 104)
GPVPVKSLQGTSFENKIFLNWKEPLDPNGIITQYEISYSSIRSFDPAVPVA
GPPQTVSNLWNSTHHVFMHLHPGTTYQFFIRASTVKGFGPATAINVTTNIS
APT;
L (additional portion of PTPR kappa);
(Fibronectin Type III Domain; SEQ ID NO: 106)
PDYEGVDASLNETATTITVLLRPAQAKGAPISAYQIVVEELHPHRTKREAG
AMECYQVPVTYQNAMSGGAPYYFAAELPPGNLP;
(additional portion of PTPR kappa; SEQ ID NO: 161)
EPAPFTVGDNRTYQGFWNPPLAPRKGYNIYFQAMSSVEKETKTQCVRIATK
AAATEEPEVIPDPAKQTDRVVKIAGISAGILVFILLLLVVILIVKKSKLAK
KRKDAMG;
(linker; SEQ ID NO: 2)
GGGGS;
(c-myc NLS; SEQ ID NO: 58)
PAAKRVKLD;
GS (linker);
(HNF1 alpha DNA-binding domain; SEQ ID NO: 112)
MVSKLSQLQTELLAALLESGLSKEALIQALGEPGPYLLAGEGPLDKGESCG
GGRGELAELPNGLGETRGSEDETDDDGEDFTPPILKELENLSPEEAAHQKA
VVETLLQEDPWRVAKMVKSYLQQHNIPQREVVDTTGLNQSHLSQHLNKGTP
MKTQKRAALYTWYVRKQREVAQQFTHAGQGGLIEEPTGDELPTKKGRRNRF
KWGPASQQILFQAYERQKNPSKEERETLVEECNRAECIQRGVSPSQAQGLG
SNLVTEVRVYNWFANRRKEEAFRHKLAM;
(linker; SEQ ID NO: 5)
GGGSGGGS;
and
(p65 Transcriptional Activation
Domain; SEQ ID NO: 114)
DEFPTMVFPSGQISQASALAPAPPQVLPQAPAPAPAPAMVSALAQAPAPVP
VLAPGPPQAVAPPAPKPTQAGEGTLSEALLQLQFDDEDLGALLGNSTDPAV
FTDLASVDNSEFQQLLNQGIPVAPHTTEPMLMEYPEAITRLVTGAQRPPDP
APAPLGAPGLPNGLLSGDEDFSSIADMDFSALLSQISS.

pCDL2247 cDNA Sequence (SEQ ID NO: 123) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCA
TTCCTCTTGATCCCT;
(Myc tag; SEQ ID NO: 44)
GAACAAAAGCTGATCAGCGAGGAGGATCTC;
(anti-CD19 scFV; SEQ ID NO: 46)
GACATCCAGATGACCCAGACCACCAGCAGCCTGAGCGCCAGCCTGGGCGAT
AGAGTGACCATCAGCTGCAGAGCCAGCCAGGACATCAGCAAGTACCTGAAC
TGGTATCAGCAGAAACCCGACGGCACCGTGAAGCTGCTGATCTACCACACC
AGCAGACTGCACAGCGGCGTGCCCAGCAGATTTTCTGGCAGCGGCTCCGGC
ACCGACTACAGCCTGACCATCTCCAACCTGGAACAGGAAGATATCGCTACC
TACTTCTGTCAGCAAGGCAACACCCTGCCCTACACCTTCGGCGGAGGCACC
AAGCTGGAAATCACAGGCGGCGGAGGATCTGGCGGAGGCGGAAGTGGCGGA
GGGGGATCTGAAGTGAAACTGCAGGAAAGCGGCCCTGGCCTGGTGGCCCCA
TCTCAGTCTCTGAGCGTGACCTGTACCGTGTCCGGCGTGTCCCTGCCTGAC
TATGGCGTGTCCTGGATCAGACAGCCCCCCAGAAAGGGCCTGGAATGGCTG
GGAGTGATCTGGGGCAGCGAGACAACCTACTACAACAGCGCCCTGAAGTCC
CGGCTGACCATCATCAAGGACAACTCCAAGAGCCAGGTGTTCCTGAAGATG
AACAGCCTGCAGACCGACGACACCGCCATCTACTACTGCGCCAAGCACTAC
TACTACGGCGGCAGCTACGCCATGGACTACTGGGGCCAGGGCACAAGCGTG
ACCGTGTCTAGC;
GATGTGCCT (additional portion of PTPR kappa);
(Fibronectin Type III Domain; SEQ ID NO: 105)
GGTCCCGTACCAGTAAAATCTCTTCAAGGAACATCCTTTGAAAATAAGATC
TTCTTGAACTGGAAAGAACCTTTGGATCCAAATGGAATCATCACTCAATAT
GAGATCAGCTATAGCAGTATAAGATCATTTGATCCTGCAGTTCCAGTGGCT
GGACCTCCCCAGACTGTATCAAATTTATGGAACAGTACACACCATGTCTTT
ATGCATCTCCACCCTGGAACCACGTACCAGTTTTTCATAAGAGCCAGCACG
GTCAAAGGCTTTGGTCCAGCCACAGCCATCAATGTCACCACCAATATCTCA
GCTCCAACT;
TTA (additional portion of PTPR kappa);
(Fibronectin Type III Domain; SEQ ID NO: 107)
CCTGACTATGAAGGAGTTGATGCCTCTCTCAATGAAACTGCCACCACAATA
ACTGTATTGTTGAGACCAGCACAAGCCAAAGGTGCTCCTATCAGTGCTTAT
CAGATTGTTGTGGAAGAACTGCACCCACACCGAACCAAGAGAGAAGCCGGA
GCCATGGAATGCTACCAGGTTCCTGTCACATACCAAAATGCCATGAGTGGG
GGTGCACCGTATTACTTTGCTGCAGAACTACCCCCGGGAAACCTACCT;
(additional portion of PTPR kappa; SEQ ID NO: 162)
GAGCCTGCCCCGTTCACTGTGGGTGACAATCGGACCTACCAAGGCTTTTGG
AACCCTCCTTTGGCTCCGCGCAAAGGATACAACATCTATTTCCAGGCGATG
AGCAGTGTGGAGAAGGAAACTAAAACCCAGTGCGTACGCATTGCTACAAAA
GCAGCAGCAACAGAAGAACCAGAAGTGATCCCAGATCCCGCCAAGCAGACA
GACAGAGTGGTGAAAATAGCAGGAATTAGTGCTGGAATTTTGGTGTTCATC
CTCCTTCTCCTAGTTGTCATATTAATTGTAAAAAAGAGCAAACTTGCTAAA
AAACGCAAAGATGCCATGGGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(HNF1 alpha DNA-binding domain; SEQ ID NO: 113)
ATGGTTTCTAAACTGAGCCAGCTGCAGACGGAGCTCCTGGCGGCCCTGCTC
GAGTCAGGGCTGAGCAAAGAGGCACTGATCCAGGCACTGGGTGAGCCGGGG
CCCTACCTCCTGGCTGGAGAAGGCCCCCTGGACAAGGGGGAGTCCTGCGGC
GGCGGTCGAGGGGAGCTGGCTGAGCTGCCCAATGGGCTGGGGGAGACTCGG
GGCTCCGAGGACGAAACGGACGACGATGGGGAAGACTTCACGCCACCCATC
CTCAAAGAGCTGGAGAACCTCAGCCCTGAGGAGGCGGCCCACCAGAAAGCC
GTGGTGGAGACCCTTCTGCAGGAGGACCCGTGGCGTGTGGCGAAGATGGTC
AAGTCCTACCTGCAGCAGCACAACATCCCACAGCGGGAGGTGGTCGATACC
ACTGGCCTCAACCAGTCCCACCTGTCCCAACACCTCAACAAGGGCACTCCC
ATGAAGACGCAGAAGCGGGCCGCCCTGTACACCTGGTACGTCCGCAAGCAG
CGAGAGGTGGCGCAGCAGTTCACCCATGCAGGGCAGGGAGGGCTGATTGAA
GAGCCCACAGGTGATGAGCTACCAACCAAGAAGGGGCGGAGGAACCGTTTC
AAGTGGGGCCCAGCATCCCAGCAGATCCTGTTCCAGGCCTATGAGAGGCAG
AAGAACCCTAGCAAGGAGGAGCGAGAGACTCTAGTGGAGGAGTGCAATAGG
GCGGAATGCATCCAGAGAGGGGTGTCCCCATCACAGGCACAGGGGCTGGGC
TCCAACCTCGTCACGGAGGTGCGTGTCTACAACTGGTTTGCCAACCGGCGC
AAAGAAGAAGCCTTCCGGCACAAGCTGGCCATG;
(linker; SEQ ID NO: 29)
GGCGGTGGAAGCGGAGGAGGTTCC;
(p65 Transcriptional Activation
Domain; SEQ ID NO: 115)
GATGAGTTTCCCACCATGGTGTTTCCTTCTGGGCAGATCAGCCAGGCCTCG
GCCTTGGCCCCGGCCCCTCCCCAAGTCCTGCCCCAGGCTCCAGCCCCTGCC
CCTGCTCCAGCCATGGTATCAGCTCTGGCCCAGGCCCCAGCCCCTGTCCCA
GTCCTAGCCCCAGGCCCTCCTCAGGCTGTGGCCCCACCTGCCCCCAAGCCC
ACCCAGGCTGGGGAAGGAACGCTGTCAGAGGCCCTGCTGCAGCTGCAGTTT
GATGATGAAGACCTGGGGGCCTTGCTTGGCAACAGCACAGACCCAGCTGTG
TTCACAGACCTGGCATCCGTCGACAACTCCGAGTTTCAGCAGCTGCTGAAC
CAGGGCATACCTGTGGCCCCCCACACAACTGAGCCCATGCTGATGGAGTAC
CCTGAGGCTATAACTCGCCTAGTGACAGGGGCCCAGAGGCCCCCCGACCCA
GCTCCTGCTCCACTGGGGGCCCCGGGGCTCCCCAATGGCCTCCTTTCAGGA
GATGAAGACTTCTCCTCCATTGCGGACATGGACTTCTCAGCCCTGCTGAGT
CAGATCAGCTCC.

pCDL2762 Protein Sequence (SEQ ID NO: 124) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKM
NSLQTDDTAIYYCAKHYYYGGSYAMDYWGQGTSVTVSS;
(Fibronectin Type III Domain 4; SEQ ID NO: 126)
PPQKVMCVSMGSTTVRVSWVPPPADSRNGVITQYSVAYEAVDGEDRGRHVV
DGISREHSSWDLVGLEKWTEYRVWVRAHTDVGPGPESSPVLVRTDED;
(additional portion of PTPR LAR; SEQ ID NO: 163)
VPSG;
(Fibronectin Type III Domain 5; SEQ ID NO: 128)
PPRKVEVEPLNSTAVHVYWKLPVPSKQHGQIRGYQVTYVRLENGEPRGLPI
IQDVMLAEAQWRPEESEDYETTISGLTPETTYSVTVAAYTTKGDGARSKPK
IVTTTGA;
(Fibronectin Type III Domain 6; SEQ ID NO: 130)
VPGRPTMMISTTAMNTALLQWHPPKELPGELLGYRLQYCRADEARPNTIDF
GKDDQHFTVTGLHKGTTYIFRLAAKNRAGLGEEFEKEIRTPEDL;
PSG (additional portion of PTPR LAR);
(Fibronectin Type III Domain 7; SEQ ID NO: 132)
FPQNLHVTGLTTSTTELAWDPPVLAERNGRIISYTVVFRDINSQQELQNIT
TDTRFTLTGLKPDTTYDIKVRAWTSKGSGPLSPSIQSRTNIPV;
(additional portion of PTPR LAR; SEQ ID NO: 164)
EQVF;
(Fibronectin Type III Domain 8; SEQ ID NO: 63)
AKNFRVAAAMKTSVLLSWEVPDSYKSAVPFKILYNGQSVEVDGHSMRKLIA
DLQPNTEYSFVLMNRGSSAGGLQHLVSIRTAPD;
(additional portion of PTPR LAR; SEQ ID NO: 151)
LLPHKPLPASAYIEDGRFDLSMPHVQDPSLVRWFYIVVVPIDRVGGSMLTP
RWSTPEELELDELLEAIEQGGEEQRRRRRQAERLKPYVAAQLDVLPETFTL
GDKKNYRGFYNRPLSPDLSYQCFVLASLKEPMDQKRYASSPYSDEIVVQVT
PAQQQEEPEMLWVTGPVLAVILIILIVIAILLFKRKRTHSPSSKDEQSIGG
G;
(linker; SEQ ID NO: 2)
GGGGS;
(c-myc NLS; SEQ ID NO: 58)
PAAKRVKLD;
GS (linker);
(GAL4 DNA-Binding Domain; SEQ ID NO: 60)
MKLLSSIEQACDICRLKKLKCSKEKPKCAKCLKNNWECRYSPKTKRSPLTR
AHLTEVESRLERLEQLFLLIFPREDLDMILKMDSLQDIKALLTGLFVQDNV
NKDAVTDRLASVETDMPLTLRQHRISATSSSEESSNKGQRQLTVS;
(linker; SEQ ID NO: 5)
GGGSGGGS;
and
(VP64 Transcription Activation
Domain; SEQ ID NO: 22)
DALDDFDLDMLGSDALDDFDLDMLGSDALDDFDLDMLGSDALDDFDLDML.

pCDL2762 cDNA Sequence (SEQ ID NO: 125) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGC
ATTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGAT
AGAGTGACCATCAGCTGCAGAGCCAGCCAGGACATCAGCAAGTACCTGAAC
TGGTATCAGCAGAAACCCGACGGCACCGTGAAGCTGCTGATCTACCACACC
AGCAGACTGCACAGCGGCGTGCCCAGCAGATTTTCTGGCAGCGGCTCCGGC
ACCGACTACAGCCTGACCATCTCCAACCTGGAACAGGAAGATATCGCTACC
TACTTCTGTCAGCAAGGCAACACCCTGCCCTACACCTTCGGCGGAGGCACC
AAGCTGGAAATCACAGGCGGCGGAGGATCTGGCGGAGGCGGAAGTGGCGGA
GGGGGATCTGAAGTGAAACTGCAGGAAAGCGGCCCTGGCCTGGTGGCCCCA
TCTCAGTCTCTGAGCGTGACCTGTACCGTGTCCGGCGTGTCCCTGCCTGAC
TATGGCGTGTCCTGGATCAGACAGCCCCCCAGAAAGGGCCTGGAATGGCTG
GGAGTGATCTGGGGCAGCGAGACAACCTACTACAACAGCGCCCTGAAGTCC
CGGCTGACCATCATCAAGGACAACTCCAAGAGCCAGGTGTTCCTGAAGATG
AACAGCCTGCAGACCGACGACACCGCCATCTACTACTGCGCCAAGCACTAC
TACTACGGCGGCAGCTACGCCATGGACTACTGGGGCCAGGGCACAAGCGTG
ACCGTGTCTAGC;
(Fibronectin Type III Domain 4; SEQ ID NO: 127)
CCACCGCAAAAAGTTATGTGTGTGAGCATGGGATCTACCACCGTCCGCGTT
TCTTGGGTTCCACCGCCTGCCGATAGTCGGAATGGCGTCATTACTCAATAC
AGCGTAGCCTACGAGGCCGTGGATGGAGAGGATAGAGGTCGCCATGTCGTA
GATGGAATTAGCCGCGAGCATAGTTCATGGGACTTAGTCGGGTTGGAAAAG
TGGACTGAATACCGTGTTTGGGTCCGAGCTCACACGGATGTTGGCCCAGGA
CCAGAGTCCAGTCCCGTTCTCGTTCGGACGGACGAGGAC;
(additional portion of PTPR LAR; SEQ ID NO: 165)
GTTCCGTCCGGT;
(Fibronectin Type III Domain 5; SEQ ID NO: 129)
CCACCCCGAAAAGTTGAGGTAGAGCCCCTGAATAGTACGGCAGTACATGTC
TATTGGAAGCTCCCTGTTCCATCAAAGCAACATGGGCAGATTAGGGGGTAC
CAAGTTACATATGTGCGATTGGAAAACGGGGAACCACGGGGCCTCCCGATC
ATCCAAGATGTTATGCTCGCAGAGGCACAGTGGCGCCCCGAGGAGTCCGAA
GATTATGAAACTACTATAAGTGGGCTTACCCCAGAGACAACCTACTCTGTA
ACTGTAGCTGCTTACACGACCAAAGGGGATGGCGCCCGAAGCAAGCCCAAA
ATTGTTACCACAACAGGGGCT;
(Fibronectin Type III Domain 6; SEQ ID NO: 131)
GTACCCGGCAGACCGACAATGATGATCAGCACGACAGCGATGAACACGGCA
CTTCTGCAATGGCATCCGCCCAAGGAATTGCCAGGGGAGTTGCTCGGCTAC
CGGCTGCAATACTGTAGGGCAGATGAAGCAAGGCCGAACACAATCGATTTC
GGAAAGGATGATCAGCACTTTACTGTTACCGGACTTCATAAAGGGACAACT
TATATCTTCCGGCTGGCGGCTAAAAATAGAGCGGGCTTGGGAGAAGAATTC
GAGAAGGAGATTAGAACCCCGGAGGATTTG;
CCTAGTGGA (additional portion of PTPR LAR);
(Fibronectin Type III Domain 7; SEQ ID NO: 133)
TTCCCGCAAAATCTCCATGTCACGGGACTCACTACTTCAACGACCGAACTC
GCTTGGGATCCTCCAGTCCTTGCCGAAAGAAACGGAAGGATTATAAGCTAC
ACCGTAGTCTTCCGAGATATTAACTCACAACAAGAACTCCAGAATATTACT
ACCGATACGCGATTCACATTGACAGGACTTAAGCCTGACACGACTTATGAT
ATAAAAGTCCGGGCTTGGACTTCCAAAGGAAGCGGACCACTGTCTCCCTCA
ATCCAATCGAGGACTATGCCAGTT;
(additional portion of PTPR LAR; SEQ ID NO: 166)
GAGCAAGTGTTT;
(Fibronectin Type III Domain 8; SEQ ID NO: 65)
GCAAAAAACTTCAGAGTAGCGGCGGCGATGAAAACTTCTGTCCTTCTGAGC
TGGGAAGTTCCGGATTCCTATAAATCCGCTGTCCCCTTCAAGATATTGTAC
AATGGTCAAAGTGTCGAGGTTGATGGACACTCCATGAGGAAACTCATTGCC
GACCTCCAACCGAACACAGAATACAGTTTTGTTTTGATGAATCGCGGTTCC
TCAGCCGGGGGCCTGCAGCACCTCGTGAGTATTAGGACCGCTCCCGAT;
(additional portion of PTPR LAR; SEQ ID NO: 152)
CTTCTTCCACATAAGCCTCTCCCCGCATCTGCGTACATAGAAGACGGGCGG
TTCGACCTGTCAATGCCCCACGTACAGGACCCTAGTCTTGTAAGATGGTTT
TATATCGTGGTCGTTCCTATAGACCGGGTTGGAGGCTCTATGCTCACCCCG
CGATGGAGTACCCCGGAAGAACTTGAGCTGGATGAGTTGTTGGAAGCAATC
GAACAGGGGGGCGAGGAACAGAGGCGGCGCCGCCGCCAAGCAGAGCGACTG
AAACCGTATGTTGCCGCTCAGTTGGATGTGTTGCCCGAAACGTTTACTTTG
GGTGACAAAAAAAATTATCGGGGGTTCTACAATAGACCTTTGAGTCCTGAT
CTTAGTTATCAGTGTTTTGTGTTGGCATCTTTGAAGGAACCGATGGACCAG
AAACGATACGCTTCCTCCCCCTATAGTGACGAGATTGTCGTTCAAGTAACA
CCCGCACAACAGCAGGAGGAGCCGGAAATGCTTTGGGTTACAGGGCCGGTA
CTTGCGGTGATCTTGATAATACTCATTGTCATAGCCATTCTGCTTTTCAAG
CGCAAACGGACCCACTCACCCTCTTCCAAGGACGAGCAGAGCATAGGGGGA
GGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-Binding Domain; SEQ ID NO: 61)
ATGAAACTCCTTAGCAGCATCGAACAGGCTTGCGACATCTGCAGGTTGAAA
AAACTCAAGTGCTCAAAAGAAAAGCCTAAGTGCGCAAAGTGCCTTAAAAAC
AATTGGGAATGTCGCTATAGCCCCAAGACAAAGCGGAGCCCTCTCACGAGA
GCACACCTGACTGAGGTAGAATCTCGCTTGGAGAGGCTGGAACAGCTTTTC
CTGCTTATCTTTCCACGCGAGGATCTCGATATGATCCTCAAAATGGACTCC
CTCCAGGACATCAAAGCTCTGCTGACTGGACTGTTTGTACAGGATAATGTG
AACAAGGACGCTGTGACAGACAGATTGGCAAGCGTGGAAACGGATATGCCC
CTGACCCTTAGACAGCACCGGATCAGTGCCACCTCTTCTAGCGAGGAAAGT
TCAAATAAAGGACAGCGCCAGCTGACGGTGAGT;
(linker; SEQ ID NO: 19)
GGCGGTGGAAGCGGAGGAGGTTCC;
and
(VP64 Transcription Activation
Domain; SEQ ID NO: 62)
GACGCTCTTGATGATTTCGATCTCGACATGCTGGGATCAGACGCTCTCGAC
GACTTCGATTTGGACATGCTTGGATCCGACGCTCTCGATGATTTCGACCTC
GACATGCTCGGATCCGATGCTCTGGATGACTTTGATCTTGATATGCTG.

pCDL2763 Protein Sequence (SEQ ID NO: 136) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKM
NSLQTDDTAIYYCAKHYYYGGSYAMDYWGQGTSVTVSS;
(additional portion of PTPR LAR; SEQ ID NO: 163)
VPSG;
(Fibronectin Type III Domain 5; SEQ ID NO: 128)
PPRKVEVEPLNSTAVHVYWKLPVPSKQHGQIRGYQVTYVRLENGEPRGLPI
IQDVMLAEAQWRPEESEDYETTISGLTPETTYSVTVAAYTTKGDGARSKPK
IVTTTGA;
(Fibronectin Type III Domain 6; SEQ ID NO: 130)
VPGRPTMMISTTAMNTALLQWHPPKELPGELLGYRLQYCRADEARPNTIDF
GKDDQHFTVTGLHKGTTYIFRLAAKNRAGLGEEFEKEIRTPEDL;
PSG (additional portion of PTPR LAR);
(Fibronectin Type III Domain 7; SEQ ID NO: 132)
FPQNLHVTGLTTSTTELAWDPPVLAERNGRIISYTVVFRDINSQQELQNIT
TDTRFTLTGLKPDTTYDIKVRAWTSKGSGPLSPSIQSRTMPV;
(SEQ ID NO: 164)
EQVF;
(Fibronectin Type III Domain 8; SEQ ID NO: 63)
AKNFRVAAAMKTSVLLSWEVPDSYKSAVPFKILYNGQSVEVDGHSMRKLIA
DLQPNTEYSFVLMNRGSSAGGLQHLVSIRTAPD;
(additional portion of PTPR LAR; SEQ ID NO: 151)
LLPHKPLPASAYIEDGRFDLSMPHVQDPSLVRWFYIVVVPIDRVGGSMLTP
RWSTPEELELDELLEAIEQGGEEQRRRRRQAERLKPYVAAQLDVLPETFTL
GDKKNYRGFYNRPLSPDLSYQCFVLASLKEPMDQKRYASSPYSDEIVVQVT
PAQQQEEPEMLWVTGPVLAVILIILIVIAILLFKRKRTHSPSSKDEQSIGG
G;
(linker; SEQ ID NO: 2)
GGGGS;
(c-myc NLS; SEQ ID NO: 58)
PAAKRVKLD;
(GAL4 DNA-Binding Domain; SEQ ID NO: 60)
GSMKLLSSIEQACDICRLKKLKCSKEKPKCAKCLKNNAVECRYSPKTKRSP
LTRAHLTEVESRLERLEQLFLLIFPREDLDMILKMDSLQDIKALLTGLFVQ
DNVNKDAVTDRLASVETDMPLTLRQHRISATSSSEESSNKGQRQLTVS;
(linker; SEQ ID NO: 5)
GGGSGGGS;
and
(VP64 Transcription Activation
Domain; SEQ ID NO: 22)
DALDDFDLDMLGSDALDDFDLDMLGSDALDDFDLDMLGSDALDDFDLDML.

pCDL2763 cDNA Sequence (SEQ ID NO: 137) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCA
TTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGAT
AGAGTGACCATCAGCTGCAGAGCCAGCCAGGACATCAGCAAGTACCTGAAC
TGGTATCAGCAGAAACCCGACGGCACCGTGAAGCTGCTGATCTACCACACC
AGCAGACTGCACAGCGGCGTGCCCAGCAGATTTTCTGGCAGCGGCTCCGGC
ACCGACTACAGCCTGACCATCTCCAACCTGGAACAGGAAGATATCGCTACC
TACTTCTGTCAGCAAGGCAACACCCTGCCCTACACCTTCGGCGGAGGCACC
AAGCTGGAAATCACAGGCGGCGGAGGATCTGGCGGAGGCGGAAGTGGCGGA
GGGGGATCTGAAGTGAAACTGCAGGAAAGCGGCCCTGGCCTGGTGGCCCCA
TCTCAGTCTCTGAGCGTGACCTGTACCGTGTCCGGCGTGTCCCTGCCTGAC
TATGGCGTGTCCTGGATCAGACAGCCCCCCAGAAAGGGCCTGGAATGGCTG
GGAGTGATCTGGGGCAGCGAGACAACCTACTACAACAGCGCCCTGAAGTCC
CGGCTGACCATCATCAAGGACAACTCCAAGAGCCAGGTGTTCCTGAAGATG
AACAGCCTGCAGACCGACGACACCGCCATCTACTACTGCGCCAAGCACTAC
TACTACGGCGGCAGCTACGCCATGGACTACTGGGGCCAGGGCACAAGCGTG
ACCGTGTCTAGC;
(additional portion of PTPR LAR; SEQ ID NO: 165)
GTTCCGTCCGGT;
(Fibronectin Type III Domain 5; SEQ ID NO: 129)
CCACCCCGAAAAGTTGAGGTAGAGCCCCTGAATAGTACGGCAGTACATGTC
TATTGGAAGCTCCCTGTTCCATCAAAGCAACATGGGCAGATTAGGGGGTAC
CAAGTTACATATGTGCGATTGGAAAACGGGGAACCACGGGGCCTCCCGATC
ATCCAAGATGTTATGCTCGCAGAGGCACAGTGGCGCCCCGAGGAGTCCGAA
GATTATGAAACTACTATAAGTGGGCTTACCCCAGAGACAACCTACTCTGTA
ACTGTAGCTGCTTACACGACCAAAGGGGATGGCGCCCGAAGCAAGCCCAAA
ATTGTTACCACAACAGGGGCT;
(Fibronectin Type III Domain 6; SEQ ID NO: 131)
GTACCCGGCAGACCGACAATGATGATCAGCACGACAGCGATGAACACGGCA
CTTCTGCAATGGCATCCGCCCAAGGAATTGCCAGGGGAGTTGCTCGGCTAC
CGGCTGCAATACTGTAGGGCAGATGAAGCAAGGCCGAACACAATCGATTTC
GGAAAGGATGATCAGCACTTTACTGTTACCGGACTTCATAAAGGGACAACT
TATATCTTCCGGCTGGCGGCTAAAAATAGAGCGGGCTTGGGAGAAGAATTC
GAGAAGGAGATTAGAACCCCGGAGGATTTG;
CCTAGTGGA (additional portion of PTPR LAR);
(Fibronectin Type III Domain 7; SEQ ID NO: 133)
TTCCCGCAAAATCTCCATGTCACGGGACTCACTACTTCAACGACCGAACTC
GCTTGGGATCCTCCAGTCCTTGCCGAAAGAAACGGAAGGATTATAAGCTAC
ACCGTAGTCTTCCGAGATATTAACTCACAACAAGAACTCCAGAATATTACT
ACCGATACGCGATTCACATTGACAGGACTTAAGCCTGACACGACTTATGAT
ATAAAAGTCCGGGCTTGGACTTCCAAAGGAAGCGGACCACTGTCTCCCTCA
ATCCAATCGAGGACTATGCCAGTT;
(additional portion of PTPR LAR; SEQ ID NO: 166)
GAGCAAGTGTTT;
(Fibronectin Type III Domain 8; SEQ ID NO: 65)
GCAAAAAACTTCAGAGTAGCGGCGGCGATGAAAACTTCTGTCCTTCTGAGC
TGGGAAGTTCCGGATTCCTATAAATCCGCTGTCCCCTTCAAGATATTGTAC
AATGGTCAAAGTGTCGAGGTTGATGGACACTCCATGAGGAAACTCATTGCC
GACCTCCAACCGAACACAGAATACAGTTTTGTTTTGATGAATCGCGGTTCC
TCAGCCGGGGGCCTGCAGCACCTCGTGAGTATTAGGACCGCTCCCGAT;
(additional portion of PTPR LAR; SEQ ID NO: 152)
CTTCTTCCACATAAGCCTCTCCCCGCATCTGCGTACATAGAAGACGGGCGG
TTCGACCTGTCAATGCCCCACGTACAGGACCCTAGTCTTGTAAGATGGTTT
TATATCGTGGTCGTTCCTATAGACCGGGTTGGAGGCTCTATGCTCACCCCG
CGATGGAGTACCCCGGAAGAACTTGAGCTGGATGAGTTGTTGGAAGCAATC
GAACAGGGGGGCGAGGAACAGAGGCGGCGCCGCCGCCAAGCAGAGCGACTG
AAACCGTATGTTGCCGCTCAGTTGGATGTGTTGCCCGAAACGTTTACTTTG
GGTGACAAAAAAAATTATCGGGGGTTCTACAATAGACCTTTGAGTCCTGAT
CTTAGTTATCAGTGTTTTGTGTTGGCATCTTTGAAGGAACCGATGGACCAG
AAACGATACGCTTCCTCCCCCTATAGTGACGAGATTGTCGTTCAAGTAACA
CCCGCACAACAGCAGGAGGAGCCGGAAATGCTTTGGGTTACAGGGCCGGTA
CTTGCGGTGATCTTGATAATACTCATTGTCATAGCCATTCTGCTTTTCAAG
CGCAAACGGACCCACTCACCCTCTTCCAAGGACGAGCAGAGCATAGGGGGA
GGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-Binding Domain; SEQ ID NO: 61)
ATGAAACTCCTTAGCAGCATCGAACAGGCTTGCGACATCTGCAGGTTGAAA
AAACTCAAGTGCTCAAAAGAAAAGCCTAAGTGCGCAAAGTGCCTTAAAAAC
AATTGGGAATGTCGCTATAGCCCCAAGACAAAGCGGAGCCCTCTCACGAGA
GCACACCTGACTGAGGTAGAATCTCGCTTGGAGAGGCTGGAACAGCTTTTC
CTGCTTATCTTTCCACGCGAGGATCTCGATATGATCCTCAAAATGGACTCC
CTCCAGGACATCAAAGCTCTGCTGACTGGACTGTTTGTACAGGATAATGTG
AACAAGGACGCTGTGACAGACAGATTGGCAAGCGTGGAAACGGATATGCCC
CTGACCCTTAGACAGCACCGGATCAGTGCCACCTCTTCTAGCGAGGAAAGT
TCAAATAAAGGACAGCGCCAGCTGACGGTGAGT;
(linker; SEQ ID NO: 29)
GGCGGTGGAAGCGGAGGAGGTTCC;
and
(VP64 Transcription Activation
Domain; SEQ ID NO: 62)
GACGCTCTTGATGATTTCGATCTCGACATGCTGGGATCAGACGCTCTCGAC
GACTTCGATTTGGACATGCTTGGATCCGACGCTCTCGATGATTTCGACCTC
GACATGCTCGGATCCGATGCTCTGGATGACTTTGATCTTGATATGCTG.

pCDL2764 Protein Sequence (SEQ ID NO: 140) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFV; SEQ ID NO: 45)
DIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKM
NSLQTDDTAIYYCAKHYYYGGSYAMDYWGQGTSVTVSS;
(Fibronectin Type III Domain 6; SEQ ID NO: 130)
VPGRPTMMISTTAMNTALLQWHPPKELPGELLGYRLQYCRADEARPNTIDF
GKDDQHFTVTGLHKGTTYIFRLAAKNRAGLGEEFEKEIRTPEDL;
PSG (additional portion of PTPR LAR);
(Fibronectin Type III Domain 7; SEQ ID NO: 132)
FPQNLHVTGLTTSTTELAWDPPVLAERNGRIISYTVVFRDINSQQELQNIT
TDTRFTLTGLKPDTTYDIKVRAWTSKGSGPLSPSIQSRTNIPV;
(additional portion of PTPR LAR; SEQ ID NO: 164)
EQVF;
(Fibronectin Type III Domain 8; SEQ ID NO: 63)
AKNFRVAAAMKTSVLLSWEVPDSYKSAVPFKILYNGQSVEVDGHSMRKLIA
DLQPNTEYSFVLMNRGSSAGGLQHLVSIRTAPD;
(additional portion of PTPR LAR; SEQ ID NO: 151)
LLPHKPLPASAYIEDGRFDLSNIPHVQDPSLVRWFYIVVVPIDRVGGSMLT
PRWSTPEELELDELLEAIEQGGEEQRRRRRQAERLKPYVAAQLDVLPETFT
LGDKKNYRGFYNRPLSPDLSYQCFVLASLKEPMDQKRYASSPYSDEIVVQV
TPAQQQEEPEMLWVTGPVLAVILIILIVIAILLFKRKRTHSPSSKDEQSIG
GG;
(linker; SEQ ID NO: 2)
GGGGS;
(c-myc NLS; SEQ ID NO: 58)
PAAKRVKLD;
GS (linker);
(GAL4 DNA-Binding Domain; SEQ ID NO: 60)
MKLLSSIEQACDICRLKKLKCSKEKPKCAKCLKNNWECRYSPKTKRSPLTR
AHLTEVESRLERLEQLFLLIFPREDLDMILKMDSLQDIKALLTGLFVQDNV
NKDAVTDRLASVETDMPLTLRQHRISATSSSEESSNKGQRQLTVS;
(linker; SEQ ID NO: 5)
GGGSGGGS;
and
(VP64 Transcription Activation
Domain; SEQ ID NO: 22)
DALDDFDLDMLGSDALDDFDLDMLGSDALDDFDLDMLGSDALDDFDLDML.

pCDL2764 cDNA Sequence (SEQ ID NO: 141) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCA
TTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGAT
AGAGTGACCATCAGCTGCAGAGCCAGCCAGGACATCAGCAAGTACCTGAAC
TGGTATCAGCAGAAACCCGACGGCACCGTGAAGCTGCTGATCTACCACACC
AGCAGACTGCACAGCGGCGTGCCCAGCAGATTTTCTGGCAGCGGCTCCGGC
ACCGACTACAGCCTGACCATCTCCAACCTGGAACAGGAAGATATCGCTACC
TACTTCTGTCAGCAAGGCAACACCCTGCCCTACACCTTCGGCGGAGGCACC
AAGCTGGAAATCACAGGCGGCGGAGGATCTGGCGGAGGCGGAAGTGGCGGA
GGGGGATCTGAAGTGAAACTGCAGGAAAGCGGCCCTGGCCTGGTGGCCCCA
TCTCAGTCTCTGAGCGTGACCTGTACCGTGTCCGGCGTGTCCCTGCCTGAC
TATGGCGTGTCCTGGATCAGACAGCCCCCCAGAAAGGGCCTGGAATGGCTG
GGAGTGATCTGGGGCAGCGAGACAACCTACTACAACAGCGCCCTGAAGTCC
CGGCTGACCATCATCAAGGACAACTCCAAGAGCCAGGTGTTCCTGAAGATG
AACAGCCTGCAGACCGACGACACCGCCATCTACTACTGCGCCAAGCACTAC
TACTACGGCGGCAGCTACGCCATGGACTACTGGGGCCAGGGCACAAGCGTG
ACCGTGTCTAGC;
(Fibronectin Type III Domain 6; SEQ ID NO: 131)
GTACCCGGCAGACCGACAATGATGATCAGCACGACAGCGATGAACACGGCA
CTTCTGCAATGGCATCCGCCCAAGGAATTGCCAGGGGAGTTGCTCGGCTAC
CGGCTGCAATACTGTAGGGCAGATGAAGCAAGGCCGAACACAATCGATTTC
GGAAAGGATGATCAGCACTTTACTGTTACCGGACTTCATAAAGGGACAACT
TATATCTTCCGGCTGGCGGCTAAAAATAGAGCGGGCTTGGGAGAAGAATTC
GAGAAGGAGATTAGAACCCCGGAGGATTTG;
CCTAGTGGA (additional portion of PTPR LAR);
(Fibronectin Type III Domain 7; SEQ ID NO: 133)
TTCCCGCAAAATCTCCATGTCACGGGACTCACTACTTCAACGACCGAACTC
GCTTGGGATCCTCCAGTCCTTGCCGAAAGAAACGGAAGGATTATAAGCTAC
ACCGTAGTCTTCCGAGATATTAACTCACAACAAGAACTCCAGAATATTACT
ACCGATACGCGATTCACATTGACAGGACTTAAGCCTGACACGACTTATGAT
ATAAAAGTCCGGGCTTGGACTTCCAAAGGAAGCGGACCACTGTCTCCCTCA
ATCCAATCGAGGACTATGCCAGTT;
(additional portion of PTPR LAR; SEQ ID NO: 166)
GAGCAAGTGTTT;
(Fibronectin Type III Domain 8; SEQ ID NO: 65)
GCAAAAAACTTCAGAGTAGCGGCGGCGATGAAAACTTCTGTCCTTCTGAGC
TGGGAAGTTCCGGATTCCTATAAATCCGCTGTCCCCTTCAAGATATTGTAC
AATGGTCAAAGTGTCGAGGTTGATGGACACTCCATGAGGAAACTCATTGCC
GACCTCCAACCGAACACAGAATACAGTTTTGTTTTGATGAATCGCGGTTCC
TCAGCCGGGGGCCTGCAGCACCTCGTGAGTATTAGGACCGCTCCCGAT;
(additional portion of PTPR LAR; SEQ ID NO: 152)
CTTCTTCCACATAAGCCTCTCCCCGCATCTGCGTACATAGAAGACGGGCGG
TTCGACCTGTCAATGCCCCACGTACAGGACCCTAGTCTTGTAAGATGGTTT
TATATCGTGGTCGTTCCTATAGACCGGGTTGGAGGCTCTATGCTCACCCCG
CGATGGAGTACCCCGGAAGAACTTGAGCTGGATGAGTTGTTGGAAGCAATC
GAACAGGGGGGCGAGGAACAGAGGCGGCGCCGCCGCCAAGCAGAGCGACTG
AAACCGTATGTTGCCGCTCAGTTGGATGTGTTGCCCGAAACGTTTACTTTG
GGTGACAAAAAAAATTATCGGGGGTTCTACAATAGACCTTTGAGTCCTGAT
CTTAGTTATCAGTGTTTTGTGTTGGCATCTTTGAAGGAACCGATGGACCAG
AAACGATACGCTTCCTCCCCCTATAGTGACGAGATTGTCGTTCAAGTAACA
CCCGCACAACAGCAGGAGGAGCCGGAAATGCTTTGGGTTACAGGGCCGGTA
CTTGCGGTGATCTTGATAATACTCATTGTCATAGCCATTCTGCTTTTCAAG
CGCAAACGGACCCACTCACCCTCTTCCAAGGACGAGCAGAGCATAGGGGGA
GGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-Binding Domain; SEQ ID NO: 61)
ATGAAACTCCTTAGCAGCATCGAACAGGCTTGCGACATCTGCAGGTTGAAA
AAACTCAAGTGCTCAAAAGAAAAGCCTAAGTGCGCAAAGTGCCTTAAAAAC
AATTGGGAATGTCGCTATAGCCCCAAGACAAAGCGGAGCCCTCTCACGAGA
GCACACCTGACTGAGGTAGAATCTCGCTTGGAGAGGCTGGAACAGCTTTTC
CTGCTTATCTTTCCACGCGAGGATCTCGATATGATCCTCAAAATGGACTCC
CTCCAGGACATCAAAGCTCTGCTGACTGGACTGTTTGTACAGGATAATGTG
AACAAGGACGCTGTGACAGACAGATTGGCAAGCGTGGAAACGGATATGCCC
CTGACCCTTAGACAGCACCGGATCAGTGCCACCTCTTCTAGCGAGGAAAGT
TCAAATAAAGGACAGCGCCAGCTGACGGTGAGT;
(linker; SEQ ID NO: 29)
GGCGGTGGAAGCGGAGGAGGTTCC;
(VP64 Transcription Activation
Domain; SEQ ID NO: 62)
GACGCTCTTGATGATTTCGATCTCGACATGCTGGGATCAGACGCTCTCGAC
GACTTCGATTTGGACATGCTTGGATCCGACGCTCTCGATGATTTCGACCTC
GACATGCTCGGATCCGATGCTCTGGATGACTTTGATCTTGATATGCTG.

pCDL2765 Protein Sequence (SEQ ID NO: 144) Comprises the Following Sequences from the N- to C-Terminus:

(CSF2RA Signal Sequence; SEQ ID NO: 41)
MLLLVTSLLLCELPHPAFLLIP;
(Myc tag; SEQ ID NO: 43)
EQKLISEEDL;
(anti-CD19 scFv; SEQ ID NO: 45)
DIQMTQTTSSLSASLGDRVTISCRASQDISKYLNWYQQKPDGTVKLLIYHT
SRLHSGVPSRFSGSGSGTDYSLTISNLEQEDIATYFCQQGNTLPYTFGGGT
KLEITGGGGSGGGGSGGGGSEVKLQESGPGLVAPSQSLSVTCTVSGVSLPD
YGVSWIRQPPRKGLEWLGVIWGSETTYYNSALKSRLTIIKDNSKSQVFLKM
NSLQTDDTAIYYCAKHYYYGGSYAMDYWGQGTSVTVSS;
SG (additional portion of PTPR LAR);
(Fibronectin Type III Domain 7; SEQ ID NO: 132)
FPQNLHVTGLTTSTTELAWDPPVLAERNGRIISYTVVFRDINSQQELQNIT
TDTRFTLTGLKPDTTYDIKVRAWTSKGSGPLSPSIQSRTMPV;
(additional portion of PTPR LAR; SEQ ID NO: 164)
EQVF;
(Fibronectin Type III Domain 8; SEQ ID NO: 63)
AKNFRVAAAMKTSVLLSWEVPDSYKSAVPFKILYNGQSVEVDGHSMRKLIA
DLQPNTEYSFVLMNRGSSAGGLQHLVSIRTAPD;
(additional portion of PTPR LAR; SEQ ID NO: 151)
LLPHKPLPASAYIEDGRFDLSMPHVQDPSLVRWFYIVVVPIDRVGGSMLTP
RWSTPEELELDELLEAIEQGGEEQRRRRRQAERLKPYVAAQLDVLPETFTL
GDKKNYRGFYNRPLSPDLSYQCFVLASLKEPMDQKRYASSPYSDEIVVQVT
PAQQQEEPEMLWVTGPVLAVILIILIVIAILLFKRKRTHSPSSKDEQSIGG
G;
(linker; SEQ ID NO: 2)
GGGGS;
(c-myc NLS; SEQ ID NO: 58)
PAAKRVKLD;
(GAL4 DNA-Binding Domain; SEQ ID NO: 60)
GSMKLLSSIEQACDICRLKKLKCSKEKPKCAKCLKNNWECRYSPKTKRSPL
TRAHLTEVESRLERLEQLFLLIFPREDLDMILKMDSLQDIKALLTGLFVQD
NVNKDAVTDRLASVETDMPLTLRQHRISATSSSEESSNKGQRQLTVS;
(linker; SEQ ID NO: 5)
GGGSGGGS;
and
(VP64 Transcription Activation
Domain; SEQ ID NO: 22)
DALDDFDLDMLGSDALDDFDLDMLGSDALDDFDLDMLGSDALDDFDLDML.

pCDL2765 cDNA Sequence (SEQ ID NO: 145) Comprises the Following Sequences from the 5′ to 3′ End:

(CSF2RA Signal Sequence; SEQ ID NO: 42)
ATGCTTCTCCTGGTGACAAGCCTTCTGCTCTGTGAGTTACCACACCCAGCA
TTCCTCTTGATCCCT;
(Myc tag; SEQ ID NO: 44)
GAACAAAAGCTGATCAGCGAGGAGGATCTC;
(anti-CD19 scFv; SEQ ID NO: 46)
GACATCCAGATGACCCAGACCACCAGCAGCCTGAGCGCCAGCCTGGGCGAT
AGAGTGACCATCAGCTGCAGAGCCAGCCAGGACATCAGCAAGTACCTGAAC
TGGTATCAGCAGAAACCCGACGGCACCGTGAAGCTGCTGATCTACCACACC
AGCAGACTGCACAGCGGCGTGCCCAGCAGATTTTCTGGCAGCGGCTCCGGC
ACCGACTACAGCCTGACCATCTCCAACCTGGAACAGGAAGATATCGCTACC
TACTTCTGTCAGCAAGGCAACACCCTGCCCTACACCTTCGGCGGAGGCACC
AAGCTGGAAATCACAGGCGGCGGAGGATCTGGCGGAGGCGGAAGTGGCGGA
GGGGGATCTGAAGTGAAACTGCAGGAAAGCGGCCCTGGCCTGGTGGCCCCA
TCTCAGTCTCTGAGCGTGACCTGTACCGTGTCCGGCGTGTCCCTGCCTGAC
TATGGCGTGTCCTGGATCAGACAGCCCCCCAGAAAGGGCCTGGAATGGCTG
GGAGTGATCTGGGGCAGCGAGACAACCTACTACAACAGCGCCCTGAAGTCC
CGGCTGACCATCATCAAGGACAACTCCAAGAGCCAGGTGTTCCTGAAGATG
AACAGCCTGCAGACCGACGACACCGCCATCTACTACTGCGCCAAGCACTAC
TACTACGGCGGCAGCTACGCCATGGACTACTGGGGCCAGGGCACAAGCGTG
ACCGTGTCTAGC;
AGTGGA (additional portion of PTPR LAR);
(Fibronectin Type III Domain 7; SEQ ID NO: 133)
TTCCCGCAAAATCTCCATGTCACGGGACTCACTACTTCAACGACCGAACTC
GCTTGGGATCCTCCAGTCCTTGCCGAAAGAAACGGAAGGATTATAAGCTAC
ACCGTAGTCTTCCGAGATATTAACTCACAACAAGAACTCCAGAATATTACT
ACCGATACGCGATTCACATTGACAGGACTTAAGCCTGACACGACTTATGAT
ATAAAAGTCCGGGCTTGGACTTCCAAAGGAAGCGGACCACTGTCTCCCTCA
ATCCAATCGAGGACTATGCCAGTT;
(additional portion of PTPR LAR; SEQ ID NO: 166)
GAGCAAGTGTTT;
(Fibronectin Type III Domain 8; SEQ ID NO: 65)
GCaAAAAACTTCAGAGTAGCGGCGGCGATGAAAACTTCTGTCCTTCTGAGC
TGGGAAGTTCCGGATTCCTATAAATCCGCTGTCCCCTTCAAGATATTGTAC
AATGGTCAAAGTGTCGAGGTTGATGGACACTCCATGAGGAAACTCATTGCC
GACCTCCAACCGAACACAGAATACAGTTTTGTTTTGATGAATCGCGGTTCC
TCAGCCGGGGGCCTGCAGCACCTCGTGAGTATTAGGACCGCTCCCGAT;
(additional portion of PTPR LAR; SEQ ID NO: 152)
CTTCTTCCACATAAGCCTCTCCCCGCATCTGCGTACATAGAAGACGGGCGG
TTCGACCTGTCAATGCCCCACGTACAGGACCCTAGTCTTGTAAGATGGTTT
TATATCGTGGTCGTTCCTATAGACCGGGTTGGAGGCTCTATGCTCACCCCG
CGATGGAGTACCCCGGAAGAACTTGAGCTGGATGAGTTGTTGGAAGCAATC
GAACAGGGGGGCGAGGAACAGAGGCGGCGCCGCCGCCAAGCAGAGCGACTG
AAACCGTATGTTGCCGCTCAGTTGGATGTGTTGCCCGAAACGTTTACTTTG
GGTGACAAAAAAAATTATCGGGGGTTCTACAATAGACCTTTGAGTCCTGAT
CTTAGTTATCAGTGTTTTGTGTTGGCATCTTTGAAGGAACCGATGGACCAG
AAACGATACGCTTCCTCCCCCTATAGTGACGAGATTGTCGTTCAAGTAACA
CCCGCACAACAGCAGGAGGAGCCGGAAATGCTTTGGGTTACAGGGCCGGTA
CTTGCGGTGATCTTGATAATACTCATTGTCATAGCCATTCTGCTTTTCAAG
CGCAAACGGACCCACTCACCCTCTTCCAAGGACGAGCAGAGCATAGGGGGA
GGG;
(linker; SEQ ID NO: 48)
GGTGGTGGGGGCTCC;
(c-myc NLS; SEQ ID NO: 59)
CCCGCCGCCAAGAGAGTGAAGCTGGAC;
(linker; SEQ ID NO: 150)
GGATCC;
(GAL4 DNA-Binding Domain; SEQ ID NO: 61)
ATGAAACTCCTTAGCAGCATCGAACAGGCTTGCGACATCTGCAGGTTGAAA
AAACTCAAGTGCTCAAAAGAAAAGCCTAAGTGCGCAAAGTGCCTTAAAAAC
AATTGGGAATGTCGCTATAGCCCCAAGACAAAGCGGAGCCCTCTCACGAGA
GCACACCTGACTGAGGTAGAATCTCGCTTGGAGAGGCTGGAACAGCTTTTC
CTGCTTATCTTTCCACGCGAGGATCTCGATATGATCCTCAAAATGGACTCC
CTCCAGGACATCAAAGCTCTGCTGACTGGACTGTTTGTACAGGATAATGTG
AACAAGGACGCTGTGACAGACAGATTGGCAAGCGTGGAAACGGATATGCCC
CTGACCCTTAGACAGCACCGGATCAGTGCCACCTCTTCTAGCGAGGAAAGT
TCAAATAAAGGACAGCGCCAGCTGACGGTGAGT;
(linker; SEQ ID NO: 29)
GGCGGTGGAAGCGGAGGAGGTTCC;
and
(VP64 Transcription Activation
Domain; SEQ ID NO: 62)
GACGCTCTTGATGATTTCGATCTCGACATGCTGGGATCAGACGCTCTCGAC
GACTTCGATTTGGACATGCTTGGATCCGACGCTCTCGATGATTTCGACCTC
GACATGCTCGGATCCGATGCTCTGGATGACTTTGATCTTGATATGCTG.

### Human T Cell Transduction

CD3⁺ Pan-T cells were obtained from healthy donors in a Human Peripheral Blood Leuko Pak, Fresh (Stemcell Technologies), and purified using RosetteSep™ Human T Cell Enrichment Cocktail (Stemcell Technologies). The cells were then frozen at a concentration of 2×10⁷ cells in 1 mL of CryoStor® CS10 (Stemcell Technologies). Twenty-four hours prior to a planned transduction, the cells were thawed and rested for 2-4 hours in hTCM (Human T Cell Media; X-VIVO 15 without Gentamicin L-Gln, Phenol Red, 1L (Lonza)+5% Human Serum (Valley Biomedical, HP1022)+10 mL per liter of 100× Glutamax (gibco)+1 mL per liter of Gentamicin 50 mg/mL (Lonza)+Premium GradeHuman IL-2 IS 50 IU/mL (Miltenyi). After resting, cells were stimulated with anti-CD3/anti-CD28 human reactive Dynabeads (Gibco) according to manufacturer's protocol overnight. The following day, the cells were transduced by adding virus onto the cells at a multiplicity of infection (MOI) of 1-2. Cells were left to transduce with the virus over a period of 72 hours, at which point the viral supernatant was removed and fresh hTCM was added. After a further 24 hours, the transduced cells were de-beaded according to manufacturer recommendation, and supplemented with fresh media. Further cell passaging to maintain a cell concentration of 1×10⁶ cells/mL was done every two days. Transduced cells were subjected to flow cytometry analysis and functional assays.

### Transduction and Activity Assay

On day 10-12, the CD3⁺ transduced cells were taken for analysis, and re-plated at 1×10⁵ cells per well for each condition in a 96-well plate. The cells were either plated with mock effector at a 3:1 ratio (CD19⁻ K562 cells), or effector cells at a 3:1 ratio (CD19⁺ Raji cells). The cells were co-cultured overnight in hTCM media. The next day, the cells were spun down and resuspended in 50 TL of Live-Dead Near IR(Invitrogen) staining buffer in PBS. The cells were incubated for 30 minutes in the dark at 4° C., and then washed twice with FACS buffer (1×PBS pH 7.4, 1L (Gibco)+5 mL of FBS per liter (Gibco)+4 mL of 0.5M EDTA pH 8.0 per liter (Invitrogen)). The cells were then resuspended in 50 TL staining mix in FACS buffer (5 TL BV510 anti-CD3 (Biolegend), 0.25 TL A674 anti-MYC (Cell Signaling)), and incubated for 30 minutes in the dark at 4° C. The cells were then washed twice in FACS buffer, and analyzed on a BD Fortessa flow cytometry machine for GFP reporter induction.

### Results

### Wave 13

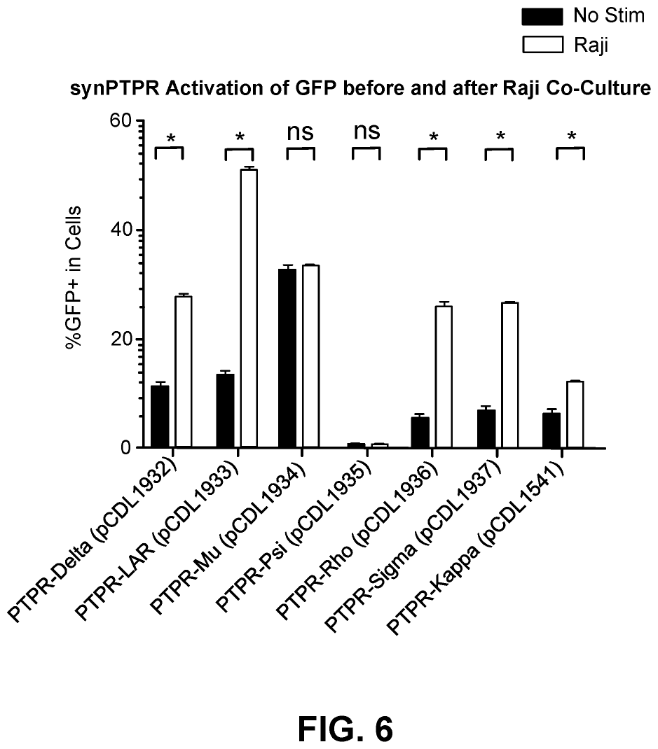

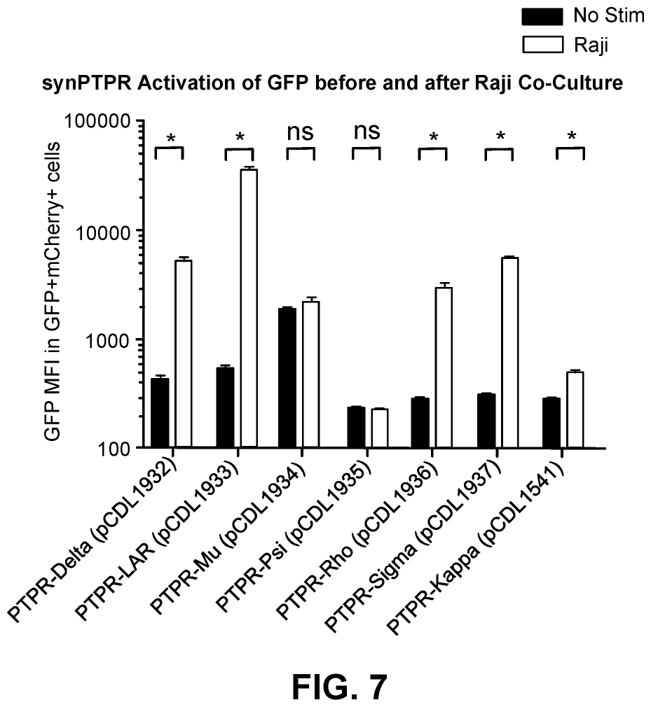

In a first set of experiments (Wave 13), the nucleic acid constructs of pCDL1932, pCDL1933, pCDL1934, pCDL1935, pCDL1936, pCDL1937, and pCDL1541 were used to transduce human CD3⁺ cells with their corresponding reporter nucleic acids (as shown in FIG. 5). The tranfected cells were either left unstimulated (cultured together with CD19⁻K562 cells) or were stimulated (cultured together with CD19⁺ Raji cells). The data show that the CD3⁺ cells transduced with pCDL1932, pCDL1933, pCDL1936, pCDL1937, and pCDL1541, when contacted with CD19-positive cells, result in cleavage of the chimeric transmembrane receptor, and result in the ability of the intracellular transcriptional regulatory domain to induce expression of green fluorescent protein (GFP) encoded by the reporter nucleic acid (FIGS. 6 and 7). pCDL1934 and pCDL1935-transduced cells showed low surface expression (6.7% and 6.6% of the cell population showing surface expression), which is thought to result in the low induction of GFP expression in these cells upon exposure to CD19 antigen. pCDL1932, pCDL1933, pCDL1936, pCDL1937, and pCDL1541 significantly higher levels of surface expression in the cells, which correlates with the CD19-induced GFP expression in these cells.

### Wave 16

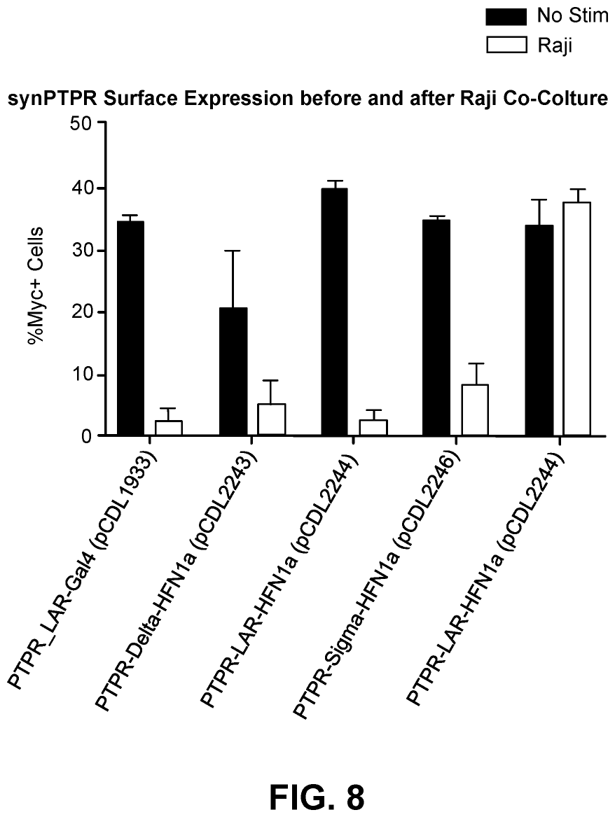

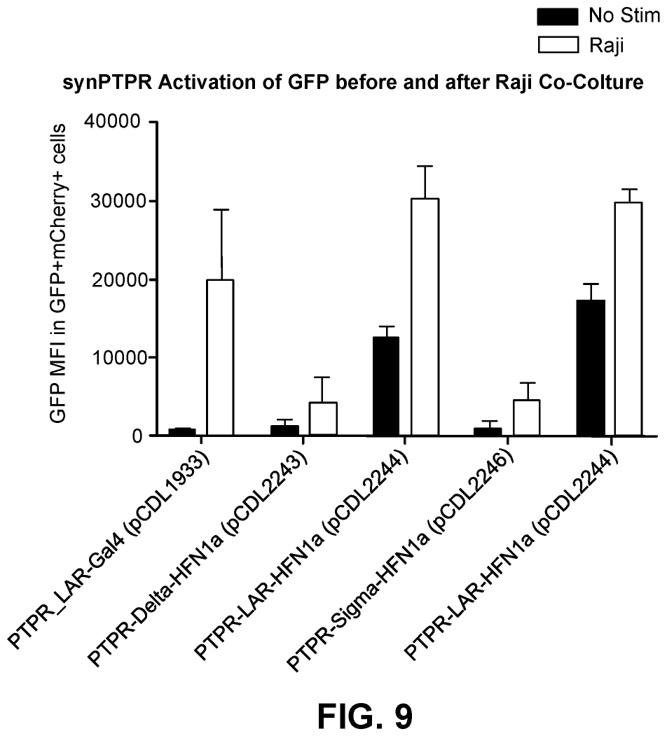

In a second set of experiments (Wave 16), the nucleic acid constructs of pCDL1932, pCDL1933, pCDL1936, pCDL1937, pCDL1541, pCDL2243, pCDL2244, pCDL2245, pCDL2246, and pCDL2247 were used to transduce human CD3+ cells with their corresponding reporter nucleic acids (as shown in FIG. 5). The tranfected cells were either left unstimulated (cultured together with CD19⁻ K562 cells) or were stimulated (cultured together with CD19⁺Raji cells). In view of the results with the Wave 13 experiments, the data resulting from the nucleic acid constructs showing surface expression on at least 10% of the transduced cells are shown. The data show that the CD3⁺ cells transduced with pCDL1933, pCDL2243, pCDL2244, pCDL2246, and pCDL2244, when contacted with CD19-positive cells, result in cleavage of the chimeric transmembrane receptor, and result in the ability of the intracellular transcriptional regulatory domain to induce expression of green fluorescent protein (GFP) encoded by the reporter nucleic acid (FIGS. 8 and 9).

### Wave 21

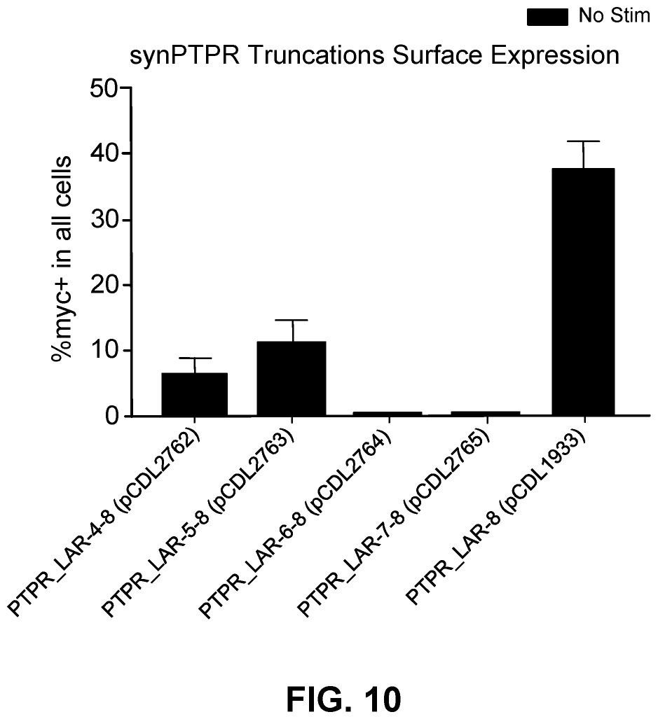

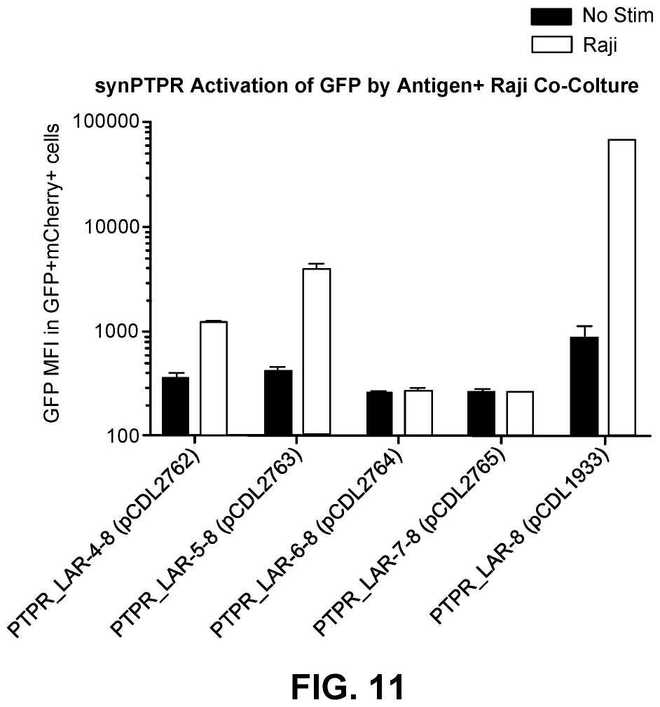

In a third set of experiments (Wave 16), the nucleic acid constructs of pCDL1933, pCDL2762, pCDL2763, pCDL2764, and pCDL2765 were used to transduce human CD3⁺ cells with their corresponding reporter nucleic acids (as shown in FIG. 5). The data show transduction with the pCDL2764 and pCDL2765 constructs results in very little surface expression of the encoded chimeric transmembrane receptor (FIG. 10). The tranfected cells were either left unstimulated (cultured together with CD19⁻ K562 cells) or were stimulated (cultured together with CD19⁺ Raji cells). The data show that the CD3⁺ cells transduced with pCDL2762, pCDL2763, and pCDL1933, when contacted with CD19-positive cells, result in cleavage of the chimeric transmembrane receptor, and result in the ability of the intracellular transcriptional regulatory domain to induce expression of green fluorescent protein (GFP) encoded by the reporter nucleic acid (FIG. 11). The low level of GFP expression resulting from cells transduced with the pCDL2764 and pCDL2765 constructs is thought to be due to the low surface expression of the encoded chimeric transmembrane receptor (as shown in FIG. 10).

1. A chimeric transmembrane receptor comprising:
an extracellular antigen-binding domain that is capable of specifically binding to a target antigen;
an extracellular integrin ligand-binding domain comprising an S2 protease cleavage site;
a transmembrane domain;
an intracellular regulatory domain comprising a gamma-secretase protease cleavage site; and
an intracellular transcriptional regulatory domain;
wherein, when the chimeric transmembrane receptor is expressed in a mammalian cell, binding of the extracellular antigen-binding domain to the target antigen induces (1) cleavage of the extracellular integrin-ligand binding domain at the S2 protease cleavage site and (2) cleavage of the intracellular regulatory domain at the gamma-secretase protease cleavage site, thereby releasing the intracellular transcriptional regulatory domain from the transmembrane domain.

2. The chimeric transmembrane receptor of claim 1, wherein the antigen-binding domain is an antibody or an antibody fragment.

3. The chimeric transmembrane receptor of claim 1, wherein the extracellular integrin ligand-binding domain is a human fibronectin III domain or a mouse fibronectin III domain.

4. The chimeric transmembrane receptor of claim 1, wherein the extracellular integrin ligand-binding domain comprises a sequence at least 80% identical to a sequence of a wild type human fibronectin III domain or a sequence of a wild type mouse fibronectin III domain.

5. The chimeric transmembrane receptor of claim 1, further comprising an additional extracellular integrin ligand-binding domain.

6. The chimeric transmembrane receptor of claim 1, wherein the transmembrane domain is a transmembrane domain present in a receptor-like tyrosine phosphatase.

7. The chimeric transmembrane receptor of claim 1, wherein the gamma-secretase cleavage site comprises a Gly-Val dipeptide amino acid sequence.

8. The chimeric transmembrane receptor of claim 1, wherein the intracellular transcriptional regulatory domain is a transcriptional activator.

9. The chimeric transmembrane receptor of claim 1, wherein the intracellular transcriptional regulatory domain is a transcriptional repressor.

10. The chimeric transmembrane receptor of claim 1, wherein the intracellular transcriptional regulatory domain comprises a transcriptional activation domain present in a polypeptide selected from the group consisting of: VP64, RelA (p65), YAP, WWTR1(TAZ), CREB3(LZIP), and MyoD.

11. A nucleic acid encoding the chimeric transmembrane receptor of claim 1.

12. A vector comprising the nucleic acid of claim 11.

13. A mammalian cell comprising the nucleic acid of claim 11.

14. The mammalian cell of claim 13, wherein the mammalian cell is an immune cell.

15. The mammalian cell of claim 13, wherein the mammalian cell further comprises a heterologous target gene comprising (i) a transcription regulatory sequence that is capable of being specifically recognized by the intracellular transcriptional regulatory domain and (ii) a nucleic acid sequence that encodes a recombinant protein,
wherein the nucleic acid sequence that encodes the recombinant protein is operably linked to the transcription regulatory sequence.

16. The mammalian cell of claim 15, wherein the recombinant protein is a chimeric antigen receptor (CAR) or a T cell receptor (TCR).

17. A pharmaceutical composition comprising a mammalian cell of claim 13.

18. A pharmaceutical composition comprising a nucleic acid of claim 11.

19. The pharmaceutical composition of claim 18, wherein the pharmaceutical composition further comprises a heterologous target gene comprising (i) a transcription regulatory sequence that is capable of being specifically recognized by the intracellular transcriptional regulatory domain and (ii) a nucleic acid sequence that encodes a recombinant protein,
wherein the nucleic acid sequence that encodes the recombinant protein is operably linked to the transcription regulatory sequence.

20. A method of treating disease in a subject in need thereof, the method comprising administering a therapeutically effective amount of a pharmaceutical composition of claim 17.
